(19) United States
(12) Patent Application Publication
(10) Pub. No.: US 2004/0018515 A1
(43) Pub. Date: Jan. 29, 2004

(54) Compositions selective for adenosine diphosphate and methods of using same

(76) Inventors: John L. Diener, Cambridge, MA (US); Jayaram Srinivasan, Murrysville, PA (US); Nobuko Hamaguchi, Framingham, MA (US); Jill Blanchard, Arlington, MA (US); Jeffrey Kurz, Somerville, MA (US); Markus Kurz, Newton, MA (US); Sharon T. Cload, Cambridge, MA (US); David Epstein, Belmont, MA (US); Charles Wilson, Concord, MA (US); Martin Stanton, Stow, MA (US)

Correspondence Address: MINTZ, LEVIN, COHN, FERRIS, GLOVSKY, AND POPEO, P.C., ONE FINANCIAL CENTER, BOSTON, MA, 02111, US

(21) Appl. No.: 10/406,027

(22) Filed: Apr. 2, 2003

Related U.S. Application Data
(60) Provisional application No. 60/369,680, filed on Apr. 3, 2002.
(60) Provisional application No. 60/370,196, filed on Apr. 5, 2002.
(60) Provisional application No. 60/437,949, filed on Jan. 3, 2003.

(51) Int. Cl. C12Q 1/68; C07H 21/04

(52) U.S. Cl. 435/6; 536/24.3

(57) ABSTRACT

Compositions which recognize and report on the concentration selectively adenosine diphosphate (ADP) and methods of making and using them are provided. The invention further relates to methods of using the compositions to monitor function of biological agents. Reagents and systems for performing the methods are also provided. The methods of the invention are useful in diagnostic applications and drug optimization.

### RELATED APPLICATIONS

This application claims priority to provisional patent applications U.S. Ser. No. 60/369,680, filed on Apr. 3, 2002, U.S. Ser. No. 60/370,196, filed on Apr. 5, 2002, and U.S. Ser. No. 60/437,949, filed on Jan. 3, 2003, each of which is incorporated herein by reference in its entirety.

### FIELD OF THE INVENTION

The invention relates to compositions which selectively recognize adenosine diphosphate (ADP). The invention further relates to methods of using the compositions to monitor the function of biological agents with, e.g., ATPase activity, kinase activity or nucleotide triphosphate hydrolase activity.

### BACKGROUND OF THE INVENTION

The human body must continuously be supplied with its own form of energy to perform its many complex functions. Aside from the energy required for muscle contraction, the body expends considerable energy for the other forms of biological work. This includes energy required for digestion, absorption, and assimilation of food nutrients and the numerous chemical compounds needed to be produced for the body to function. During all chemical reactions, energy transformations occur.

Phosphorylated adenosine-based nucleosides, e.g., adenosine triphosphate (ATP) and adenosine diphosphate (ADP) are energy-rich compounds with an important role in the metabolism of living organisms. The Krebs cycle is a cyclic sequence of reactions by which most living cells generate energy during the process of aerobic respiration. The Krebs cycle, which converts ADP to ATP, takes place in the mitochondria, using up oxygen and producing carbon dioxide and water as waste products.

ADP and ATP play a central role in endergonic reactions through a process called reaction coupling. Hydrolysis of ATP is the common currency used to do all kinds of work in living systems, including biosynthesis, excretion, muscle contraction, active transport, etc. It is important that living systems be capable of both manufacturing and conserving these molecules. Once produced as a result of hydrolysis, both ADP and AMP can be “recycled” within cells by processes of phosphorylation (substrate level phosphorylation, oxidative phosphorylation or photosynthetic phosphorylation), whereby a phosphate is attached to make either ADP or ATP.

Substrate level phosphorylation occurs in certain exergonic reactions, when the free energy released exceeds that required for phosphorylation of either AMP or ADP (>7.3 kcal/mole). When such a quantity of energy is released, the cell may “capture” the energy by using it to phosphorylate a molecule of AMP or ADP, thus producing ADP or ATP, respectively.

In both oxidative and photosynthetic phosphorylation, an electrochemical gradient is built up due to the pumping of protons across a cell membrane. This “chemiosmotic” gradient serves as the potential energy used to phosphorylate ADP. Quantitatively, chemiosmosis is the most important mechanism for making ATP in cells. In eukaryotic cells, chemiosmotic ATP synthesis occurs within the mitochondrion and chloroplast; in prokaryotic cells, the process occurs at the outer plasma membrane.

Protein kinases are ATP-dependent enzymes involved in a number of biological processes, including signal transduction in a variety of different cell types and the initiation and timing of various events (e.g., DNA synthesis and mitosis) in the cell cycle. Because kinases are essential cellular signaling molecules, mutations which affect kinase activity can lead to diseases and disorders, including Hirschsprung's disease (aganglionic megacolon), agammaglobulinemia; non-insulin dependent diabetes mellitus (NIDDM); mastocytocis; hypochondroplasia; and other immunodeficiencies, cancers, and endocrine disorders. Thus, detection of kinase activity, and the ability to determine compounds which modulate it are important in the diagnosis and treatment of kinase-related diseases and disorders.

Protein kinases are enzymes that catalyze the transfer of a phosphate group from a nucleoside triphosphate (usually ATP) to a protein substrate to yield a phosphorylated protein and a nucleoside diphosphate (ADP). In a phosphorylation reaction, kinases transfer a phosphate group to a hydroxyl moiety on a side chain of a serine, threonine, or tyrosine amino acid in an esterification reaction. In rare cases, a histidine amino acid is phosphorylated. Approximately two thirds of known kinases are specific for serine or threonine residues, and the remaining ones are specific for tyrosine residues.

Most biochemical assays to detect protein kinase activity monitor the phosphorylation of the protein substrate. Often, however, the substrate used in these assays is not the natural substrate for the kinase but is an engineered peptide substrate. Thus, the peptide used in the assay must be optimized for each particular kinase and even an optimized peptide may not be as effectively phosphorylated as the natural protein substrate, making accurate measurement of kinase activity difficult. Moreover, the peptide substrates are not universal in that they may not be a substrate at all for a given kinase.

One peptide-based assay monitors kinase activity by fluorescence resonance energy transfer (FRET). In this assay, a peptide substrate is synthesized with a donor fluorophore at one end and a quencher or FRET acceptor at the other end of the peptide. The peptide is engineered to contain a protease cleavage site which includes the amino acid to be phosphorylated by the kinase. When the peptide has been phosphorylated, it is no longer a substrate for the protease. After the kinase reaction is run, the protease is added as a second reaction. Upon peptide cleavage, an increase in donor fluorescence and/or decrease in acceptor fluorescence is observed. This assay is impractical for high throughput screening of kinase inhibitors as it requires two enzymatic reactions (which can be expensive) and can also lead to inaccurate results, for example if the inhibitor inhibits the protease and not the kinase.

Another peptide-based assay uses fluorescence anisotropy. In this assay, a fluorescently labeled phosphopeptide is bound to an anti-phosphotyrosine antibody. The large size of the antibody-peptide complex relative to the free peptide causes a large increase in the fluorescence anisotropy of the labeled peptide. A kinase is used to phosphorylate an unlabeled, unphosphorylated version of the same peptide. As the unlabeled phosphopeptide is generated, it competes with the labeled peptide for antibody binding, and when unlabeled peptide displaces labeled peptide from the antibody complex, a decrease in the fluorescence anisotropy is observed. However, this assay is limited by the lack of adequate anti-phosphoserine or anti-phosphothreonine antibodies.

Another peptide-based assay is the standard scintillation proximity assay (SPA). In this assay, a biotinylated peptide substrate is reacted with γ-³³ATP and the kinase of interest. Upon completion of the reaction, the mixture is transferred to a streptavidin coated flash plate containing a scintilant imbedded into the surface of the plate. The peptide substrates are immobilized on the surface of the plate and the greater the amount of ³³P-phosphopeptide, the greater the SPA signal. If an inhibitor screen is being done, then a successful inhibitor or “hit” will cause a significant decrease in SPA signal relative to a control reaction without inhibitors. There are three problems with this assay in general. First, it relies on a peptide substrate which at the least may need to be optimized for a specific kinase, or at worst may not be a substrate at all for a given kinase. Second, the reaction requires transfer to a second plate, doubling the amount of radioactive waste for disposal. This can be a significant expense when screening a very large number of compounds. Finally, because the assay is done in two steps, only endpoint measurements may be taken from a single reaction (no kinetic data is obtained).

One assay is currently available which directly monitors the transformation of ATP to ADP, and is thus both kinase- and substrate-independent. This enzymatic assay includes the kinase of interest, its substrate and two additional enzymes, lactate dehydrogenase and pyruvate kinase. In this assay, pyruvate kinase converts phosphoenol pyruvate and ADP into ATP and pyruvate. Lactate dehydrogenase then converts pyruvate and NADH to lactate and NAD. Conversion of NADH to NAD is accompanied by a colorimetric change of the solution. There are three major drawbacks to using this type of assay: it requires the addition of two extra enzymes to the reaction (which can be expensive); colorimetric assays are typically less sensitive than fluorescence- or radioactivity-based assays; and inaccurate identification of kinase inhibitors may result from modulation of lactate dehydrogenase and/or pyruvate kinase activity instead of protein kinase activity.

Thus, a need remains in the art for accurate, efficient, cost-effective methods of monitoring kinase activity, and the determination of compositions which modulate such activity.

### SUMMARY OF THE INVENTION

The nucleic acid compositions of the present invention are used to monitor the activity of biological agents by-detecting reagents involved as starting material, byproduct, or product of such activity. The invention also provides for accurate, efficient, cost-effective methods for detecting compositions which specifically modulate the activity of biological agents which consume or generate materials detectable by the methods of the invention.

The present invention includes nucleic acid compositions, referred to as nucleic acid sensor molecules (“NASMs”), which have a target modulation domain, a linker domain and a catalytic domain. In one embodiment of the invention, the target modulation domain of the NASM recognizes adenosine diphosphate (ADP). The NASMs of the present invention can be made from RNA, DNA, or a combination of RNA and DNA. NASMs according to the present invention can also include at least one modified nucleotide.

The catalytic domain of the NASMs according to the present invention can include a unit that generates an optical signal. In some embodiments, this unit can include a first optical signaling moiety, such as fluorescent donor, and a second signaling moiety, such as a fluorescent quencher. In embodiments having first and second optical signaling moieties, recognition of a target by the target modulation domain can change the proximity between the optical moieties. For example, in embodiments having a fluorescent donor and a fluorescent quencher, recognition of a target by the target modulation domain can result in an increase in the detectable fluorescence of the fluorescent donor. In other embodiments, recognition of a target by the target modulation domain can result in a conformational change in the optical signaling moiety, thereby resulting in a detectable optical signal.

In another embodiment, the catalytic domain of the NASMs of the present invention can include a ribozyme. For example, the catalytic domain can include an endonucleolytic ribozyme, such as a cis-endonucleolytic ribozyme or a trans-endonucleolytic ribozyme. In a preferred embodiment, the endonucleolytic ribozyme of the catalytic domain is a hammerhead ribozyme. In other embodiments, the catalytic domain of the NASMs can include a self-ligating ribozyme, such as for example, a cis-ligase ribozyme, a trans-ligase, a 1-piece ligase, a 2-piece ligase, a 3-piece ligase or any combination thereof.

NASMs of the present invention can also include an additional label. In one embodiment, the NASM can include a detectable label. For example, the detectable label can include at least one radioactive moiety, or a fluorescent label, such as for example, fluorescein, DABCYL, or a green fluorescent protein (GFP) moiety. In another embodiment, the NASM of the present invention can include an affinity capture tag label.

NASMs according to the present invention can be used to form compositions. In some embodiments, these compositions can also include an RNase inhibitor, such as for example, Va-riboside, vanadyl, tRNA, polyU, RNaseln or RNaseOut. In these embodiments, the compositions can be substantially RNase-free. In another embodiment, at least one NASM in the compositions according to the present invention can be affixed to a substrate, such as for example, glass, gold or other metal(s), silicon or other semiconductor material(s), nylon or plastic. These compositions can be attached to the substrate either covalently or non-covalently. In one embodiment, one or more NASM according to the present invention can be immobilized to the substrate by hybridization of an end portion of the NASM to an oligonucleotide that is attached to the surface of the substrate. In this embodiment, virtually any number of NASMs can be immobilized via hybridization, but in a preferred embodiment, at least 50 NASMs are attached to the substrate, and more preferably, at least 250 NASMs are attached to the substrate.

The present invention also provides systems, diagnostic systems and methods for detecting ADP in a sample using a NASM composition according to the present invention and a detector that is in communication with the composition. In this embodiment, the detector is capable of detecting a signal that is generated by the composition when the NASM recognizes a target molecule. In some embodiments, the systems and methods can also include a processor for processing the optical signals detected by the detector. The change in signal generated by the NASM composition can be used to quantify the amount of ADP in a sample. These systems and methods for detecting ADP can be used in conjunction with environmental samples, biohazard samples, organic sample, drugs and toxin, flavors, fragrances, or biological samples. Biological samples can include cells, cell extracts, cell lysates, tissues, tissue extracts, bodily fluids, serum, blood and blood products.

In another embodiment of the invention, the NASMs of the present invention can be used in methods for detecting the activity of a biological agent that produces or consumes ADP in a reaction. These methods include the steps of contacting a sample that contains the biological agent with a NASM of the present invention and detecting the change in signal generated by the optical signal generating unit or detectable label of the NASM, wherein a change in signal indicates activity of the biological agent in the sample. In yet another embodiment, NASMs according to the present invention can also be used in methods for identifying a modulator of the activity of a biological agent that produces or consumes ADP in a reaction. In this embodiment, a test agent is contacted with a biological agent and a NASM that includes a target recognition domain that recognizes ADP.

In these embodiments, suitable biological agents for use in these methods include, but are not limited to, an ATP synthase, an ATPase, or a kinase. Kinases used in these methods can include an RAF kinase, or a MAP kinase (MEK), such as for example, ERK1, ERK2, JNK or P38 MAP kinase, or a MAP kinase kinase (MEKK), or a MAP kinase kinase kinase (MEKKK). In these embodiments, the catalytic domain of the NASM can include a cis-ligase ribozyme or a trans-ligase ribozyme.

The present invention also include NASMs that are at least 10, 100, 1000, 10,000 times more specific for ADP than ATP, and NASMs that recognize ADP in at least a 10, 100, 1000, 10,000 fold excess of ATP.

In another aspect, the present invention includes ADP-specific aptamers. The ADP-specific aptamers of the present invention can be combined with a buffer to create compositions. In some embodiments, the compositions can also comprise an RNase inhibitor. In some embodiments, the compositions can be substantially RNase-free.

These compositions can also include at least one ADP-specific aptamer that is affixed to a substrate. Suitable substrates for use in the present invention include, but are not limited to, glass, gold or other metal(s), silicon or other semiconductor material(s), nitrocellulose, nylon or plastic. In one embodiment, the substrate can be a multiwell plate that has a scintillant imbedded in the surface of the plate.

The ADP-specific aptamer can be covalently or noncovalently attached to the substrate. In one embodiment, one or more ADP-specific aptamer according to the present invention can be immobilized to the substrate by hybridization of an end portion of the aptamer to an oligonucleotide that is attached to the surface of the substrate. In another embodiment, the ADP-specific aptamer can be biotinylated, and the surface of the plate can be coated with streptavidin. In yet another embodiment, at least one ADP-specific aptamer can be immobilized within the wells of a multi-well plate that has scintillant imbedded in the surface of the plate.

In this embodiment, virtually any number of ADP-specific aptamers can be immobilized via hybridization, but in a preferred embodiment, at least 50 ADP-specific aptamers are attached to the substrate, and more preferably, at least 250 ADP-specific aptamers are attached to the substrate.

The present invention also provides systems, diagnostic systems and methods for detecting ADP in a sample using an ADP-specific aptamer composition according to the present invention and a detector that is in communication with the composition. In these methods, a sample containing detectably labeled ADP is contacted with an ADP-specific aptamer composition of the present invention, and the signal generated by the detectable label indicates the presence of ADP in the sample. In some embodiments, the systems and methods of the present invention can also include a processor for processing the optical signals detected by the detector. The change in signal generated by the ADP-specific aptamer composition can be used to quantify the amount of ADP in a sample. These systems and methods for detecting ADP can be used in conjunction with environmental samples, biohazard samples, organic sample, drugs and toxin, flavors, fragrances, or biological samples. Biological samples can include cells, cell extracts, cell lysates, tissues, tissue extracts, bodily fluids, serum, blood and blood products.

In another embodiment of the invention, the ADP-specific aptamers of the present invention can be used in methods for detecting the activity of a biological agent that produces or consumes ADP in a reaction. These methods include the steps of contacting a sample that contains the biological agent with a ADP-specific aptamer of the present invention and detecting the change in signal generated by the optical signal generating unit or detectable label of the ADP-specific aptamer, wherein a change in signal indicates activity of the biological agent in the sample. In yet another embodiment, ADP-specific aptamers according to the present invention can also be used in methods for identifying a modulator of the activity of a biological agent that produces or consumes ADP in a reaction. In this embodiment, a test agent is contacted with a biological agent and a ADP-specific aptamer recognizes ADP. According to these methods, recognition of the ADP by the ADP-specific aptamer results in a change in signal generated by the detectable label, and a change in the signal generated indicates that the test agent is a modulator of the activity of the biological agent. Suitable biological agents which produce or consume ADP and can be used with these methods include, but are not limited to, an ATP synthase, a kinase or an ATPase.

The present invention also include ADP-specific aptamers that are at least 10, 100, 1000, 10,000 times more specific for ADP than ATP, and ADP-specific aptamers that recognize ADP in at least a 10, 100, 1000, 10,000 fold excess of ATP.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### BRIEF DESCRIPTION OF THE DRAWINGS

FIG. 1A is a schematic representation of secondary structure representation of 3-piece NASM construct. FIG. 1B is a schematic representation of a 1-piece NASM construct which is a slightly modified version of 3-piece system where the effector and substrate regions are replaced by a stable GNRA tetraloop.

FIG. 2 is a schematic representation of a secondary structure representation of two 2-piece NASMs with their oligonucleotide substrate.

FIG. 3 is a flow diagram showing a gel-based method for selecting nucleic acid sensor molecules having a target molecule activatable endonuclease activity.

FIG. 4 is a flow diagram showing a method for selecting nucleic acid sensor molecules having a target molecule activatable ligase activity.

FIG. 5 is a flow diagram showing a method for selecting nucleic acid sensor molecules having a target molecule activatable self-cleavage activity.

FIG. 6 is a schematic representation of various FRET formats in hammerhead ribozymes.

FIG. 7A is a schematic representation of an example of a self-cleaving nucleic acid sensor molecule bound to a solid support when used in an epi-illuminated FRET detection scheme. FIG. 7B is a schematic representation of the same sensor in an epi-illuminated beacon configuration, with the acceptor fluorophore replaced by a quencher group. FIG. 7C is a schematic representation of the same sensor in an TIR-illuminated beacon configuration.

FIG. 8 is a schematic representation of the conversion of a core hammerhead NASM into optical NASMs useful for FRET.

FIG. 9 is a schematic representation of stem I-modified NASMs useful for FRET.

FIG. 10 is a schematic representation of immobilized hammerhead NASMs useful for FRET.

FIG. 11A is a graph depicting fluorescence intensity vs. time for cleavage in optical hammerhead NASM as measured by FRET. FIG. 11B is a line graph of first order kinetic analysis of cleavage rate as measured by FRET.

FIG. 12 is a schematic representation of the use of beads in a homogeneous assay format utilizing a self-ligating nucleic acid sensor. FIG. 12A is a schematic representation of the beads prior to target binding and ligation (no emission from acceptor). FIG. 12B is a schematic representation of the beads after target binding and ligation (emission from acceptor detected).

FIG. 13A is a schematic representation of an example of a self-ligating nucleic acid sensor molecule bound to a solid support when used in a TIR-illuminated detection scheme where there is a signal increase upon target binding. FIG. 13B is a schematic representation of the same sensor in an epi-illuminated configuration, where target binding is detected by monitoring changes of the fluorophore bound to the substrate at the surface of the array. FIG. 13C is a schematic representation of the same epi-illuminated configuration, where target binding is detected by monitoring changes in the fluorescence polarization.

FIG. 14 is a schematic representation of a NASM of a ligase ribozyme tethered to a chip by a capture oligonucleotide.

FIG. 15 is a schematic representation of a solid phase self-ligating NASM-ECD chip used for electrochemical detection.

FIG. 16 is a schematic representation of a solid-phase self-cleaving NASM-ECD ship used for electrochemical detection.

FIG. 17 is a schematic representation of a peak in the faradaic current, centered at the redox potential of the electron donor species (specified for a given reference electrode) and superimposed on top of the capacitive current baseline which is observed in the absence of surface-immobilized signaling probes.

FIG. 18 is a schematic representation of an ADP SPA using an ADP aptamer.

FIG. 19 is a flow chart describing the negative incubation and denaturation scheme for ADP sensor generation.

FIG. 20 is a flow chart detailing the in vitro ADP aptamer selection strategies utilized herein.

FIG. 21 depicts the elution profiles of RNA molecules obtained by affinity chromatography during specified rounds of selection for ADP aptamer. FIGS. 2A, 2B and 2C are graphs showing the elution profiles obtained in round 1, round 4, and round 5 of selection, respectively.

FIG. 22 is a graphic depiction of the enrichment of ADP-binding aptamer candidates over five rounds (1-5) of selection.

FIG. 23 shows the elution profiles of RNA molecules obtained by affinity chromatography during specified rounds of selection for ADP aptamer; FIGS. 4A, 4B, 4C and 4D are graphs showing the elution profiles obtained in round 6, round 8, round 9, and round 10 of selection, respectively.

FIG. 24 is a graph depicting the enrichment of ADP selective aptamer candidates over five rounds (6-10) of selection.

FIG. 25 is a bar graph indicating the enrichment of ADP aptamer candidates over all rounds of the selection.

FIGS. 26A and 26B are elution profiles of RNA molecules obtained by affinity chromatography during specified rounds of selection for ADP aptamer. FIGS. 7A and 7B are the elution profile obtained in round 16 and 6C of selection, respectively.

FIG. 27 is a bar graph indicating the relative ADP binding activity of pooled as well as individual ADP aptamer candidates.

FIG. 28A through 28F are graphs indicating that ADP aptamer candidate clone selectivity for ADP or ATP binding.

FIG. 29 is a graph of competitive binding of ADP aptamer candidate clone F01 (#11 of FIG. 9) selectivity for adenosine nucleoside derivatives.

FIG. 30 depicts various targets for the ADP aptamer and their respective binding constants indicating the specificity of clone F01 for ADP as compared to the specificity of known aptamers for select nucleosides and nucleoside analogs.

FIG. 31 is a graph showing the effect of ATP purity on the specificity of ADP aptamer clone F01.

FIG. 32 is a schematic representation of ppERK kinase SPA using an ADP aptamer.

FIG. 33 depicts the kinetics of ppERK activity. FIG. 14A is a graph of radiometric determination of kinase-mediated phosphorylation of MBP. FIG. 14B is a graph of ppERK activity kinase SPA using an ADP aptamer (FIG. 14B).

FIG. 34 is a bar graph showing the effect of staurosporine on ppERK activity using a kinase SPA incorporating ADP aptamer.

FIG. 35 is a graph showing the effect of staurosporine, SB220025, and olomoucine concentration on ppERK activity in a SPA using an ADP aptamer.

FIG. 36 is a graph showing the kinetics of ADP generation in a HTS 96-well ppERK kinase SPA using an ADP aptamer. FIG. 17A is a graph showing the time course of ADP generation. FIG. 17B is a graph showing the time-dependent signal observed in the ppERK kinase SPA.

FIG. 37 depicts the results of a 96-well high throughput screening (HTS) ppERK kinase assay. FIG. 37A is a graph depicting the ppERK kinase inhibitory activity observed in a compound library using a HTS 96-well ppERK kinase SPA. FIG. 37B is a graph depicting the results of the ADP-HH screening assay. FIG. 37C is a graph depicting the results of the ADP-SPA screening (2nd) assay.

FIG. 38 is a gel depicting the structural characterization of ADP aptamer candidate clone F01 using partial alkaline hydrolysis.

FIG. 39 is a schematic of the ADP candidate clone F01 secondary structure from structural characterization studies.

FIGS. 40A and 40B are schematic representations of predicted structures for ADP aptamer candidate clone F01.

FIGS. 41A and 41B are schematic representations of minimized ADP aptamers constructed from clone F01.

FIG. 42 is a schematic of one embodiment of a minimized ADP aptamer incorporating a capture probe sequence.

FIG. 43 is a graph showing the kinetics of ppERK activity observed in a kinase SPA using an ADP aptamer incorporating a capture probe.

FIG. 44 is a graph showing the effect of staurosporine on ppERK activity in a HTS 96-well ppERK kinase SPA using an ADP aptamer capture probe.

FIG. 45 is a graph showing the effect of SB220025 on ppERK activity in a HTS 96-well ppERK kinase SPA using an ADP aptamer capture probe.

FIG. 46 is a graph showing the effect of ITU on ppERK activity in a HTS 96-well ppERK kinase SPA using an ADP aptamer.

FIGS. 47A and 47B are schematic representations depicting the pool design for ADP dependent hammerhead selections.

FIG. 48 is a bar graph showing the ADP dependence of ADP sensor candidate pools in selection rounds.

FIG. 49 is an autoradiograph of a gel indicating the results of cleavage assay analysis of individual ADP sensor candidates.

FIG. 50 is a schematic representation of functional ADP sensor sequences derived from stem selection studies.

FIGS. 51A and 51B are a schematic representations of functional ADP sensor (A) and the structure of ADP sensor prior to preparation for FRET (B).

FIG. 52 is a schematic of an ADP FRET assay incorporating an ADP sensor.

FIG. 53 is a graph depicting the kinetics of FRET using an ADP sensor.

FIG. 54 is a graph showing the effect of ADP concentration on the kinetics of FRET using an ADP sensor.

FIG. 55 is a graph indicating the linear response of FRET to increasing ADP concentration using an ADP sensor.

FIG. 56 is a graph comparing the time-dependent ADP accumulation observed in conventional radiometric kinase assays using ³²P-γ-ATP or ³²P-α-ATP, and an ADP FRET assay.

FIG. 57 is a schematic representation of the compounds included in the compound library used for inhibitor screening as depicted in FIGS. 37A, 37B and 37C.

### DETAILED DESCRIPTION OF THE INVENTION

The invention is drawn to aptamer nucleic acid molecules (“aptamers”) which selectively recognize ADP. The invention also relates to catalytic nucleic acid sensor molecules (also known as allosteric ribozymes, aptazymes, and the like) and to optical nucleic acid sensor molecules which selectively recognize ADP.

Catalytic nucleic acid sensor molecules (NASMs) can be generated in a number of ways, including use of an aptamer derived target modulation domain joined to a catalytic domain by a linker region. Optical NASMs are generated from catalytic NASMs by addition of an optical signal generating unit. In general, optical NASMs generate a detectable optical signal upon recognition of a target molecule.

The invention also includes methods by which a change in the conformation of a nucleic acid composition of the invention upon recognition of a specific target molecule can be coupled to a quantifiable, measurable signal.

The invention also includes methods which allow one to assay the activity of a biological agent by detecting the starting material, byproduct, or product which is generated or consumed in a reaction carried out by the biological agent. Assays can be carried out in a variety of formats, including in vitro biochemical assays on chips or other substrates or in live cells. These assays have applications in all phases of drug discovery, including target validation, drug discovery and development, as well as high throughput screening.

The invention also includes methods which allow one to test the modulatory activity of compounds on biochemical agents. For example, the activity of a biological agent can be assayed in the presence and absence of a modulatory compound, and the reaction products measured and compared for each. Assays can be carried out in a variety of formats, including in vitro biochemical assays on chips or other substrates or in live cells. These assays have applications in all phases of drug discovery, including target validation, drug discovery and development, as well as high throughput screening.

High throughput screening methods are also provided. A plurality of nucleic acid molecules of the invention are immobilized at discrete sites on a substrate, e.g., on a 96- or 384-well plate. Such a plurality of immobilized nucleic acid compositions can be used to detect the products of a variety of biochemical reactions simultaneously, or can be used to monitor the effects of different reaction conditions (e.g., buffers, or the presence of modulatory compounds) on a particular biochemical reaction.

Nucleic acid compositions of the invention (aptamers and nucleic acid sensor molecules) are RNAs, DNAs, RNA/DNA hybrids, or derivatives or analogs of nucleic acids that catalyze a chemical reaction and/or undergo a conformational change upon the recognition of a specific target molecule.

Nucleic acid compositions of the invention can be generated or selected by a variety of methods both disclosed herein and known in the art. For examples, see WO98/27104, WO01/96559, and WO 00/26226, each of which is incorporated herein by reference.

Definitions

In order to more clearly and concisely describe and point out the subject matter of the claimed invention, the following definitions are provided for specific terms which are used in the following written description and the appended claims.

As defined herein, “nucleic acid” means either DNA, RNA, single-stranded or double-stranded, and any chemical modifications thereof. Modifications include, but are not limited to, those which provide other chemical groups that incorporate additional charge, polarizability, hydrogen bonding, electrostatic interaction, and fluxionality to the nucleic acid ligand bases or to the nucleic acid ligand as a whole. Such modifications include, but are not limited to, 2′-position sugar modifications, 5-position pyrimidine modifications, 8-position purine modifications, modifications at exocyclic amines, substitution of 4-thiouridine, substitution of 5-bromo or 5-iodo-uracil; backbone modifications, methylations, unusual base-pairing combinations such as the isobases isocytidine and isoguanidine and the like. Modifications can also include 3′ and 5′ modifications such as capping.

As defined herein, an “oligonucleotide” is used interchangeably with the term “nucleic acid” and includes RNA or DNA (or RNA/DNA) sequences of more than one nucleotide in either single strand or double-stranded form. A “modified oligonucleotide” includes at least one nucleotide residue with any of: an altered internucleotide linkage(s), altered sugar(s), altered base(s), or combinations thereof.

As defined herein, “target” means any compound or molecule of interest for which a diagnostic test is desired and where a nucleic acid ligand is known or can be identified. A “target” is any molecule to be detected, and is any molecule for which a nucleic acid ligand exists or can be generated. A target molecule can be naturally occurring or artificially created, including a protein, peptide, carbohydrate, polysaccharide, glycoprotein, hormone, receptor, antigen, antibody, virus, substrate, metabolite, transition state analog, cofactor, inhibitor, drug, dye, nutrient, growth factor, etc. without limitation.

As defined herein, a molecule which “naturally binds to DNA or RNA” is one which is found within a cell in an organism found in nature.

As defined herein, a “random sequence” or a “randomized sequence” is a segment of a nucleic acid having one or more regions of fully or partially random sequences. A fully random sequence is a sequence in which there is an approximately equal probability of each base (A, T, C, and G) being present at each position in the sequence. In a partially random sequence, instead of a 25% chance that an A, T, C, or G base is present at each position, there are unequal probabilities.

As defined herein, a “fixed region” is a nucleic acid sequence which is known.

As defined herein, a “signal” is a detectable physical quantity, impulse or object.

As defined herein, an “optical signal” is a signal the optical properties of which can be detected.

As defined herein, a “biological agent” is a substance produced by or found within a living organism.

As defined herein, a “modulatory compound” is a compound that affects the function of a substance having activity, such as a biological agent or a nucleic acid sensor molecule.

As defined herein, the “starting material of a biological reaction” is a substance(s) consumed by a chemical process which can be conducted in a living system.

As defined herein, a “product of a biological reaction” is a substance(s) which is the main substance produced by a chemical process which can be conducted in a living system.

As defined herein, a “byproduct of a biological reaction” is a substance(s), other than the main product, produced by a chemical process which can be conducted in a living system.

As defined herein, “bodily fluid” refers to a mixture of molecules obtained from an organism. This includes, but is not limited to, whole blood, blood plasma, urine, semen, saliva, lymph fluid, meningal fluid, amniotic fluid, glandular fluid, sputum, and cerebrospinal fluid. This also includes experimentally separated fractions of all of the preceding. Bodily fluid also includes solutions or mixtures containing homogenized solid material, such as feces, tissues, and biopsy samples.

As defined herein, “test mixture” refers to any sample that contains a plurality of molecules. This includes, but is not limited to, bodily fluids as defined above, and any sample for environmental and toxicology testing such as contaminated water and industrial effluent.

As defined herein, “fluorescent group” refers to a molecule that, when excited with light having a selected wavelength, emits light of a different wavelength. Fluorescent groups include, but are not limited to, fluorescein, tetramethylrhodamine, Texas Red, BODIPY, 5-[(2-aminoethyl)amino]napthalene-1-sulfonic acid (EDANS), and Lucifer yellow. Fluorescent groups may also be referred to as “fluorophores”.

As defined herein, “fluorescence-modifying group” refers to a molecule that can alter in any way the fluorescence emission from a fluorescent group. A fluorescence-modifying group generally accomplishes this through an energy transfer mechanism. Depending on the identity of the fluorescence-modifying group, the fluorescence emission can undergo a number of alterations, including, but not limited to, attenuation, complete quenching, enhancement, a shift in wavelength, a shift in polarity, a change in fluorescence lifetime. One example of a fluorescence-modifying group is a quenching group.

As defined herein, “energy transfer” refers to the process by which the fluorescence emission of a fluorescent group is altered by a fluorescence-modifying group. If the fluorescence-modifying group is a quenching group, then the fluorescence emission from the fluorescent group is attenuated (quenched). Energy transfer can occur through fluorescence resonance energy transfer, or through direct energy transfer. The exact energy transfer mechanisms in these two cases are different. It is to be understood that any reference to energy transfer in the instant application encompasses all of these mechanistically-distinct phenomena.

As defined herein, “energy transfer pair” refers to any two molecules that participate in energy transfer. Typically, one of the molecules acts as a fluorescent group, and the other acts as a fluorescence-modifying group. The preferred energy transfer pair of the instant invention comprises a fluorescent group and a quenching group. In some cases, the distinction between the fluorescent group and the fluorescence-modifying group may be blurred. For example, under certain circumstances, two adjacent fluorescein groups can quench one another's fluorescence emission via direct energy transfer. For this reason, there is no limitation on the identity of the individual members of the energy transfer pair in this application. All that is required is that the spectroscopic properties of the energy transfer pair as a whole change in some measurable way if the distance between the individual members is altered by some critical amount.

“Energy transfer pair” is used to refer to a group of molecules that form a single complex within which energy transfer occurs. Such complexes may comprise, for example, two fluorescent groups which may be different from one another and one quenching group, two quenching groups and one fluorescent group, or multiple fluorescent groups and multiple quenching groups. In cases where there are multiple fluorescent groups and/or multiple quenching groups, the individual groups may be different from one another e.g., one complex contemplated herein comprises fluorescein and EDANS as fluorescent groups, and DABCYL as a quenching agent.

As defined herein, “quenching group” refers to any fluorescence-modifying group that can attenuate at least partly the light emitted by a fluorescent group. We refer herein to this attenuation as “quenching”. Hence, illumination of the fluorescent group in the presence of the quenching group leads to an emission signal that is less intense than expected, or even completely absent. Quenching occurs through energy transfer between the fluorescent group and the quenching group. The preferred quenching group of the invention is (4-dimethylamino-phenylazo)benzoic acid (DABCYL).

As defined herein, “fluorescence resonance energy transfer” or “FRET” refers to an energy transfer phenomenon in which the light emitted by the excited fluorescent group is absorbed at least partially by a fluorescence-modifying group. If the fluorescence-modifying group is a quenching group, then that group can either radiate the absorbed light as light of a different wavelength, or it can dissipate it as heat. FRET depends on an overlap between the emission spectrum of the fluorescent group and the absorption spectrum of the quenching group. FRET also depends on the distance between the quenching group and the fluorescent group. Above a certain critical distance, the quenching group is unable to absorb the light emitted by the fluorescent group, or can do so only poorly.

As defined herein, “direct energy transfer” refers to an energy transfer mechanism in which passage of a photon between the fluorescent group and the fluorescence-modifying group does not occur. Without being bound by a single mechanism, it is believed that in direct energy transfer, the fluorescent group and the fluorescence-modifying group interfere with each others electronic structure. If the fluorescence-modifying group is a quenching group, this will result in the quenching group preventing the fluorescent group from even emitting light.

In general, quenching by direct energy transfer is more efficient than quenching by FRET. Indeed, some quenching groups that do not quench particular fluorescent groups by FRET (because they do not have the necessary spectral overlap with the fluorescent group) can do so efficiently by direct energy transfer. Furthermore, some fluorescent groups can act as quenching groups themselves if they are close enough to other fluorescent groups to cause direct energy transfer. For example, under these conditions, two adjacent fluorescein groups can quench one another's fluorescence effectively. For these reasons, there is no limitation on the nature of the fluorescent groups and quenching groups useful for the practice of this invention.

As defined herein, an “aptamer” is a nucleic acid which binds to a non-nucleic acid target molecule or a nucleic acid target through non-Watson-Crick base pairing.

As defined herein, an aptamer nucleic acid molecule which “recognizes a target molecule” is a nucleic acid molecule which specifically binds to a target molecule.

As defined herein, a “nucleic acid sensor molecule” or “NASM” refers to either or both of a catalytic nucleic acid sensor molecule and an optical nucleic acid sensor molecule.

As defined herein, a “catalytic nucleic acid sensor molecule” is a nucleic acid sensor molecule comprising a target modulation domain, a linker region, and a catalytic domain.

As defined herein, an ‘optical nucleic acid sensor molecule” is a catalytic nucleic acid sensor molecule wherein the catalytic domain has been modified to emit an optical signal as a result of and/or in lieu of catalysis by the inclusion of an optical signal generating unit.

As defined herein, a “nucleic acid ligand” refers to either or both an aptamer or NASM.

As defined herein, a “target modulation domain” (TMD) is the portion of a nucleic acid sensor molecule which recognizes a target molecule. The target modulation domain is also sometimes referred to herein as the “target activation site” or “effector modulation domain”.

As defined herein, a “catalytic domain” is the portion of a nucleic acid sensor molecule possessing catalytic activity which is modulated in response to binding of a target molecule to the target modulation domain.

As defined herein, a “linker region” or “linker domain” is the portion of a nucleic acid sensor molecule by or at which the “target modulation domain” and “catalytic domain” are joined. Linker regions include, but are not limited to, oligonucleotides of varying length, base pairing phosphodiester, phosphothiolate, and other covalent bonds, chemical moieties (e.g., PEG), PNA, formacetal, bismaleimide, disulfide, and other bifunctional linker reagents. The linker domain is also sometimes referred to herein as a “connector” or “stem”.

As defined herein, an “optical signal generating unit” is a portion of a nucleic acid sensor molecule comprising one or more nucleic acid sequences and/or non-nucleic acid molecular entities, which change optical or electrochemical properties or which change the optical or electrochemical properties of molecules in close proximity to them in response to a change in the conformation or the activity of the nucleic acid sensor molecule following recognition of a target molecule by the target modulation domain.

As defined herein, a nucleic acid sensor molecule which “recognizes a target molecule” is a nucleic acid molecule whose activity is modulated upon binding of a target molecule to the target modulation domain to a greater extent than it is by the binding of any non-target molecule or in the absence of the target molecule. The recognition event between the nucleic acid sensor molecule and the target molecule need not be permanent during the time in which the resulting allosteric modulation occurs. Thus, the recognition event can be transient with respect to the ensuing allosteric modulation (e.g., conformational change) of the nucleic acid sensor molecule.

As defined herein, a “cleavage substrate” is an oligonucleotide or portion of an oligonucleotide cleaved upon target molecule recognition by a target modulation domain of an endonucleolytic nucleic acid sensor molecule.

As defined herein, an “oligonucleotide substrate” is an oligonucleotide that is acted upon by the catalytic domain of a nucleic acid sensor molecule with ligase activity.

As defined herein, an “effector oligonucleotide” is an oligonucleotide that base pairs with the effector oligonucleotide binding domain of a nucleic acid sensor molecule with ligase activity.

As defined herein, an “effector oligonucleotide binding domain” is the portion of the nucleic acid sensor molecule with ligase activity which is complementary to the effector oligonucleotide.

As defined herein, a “capture oligonucleotide” is an oligonucleotide that is used to attach a nucleic acid sensor molecule to a substrate by complementarity and/or hybridization.

As defined herein, an “oligonucleotide substrate binding domain” is the portion on the nucleic acid sensor molecule with ligase activity that is complementary to and can base pair with an oligonucleotide substrate.

As defined herein, a “oligonucleotide supersubstrate” is an oligonucleotide substrate that is complementary to and can base pair with the oligonucleotide substrate binding domain and to the effector oligonucleotide binding domain of a nucleic acid sensor molecule with ligase activity. The oligonucleotide supersubstrate may or may not carry an affinity tag.

As defined herein, a “oligonucleotide supersubstrate binding domain” is the region of a nucleic acid sensor molecule with ligase activity that is complementary to and can base pair with the oligonucleotide supersubstrate.

As defined herein, “switch factor” is the enhancement observed in the catalytic activity and/or catalytic initial rate of a nucleic acid sensor molecule upon recognition of a target molecule by the target modulation domain.

As defined herein, an “amplicon” is the sequence of a nucleic acid sensor molecule with ligase activity covalently ligated to an oligonucleotide substrate.

As defined herein, “amplicon dependent nucleic acid amplification“refers to a technique by which one can amplify the signal of a nucleic acid sensor molecule by use of standard RT/PCR or Real-Time RT-PCR methods.”

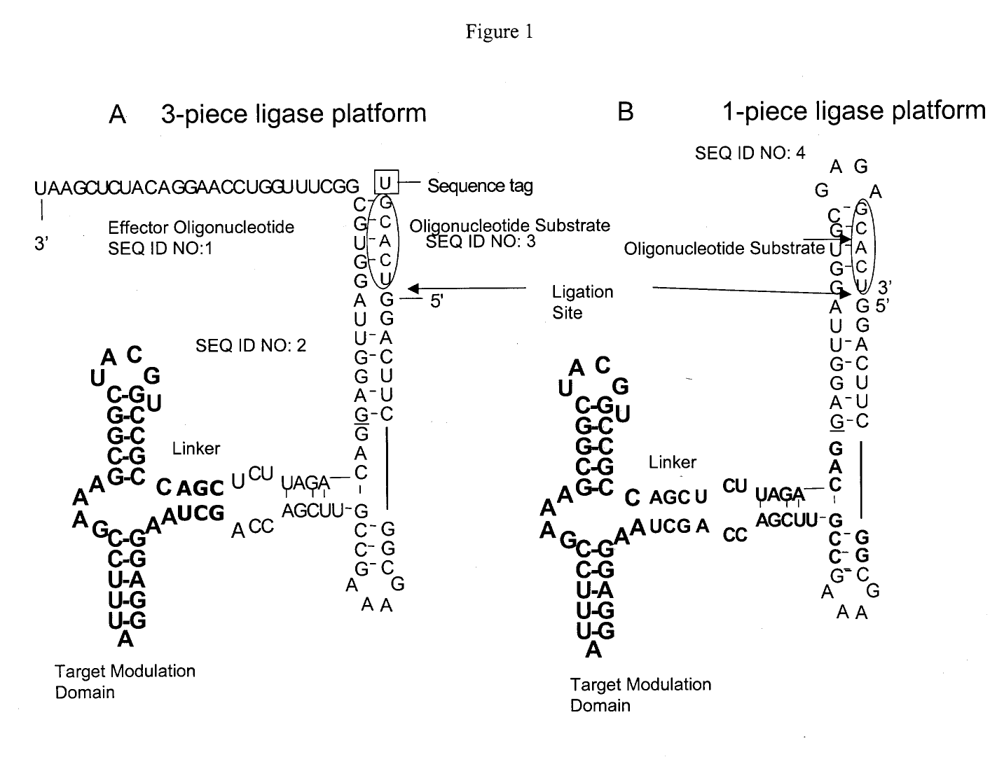

As defined herein, a “3-piece ligase” is a 3-component trans-ligase ribozyme. The first component consists of the catalytic domain, the linker, the target modulation domain, the substrate binding domain and the effector oligonucleotide binding domain. The second component is the effector oligonucleotide that is complementary to the effector oligonucleotide binding domain. The third component is the oligonucleotide substrate that is complementary to the substrate binding domain. This system follows the format of the 3-piece ligase platform shown in FIG. 1A.

As defined herein, a “cis-ligase ribozyme” is a ligase ribozyme that ligates its 3′ end to its 5′ end. The cis-ligase ribozyme is also referred herein as “1-piece ligase” and is a 1-component system where oligonucleotide substrate, oligonucleotide substrate binding domain, catalytic domain, effector oligonucleotide and effector oligonucleotide binding domains are fused in the format shown in FIG. 1B.

As defined herein, a “trans-ligase ribozyme” is a ligase ribozyme that ligates its 5′ end to the 3′ end of an oligonucleotide substrate.

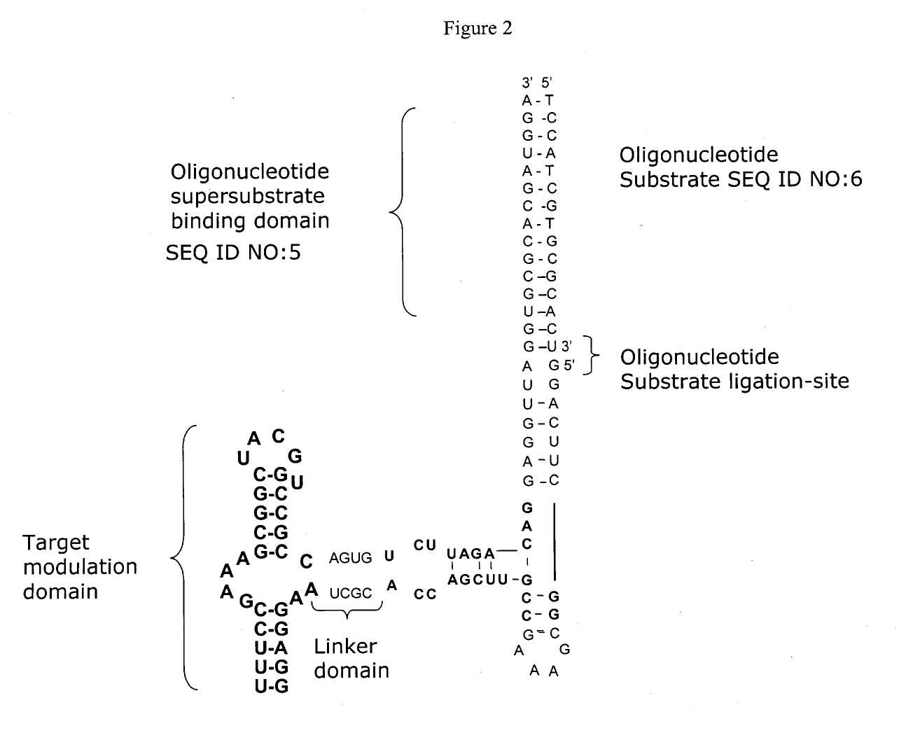

As defined herein, a “2-piece ligase” is a 2-component trans-ligase ribozyme. The first component consists of the catalytic domain, the linker region, the target modulation domain, the substrate binding domain and the effector oligonucleotide binding domain. The second component is the oligonucleotide substrate that is complementary to the substrate binding domain and the effector oligonucleotide binding domain. This system follows the format shown in FIG. 2.

As defined herein, “stem selection” refers to a process performed on a pool of nucleic molecules comprising a target modulation domain, a catalytic domain and an oligonucleotide linker region wherein the linker region is fully or partially randomized.

As defined herein, “rational design/engineering” refers to a technique used to construct nucleic acid sensor molecules in which a non-conserved region of a ribozyme is replaced with a target modulation domain and joined to the catalytic domain of the ribozyme by an oligonucleotide linker region.

As defined herein, a “biosensor” comprises a plurality of nucleic acid ligands.

As defined herein, “substrate” means any physical supporting surface, whether rigid, flexible, solid, porous, gel-based, or of any other material or composition. A substrate includes a microfabricated solid surface to which molecules may be attached through either covalent or non-covalent bonds. This includes, but is not limited to, Langmuir-Bodgett films, functionalized glass, membranes, charged paper, nylon, germanium, silicon, PTFE, polystyrene, gallium arsenide, gold, and silver. Any other material known in the art that is capable of having functional groups such as amino, carboxyl, thiol or hydroxyl incorporated on its surface, is contemplated. This includes surfaces with any topology, such spherical surfaces and grooved surfaces.

As defined herein, an “array” or “microarray” refers to a biosensor comprising a plurality of nucleic acid sensor molecules immobilized on a substrate.

As defined herein, “specificity” refers to the ability of a nucleic acid of the present invention to recognize and discriminate among competing or closely-related targets or ligands. The degree of specificity of a given nucleic acid is not necessarily limited to, or directly correlated with, the binding affinity of a given molecule. For example, hydrophobic interaction between molecule A and molecule B has a high binding affinity, but a low degree of specificity. A nucleic acid that is 100 times more specific for target A relative to target B will preferentially recognize and discriminate for target A 100 times better than it recognizes and discriminates for target B.

As defined herein, “selective” refers to a molecule that has a high degree of specificity for a target molecule.

I. Nucleic Acid Compositions

In addition to carrying genetic information, nucleic acids can adopt complex three-dimensional structures. These three-dimensional structures are capable of specific recognition of target molecules and, furthermore, of catalyzing chemical reactions. Nucleic acids will thus provide candidate detection molecules for diverse target molecules, including those which do not naturally recognize or bind to DNA or RNA.

In aptamer selection, combinatorial libraries of oligonucleotides are screened in vitro to identify oligonucleotides which bind with high affinity to pre-selected targets. In NASM selection, on the other hand, combinational libraries of oligonucleotides are screened in vitro to identify oligonucleotides which exhibit increased catalytic activity in the presence of targets. Possible target molecules for both aptamers and NASMS include natural and synthetic polymers, including proteins, polysaccharides, glycoproteins, hormones, receptors, and cell surfaces, and small molecules such as drugs, metabolites, transition state analogs, specific phosphorylation states, and toxins. Small biomolecules, e.g., amino acids, nucleotides, NAD, S-adenosyl methionine, chloramphenicol, and large biomolecules, e.g., thrombin, Ku, DNA polymerases, are effective targets for aptamers, catalytic RNAs (ribozymes) discussed herein (e.g., hammerhead RNAs, hairpin RNAs) as well as NASMs.

In preferred embodiments, the aptamers and NASMs of the invention specifically recognize ADP. The nucleic acids of the invention are therefore useful in the detection of biological agents which consume or produce ADP as a starting material, product, or byproduct of their activity.

While the aptamer selection processes described identifies aptamers through affinity-based (binding) selections, the selection processes as described for NASMs identifies nucleic acid sensor molecules through target modulation of the catalytic core of a ribozyme. In NASM selection, selective pressure on the starting population of NASMs (starting pool size is as high as 10¹⁴ to 10¹⁷ molecules) results in nucleic acid sensor molecules with enhanced catalytic properties, but not necessarily in enhanced binding properties. Specifically, the NASM selection procedures place selective pressure on catalytic effectiveness of potential NASMS by modulating both target concentration and reaction time-dependence. Either parameter, when optimized throughout the selection, can lead to nucleic acid molecular sensor molecules which have custom-designed catalytic properties, e.g., NASMs that have high switch factors, and or NASMs that have high specificity.

II. Selection and Generation of a Target Specific Nucleic Acid Aptamer

Systematic Evolution of Ligands by Exponential Enrichment, “SELEX™,” is a method for making a nucleic acid ligand for any desired target, as described, e.g., in U.S. Pat. Nos. 5,475,096 and 5,270,163, and PCT/US91/04078, each of which is specifically incorporated herein by reference.

SELEX™ technology is based on the fact that nucleic acids have sufficient capacity for forming a variety of two- and three-dimensional structures and sufficient chemical versatility available within their monomers to act as ligands (i.e., form specific binding pairs) with virtually any chemical compound, whether large or small in size.

The method involves selection from a mixture of candidates and step-wise iterations of structural improvement, using the same general selection theme, to achieve virtually any desired criterion of binding affinity and selectivity. Starting from a mixture of nucleic acids, preferably comprising a segment of randomized sequence, the SELEX™ method includes steps of contacting the mixture with the target under conditions favorable for binding, partitioning unbound nucleic acids from those nucleic acids which have bound to target molecules, dissociating the nucleic acid-target pairs, amplifying the nucleic acids dissociated from the nucleic acid-target pairs to yield a ligand-enriched mixture of nucleic acids, then reiterating the steps of binding, partitioning, dissociating and amplifying through as many cycles as desired.

Within a nucleic acid mixture containing a large number of possible sequences and structures, there is a wide range of binding affinities for a given target. A nucleic acid mixture comprising, for example a 20 nucleotide randomized segment can have 4²⁰ candidate possibilities. Those which have the higher affinity constants for the target are most likely to bind to the target. After partitioning, dissociation and amplification, a second nucleic acid mixture is generated, enriched for the higher binding affinity candidates. Additional rounds of selection progressively favor the best ligands until the resulting nucleic acid mixture is predominantly composed of only one or a few sequences. These can then be cloned, sequenced and individually tested for binding affinity as pure ligands.

Cycles of selection and amplification are repeated until a desired goal is achieved. In the most general case, selection/amplification is continued until no significant improvement in binding strength is achieved on repetition of the cycle. The method may be used to sample as many as about 10¹⁸ different nucleic acid species. The nucleic acids of the test mixture preferably include a randomized sequence portion as well as conserved sequences necessary for efficient amplification. Nucleic acid sequence variants can be produced in a number of ways including synthesis of randomized nucleic acid sequences and size selection from randomly cleaved cellular nucleic acids. The variable sequence portion may contain fully or partially random sequence; it may also contain subportions of conserved sequence incorporated with randomized sequence. Sequence variation in test nucleic acids can be introduced or increased by mutagenesis before or during the selection/amplification iterations.

In one embodiment of SELEX™, the selection process is so efficient at isolating those nucleic acid ligands that bind most strongly to the selected target, that only one cycle of selection and amplification is required. Such an efficient selection may occur, for example, in a chromatographic-type process wherein the ability of nucleic acids to associate with targets bound on a column operates in such a manner that the column is sufficiently able to allow separation and isolation of the highest affinity nucleic acid ligands.

In many cases, it is not necessarily desirable to perform the iterative steps of SELEX™ until a single nucleic acid ligand is identified. The target-specific nucleic acid ligand solution may include a family of nucleic acid structures or motifs that have a number of conserved sequences and a number of sequences which can be substituted or added without significantly affecting the affinity of the nucleic acid ligands to the target. By terminating the SELEX™ process prior to completion, it is possible to determine the sequence of a number of members of the nucleic acid ligand solution family.

A variety of nucleic acid primary, secondary and tertiary structures are known to exist. The structures or motifs that have been shown most commonly to be involved in non-Watson-Crick type interactions are referred to as hairpin loops, symmetric and asymmetric bulges, pseudoknots and myriad combinations of the same. Almost all known cases of such motifs suggest that they can be formed in a nucleic acid sequence of no more than 30 nucleotides. For this reason, it is often preferred that SELEX procedures with contiguous randomized segments be initiated with nucleic acid sequences containing a randomized segment of between about 20-50 nucleotides.

The basic SELEX™ method has been modified to achieve a number of specific objectives. For example, U.S. Pat. No. 5,707,796 describes the use of SELEX™ in conjunction with gel electrophoresis to select nucleic acid molecules with specific structural characteristics, such as bent DNA. U.S. Pat. No. 5,763,177 describes a SELEX™ based methods for selecting nucleic acid ligands containing photoreactive groups capable of binding and/or photocrosslinking to and/or photoinactivating a target molecule. U.S. Pat. No. 5,567,588 and U.S. application Ser. No. 08/792,075, filed Jan. 31, 1997, entitled “Flow Cell SELEX”, describe SELEX™ based methods which achieve highly efficient partitioning between oligonucleotides having high and low affinity for a target molecule. U.S. Pat. No. 5,496,938 describes methods for obtaining improved nucleic acid ligands after the SELEX™ process has been performed. U.S. Pat. No. 5,705,337 describes methods for covalently linking a ligand to its target. Each of these patents and applications is specifically incorporated herein by reference.

SELEX™ can also be used to obtain nucleic acid ligands that bind to more than one site on the target molecule, and to nucleic acid ligands that include non-nucleic acid species that bind to specific sites on the target. SELEX™ provides means for isolating and identifying nucleic acid ligands which bind to any envisionable target, including large and small biomolecules including proteins (including both nucleic acid-binding proteins and proteins not known to bind nucleic acids as part of their biological function) cofactors and other small molecules. See U.S. Pat. No. 5,580,737 for a discussion of nucleic acid sequences identified through SELEX™ which are capable of binding with high affinity to caffeine and the closely related analog, theophylline.

Counter-SELEX™ is a method for improving the specificity of nucleic acid ligands to a target molecule by eliminating nucleic acid ligand sequences with cross-reactivity to one or more non-target molecules. Counter-SELEX™ is comprised of the steps of a) preparing a candidate mixture of nucleic acids; b) contacting the candidate mixture with the target, wherein nucleic acids having an increased affinity to the target relative to the candidate mixture may be partitioned from the remainder of the candidate mixture; c) partitioning the increased affinity nucleic acids from the remainder of the candidate mixture; d) contacting the increased affinity nucleic acids with one or more non-target molecules such that nucleic acid ligands with specific affinity for the non-target molecule(s) are removed; and e) amplifying the nucleic acids with specific affinity to the target molecule to yield a mixture of nucleic acids enriched for nucleic acid sequences with a relatively higher affinity and specificity for binding to the target molecule.

For example, a heterogeneous population of oligonucleotide molecules comprising randomized sequences is generated and selected to identify a nucleic acid molecule having a binding affinity which is selective for a target molecule. (U.S. Pat. Nos. 5,475,096; 5,476,766; and 5,496,938) each of is incorporated herein by reference. In some examples, a population of 100% random oligonucleotides is screened. In others, each oligonucleotide in the population comprises a random sequence and at least one fixed sequence at its 5′ and/or 3′ end. The oligonucleotide can be RNA, DNA, or mixed RNA/DNA, and can include modified or nonnatural nucleotides or nucleotide analogs. (U.S. Pat. Nos. 5,958,691; 5,660,985; 5,958,691; 5,698,687; 5,817,635; and 5,672,695, PCT publication WO 92/07065).

The random sequence portion of the oligonucleotide is flanked by at least one fixed sequence which comprises a sequence shared by all the molecules of the oligonucleotide population. Fixed sequences include sequences such as hybridization sites for PCR primers, promoter sequences for RNA polymerases (e.g., T3, T4, T7, SP6, and the like), restriction sites, or homopolymeric sequences, such as poly A or poly T tracts, catalytic cores (described further below), sites for selective binding to affinity columns, and other sequences to facilitate cloning and/or sequencing of an oligonucleotide of interest.

In one embodiment, the random sequence portion of the oligonucleotide is about 15-70 (e.g., about 30-40) nucleotides in length and can comprise ribonucleotides and/or deoxyribonucleotides. Random oligonucleotides can be synthesized from phosphodiester-linked nucleotides using solid phase oligonucleotide synthesis techniques well known in the art (Froehler et al., Nucl. Acid Res. 14:5399-5467 (1986); Froehler et al., Tet. Lett. 27:5575-5578 (1986)). Oligonucleotides can also be synthesized using solution phase methods such as triester synthesis methods (Sood et al., Nucl. Acid Res. 4:2557 (1977); Hirose et al., Tet. Lett., 28:2449 (1978)). Typical syntheses carried out on automated DNA synthesis equipment yield 10¹⁵-10¹⁷ molecules. Sufficiently large regions of random sequence in the sequence design increases the likelihood that each synthesized molecule is likely to represent a unique sequence.

To synthesize randomized sequences, mixtures of all four nucleotides are added at each nucleotide addition step during the synthesis process, allowing for random incorporation of nucleotides. In one embodiment, random oligonucleotides comprise entirely random sequences; however, in other embodiments, random oligonucleotides can comprise stretches of nonrandom or partially random sequences. Partially random sequences can be created by adding the four nucleotides in different molar ratios at each addition step.

The SELEX method encompasses the identification of high-affinity nucleic acid ligands containing modified nucleotides conferring improved characteristics on the ligand, such as improved in vivo stability or improved delivery characteristics. Examples of such modifications include chemical substitutions at the ribose and/or phosphate and/or base positions. SELEX-identified nucleic acid ligands containing modified nucleotides are described in U.S. Pat. No. 5,660,985, which describes oligonucleotides containing nucleotide derivatives chemically modified at the 5′ and 2′ positions of pyrimidines. U.S. Pat. No. 5,756,703 describes oligonucleotides containing various 2′-modified pyrimidines. U.S. Pat. No. 5,580,737 describes highly specific nucleic acid ligands containing one or more nucleotides modified with 2′-amino (2′-NH₂), 2′-fluoro (2′-F), and/or 2′-O-methyl (2′-OMe) substituents.

The SELEX method encompasses combining selected oligonucleotides with other selected oligonucleotides and non-oligonucleotide functional units as described in U.S. Pat. No. 5,637,459 and U.S. Pat. No. 683,867. The SELEX method further encompasses combining selected nucleic acid ligands with lipophilic or non-immunogenic high molecular weight compounds in a diagnostic or therapeutic complex, as described in U.S. Pat. No. 6,011,020. VEGF nucleic acid ligands that are associated with a lipophilic compound, such as diacyl glycerol or dialkyl glycerol, in a diagnostic or therapeutic complex are described in U.S. Pat. No. 5,859,228.

VEGF nucleic acid ligands that are associated with a lipophilic compound, such as a glycerol lipid, or a non-immunogenic high molecular weight compound, such as polyalkylene glycol are further described in U.S. Pat. No. 6,051,698. VEGF nucleic acid ligands that are associated with a non-immunogenic, high molecular weight compound or a lipophilic compound are further described in PCT Publication No. WO 98/18480. These patents and applications allow the combination of a broad array of shapes and other properties, and the efficient amplification and replication properties, of oligonucleotides with the desirable properties of other molecules. Each of the above references, which describe modifications of the basic SELEX procedure are specifically incorporated by reference in its entirety.

The identification of nucleic acid ligands to small, flexible peptides via the SELEX method has been explored. Small peptides have flexible structures and usually exist in solution in an equilibrium of multiple conformers, and thus it was initially thought that binding affinities may be limited by the conformational entropy lost upon binding a flexible peptide. However, the feasibility of identifying nucleic acid ligands to small peptides in solution was demonstrated in U.S. Pat. No. 5,648,214. In this patent, high affinity RNA nucleic acid ligands to substance P, an 11 amino acid peptide, were identified. This reference is specifically incorporated by reference in its entirety.

To generate oligonucleotide populations which are resistant to nucleases and hydrolysis, modified oligonucleotides can be used and can include one or more substitute internucleotide linkages, altered sugars, altered bases, or combinations thereof. In one embodiment, oligonucleotides are provided in which the P(O)O group is replaced by P(O)S (“thioate”), P(S)S (“dithioate”), P(O)NR₂ (“amidate”), P(O)R, P(O)OR′, CO or CH₂ (“formacetal”) or 3′-amine (—NH—CH₂—CH₂—), wherein each R or R′ is independently H or substituted or unsubstituted alkyl. Linkage groups can be attached to adjacent nucleotide through an —O—, —N—, or —S— linkage. Not all linkages in the oligonucleotide are required to be identical.

In further embodiments, the oligonucleotides comprise modified sugar groups, for example, one or more of the hydroxyl groups is replaced with halogen, aliphatic groups, or functionalized as ethers or amines. In one embodiment, the 2′-position of the furanose residue is substituted by any of an O-methyl, O-alkyl, O-allyl, S-alkyl, S-allyl, or halo group. Methods of synthesis of 2′-modified sugars are described in Sproat, et al., Nucl. Acid Res. 19:733-738 (1991); Cotten, et al., Nucl. Acid Res. 19:2629-2635 (1991); and Hobbs, et al., Biochemistry 12:5138-5145 (1973). The use of 2-fluoro-ribonucleotide oligomer molecules can increase the sensitivity of a nucleic acid sensor molecule for a target molecule by ten- to- one hundred-fold over those generated using unsubstituted ribo- or deoxyribooligonucleotides (Pagratis, et al., Nat. Biotechnol. 15:68-73 (1997)), providing additional binding interactions with a target molecule and increasing the stability of the secondary structure(s) of the nucleic acid sensor molecule (Kraus, et al., Journal of Immunology 160:5209-5212 (1998); Pieken, et al., Science 253:314-317 (1991); Lin, et al., Nucl. Acids Res. 22:5529-5234 (1994); Jellinek, et al. Biochemistry 34:11363-11372 (1995); Pagratis, et al., Nat. Biotechnol 15:68-73 (1997)).

Nucleic acid aptamer molecules are generally selected in a 5 to 20 cycle procedure. In one embodiment, heterogeneity is introduced only in the initial selection stages and does not occur throughout the replicating process.

The starting library of DNA sequences is generated by automated chemical synthesis on a DNA synthesizer. This library of sequences is transcribed in vitro into RNA using T7 RNA polymerase and purified. In one example, the 5′-fixed:random:3′-fixed sequence is separated by a random sequence having 30 to 50 nucleotides.

1) ADP Aptamers

Sorting among the billions of aptamer candidates to find the desired molecules starts from the complex sequence pool, whereby desired ADP aptamers are isolated through an iterative in vitro selection process. The selection process removes both non-specific and non-binding types of contaminants. In a following amplification stage, thousands of copies of the surviving sequences are generated to enable the next round of selection. During amplification, random mutations can be introduced into the copied molecules—this ‘genetic noise’ allows functional nucleic acid aptamer molecules to continuously evolve and become even better adapted. The entire experiment reduces the pool complexity from 10¹⁷ molecules down to around 100 ADP aptamer candidates that require detailed characterization.

Aptamer selection is accomplished by passing a solution of oligonucleotides through a column containing the target molecule (e.g., ADP). The flow-through, containing molecules which are incapable of binding target, is discarded. The column is washed, and the wash solution is discarded. Oligonucleotides which bound to the column are then specifically eluted, reverse transcribed, amplified by PCR (or other suitable amplification techniques), transcribed into RNA, and then reapplied to the selection column. Successive rounds of column application are performed until a pool of aptamers enriched in target binders is obtained.

Negative selection steps can also be performed during the selection process. Addition of such selection steps is useful to remove aptamers which bind to a target in addition to the desired target. Additionally, where the target column is known to contain an impurity, negative selection steps can be performed to remove from the binding pool those aptamers which bind selectively to the impurity, or to both the impurity and the desired target. For example, where the desired target is ADP, care must be taken so as to remove aptamers which bind to closely related molecules such as ATP. Examples of negative selection steps include, for example, incorporating column washing steps with ATP in the buffer, or the addition of an ATP column before the ADP selection column (e.g., the flow through from the ATP column will contain aptamers which do not bind ATP).

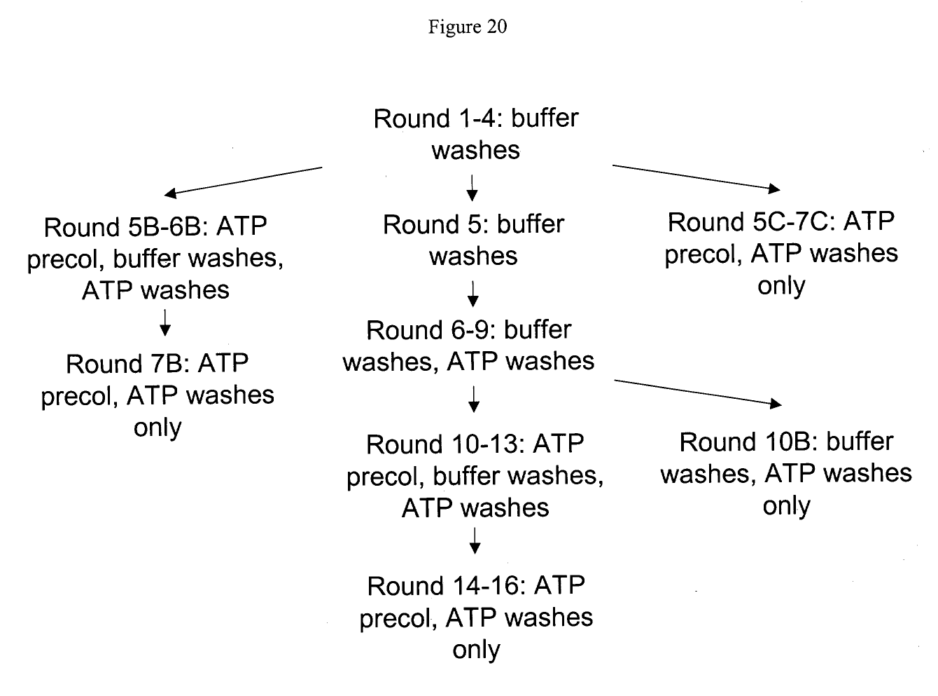

FIG. 20 summarizes the selection strategies tested in studies to optimize the ADP aptamer selection protocol. These strategies included washes with selection buffer and then washes with ATP in selection buffer. Washes only with ATP in selection buffer were also tested. The use of an ATP precolumn was tested. Further, starting material from the initial elution peak from round 4 was used. RNA specifically eluted from the ADP column was ethanol precipitated, reverse transcribed, PCR amplified and ultimately transcribed again into RNA for the next round of selection.

After the completion of selection, the ADP-specific aptamers are reverse transcribed into DNA, cloned, sequenced, and/or resynthesized using natural or modified nucleotides, and amplified.

2) Core Uses of ADP Aptamers

Protein kinases are involved in a number of biological processes, including signal transduction in a variety of different cell types and the initiation and timing of various events (e.g., DNA synthesis and mitosis) in the cell cycle. Because kinases are essential cellular signaling molecules, mutations which affect kinase activity can lead to diseases and disorders, including Hirschsprung's disease (aganglionic megacolon), agammaglobulinemia; non-insulin dependent diabetes mellitus (NIDDM); mastocytocis; hypochondroplasia; and other immunodeficiencies, cancers, and endocrine disorders. Thus, detection of kinase activity, and the ability to identify compounds which modulate it are important in the diagnosis and treatment of kinase-related diseases and disorders.

The typical process by which compounds able to modulate the activity of kinases are identified is a high throughput screen. A high throughput screen is typically a biochemical reaction configured to produce a detectible signal that is correlated to the extent of the reaction. In the case of a protein kinase, which converts ATP into ADP and phosphorylates a protein or peptide substrate in the process, a detectible signal that is correlated to the amount of ADP produced would yield information on the extent of reaction and thus on activity of the kinase. In a high throughput screen, the same kinase reaction is repeated thousands to millions of times in exactly the same way. The only difference between the reactions is that each reaction will have a different compound (potential inhibitor or activator) or set of compounds added to it. Reactions whose detectible kinase activity is unchanged relative to control reactions without compound, contain inactive compounds and are called “misses”. Reactions whose detectible kinase activity is significantly changed relative to control reactions without compound, contain active compounds and are called “hits”. These “hits” represent compounds that are potential drug leads which are sorted out of the hits using a variety of assays to determine, e.g., activity, specificity, or affinity, well known to those of ordinary skill in the art. In addition, the kinase assay described above can itself be used to identify drug leads among the hits by determining which candidate drugs effectively modulate kinase activity.

Because the process of high throughput screening requires thousands to millions of assays, each assay will ideally be very reliable to prevent both false hits and false misses. The assay should also require minimal manipulation and additional reagents to keep the cost per assay as low as possible.

Thus, the ADP aptamers of the invention are useful for direct ADP detection, indirect ATP detection by detection of a change in ADP, detection of kinase activity by monitoring the production of ADP as a byproduct of the kinase reaction, and monitoring the effect of various kinase inhibitors by monitoring the effect on the production of ADP as a byproduct of the kinase reaction.

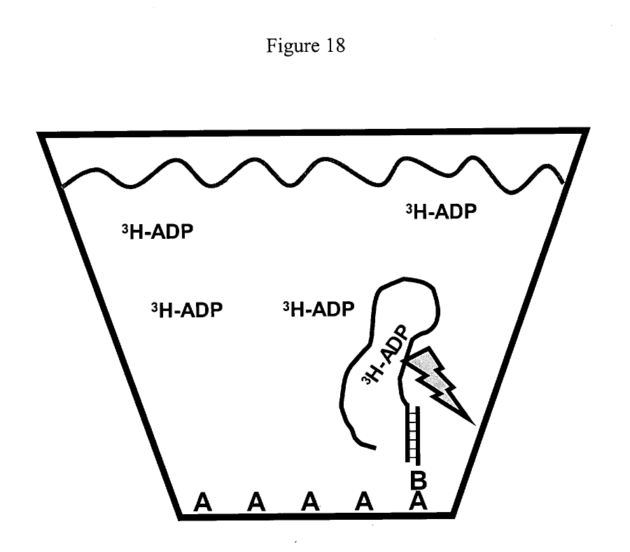

To facilitate use of the aptamers in high throughput screening assays, an aptamer can be generated with a 3′ sequence tag which specifically hybridizes with a biotinylated capture oligo. Such a capture oligo then can be used to immobilize the aptamer on a streptavidin coated substrate through the biotin-streptavidin binding. When such a streptavidin coated substrate is a flash plate (e.g., a plate containing a scintillant imbedded therein), a surface immobilized aptamer that binds to ³H-ADP will concentrate the tritiated nucleotide on the surface of the flash plate and generate a detectable scintillation proximity signal. See FIG. 18.

Using this methodology, aptamers can be analyzed for the ability to yield ADP-mediated signal in the ADP SPA. Additionally, the aptamers can be analyzed for the ability to discriminate between ADP and ATP, or other ADP analogs.

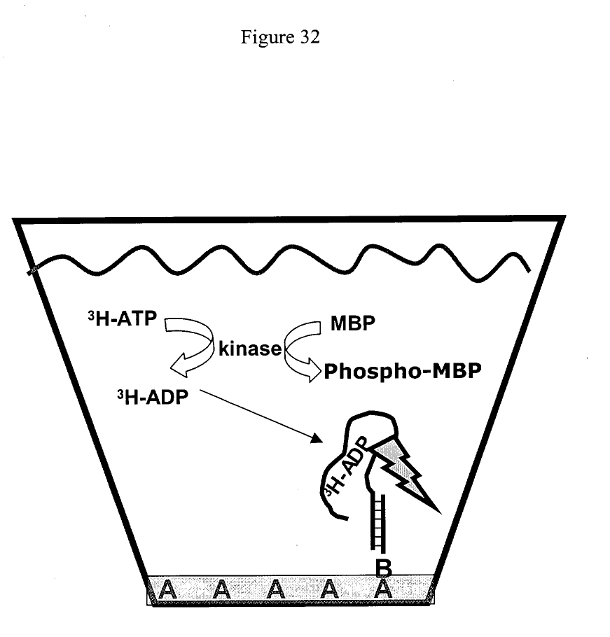

As shown in FIG. 32, a surface immobilized aptamer that binds to ³H-ADP can be utilized to measure kinase-mediated protein phosphorylation. An ADP aptamer will concentrate the tritiated ADP released by kinase on the surface of the flash plate and generate a detectable scintillation proximity signal.

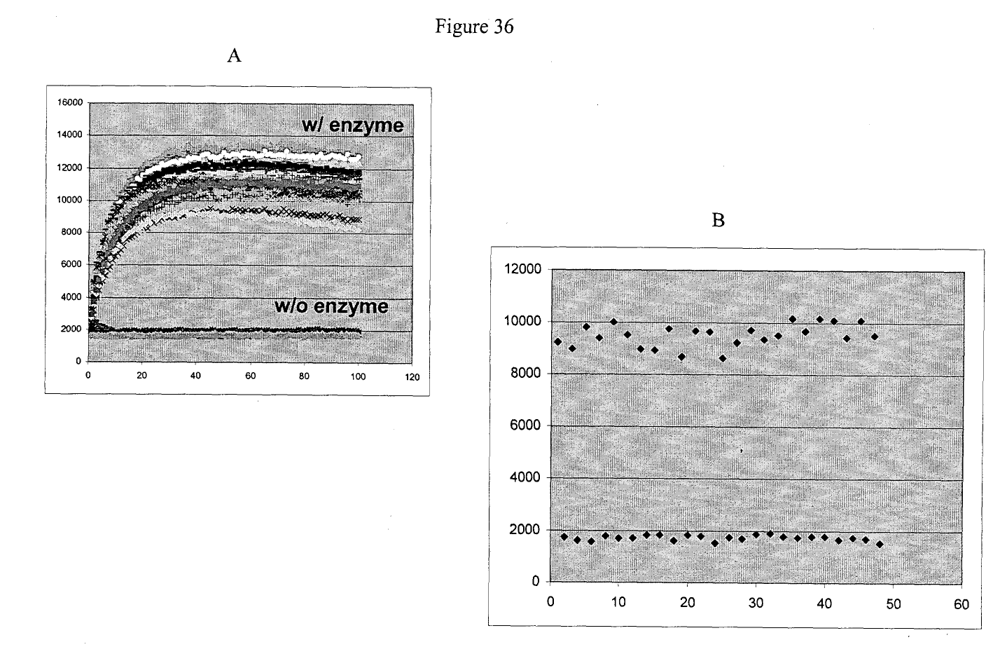

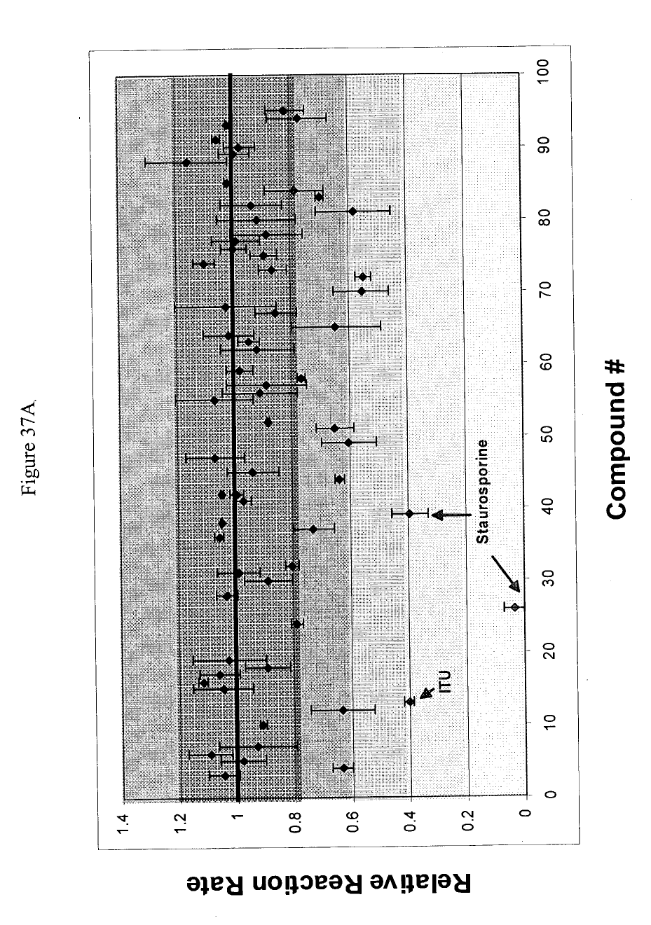

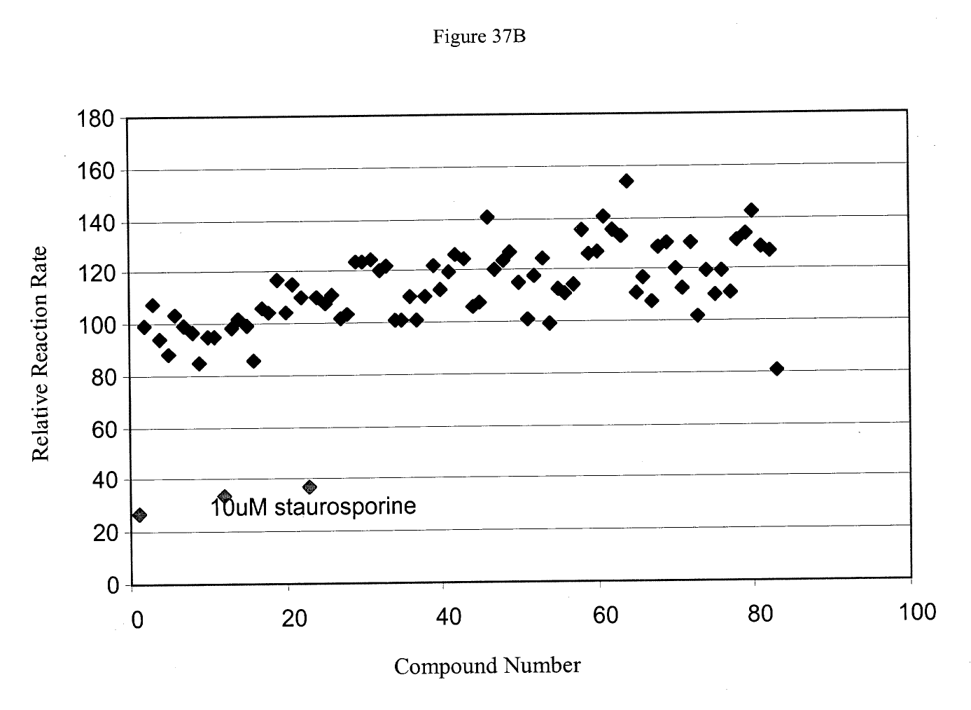

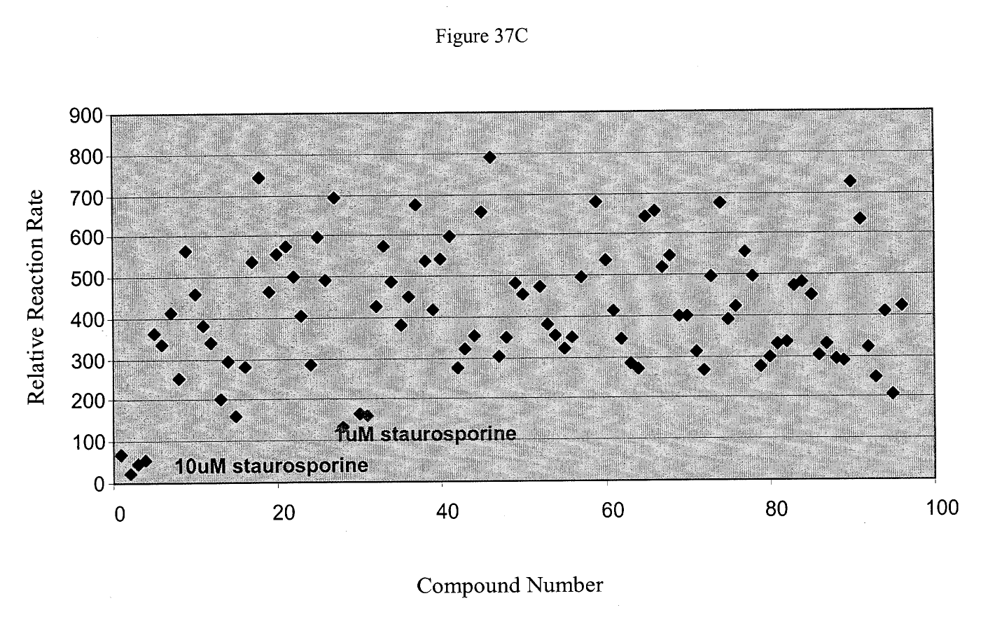

A 96-well high throughput screening (HTS) kinase assay demonstrating the effect of various kinase inhibitors on ppERK activity is shown in FIGS. 36 and 37. In this assay, the effect of inhibitor on ADP production is determined as measured by ADP aptamer SPA.

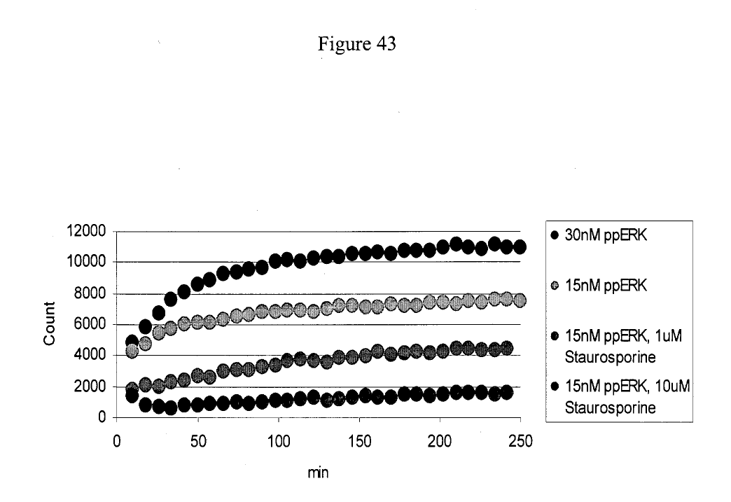

FIG. 43 shows the use of an ADP aptamer to detect ppERK-mediated phosphorylation of Myelin basic protein (MBP) in an ADP SPA. The phosphorylation of ppERK is reflected in the time-dependent and ppERK-dependent increase in assay signal observed using the ADP SPA incorporating the minimized, directly biotinylated ADP aptamer. Furthermore, as shown in FIG. 43, concentration-dependent inhibition of ppERK by staurosporine using this ADP SPA is consistent with the known kinase inhibitory activity of staurosporine.

3) Other Uses of ADP Aptamers

Target Discovery/Validation by ATPase Mining and Kinase Mining

The ADP aptamers of the present invention can be used to detect ADP generation or disappearance associated with a variety of different biological processes, such as various diseases and disorders. In one embodiment, ADP or by inference ATP levels in a cell, tissue or organ sample associated with a pathological condition can be detected using the ADP aptamers of the present invention. Detection of binding of the ADP aptamer to its target and by inference the ADP/ATP level itself provides a means of diagnosing the condition.

Drug Discovery

Generally, methods of drug discovery comprise steps of 1) identifying target(s) molecules associated with a disease; 2) validating target molecules (e.g., mimicking the disease in an animal or cellular model); 3) developing assays to identify lead compounds which affect that target (e.g., using libraries to assay the ability of a compound to bind to the target); 4) prioritizing and modifying lead compounds identified through biochemical and cellular testing; 5) testing in animal models; and 6) testing in humans (clinical trials). Through the power of genomics and combinatorial chemistry, large numbers of lead compounds can be identified in high throughput assays (step 3); however, a bottleneck occurs at step 4 because of the lack of efficient ways to prioritize and optimize lead compounds and to identify those which actually offer potential for clinical trials.

The aptamers according to the present invention offer a way to solve this problem by providing reagents which can be used at each step of the drug development process. Most importantly, the nucleic acid compositions according to the present invention offer a way to correlate biochemical data, from in vitro biochemistry and cellular assays, with the effect of a drug on physiological response from a biological assay.

In one embodiment of the invention, a method for identifying a drug compound is provided, comprising identifying a profile of ATP consuming-ADP generating biological agents associated with a disease trait in a patient or test sample, administering a candidate compound to the patient, and monitoring changes in activity of the biological agents in the profile. In one embodiment, the APT consuming-ADP generating biological agents are protein kinases which utilize ATP to phosphorylate partner proteins in a signal transduction cascade, thereby regulating biochemical function of the partner, and the ADP aptamer is used to identify inhibitors of kinase activity. In one embodiment of the invention, the ATP consuming-ADP generating biological agents are helicases which utilize ATP to unwind DNA, and the ADP aptamer is used to identify inhibitors of helicase activity. In general, it is thought the human proteome is comprised of around two thousand protein kinases and a significantly greater number of proteins with ATPase activity. Hence, in another embodiment of the invention, the ADP aptamer is used to identify inhibitors of all enzymes that utilize ATP to generate ADP.

In one embodiment of the present invention, an ADP aptamer is used to identify, or mine, all proteins in a tissue or patient sample that-have ATPase activity or that have kinase activity. Thus, in one embodiment of the invention, the ADP aptamer is used as a kinase mining (or profiling), or ATPase mining tool. In another embodiment, this kinase or ATPase activity of the monitored profile is compared with a profile of a healthy patient or population of healthy patients, and a compound which generates a profile which is substantially similar to the profile of biological agents in the healthy patient(s) (based on routine statistical testing) is identified as a drug.

Aptamers for Use in Identifying Lead Compounds

In one embodiment, the ADP aptamer is used to identify the ATP utilizing agent (an ATPase or a protein kinase) as described above. ATP utilizing agents are provided and are validated by testing against multiple patient samples in vitro to verify that the signal generated by the binding of these molecules is diagnostic of a particular disease. Validation can also be performed ex vivo, e.g. in cell culture, using microscope-based detection systems and other optical systems as described in U.S. Pat. Nos. 5,843,658, 5,776,782, 5,648,269, and 5,585,245 and/or in vivo.

In one embodiment, the same methods which are used to validate the diagnostic value of particular sets of target molecule/aptamer combinations are used to identify lead compounds which can function as drugs. Thus, in one embodiment, the effects of a compound on target binding is monitored to identify changes in a profile arising as a result of treatment with a candidate compound.

In one embodiment, samples from a treated patient are tested in vitro; however, samples can also be treated ex vivo or in vivo. When the diagnostic profile identified by the biosensor changes from a profile which is a signature of a disease to one which is substantially similar to the signature of a wild type state (e.g., as determined using routine statistical tests), the lead compound is identified as a drug. Target molecules which activate the biosensor can comprise molecules with characterized activity and/or molecules with uncharacterized activity. Because a large number of target molecules can be monitored simultaneously, the method provides a way to assess the affects of compounds on multiple drug targets simultaneously, allowing identification of the most sensitive drug targets associated with a particular trait (e.g., a disease or a genetic alteration).

III. Selection and Generation of a Target Specific-Nucleic Acid Sensor Molecule

1) Generation and Selection of NASMs

Nucleic acid-based detection schemes have exploited the ligand-sensitive catalytic properties of some nucleic acids, e.g., such as ribozymes. Ribozyme-based nucleic acid sensor molecules have been designed both by engineering and by in vitro selection methods. Some engineering methods exploit the apparently modular nature of nucleic acid structures by coupling molecular recognition to signaling by simply joining individual target-modulation and catalytic domains using, e.g., a double-stranded or partially double-stranded linker. ATP sensors, for example, have been created by appending the previously-selected, ATP-selective TMD sequences (see, e.g., Sassanfar et al., Nature 363:550-553 (1993)) to either the self-cleaving hammerhead ribozyme (see, e.g., Tang et al., Chem. Biol. 4:453-459 (1997)) as a hammerhead-derived sensor, or the L1 self-ligating ribozyme (see, e.g., Robertson et al., Nucleic Acids Res. 28:1751-1759 (2000)) as a ligase-derived sensor. Hairpin-derived sensors are also contemplated. In general, the target modulation domain is defined by the minimum number of nucleotides sufficient to create a three-dimensional structure which recognizes a target molecule.

Catalytic nucleic acid sensor molecules (NASMs) are selected which have a target molecule-sensitive catalytic activity (e.g., self-cleavage) from a pool of randomized or partially randomized oligonucleotides. The catalytic NASMs have a target modulation domain which recognizes the target molecule and a catalytic domain for mediating a catalytic reaction induced by the target modulation domain's recognition of the target molecule. Recognition of a target molecule by the target modulation domain triggers a conformational change and/or change in catalytic activity in the nucleic acid sensor molecule. In one embodiment, by modifying (e.g., removing) at least a portion of the catalytic domain and coupling it to an optical signal generating unit, an optical nucleic acid sensor molecule is generated whose optical properties change upon recognition of the target molecule by the target modulation domain. In one embodiment, the pool of randomized oligonucleotides comprises the catalytic site of a ribozyme.

A heterogeneous population of oligonucleotide molecules comprising randomized sequences is screened to identify a nucleic acid sensor molecule having a catalytic activity which is modified (e.g., activated) upon interaction with a target molecule. As with the aptamer nucleic acids, the oligonucleotide can be RNA, DNA, or mixed RNA/DNA, and can include modified or nonnatural nucleotides or nucleotide analogs.

Each oligonucleotide in the population comprises a random sequence and at least one fixed sequence at its 5′ and/or 3′ end. In one embodiment, the population comprises oligonucleotides which include as fixed sequences an aptamer known to specifically bind a particular target and a catalytic ribozyme or the catalytic site of a ribozyme, linked by a randomized oligonucleotide sequence. In a preferred embodiment, the fixed sequence comprises at least a portion of a catalytic site of an oligonucleotide molecule (e.g., a ribozyme) capable of catalyzing a chemical reaction.

Catalytic sites are well known in the art and include, e.g., the catalytic core of a hammerhead ribozyme (see, e.g., U.S. Pat. No. 5,767,263; U.S. Pat. No. 5,700,923) or a hairpin ribozyme (see, e.g., U.S. Pat. No. 5,631,359). Other catalytic sites are disclosed in U.S. Pat. No. 6,063,566; Koizumi et al., FEBS Lett. 239: 285-288 (1988); Haseloff and Gerlach, Nature 334: 585-59 (1988); Hampel and Tritz, Biochemistry 28: 4929-4933 (1989); Uhlenbeck, Nature 328: 596-600 (1987); and Fedor and Uhlenbeck, Proc. Natl. Acad. Sci. USA 87: 1668-1672 (1990).

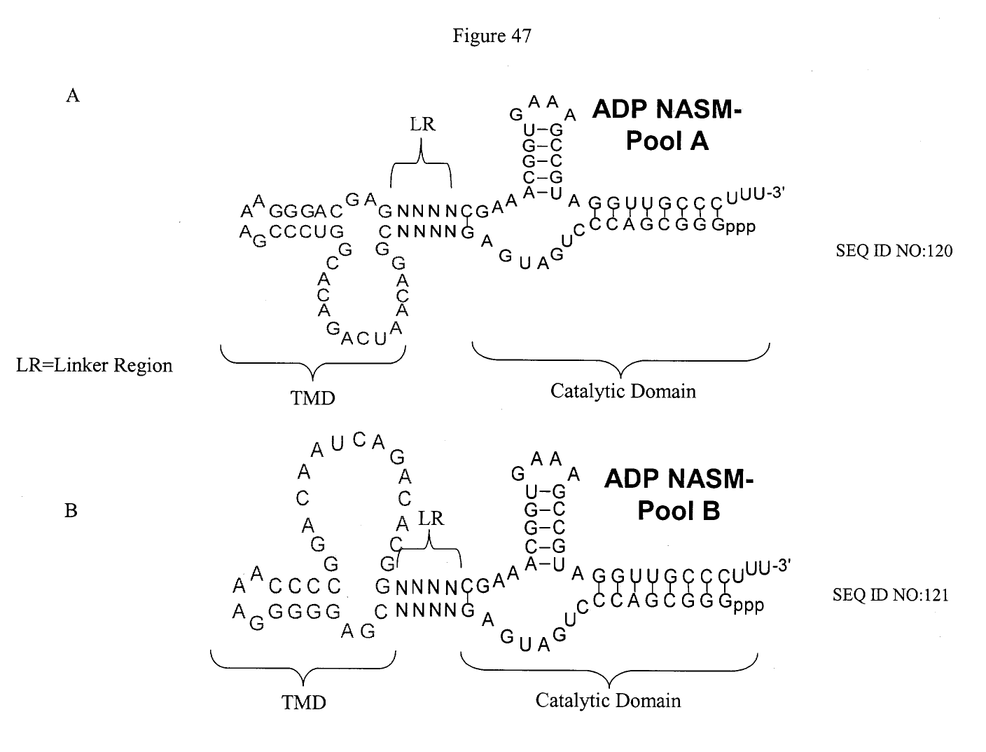

In some embodiments, a population of partially randomized oligonucleotides is generated from known aptamer and ribozyme sequences joined by the randomized oligonucleotides. Most molecules in this pool are non-functional, but a handful will respond to a given target and be useful as nucleic acid sensor molecules. Catalytic NASMs are isolated by the iterative process described above. Two examples of catalytic NASMs generated in this manner are shown as SEQ ID NOS: 120 and 121, in FIG. 47. In all embodiments, during amplification, random mutations can be introduced into the copied molecules—this ‘genetic noise’ allows functional NASMs to continuously evolve and become even better adapted as target-activated molecules.

In another embodiment, the population comprises oligonucleotides which include a randomized oligonucleotide linked to a fixed sequence which is a catalytic ribozyme, the catalytic site of a ribozyme or at least a portion of a catalytic site of an oligonucleotide molecule (e.g., a ribozyme) capable of catalyzing a chemical reaction. The starting population of oligonucleotides is then screened in multiple rounds (or cycles) of selection for those molecules exhibiting catalytic activity or enhanced catalytic activity upon recognition of the target molecule as compared to the activity in the presence of other molecules, or in the absence of the target.

The nucleic acid sensor molecules identified through in vitro selection, e.g., as described above, comprise a catalytic domain (i.e., a signal generating moiety), coupled to a target modulation domain, (i.e., a domain which recognizes a target molecule and which transduces that molecular recognition event into the generation of a detectable signal). In addition, the nucleic acid sensor molecules of the present invention use the energy of molecular recognition to modulate the catalytic or conformational properties of the nucleic acid sensor molecule.

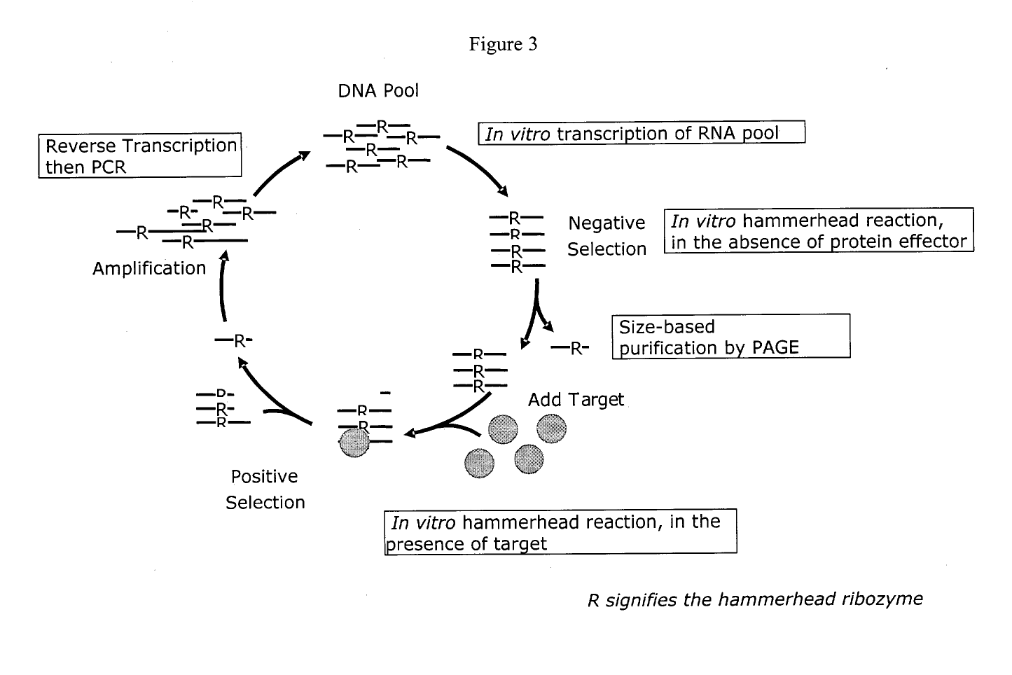

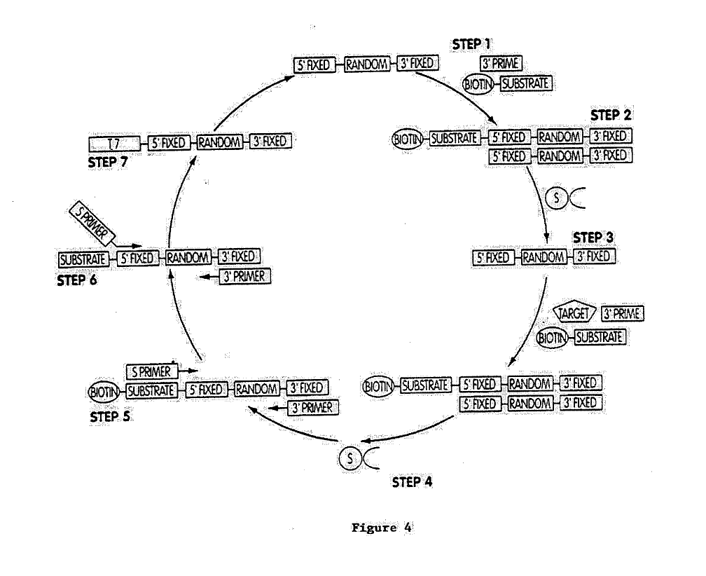

Nucleic acid sensor molecules are generally selected in a 5 to 20 cycle procedure. In one embodiment, heterogeneity is introduced only in the initial selection stages and does not occur throughout the replicating process. FIG. 4 shows a schematic diagram in which the oligonucleotide population is screened for a nucleic acid sensor molecule which comprises a target molecule activatable ligase activity. FIG. 3 shows the hammerhead nucleic acid sensor molecule selection methodology. Each of these methods are readily modified for the selection of NASMs with other catalytic activities.

Additional procedures may be incorporated in the various selection schemes, including: pre-screening, negative selection, etc. For example, individual clones isolated from selection experiments are tested early for allosteric activation in the presence of target-depleted extracts as a pre-screen, and molecules that respond to endogenous non-specific activators are eliminated from further consideration as target-modulated NASMs; to the extent that all isolated NASMs are activated by target-depleted extracts, depleted extracts are included in a negative selection step of the selection process; commercially available RNase inhibitors and competing RNAse substrates (e.g. tRNA) may added to test samples to inhibit nucleases; or by carrying out selection in the presence of nucleases (e.g. by including depleted extracts during a negative selection step) the experiment intrinsically favors those molecules that are resistant to degradation; covalent modifications to RNA that can render it highly nuclease-resistant can be performed (e.g., 2′-O-methylation) to minimize non-specific cleavage in the presence of biological samples (see, e.g., Usman et al.). Clin. Invest. 106:1197-202 (2000).

In one embodiment, nucleic acid sensor molecules are selected which are activated by target molecules comprising molecules having an identified biological activity (e.g., a known enzymatic activity, receptor activity, or a known structural role); however, in another embodiment, the biological activity of at least one of the target molecules is unknown (e.g., the target molecule is a polypeptide expressed from the open reading frame of an EST sequence, or is an uncharacterized polypeptide synthesized based on a predicted open reading frame, or is a purified or semi-purified protein whose function is unknown).

Although in one embodiment the target molecule does not naturally bind to nucleic acids, in another embodiment, the target molecule does bind in a sequence specific or non-specific manner to a nucleic acid ligand. In a further embodiment, a plurality of target molecules binds to the nucleic acid sensor molecule. Selection for NASMs specifically responsive to a plurality of target molecules (i.e. not activated by single targets within the plurality) may be achieved by including at least two negative selection steps in which subsets of the target molecules are provided. Nucleic acid sensor molecules can be selected which bind specifically to a modified target molecule but which do not bind to closely related target molecules. Stereochemically distinct species of a molecules can also be targeted.

A Target Modulation Domain with Endonucleolytic Activity

FIG. 3 shows the hammerhead nucleic acid sensor molecule selection methodology. As shown in FIG. 3, selection of an endonucleolytic nucleic acid sensor molecule (e.g., a hammerhead-derived NASM) begins with the synthesis of a ribozyme sequence on a DNA synthesizer. Alternatively, synthesis occurs on a RNA synthesizer. Random nucleotides are incorporated generating pools of roughly 10¹⁶ molecules. Most molecules in this pool are non-functional, but a handful will respond to a given target and be useful as nucleic acid sensor molecules. Sorting among the billions of species to find the desired molecules starts from the complex sequence pool. Nucleic acid sensor molecule are isolated by an iterative process: in addition to the target-activated ribozymes that one desires, the starting pool is usually dominated by either constitutively active or completely inactive ribozymes. The selection process removes both types of contaminants by incorporating both negative and positive selection incubation steps. In the following-amplification stage, thousands of copies of the surviving sequences are generated to enable the next round of selection. During amplification, random mutations can be introduced into the copied molecules—this ‘genetic noise’ allows functional NASMs to continuously evolve and become even better adapted as target-activated molecules. The entire experiment reduces the pool complexity from 10¹⁶ down to <100.

The starting library of DNA sequences (the “pool”) is generated by automated chemical synthesis on a DNA synthesizer. This library of sequences is transcribed in vitro into RNA using T7 RNA polymerase and subsequently purified. Alternatively, the pool is generated in a RNA synthesizer. In the absence of the desired target molecule of interest, the RNA library is incubated together with the binding buffer alone as a negative selection incubation. During this incubation, non-allosteric (or non-target activated) ribozymes are expected to undergo a catalytic reaction, in this case, cleavage. Undesired members of the hammerhead pool, those that are constitutively active in the absence of the target molecule, are removed from the unreacted members by size-based purification, e.g., by PAGE-chromatography; 7 M Urea, 8-10% acrylamide, 1×TBE. Higher molecular weight species are eluted as a single broad band from the gel matrix into TBE buffer, then purified for subsequent steps in the selection cycle. The remaining RNA pool is then incubated under identical conditions but now in the presence of the target molecule of interest in binding buffer, as a positive selection incubation. In another size-based purification, desired members of the hammerhead pool, those that are only active in the presence of the target molecule, are removed from the remaining unreacted members by PAGE-chromatography; 7 M Urea, 8-10% acrylamide, 1×TBE. In this step, lower molecular weight species are eluted as a single broad band from the gel matrix into TBE buffer, then purified for subsequent steps in the selection cycle. RT-PCR amplified DNA is then purified and transcribed to yield an enriched pool for a subsequent round of reselection. Rounds of selection and amplification are repeated until functional members sufficiently dominate the resultant library.

B. Target Modulation Domain with Ligase Activity

FIG. 4 shows a schematic diagram in which the oligonucleotide population is screened for a nucleic acid sensor molecule which comprises a target molecule activatable ligase activity. In the embodiment shown in FIG. 4, the ligation reaction involves covalent attachment of an oligonucleotide substrate to the 5′-end of the NASM through formation of a phosphodiester linkage. Other ligation chemistries can form the basis for selection of NASMs (e.g., oligonucleotide ligation to the 3′-end, alkylations (see, e.g., Wilson et al., Nature 374 (6525):777-782 (1995)), peptide bond formation (see, e.g., Zhang et al., Nature 390 (6655):96-100 (1997)), Diels-Alder reactions to couple alkenes and dienes (see, e.g., Seelig et al., Chemistry and Biology 3:167-176 (1999)). For some chemistries, the chemical functional groups that constitute the reactants in the ligation reaction may not naturally appear within nucleic acids. Thus, it may be necessary to synthesize an RNA pool in which one of the ligation reactants is covalently attached to each member of the pool (e.g., attaching a primary amine to the 5′-end of an RNA to enable selection for peptide bond formation).

In this embodiment, the oligonucleotide population from which the NASMs are selected is initially screened in a negative selection procedure to eliminate any molecules which have ligase activity even in the absence of target molecule binding. A solution of oligonucleotides (e.g., 100 pM) comprising a 5′ and 3′ fixed sequence (“5′-fixed: random: 3′-fixed”) is denatured with a 3′ primer sequence (“3′ prime”) (e.g., 200 pM) which binds to at least a portion of the 3′ fixed sequence. Ligation buffer (e.g., 30 mM Tris HCl, pH 7.4, 600 mM NaCl, 1 mM EDTA, 1% NP-40, 60 mM MgCl₂) and a tagged oligonucleotide substrate sequence (“tag-substrate”) (e.g., Tag-UGCCACU) are added and the mixture is incubated for about 16 to about 24 hours at 25° C. in the absence of target molecule (STEP 1). Tags encompassed within the scope include, e.g., radioactive labels, fluorescent labels, a chemically reactive species such as thiophosphate, the first member of a binding pair comprising a first and second binding member, each member bindable to the other (e.g., biotin, an antigen recognized by an antibody, or a tag nucleic acid sequence). The reaction is stopped by the addition of EDTA. Alternatively, the reaction can be terminated by removal of the substrate or addition of denaturants (e.g., urea or formamide).

Ligated molecules are removed from pool of selectable molecules (STEP 2), generating a population of oligonucleotides substantially free of ligated molecules (as measured by absence of the tag sequence in the solution). In the embodiment shown in FIG. 4, the tag is the first member of a binding pair (e.g., biotin) and the ligated molecules (“biotin-oligonucleotide substrate:5′-fixed:random:3′-fixed”) are physically removed from the solution by contacting the sample to a solid support to which the second member of the binding pair is bound (“S”) (e.g., streptavidin). The eluant collected comprises a population of oligonucleotides enriched for non-ligated molecules (5′-fixed:random:3′-fixed). This step can be repeated multiple times until the oligonucleotide population is substantially free of molecules having target-insensitive ligase activity.

This step allows for suppression of the ability of constitutively active molecules to be carried through to the next cycle of selection. Physical separation of ligated and unligated molecules is one mechanism by which this can be achieved. Alternatively, the negative selection step can be configured such that catalysis converts active molecules to a form that blocks their ability to be either retained during the subsequent positive selection step or to be amplified for the next cycle of selection. For example, the oligonucleotide substrate used for ligation in the negative selection step can be synthesized without a capture tag. Target-independent ligases covalently self-attach the untagged oligonucleotide substrate during the negative selection step and are then unable to accept a tagged form of the oligonucleotide substrate provided during the positive selection step that follows. In another embodiment, the oligonucleotide substrate provided during the negative selection step has a different sequence from that provided during the positive selection step. When PCR is carried out using a primer complementary to the positive selection oligonucleotide substrate, only target-activated ligases will be capable of amplification.

A positive selection phase follows. In this phase, more 3′ primer and tagged oligonucleotide substrate are added to the pool resulting from the negative selection step. Target molecules are then added to form a reacted solution and the reacted solution is incubated at 25° C. for about 2 hours (STEP 3). Target molecules encompassed within the scope include, e.g., proteins or portions thereof (e.g., receptors, antigen, antibodies, enzymes, growth factors), peptides, enzyme inhibitors, hormones, carbohydrates, polysaccharides, glycoproteins, lipids, phospholipids, metabolites, metal ions, cofactors, inhibitors, drugs, dyes, vitamins, nucleic acids, membrane structures, receptors, organelles, and viruses. Target molecules can be free in solution or can be part of a larger cellular structure (e.g., such as a receptor embedded in a cell membrane). In one embodiment, a target molecule is one which does not naturally bind to nucleic acids.

The reacted solution is enriched for ligated molecules (biotin-oligonucleotide substrate: 5′- fixed :random:3′-fixed) by removing non-tagged molecules (5′-fixed:random:3′-fixed) from the solution. For example, in one embodiment, the tagged oligonucleotide substrate comprises a biotin tag and ligated molecules are isolated by passing the reacted solution over a solid support to which streptavidin (S) is bound (STEP 4). Eluant containing non-bound, non-ligated molecules (5′-fixed:random:3′-fixed) is discarded and bound, ligated molecules (biotin-oligonucleotide substrate: 5′-fixed:random:3′-fixed) are identified as nucleic acid sensor molecules and released from the support by disrupting the binding pair interaction which enabled capture of the catalytically active molecules. For example, heating to 95° C. in the presence of 10 mM biotin allows release of biotin-tagged catalysts from an immobilized streptavidin support. In another embodiment, the captured catalysts remain attached to a solid support and are directly amplified (described below) while immobilized. Multiple positive selection phases can be performed (STEPS 3 and 4). In one embodiment, the stringency of each positive selection phase is increased by decreasing the incubation time by one half.

Physically removing inactive species from the pool adds stringency to the selection process. However, to the extent that the ligation reaction increases the amplification potential of the NASMs, this step may be omitted. In the illustrated embodiment, for example, ligation of an oligonucleotide to the active species provides a primer binding site that enables subsequent PCR amplification using an oligonucleotide substrate complementary to the original oligonucleotide substrate. Unligated species do not necessarily need to be physically separated from other species because they are less likely to amplify in the absence of a covalently tethered primer binding site. Selected nucleic acid sensor molecules are amplified (or in the case of RNA molecules, first reverse transcribed, then amplified) using an oligonucleotide substrate primer (“S primer”) which specifically binds to the ligated oligonucleotide substrate sequence (STEP 5). In one embodiment, amplified molecules are further amplified with a nested PCR primer that regenerates a T7 promoter (“T7 Primer”) from the 5′ fixed and the litigated oligonucleotide substrate sequence (STEP 6). Following transcription with T7 RNA polymerase (STEP 7), the oligonucleotide pool may be further selected and amplified to eliminate any remaining unligated sequences (5′-fixed:random:3′-fixed) by repeating STEPS 3-7. It should be obvious to those of skill in the art that in addition to PCR, and RT-PCR, any number of amplification methods can be used (either enzymatic, chemical, or replication-based, e.g., such as by cloning), either singly, or in combination. Exemplary amplification methods are disclosed in Saiki, et al., Science 230:1350-1354 (1985); Saiki, et al., Science 239:481-491 (1988); Kwoh, et al., Proc. Natl. Acad. Sci. 86:1173 (1989); Joyce, Molecular Biology of RNA: UCLA Symposia on Molecular and Cellular Biology, T. R. Cech (ed.) pp. 361-371 (1989); and Guatelli, et al., Proc. Natl. Acad. Sci. 87:1874 (1990).

Because the 3′ primer (3′ prime) (see STEP 3 in FIG. 4) is included in the ligation mixture, selected nucleic acid sensor molecules may require this sequence for activation. In cases where this is undesirable, the 3′ primer may be omitted from the mix. Alternatively, the final nucleic acid sensor molecule can be modified by attaching the 3′ primer via a short sequence loop or a chemical linker to the 3′ end of the nucleic acid sensor molecule, thereby eliminating the requirement for added primer, allowing 3′ primer sequence to self-prime the molecule.

C. Target Modulation Domain with Self-Cleaving Activity

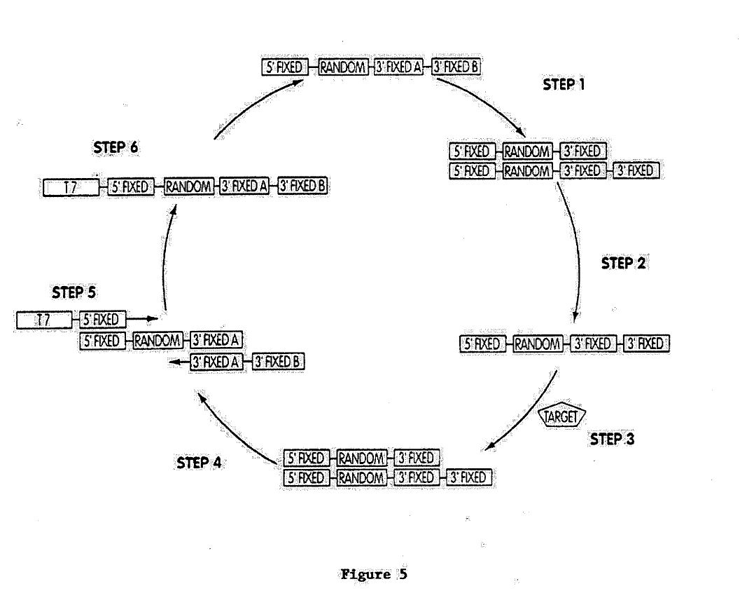

In another embodiment, as shown in FIG. 5, an oligonucleotide population is screened for a nucleic acid sensor molecule which comprises a target molecule having activatable self-cleaving activity. In this embodiment, the starting population of oligonucleotide molecules comprises 5′ and 3′ fixed regions (“5′-fixed and 3′ fixed A-3′fixed B”) and at least one of the fixed regions, in this example, the 3′ fixed region, comprises a ribozyme catalytic core including a self cleavage site (the junction between 3′ fixed A-3′fixed B). The population of oligonucleotide molecules comprising random oligonucleotides flanked by fixed 5′ and 3′ sequences (5′-fixed:random:3′-fixed A: 3′ fixed B) are negatively selected to remove oligonucleotides which self-cleave (i.e., 5′-fixed:random:3′-fixed-A molecules) even in the absence of target molecules. The oligonucleotide pool is incubated in reaction buffer (e.g., 50 mM Tris HCl, pH 7.5, 20 mM MgCl₂) for 5 hours at 25° C., punctuated at one hour intervals by incubation at 60° C. for one minute (STEP 1). In one embodiment, the uncleaved fraction of the oligonucleotide population (containing 5′-fixed and 3′ fixed A-3′-fixed B molecules) is purified by denaturing 10% polyacrylamide gel electrophoresis (PAGE) (STEP 2). Target molecule dependent cleavage activity is then selected in the presence of target molecules in the presence of reaction buffer by incubation at 23° C. for about 30 seconds to about five minutes (STEP 3). Cleaved molecules (5′-fixed:random:3′fixed-A molecules) are identified as nucleic acid sensor molecules and are purified by PAGE (STEP 4).

Amplification of the cleaved molecule is performed using primers which specifically bind the 5′-fixed and the 3′-fixed A sequences, regenerating the T7 promoter and the 3′-fixed B site (STEP 5), and the molecule is further amplified further by RNA transcription using T7 polymerase (STEP 6). In one embodiment, the process (STEPS 1-6) is repeated until the starting population is reduced to about one to five unique sequences.

Alternative methods for separating cleaved from uncleaved RNAs can be used. Tags can be attached to the 3′-fixed B sequence and separation can be based upon separating tagged sequences from non-tagged sequences at STEP 4. Chromatographic procedures that separate molecules on the basis of size (e.g., gel filtration) can be used in place of electrophoresis. One end of each molecule in the RNA pool can be attached to a solid support and catalytically active molecules isolated upon release from the support as a result of cleavage. Alternate catalytic cores may be used. These alternate catalytic cores and methods using these cores are also are encompassed within the scope of the invention.

D. Other Target Modulation Domains

Nucleic acid sensor molecules which utilize other catalytic actions or which combine both cleavage and ligase activities in a single molecule can be isolated by using one or a combination of both of the selection strategies outlined independently above for ligases and endonucleases. For example, the hairpin ribozyme is known to catalyze cleavage followed by ligation of a second oligonucleotide substrate (Berzal-Herranz et al., Genes and Development 1: 129-134 (1992)). Target activated sensor molecules based on the hairpin activity can be isolated from a pool of randomized sequence RNAs. Hairpin-based NASMs can be isolated on the basis of target molecule dependent release of the fragment in the same way that hammerhead-based NASMs are isolated (e.g., target molecule dependent increase in electrophoretic mobility or target molecule dependent release from a solid support). Alternatively, nucleic acid sensor molecules can be selected on the basis of their ability to substitute the 3′-sequence released upon cleavage for another sequence as described in an target molecule independent manner by Berzal-Herranz et al., Genes and Development 1:129-134 (1992). In this scheme, the original 3′-end of the NASM is released in an initial cleavage event and an exogenously provided oligonucleotide substrate with a free 5′-hydroxyl is ligated back on. The newly attached 3′-end provides a primer binding site that can form the basis for preferential amplification of catalytically active molecules. Constitutively active molecules that are not activated by a provided target molecule can be removed from the pool by (1) separating away molecules that exhibit increased electrophoretic mobility in the absence of an exogenous oligonucleotide substrate or in the absence of target molecule, or (2) capturing molecules that acquire an exogenous oligonucleotide substrate (e.g., using a 3′-biotinylated substrate and captured re-ligated species on an avidin column).

Like the hairpin ribozyme, the group I intron self-splicing ribozymes combine cleavage and ligation activities to promote ligation of the exons that flank it. In the first step of group I intron-catalyzed splicing, an exogenous guanosine cofactor attacks the 5′-splice site. As a result of an intron-mediated phosphodiester exchange reaction, the 5′-exon is released coincident with attachment of the guanosine cofactor to the ribozyme. In a second chemical step, the 3′-hydroxyl at the end of the 5′-exon attacks the phosphodiester linkage between the intron and the 3′-exon, leading to ligation of the two exons and release of the intron. Group I intron-derived NASMs can be isolated from degenerate sequence pools by selecting molecules on the basis of either one or both chemical steps, operating in either a forward or reverse direction. NASMs can be isolated by specifically enriching those molecules that fail to promote catalysis in the absence of target molecule but which are catalytically active in its presence. Specific examples of selection schemes follow. In each case, a pool of RNAs related in sequence to a representative group I intron (e.g., the Tetrahymena thermophila pre-rRNA intron or the phage T4 td intron) serves as the starting point for selection. Random sequence regions can be embedded within the intron at sites known to be important for proper folding and activity (e.g., substituting the P5abc domain of the Tetrahymena intron, Williams et al., Nucl. Acid Res. 22(11):2003-2009 (1994)). Intron nucleic acid sensor molecules, in this case, sensitive to thio-GMP can be generated as follows.

In the first step, forward direction, the intron is synthesized with a short 5′-exon. In the negative selection step, a guanosine cofactor is provided and constitutively active molecules undergo splicing. In the positive selection step, the target molecule is provided together with thio-GMP. Molecules responsive to the target undergo activated splicing and as a result acquire a unique thiophosphate at their 5′-termini. Thio-tagged NASMs can be separated from untagged ribozymes by their specific retention on mercury gels or activated thiol agarose columns.

The first step, reverse direction method is performed as described in Green & Szostak. An intron is synthesized with a 5′-guanosine and no 5′-exon. An oligonucleotide substrate complementary to the 5′-internal guide sequence is provided during the negative selection step and constitutively active molecules ligate the substrate to their 5′-ends, releasing the original terminal guanosine. A second oligonucleotide substrate with a different 5′-sequence is provided together with target in the positive selection step. NASMs specifically activated by the target molecule ligate the second oligonucleotide substrate to their 5′-ends. PCR amplification using a primer corresponding to the second substrate can be carried out to preferentially amplify target molecule sensitive nucleic acid sensor molecules.

The second step, reverse direction method is performed as described in Nature 344:467-468 (1990). The intron is synthesized with no flanking exons. During the negative selection step, pool RNAs are incubated together with a short oligonucleotide substrate under conditions which allow catalysis to proceed. During the positive selection step, a second oligonucleotide substrate with a different 3′-sequence is provided together with the sensor target. NASMs are activated and catalyze ligation of the 3′-end of the second substrate. Reverse transcription carried out using a primer complementary to the 3′-end of the second substrate specifically selects NASMs for subsequent amplification.

To generate an ADP NASM from an ADP aptamer, requires that the minimal secondary structure elements of the aptamer be known as the core ligand binding element of the aptamer must be appended directly to the randomized stem used in the catalytic NASM stem selection. Two structural analytical methods were used to determine the minimal secondary structure of the ADP F01 aptamer, 3′-end mapping and doped RNA reselection.

2) Characterization of NASMs

Once particular aptamers or nucleic acid sensor molecules have been selected, they can be isolated, cloned, sequenced, and/or resynthesized using natural or modified nucleotides. Accordingly, synthesis intermediates of nucleic acid compositions are also encompassed within the scope of the invention, as are replicatable sequences (e.g., plasmids) comprising the nucleic acid compositions of the invention.

The pool of NASMs is cloned into various plasmids transformed, e.g., into E. coli. Individual NASM encoded DNA clones are isolated, PCR amplified and to generate NASM RNA. The NASM RNAs are then tested in target modulation assays which determine the rate or extent of ribozyme modulation. For hammerhead NASMs, the extent of target dependent and independent reaction is determined by quantifying the extent of endonucleolytic cleavage of an oligonucleotide substrate. The extent of reaction can be followed by electrophoresing the reaction products on a denaturing PAGE gel, and subsequently analyzed by standard radiometric methods. For ligase NASMs, the extent of target dependent and independent reaction is determined by quantifying the extent of ligation of an oligonucleotide substrate, resulting in an increase in NASM molecular weight, as determined in denaturing PAGE gel electrophoresis.

Individual NASM clones which display high target dependent switch factor values, or high k_{act} rate values are subsequently chosen for further modification and evaluation.

Hammerhead-derived NASM clones are then further modified to render them suitable for the optical detection applications that are described in detail below. These NASMs are used as fluorescent biosensors affixed to solid supports, as fluorescent biosensors in homogeneous (solution) FRET-based assays, and as biosensors in SPA applications.

Ligase and intron-derived NASM clones are further modified to render them suitable for a number of detection platforms and applications, including, but not limited to, PCR and nucleotide amplification detection methods; fluorescent-based biosensors detectable in solution and chip formats; and as in vivo, intracellular detection biosensors.

An important kinetic consideration in NASM characterization is the fact that RNAse-mediated degradation of the nucleic acid sensor molecule proceeds at a rate in competition with the rate of nucleic acid sensor molecule catalysis. As such, nucleic acid sensor molecules with fast turnover rates can be assayed for shorter times and are thus less susceptible to RNAse problems. Nucleic acid sensor molecules with fast turnover can be obtained by (1) reducing the length of the incubation during the positive selection step, and/or (2) choosing fast nucleic acid sensor molecules (potentially with less favorable allosteric activation ratios) when screening individual clones emerging from the selection experiment.

The relative stabilities of the activated and unactivated forms of the nucleic acid sensor molecules can be optimized to achieve the highest sensitivity of detection of target molecule. In one embodiment, the nucleic acid sensor molecule is further engineered to enhance the stability of one form over another, such as favoring the formation of the target molecule activated form. As in the case where certain bases do not form base pairs when the nucleic acid sensor molecule is unactivated, the unactivated form is not stabilized.

A number of methods can be used to evaluate the relative stability of different conformations of the nucleic acid sensor molecule. In one embodiment, the free energy of the structures formed by the nucleic acid sensor molecule is determined using software programs such as mfold®, which can be found on the Rensselaer Polytechnic Institute (RPI) web site (www.rpi.edu/dept.).

In another embodiment, a gel assay is performed which permits detection of different conformations of the nucleic acid sensor molecule. In this embodiment, the nucleic acid sensor molecule is allowed to come to equilibrium at room temperature or the temperature at which the nucleic acid sensor molecule will be used. The molecule is then cooled to 4° C. and electrophoresed on a native (non-denaturing) gel at 4° C. Each of the conformations formed by the nucleic acid sensor molecule will run at a different position on the gel, allowing visualization of the relative concentration of each conformation. Similarly, the conformation of nucleic acid sensor molecules which form in the presence of target molecule is then determined by a method such as circular dichroism (CD). By comparing the conformation of the nucleic acid sensor molecule formed in the presence of target molecule with the conformations formed in the absence of target molecule, the conformation which corresponds to the activated conformation can be identified in a sample in which there is no target molecule. The nucleic acid sensor molecule can then be engineered to minimize the formation of the activated conformation in the absence of target molecule. The sensitivity and specificity of nucleic acid sensor molecule can be further tested using target molecule modulation assays with known amounts of target molecules.

Modifications to stabilize one conformation of the nucleic sensor molecule over another may be identified using the mfold program or native gel assays discussed above. A labeled nucleic acid sensor molecule is generated by coupling a first signaling moiety (F) to a first nucleotide and a second signaling moiety (D) to a second nucleotide as discussed above. As above, the sensitivity and specificity of the nucleic acid sensor molecule can be further assayed by using target molecule modulation assays with known amounts of target molecules.

3) Converting a Catalytic NASM to an Optical NASM

During or after synthesis of the NASM, an optical signal generating unit is either added or inserted into the oligonucleotide sequence comprising the derived nucleic acid sensor molecule. In one embodiment, in order to convert a catalytic nucleic acid sensor molecule into an optical nucleic acid sensor molecule, at least a portion of the catalytic domain is modified (e.g., deleted). In one embodiment, the deletion enhances the conformational stability of the optical nucleic acid sensor molecule in either the bound or unbound forms. In one embodiment, deletion of the entire catalytic domain of the catalytic NASM stabilizes the unbound form of the nucleic acid sensor molecule. In another embodiment, the deletion may be chosen so as to take advantage of the inherent fluorescence-quenching properties of unpaired guanosine (G) residues (Walter and Burke, RNA 3:392 (1997)).

In another embodiment, the target modulation domain from a previously identified nucleic acid sensor molecule is incorporated into an oligonucleotide sequence that changes conformation upon target recognition. Nucleic acid sensor molecules of this type can be derived from allosteric ribozymes, such as those derived from the hammerhead, hairpin, L1 ligase, or group 1 intron ribozymes and the like, all of which transduce molecular recognition into a detectable signal. For example, 3′,5′-cyclic nucleotide monophosphate (cNMP)-dependent hammerhead ribozymes were reengineered into RNA molecules which specifically bound to cNMP (Soukup et al., RNA 7:524 (2001)). The catalytic cores for hammerhead ribozymes were removed and replaced with 5-base duplex forming sequences. The binding of these reengineered RNA sensor molecules to cNMP was then confirmed experimentally. By adjusting the duplex length, sensor molecules can be redesigned to undergo significant conformational changes. The conformational changes can then be coupled to detection via FRET or simply changes in fluorescence intensity (as in the case of a molecular beacon). For example, by adding an appropriate probe on each end of the duplex, the stabilization of duplex by target binding can be monitored with the change in fluorescence.

While the above experimental example is performed in solution and utilizes a cuvette-based fluorescence spectrometer, in alternative embodiments the methods are performed in microwell multiplate readers (e.g., the Packard Fusion, or the Tecan Ultra) for high-throughput solution phase measurements.

In one embodiment, after deletion of at least a portion of the catalytic site from a catalytic nucleic acid sensor molecule, an optical signaling unit is either added to, or inserted within, the nucleic sensor molecule, generating a sensor molecule whose optical properties change in response to binding of the target molecule to the target modulation domain. In one embodiment, the optical signaling unit is added by exposing at least a 5′ or 3′ nucleotide that was not previously exposed. The 5′ nucleotide or a 5′ subterminal nucleotide (e.g., an internal nucleotide) of the molecule is couplable to a first signaling moiety while the 3′ nucleotide or 3′ subterminal nucleotide is couplable to a second signaling moiety. Target molecule recognition by the optical nucleic acid sensor molecule alters the proximity of the 5′ and 3′ nucleotide (or subterminal nucleotides) with respect to each other, and when the first and second signaling moieties are coupled to their respective nucleotides, this change in proximity results in a target sensitive change in the optical properties of the nucleic acid sensor molecule. Detection of changes in the optical properties of the nucleic acid sensor molecule can therefore be correlated with the presence and/or quantity of a target molecule in a sample.

In another embodiment, optical NASMs are generated by adding first and second signaling moieties, that are coupled to the 5′ terminal or subterminal sequences, and 3′-1 terminal and subterminal sequences respectively, of the catalytic NASM. Signaling molecules can be coupled to nucleotides which are already part of the nucleic acid sensor molecule or may be coupled to nucleotides which are inserted into the nucleic acid sensor molecule, or can be added to a nucleic acid sensor molecule as it is synthesized. Coupling chemistries to attach signaling molecules are well known in the art (see, e.g., The Molecular Probes Handbook, R. Haughland). Suitable chemistries include, e.g., derivatization of the 5-position of pyrimidine bases (e.g., using 5′-amino allyl precursors), derivatization of the 5′-end (e.g., phosphoroamidites that add a primary amine to the 5′-end of chemically-synthesized oligonucleotide) or the 3′-end (e.g., periodate treatment of RNA to convert the 3′-ribose into a dialdehyde which can subsequently react with hydrazide-bearing signaling molecules).

In another embodiment, a single signaling moiety is either added to, or inserted within, the catalytic nucleic sensor molecule. In this embodiment, binding of the target molecule results in changes in both the conformation and physical aspect (e.g., molecular volume, and thus rotational diffusion rate, etc.) of the optical nucleic acid sensor molecule. Conformational changes in the optical nucleic acid sensor molecule upon target recognition will modify the chemical environment of the signaling moiety, while changes in the physical aspect of the nucleic acid sensor molecule will alter the kinetic properties of the signaling moiety. In both cases, the result will be a detectable change in the optical properties of the nucleic acid sensor molecule.

In one embodiment, the optical nucleic acid sensor molecule is prepared without a quencher group. Instead of a quencher group, a moiety with a free amine group can be added. This free amine group allows the sensor molecule to be attached to an aldehyde-derivatized glass surface via standard protocols for Schiff base formation and reduction. The nucleic acid sensor molecules can be bound in discrete regions or spots to form an array, or uniformly distributed to cover an extended area. In the absence of target, the optical nucleic acid sensor molecule will diffusionally rotate about its point of attachment to the surface at a rate characteristic of its molecular volume and mass. After target recognition and modulation of the structure of the NASM, the optical NASM-target complex will have a correspondingly larger volume and mass. This change in molecular volume (mass) will slow the rate of rotational diffusion, and result in a measurable change in the polarization state of the fluorescence emission from the fluorophore.

In one embodiment of the invention, a single signaling moiety is attached to a portion of a catalytic NASM that is released as a result of catalysis (e.g., either end of a self-cleaving ribozyme or the pyrophosphate at the 5′-end of a ligase). Target molecule-activated catalysis leads to release of the signaling moiety from the optical NASM to generate a signal correlated with the presence of the target. Release can be detected by either (1) changes in the intrinsic optical properties of the signaling moiety (e.g., decreased fluorescence polarization as the released moiety is able to tumble more freely in solution), or (2) changes in the partitioning of the signaling moiety (e.g., release of a fluorophore from a chip containing immobilized ribozymes such that the total fluorescence of the chip is reduced following washing).

In another embodiment of the invention, the catalytic nucleic acid sensor molecule is umnodified and the optical signaling unit is provided as a substrate for the NASM. One example of this embodiment includes a fluorescently tagged oligonucleotide substrate which can be joined to a NASM with ligase activity. In a heterogeneous assay using the ligase as a sensor molecule, analyte-containing samples are incubated with the fluorescent oligonucleotide substrate and the ligase under conditions that allow the ligase to function. Following an incubation period, the ligase is separated from free oligonucleotide substrate (e.g., by capturing ligases onto a solid support on the basis of hybridization to ligase-specific sequences or by pre-immobilizing the ligases on a solid support and washing extensively).

Quantitation of the captured fluorescence signal provides a means for inferring the concentration of analyte in the sample. In a second example of this embodiment, catalytic activity alters the fluorescence properties of a oligonucleotide substrate without leading to its own modification. Fluorophore pairs or fluorophore/quencher pairs can be attached to nucleotides flanking either side of the cleavage site of an oligonucleotide substrate for a trans-acting endonuclease ribozyme (Jenne et al., Nature Biotechnology 19(l):56-61 (2001)). Target activated cleavage of the substrate leads to separation of the pair and a change in its optical properties.

In another embodiment of the invention, the ligase catalytic NASM and its oligonucleotide substrates are unmodified and detection relies on catalytically-coupled changes in the ability of the NASM to be enzymatically amplified. In one example, a target-activated ligase is incubated together with oligonucleotide substrate and an analyte-containing sample under conditions which allow the ligase to function. Following an incubation period, the reaction is quenched and the mixture subjected to RT/PCR amplification using a primer pair that includes the oligo sequence corresponding to the ligation substrate. Amplification products can be detected by a variety of generally practiced methods (e.g. Taqman®). Only those ribozymes that have self-ligated an oligonucleotide substrate are capable of amplification under these conditions and will generate a signal that can be coupled to the concentration of the sensor target.

4) Detection of Optical NASMs

i) Proximity Dependent Signaling Moieties

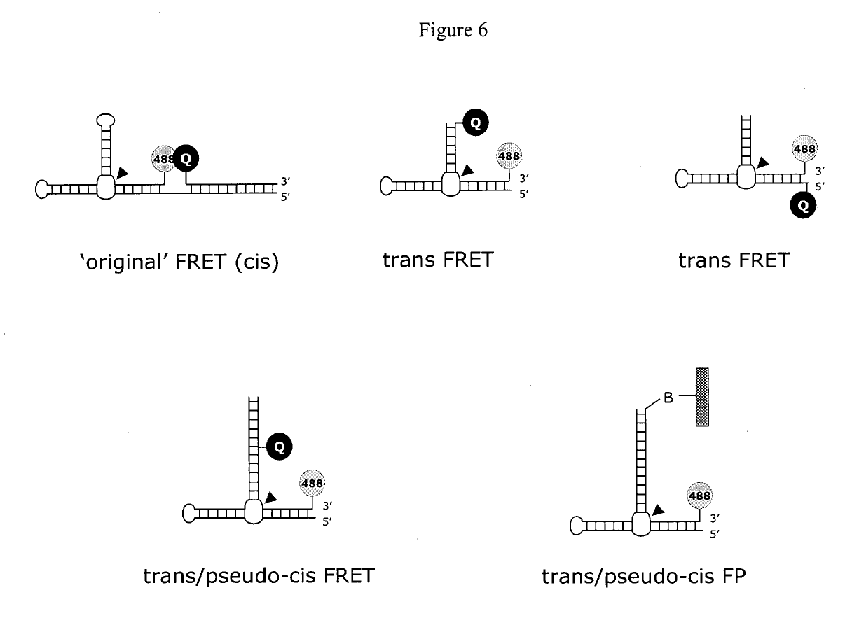

Many proximity dependent signaling moieties are known in the art and are encompassed within the scope of the present invention (Morrison, Nonisotopic DNA Probe Techniques, Kricka, ed., Academic Press, Inc., San Diego, Calif., chapter 13; Heller et al., Academic Press, Inc. pp. 245-256 (1985)). Systems using these signaling moieties rely on the change in fluorescence that occurs when the moieties are brought into close proximity. Such systems are described in the literature as fluorescence energy transfer (FET), fluorescence resonance energy transfer (FRET), nonradiative energy transfer, long-range energy transfer, dipole-coupled energy transfer, or Forster energy transfer (U.S. Pat. No. 5,491,063, Wu et al., Anal. Biochem. 218:1 (1994)). The arrangement of various fluorophore-quencher pairs is shown in FIG. 6. (See Jenne et al., Nature Biotechnology 1:56-61 (2001); Singh et al., RNA 5:1348 (1999); Frauendorf et al., Bioorg Med. Chem. 10:2521-2524 (2001); Perkins et al., Biochemistry 35(50):16370-16377 (1996)), and WO 99/47704 for discussion of various FRET formats.

Suitable fluorescent labels are known in the art and commercially available from, for example, Molecular Probes (Eugene, Oreg.). These include, e.g., donor/acceptor (i.e., first and second signaling moieties) molecules such as: fluorescein isothiocyanate (FITC)/tetramethylrhodamine isothiocyanate (TRITC), FITC/Texas Red), FITC/N-hydroxysuccinimidyl 1-pyrenebutyrate (PYB), FITC/eosin isothiocyanate (EITC), N-hydroxysuccinimidyl 1 -pyrenesulfonate (PYS)/FITC, FITC/Rhodamine X (ROX), FITC/tetramethylrhodamine (TAMRA), and others. In addition to the organic fluorophores already mentioned, various types of nonorganic fluorescent labels are known in the art and are commercially available from, for example, Quantum Dot Corporation, Inc. (Hayward, Calif.). These include, e.g., donor/ acceptor (i.e., first and second signaling moieties) semiconductor nanocrystals (i.e., ‘quantum dots’) whose absorption and emission spectra can be precisely controlled through the selection of nanoparticle material, size, and composition (see, e.g., Bruchez et al., Science 281:2013 (1998); Chan et al., J. Colloid and Interface Sci. 203:197 (1998), Han et al., Nature Biotechnol 19:631 (2001)).

The selection of a particular donor/acceptor pair is not critical to practicing the invention provided that energy can be transferred between the donor and the acceptor. P-(dimethyl aminophenylazo) benzoic acid (DABCYL) is one example of a non-fluorescent acceptor dye which effectively quenches fluorescence from an adjacent fluorophore, e.g., fluorescein or 5-(2′-aminoethyl) aminonaphthalene (EDANS).

The first and second signaling moieties can be attached to terminal or to non-terminal sequences. The position of the non-terminal sequences coupled to signaling moieties is limited to a maximal distance from the 5′ or 3′ nucleotide which still permits proximity dependent changes in the optical properties of the molecule. Coupling chemistries are routinely practiced in the art, and oligonucleotide synthesis services provided commercially (e.g., Integrated DNA Technologies, Coralville, Iowa) can also be used to generate labeled molecules. In a further embodiment, the nucleic acid sensor molecule is used, either tethered to a solid support or free in solution, to detect the presence and concentration of target molecules in a complex biological fluid.

For example, the first signaling moiety (F) can be fluorescein molecule coupled to the 5′ end and the second signaling molecule (D) can be a DABCYL molecule (a quenching group) coupled to the 3′ end. When the nucleic acid sensor molecule is not activated by target molecule, the fluorescent group and the quenching group are in close proximity and little fluorescence is detectable from the fluorescent group. Addition of target molecule causes a change in the conformation of the optical nucleic acid sensor molecule. When the molecule is activated by target recognition, and the first and second signaling moieties (F and D, respectively) are no longer in sufficient proximity for the quenching group to quench the fluorescence of the fluorescent group, the result is a detectable fluorescent signal being produced upon recognition of the target molecule.

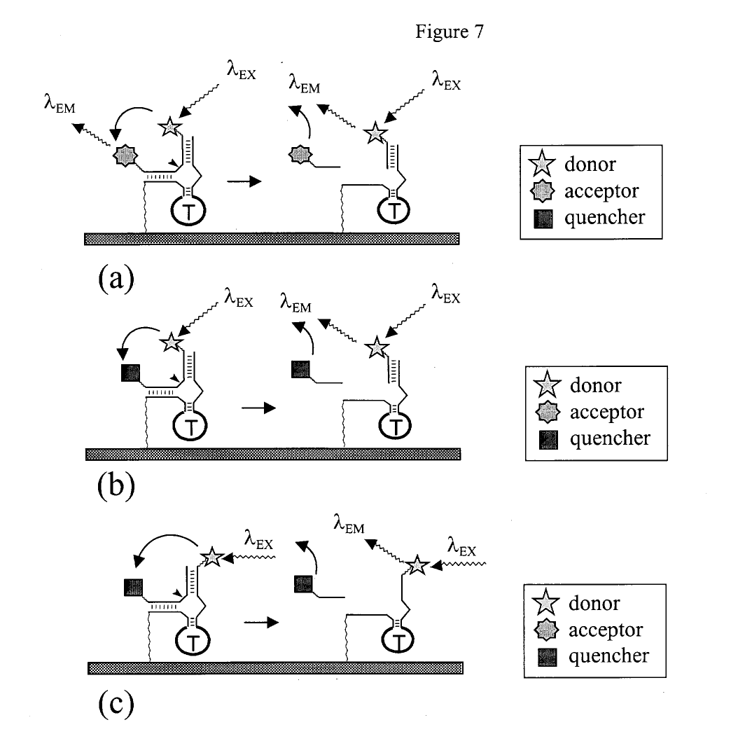

One general method for implementing a FRET-based (fluorescence resonance energy transfer) assay utilizing nucleic acid sensor molecules is described for a hammerhead nucleic acid sensor molecule, wherein the nucleic acid sensor molecule is immobilized on a solid substrate, e.g., within a microtiter plate well, on a membrane, on a glass or plastic microscope slide, etc. In the embodiment shown in FIGS. 7A, B, and C, a self-cleaving ribozyme such as the hammerhead (in this case attached to a solid support via a linker molecule is shown) is labeled with a fluorophore. In FIG. 7A, the labeled NASM in the unactivated state comprises two oligonucleotides including a transacting cleavage substrate which bears a first and second fluorescent label. In the unactivated state, i.e., in the absence of target molecule, the donor fluorophore and the acceptor fluorophore are in sufficiently close proximity for FRET to occur; thus, minimal fluorescent emission is detected from the donor fluorophore at wavelength 3, λ3, upon epi-illumination excitation at the excitation wavelength, λ_{EX}. Upon target molecule recognition, the cleavage fragment of the cleavage substrate bearing the acceptor fluorophore dissociates from the NASM-target complex. Once separated from the acceptor fluorophore, the donor fluorophore can no longer undergo de-excitation via FRET, resulting in a detectable increase in its fluorescent emission at wavelength, λ_{EM} (see, e.g., Singh. et al., RNA 5:1348 (1999); Wu et al., Anal. Biochem. 218:1 (1994); Walter et al., RNA 3:392 (1997); Walter et al., The EMBO Journal 17(8):2378 (1998)). In a further embodiment, the change in the polarization state of the fluorescent emission from the donor fluorophore (due to the increased diffusional rotation rate of the smaller cleavage fragment) can be detected/monitored in addition to changes in fluorescent emission intensity (see, e.g., Singh et al., Biotechniques 29:344 (2000)). In a further embodiment, the NASMs are free in solution.

In another embodiment, shown in FIG. 7B, the acceptor fluorophore attached to the cleavage substrate is replaced by a quencher group. This replacement will also result in minimal fluorescent donor emission at wavelength λ_{EX} when the NASM is in the unbound state under epi-illumination excitation at wavelength λ_{EX}. Upon target molecule recognition, the cleavage fragments of the cleavage substrate bearing the donor and quencher groups dissociate from the NASM-target molecule complex. Once separated from the quencher, the donor fluorophore will exhibit a detectable increase in its fluorescent emission at wavelength λ_{EM}. In a further embodiment, the change in the polarization state of the fluorescent emission from the donor fluorophore (due to the increased diffusional rotation rate of the smaller cleavage fragment) can be detected/monitored in addition to changes in fluorescent emission intensity. In a further embodiment, NASMs are free in solution.

In a different embodiment, the optical configuration is designed to provide excitation via total internal reflection (TIR)-illumination, as shown in FIG. 7C. Also, the donor fluorophore is attached to the NASM body while the quencher is attached to the cleavage substrate. In this configuration, with the surface-immobilized NASM in the unbound state, the fluorescent donor emission at wavelength λ_{EM} will be minimal. Upon target module recognition, the cleavage fragment of the cleavage substrate bearing the quencher group dissociates from the NASM-target module complex. Once separated from the quencher, the donor fluorophore will exhibit a detectable increase in its fluorescent emission at wavelength λ_{EM}. In an alternative embodiment to that shown in shown in FIG. 7C, the quencher group can be replaced with an acceptor fluorophore. In yet another alternative embodiment to those shown in FIGS. 7A, B, and C, the donor fluorophore is coupled to the cleavage fragment of the cleavage substrate and the acceptor fluorophore or quencher group is deleted. Upon target molecule recognition and dissociation of the cleavage fragment, the polarization state of the fluorescent emission from the donor fluorophore will undergo a detectable change due to the difference in the diffusional rotation rates of the surface-bound NASM target complex and the free cleavage fragment.

In one embodiment, a universal FRET trans-substrate is synthesized for all NASMs derived from self-cleaving allosteric ribozymes. This substrate would have complementary optical signaling units (i.e., donor and acceptor groups) coupled to opposite ends of the synthetic oligonucleotide sequence. Such a universal substrate would obviate the need for coupling optical signaling units to the sensor (i.e., ribozyme) molecule itself.

In addition to the herein described methods, any additional proximity dependent signaling system known in the art can be used to practice the method according to the invention, and are encompassed within the scope.

In one specific embodiment described here, a first oligonucleotide of the nucleotide sensor molecule is 3′-labeled with an acceptor or quencher fluorophore, such as TAMRA, AlexaFluor 568, or DABCYL, via specific periodate oxidation. A second oligonucleotide of the nucleic acid sensor molecule, complementary to at least part of the first oligo portion of the NASM, is labeled with a 3′ biotin and a 5′ donor fluorophore, such as fluorescein (FAM, FITC, etc.). These two nucleic oligonucleotides are heat-denatured in solution and allowed to anneal/hybridize during cooling to room temperature. After hybridization, the NASM solution is applied to a surface which has been coated with some type of avidin (streptavidin, neutravidin, avidin, etc.). This surface could include a microtiter plate well, a streptavidin-impregnated membrane, a glass or plastic microscope slide, etc. In any case, the ribozyme-oligo complex is specifically immobilized via the 3′ biotin on the donor oligo, leaving the binding domain free to interact with the target effector molecule.

The donor and acceptor fluorophores form an efficient FRET-pair; that is, upon excitation of the donor fluorophore near its spectral absorption maxima, the incident electromagnetic energy is efficiently transferred (nonradiatively) via resonant electric dipole coupling from the donor fluorophore to the acceptor fluorophore. The efficiency of this resonant energy transfer is strongly dependent on the separation between the donor and acceptor fluorophores, the transfer rate being proportional to 1I/R⁶, where R is the intermolecular separation. Therefore, when the donor and acceptor are in close proximity, i.e., a few bond-lengths or roughly 10-50 Angstroms, the fluorescent emission from donor species will be reduced relative to its output in an isolated configuration, while the emission from the acceptor species, through indirect excitation by the donor, will be detectable. Upon separation of the donor and acceptor, the donor fluorescence emission signal will increase strongly, while the acceptor emission signal will show a commensurate decrease in intensity. After effector-mediated cleavage at room temperature, the cleavage fragment will rapidly dissociate from the ribozyme body and diffuse away into solution.

This target-activated nucleic acid sensor molecule system constitutes a highly sensitive real-time sensor for detecting and quantitating the concentration of the target molecule present in an unknown sample solution. The ultimate limit of detection (LOD) for this system is determined by the switch factor, defined as the ratio of the catalytic rate (in this example, the rate of cleavage) of the ribozyme sensor in the presence of its target to that of the ribozyme in the absence of its target. The dynamic range of the ribozyme sensor will be determined by the switch factor and the dissociation constant, K_{d}, for the interaction of the ribozyme binding domain with the target molecule. In theory, the effective dynamic range over which the rate-response of the NASM is linear in the target concentration has K_{d} as an upper bound.

In practice, concentration measurements up to 1 mM are possible with this sensor in solution-phase measurements. The absolute precision of measurements made with this NASM will depend on the amount of background catalytic activity (i.e., in the absence of target) and baseline drift of the fluorescence signals from both sample and controls due to physical factors, such as liquid handling errors, reagent adhesion, evaporation, or mixing. After some optimization, run-to-run CVs of a few percent are possible with FRET-based NASMs measured in solution. Immobilization of the NASM does not degrade its catalytic activity, although it may limit the effective availability of the target-binding domain for interaction with target molecules. The locally high concentration of surface-immobilized NASM will tend to offset this effect by driving the equilibrium for the association (and subsequent catalytic) reactions toward formation of ribozyme-target complex. Detection of the fluorescent signals can be accomplished by a microplate fluorescence reader equipped with the appropriate lamps, optics, filters, and optical detectors (PMT) manufactured by Packard Instrument Co.

Such a sensor array could be used to detect and quantify the presence of an arbitrary target molecule in a complex solution, e.g., crude cell extract or biological fluid, in real time. In addition, this general NASM strategy could be extended to accomplish multiplexed detection of multiple analytes in a sample simultaneously, by using NASMs labeled with fluorophores having different emission wavelengths. In all of these scenarios, optical detection of the FRET signals could be accomplished using a commercially available microarray imager or scanning fluorescence microscope.

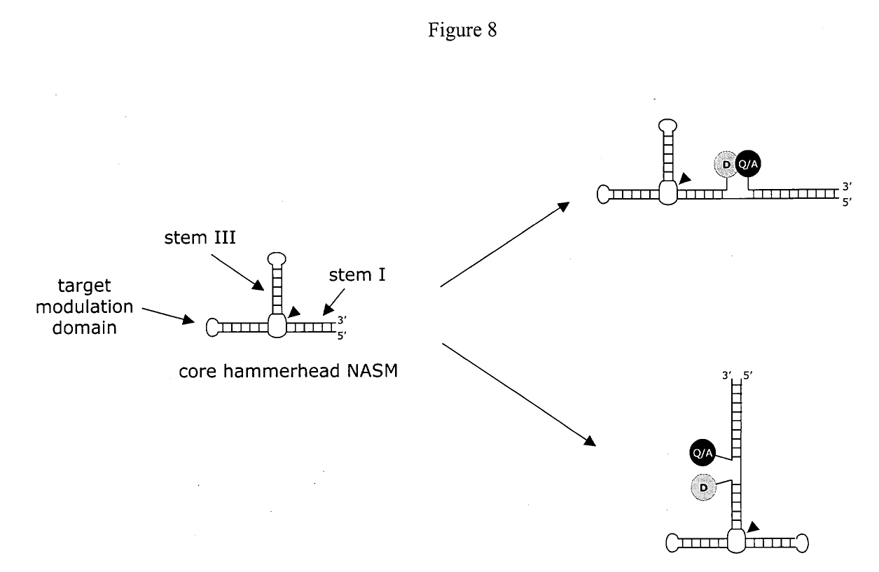

For example, fluorescence energy resonance transfer (FRET) can be used as a general detection method for hammerhead ribozyme or effector-dependent hammerhead ribozyme activity. Hammerhead NASMs typically consist of a catalytic domain responsible for RNA phosphodiester cleavage activity, plus a target modulation domain which, upon binding of an analyte molecule, triggers a structural change within the NASM and leads to the cleavage reaction. In one specific embodiment, described herein, such core hammerhead NASMs are modified to contain a donor fluorophore (D) covalently attached to the 3′-end of the NASM. In addition, a sequence domain to which a fluorescence quencher/acceptor dye (Q/A) containing auxiliary oligonucleotide can be hybridized is attached adjacent to either stem I or stem III (FIG. 8). The fluorophores are chosen to form an efficient FRET-pair; that is, upon excitation of the first, or donor fluorophore near its spectral absorption maxima, the incident electromagnetic energy is efficiently transferred (nonradiatively) via resonant electric dipole coupling from the donor fluorophore to the second, or acceptor fluorophore. The efficiency of this resonant energy transfer is strongly dependent on the separation between the donor and acceptor fluorophores, the transfer rate being proportional to 1/R⁶, where R is the intermolecular separation. Therefore, when the donor and acceptor are in close proximity, i.e., a few bond-lengths or roughly 10-50 Angstroms, the fluorescent emission from donor species will be reduced relative to its output in an isolated configuration, while the emission from the acceptor species, through indirect excitation by the donor, will be detectable. Therefore the relative positioning of the fluorescence-labeled NASM 3′-terminus and the second fluorophore should be in close proximity to allow for such an energy transfer.

One example of FRET pairs are fluorescein as donor and TAMRA as acceptor. Alternatively, the acceptor can be replaced by a so-called dark quencher, such as DABCYL or QSY-7. Either relative orientation of the fluorophores (donor/acceptor and NASM/auxiliary oligo) can be chosen. The exact distance is governed by the number of unpaired nucleotides connecting stem I or III and the hybridization domain for the second oligo, and preferably is between 2 and 4 nucleotides long. The stem involving the 3′-terminus must be long enough to ensure proper folding into a hammerhead structure, but not too long to prevent rapid dissociation after hammerhead cleavage, and is preferably between 5 and 8 nucleotides. The attachment of the first fluorophore to the NASM 3′-terminus can be done by a variety of methods such as enzymatic ligation of a fluorescent nucleotide using terminal transferase or RNA ligase, or by oxidizing the terminal ribonucleotide with sodium periodate, followed by reaction with a fluorophore amine in the presence of sodium borohydride/cyanoborohydride, or a fluorophore hydrazide, semicarbazide or thiocarbazide (Agrawal in Protocols for Oligonucleotide Conjugates, Humana Press, Totowa, 1994, 26, 93; Wu et al., Nucleic Acids Research 24(17):3472 (1996)). Notably, apart from the 3′-modifications, the NASMs can be synthesized entirely through in simple vitro transcription reactions and do not have to contain any other internal or 5′ chemical modifications that are potentially difficult to introduce. The auxiliary oligonucleotide can be of any nucleotide sequence or composition (e.g., DNA, RNA, 2′-OMe-RNA, 2′-F-RNA or combination thereof), with a length ensuring tight hybridization to the complementary NASM domain, preferably between 20 to 30 nucleotides. Conversely the length and sequence of the corresponding NASM domain can be freely chosen to accommodate the auxiliary oligonucleotide.

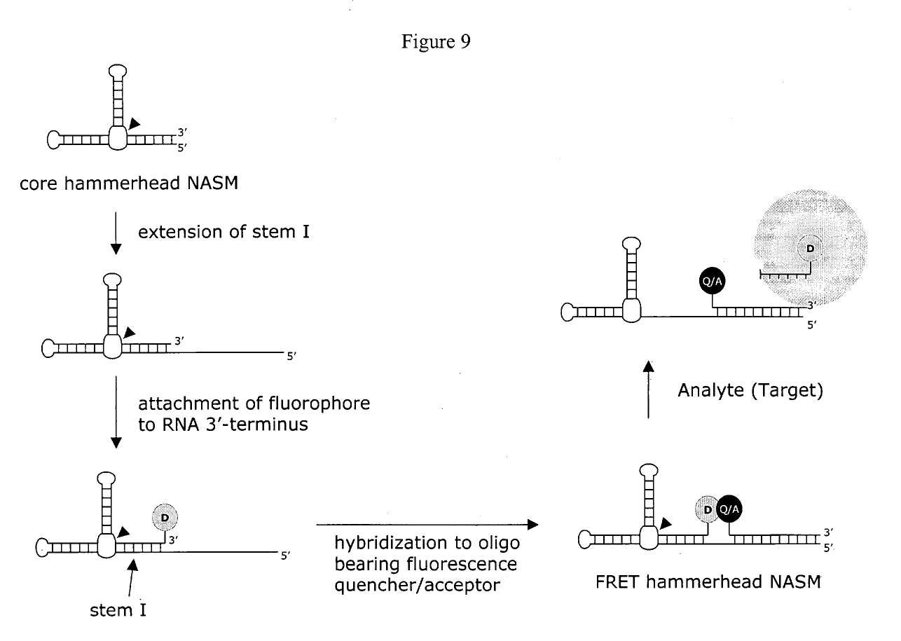

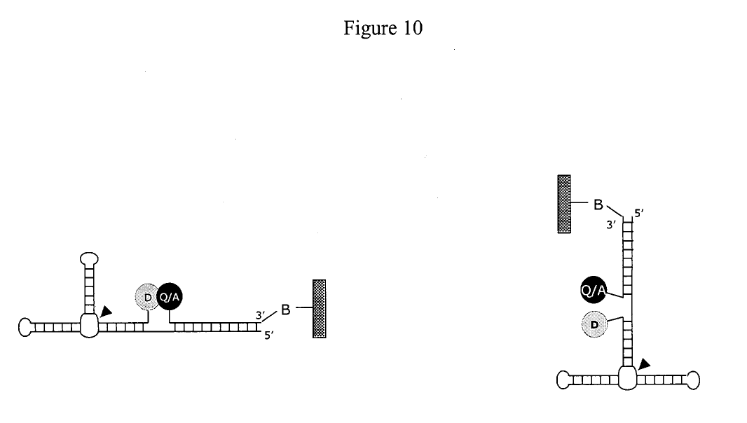

An example of a stem I-modified FRET hammerhead NASM is illustrated in FIG. 9. In addition, the NASM can be immobilized on a solid support via its auxiliary oligonucleotide, for example through incorporation of a biotin and capture on a streptavidin surface (FIG. 10). This surface could include a microtiter plate well, a streptavidin-impregnated membrane, a glass or plastic microscope slide, etc. Preferably immobilization takes place though the remote end of the auxiliary oligo, exposing the NASM core to the solution and not restricting it's accessibility or activity. The generalization of this application of surface-immobilized ribozyme sensors with FRET detection to a micro- or macro-arrayed format on an extended substrate such as glass or plastic is easily envisioned. Such a sensor array could be used to detect and quantify the presence of an arbitrary target molecule in a complex solution, e.g., crude cell extract or biological fluid, in real time. In this scenario, optical detection of the FRET signals could be accomplished using a commercially available microarray imager or scanning fluorescence microscope.

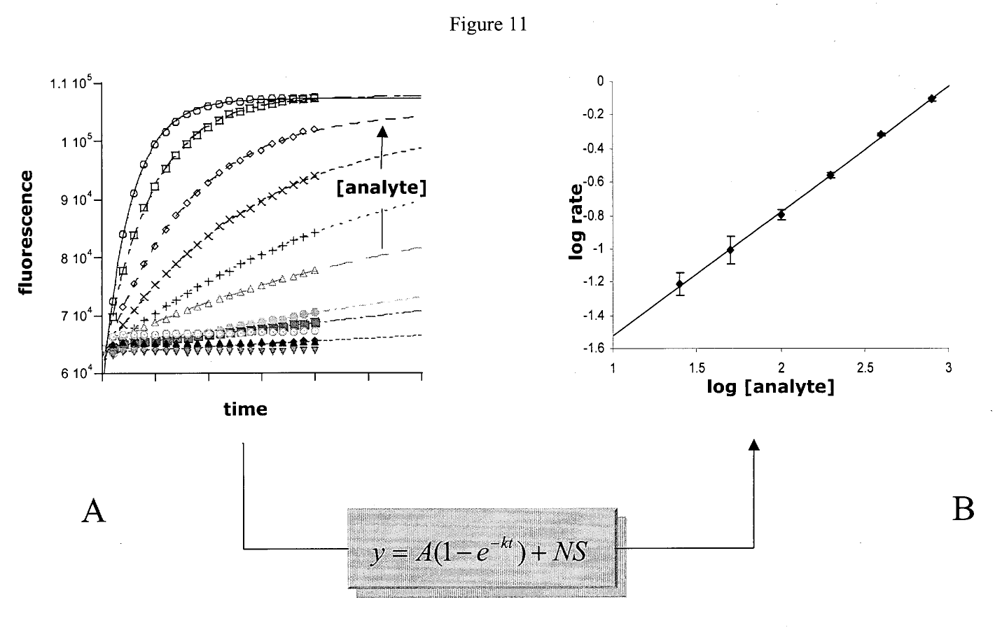

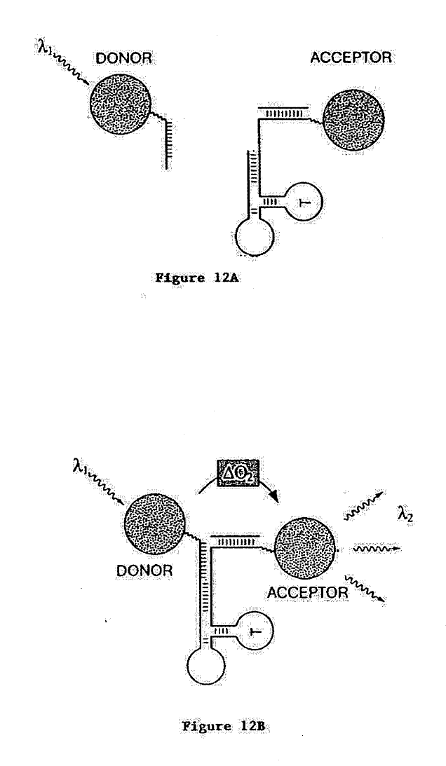

Upon effector-mediated cleavage of the hammerhead NASM, the 3′-terminus that contains one of the dye modifications is separated and dissociates away from the core NASM (FIG. 9). Thereby the donor and acceptor fluorophores are separated, leading to a strong increase in the donor fluorescence emission signal, while the acceptor emission signal will show a commensurate decrease in intensity. The increase or decrease in fluorescence can be recorded as a function of reaction time. Since the hammerhead NASM construct described herein exerts cis-cleavage activity, they follow a first-order cleavage kinetic model which allows the calculation of reaction rates after analysis of the resulting fluorescence vs. time curves (FIGS. 11A and 11B). Typically, within a certain range, the catalytic rate is a function of the effector concentration and can therefore be used to calculate an unknown effector concentration based on a measured rate value. This type of 1st order kinetic analysis in completely independent on the absolute fluorescent signal values, but relies only on their relative change over time. This makes this system particularly robust against signal fluctuations due to pipetting errors etc. compared to other, trans-reacting systems (i.e., hammerhead ribozymes acting on a separate substrate molecule).

To perform fluorescence resonance energy transfer (FRET) measurements, fluorescein-labeled RNA and quencher oligo are mixed to form the nucleic acid sensor cleavage solution. Cleavage reactions are performed in black 96-well microplates, and are started by mixing the nucleic acid sensor solution with target molecule in assay buffer. The fluorescence signals are monitored in a Fusion™ a-FP plate reader and the obtained fluorescence (rfu) values are plotted against time. The apparent reaction rates can be calculated assuming the 1st order kinetic model equation y=A(1−e^{-kt})+NS (A: signal amplitude; k: observed catalytic rate; NS: nonspecific background signal) using a curve fit algorithm (KaleidaGraph, Synergy Software, Reading, Pa.), as shown in FIG. 11. Dose-response curves are generated by plotting the calculated rates vs. the corresponding target concentrations.

ii) Indirect Energy Transfer

Other proximity-dependent signaling systems that do not rely on direct energy transfer between signaling moieties are also known in the art and can be used in the methods described herein. These include, e.g., systems in which a signaling moiety is stimulated to fluoresce or luminesce upon activation by the target molecule. This activation may be direct (e.g., as in the case of scintillation proximity assays (SPA), via a photon or radionucleide decay product emitted by the bound target), or indirect (e.g., as in the case of AlphaScreen™ assays, via reaction with singlet oxygen released from a photosensitized donor bead upon illumination). In both scenarios, the activation of detected signaling moiety is dependent on close proximity of the signaling moiety and the activating species. In general, for both fluorescence, fluorescence polarization, and scintillation-proximity-type assays, the nucleic acid sensor molecule may be utilized in either solution-phase or solid-phase formats. That is, in functional form, the nucleic acid sensor molecule may be tethered (directly, or via a linker) to a solid support or free in solution.

In one embodiment of an SPA assay, nucleic acid sensor molecules which ligate an oligonucleotide substrate in the presence of a target molecule (ADP), are bound to a scintillant-impregnated microwell plate (e.g., FlashPlates, NEN Life Sciences Products, Boston, Mass.) coated with, for example, streptavidin via a (biotin) linker attached to the 5′ end of a capture oligonucleotide sequence. The various plate-sensor coupling chemistries are determined by the type and manufacturer of the plates, and are well-known in the art. Upon the addition of a solution containing target molecule and excess radiolabeled (e.g., ³⁵S) oligonucleotide substrate in ligation buffer, the NASMs hybridize and ligate the substrate oligonucleotide. Some fraction of the radiolabeled oligonucleotide substrate will be ligated to surface-immobilized NASMs on the plate, while unligated oligonucleotide substrate will be free in solution. Only those oligonucleotide substrates ligated to surface-immobilized NASMs on the plate will be in close enough proximity to the scintillant molecules embedded in the plate to excite them, thereby stimulating luminescence which can be easily detected using a luminometer (e.g., the TopCount luminescence plate reader, Packard Biosciences, Meriden, Conn.). This type of homogeneous assay format provides straightforward, real-time detection, quantification, and kinetic properties of target molecule binding.

In another embodiment, a similar SPA assay format is performed using scintillant-impregnated beads (e.g., Amersham Pharmacia Biotech, Inc., Piscataway, N.J.). In this embodiment, NASMs which ligate on an oligonucleotide substrate in the presence of a target molecule are coupled to scintillant-impregnated beads which are suspended in solution in, for example, a microwell plate. The various bead-sensor coupling chemistries are determined by the type and manufacturer of the beads, and are well-known in the art. Upon the addition of a solution containing target molecule and excess radiolabeled (e.g., ³⁵S) oligonucleotide substrate in ligation buffer, the NASMs hybridize and ligate the oligonucleotide substrate. Some fraction of the radiolabeled substrate will be ligated to surface-immobilized NASMs on the beads, while unligated substrate will be free in solution. Only those substrates ligated to surface-immobilized NASMs on the beads will be in close enough proximity to the scintillant molecules embedded in the beads to excite them, thereby stimulating luminescence which can be easily detected using a luminometer (e.g., the TopCount luminescence plate reader, Packard Biosciences, Meriden, Conn.). In addition to enabling real-time target detection and quantification, this type of homogeneous assay format can be used to investigate cellular processes in situ in real time. This could be done by culturing cells directly onto a microwell plate and allowing uptake of scintillant beads and radioisotope by cells. Biosynthesis, proliferation, drug uptake, cell motility, etc. can then be monitored via the luminescence signal generated by beads in presence of selected target molecules (see, e.g., Cook et al., Pharmaceutical Manufacturing International pp. 49-53 (1992) or Heath et al., Cell Signaling: Experimental Strategies pp. 193-194 (1992)).

FIGS. 12A and 12B show an exemplary embodiment of a non-isotopic proximity assay based on nucleic acid sensor molecules used in conjunction with AlphaScreen™ beads (Packard Biosciences, Meriden, Conn.). In this embodiment, the nucleic acid sensor molecules, which ligate an oligonucleotide substrate in the presence of a target molecule, are bound to a chemiluminescent compound-impregnated acceptor bead coated with, for example, streptavidin, via a (biotin) linker attached to the 5′ end of the effector oligonucleotide sequence. The various bead-sensor coupling chemistries are determined by the type and manufacturer of the beads, and are well-known in the art. The oligonucleotide substrate is coupled to a photosensitizer-impregnated donor bead coated with, for example, streptavidin, via a (biotin) linker attached to the 3′ end of the substrate. The donor (substrate) and acceptor (ribozyme) beads and target molecules are then combined in solution in a microwell plate, some of the NASMs hybridize and ligate the oligonucleotide substrate, bringing the donor and acceptor beads into close proximity (<200 nm). Upon illumination at 680 nm, the photosensitizer in the donor bead converts ambient oxygen into the singlet state at a rate of approximately 60,000/second per bead. The singlet oxygen will diffuse a maximum distance of approximately 200 nm in solution; if an acceptor bead containing a chemiluminescent compound is within this range, i.e., if ligation has occurred in the presence of the target molecule, chemiluminescence at 370 nm is generated. This radiation is immediately converted within the acceptor bead to visible luminescence at 520-620 nm with a decay half-life of 0.3 sec. The visible luminescence at 520-620 nm is detected using a time-resolved fluorescence/luminescence plate reader (e.g., the Fusion multifunction plate reader, Packard Biosciences, Meriden, Conn.). This type of nonisotopic homogeneous proximity assay format provides highly sensitive detection and quantification of target molecule concentrations in volumes <25 microliters for high throughput screening (see, e.g., Beaudet et al., Genome Res. 11:600 (2001)).

SPA assays can be performed with any type of NASM (i.e., endonucleases as well as ligases). This type of assay can also be used with the aptamers of the invention to monitor the presence or concentration of target in a solution. FIG. 18 depicts the use of a surface-bound ADP aptamer to monitor the presence or concentration of radiolabeled ADP in solution. FIG. 32 depicts the use of this assay to monitor kinase function by measuring ADP production. In an aptamer SPA, a kinase is reacted with γ-³³P-ATP and a substrate in the presence of a biotinylated ADP aptamer which is bound to a streptavidin coated flash plate containing a scintillant imbedded into the surface of the plate. The greater the amount of ³³P-ADP generated by the kinase reaction and bound to the ADP aptamer, the greater the SPA signal. If a kinase inhibitor screen is being done, a successful inhibitor will cause a significant decrease in SPA signal relative to a control reaction without inhibitors.

iii) Optical Signal Generating Units With Single Signaling Moieties

In one embodiment, the optical nucleic acid sensor molecule comprises an optical signaling unit with a single signaling moiety introduced at either an internal or terminal position within the nucleic acid sensor molecule. In this embodiment, binding of the target molecule results in changes in both the conformation and physical aspect (e.g., molecular volume or mass, rotational diffusion rate, etc.) of the nucleic acid sensor molecule. Conformational changes in the nucleic acid sensor molecule upon target recognition will modify the chemical environment of the signaling moiety. Such a change in chemical environment will in general change the optical properties of the signaling moiety. Suitable signaling moieties are described in Jhaveri et al., Am. Chem. Soc. 122:2469-2473 (2000), and include, e.g., fluorescein, acridine, and other organic and nonorganic fluorophores.

In one embodiment, a signaling moiety is introduced at a position in the catalytic nucleic acid molecule near the target activation site (identifiable by footprinting studies, for example). Binding of the target molecule will (via a change in conformation of the nucleic acid molecule) alter the chemical environment and thus affect the optical properties of the signaling moiety in a detectable manner.

Recognition of the target molecule by the NASM will result in changes in the conformation and physical aspect of the nucleic acid sensor molecule, and will thus alter the kinetic properties of the signaling moiety. In particular, the changes in conformation and mass of the sensor-target complex will reduce the rotational diffuision rate for the sensor-target complex, resulting in a detectable change in the observed steady state fluorescence polarization (FP) from the signaling moiety. The expected change in FP signal with target concentration can be derived using a modified form of the well-known Michaelis-Menten model for ligand binding kinetics (see, e.g., Lakowicz, J. R., Principles of Fluorescence Spectroscopy, Second Edition, 1999, Kluwer Academic/Plenum Publishers, New York). FP is therefore a highly sensitive means of detecting and quantitatively determining the concentration of target molecules in a sample solution (Jameson et al., Methods in Enzymology 246:283 (1995); Jameson et al., METHODS 19:222 (1999); Jolley, Comb. Chem. High Throughput Screen 2(4):177 (1999); Singh, et al., BioTechniques 29:344 (2000); Owicki et al., Genetic Engineering News 17(19) (1997)). FP methods are capable of functioning in both solution- and solid-phase implementations.

Numerous additional methods can be used that, e.g., make use of a single fluorescent label and an unpaired guanosine residue (instead of a quencher group), to enable the use of FRET in target detection and quantitation as described in the embodiments above (see, e.g., Walter et al., RNA 3:392 (1997)).

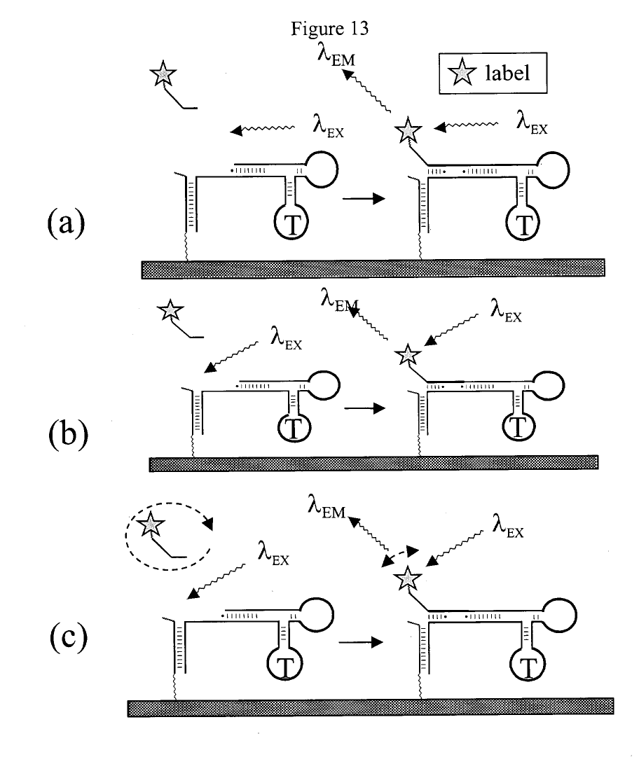

In a further embodiment, shown in FIGS. 13A, B, and C, an unlabeled ligating NASM such as the lysozyme-dependent L1 ligase is shown (see, e.g., Robertson et al., Nucleic Acids Res. 28:1751-1759 (2000)). In the unactivated state, i.e., in the absence of target, no fluorescent emission is detected from the surface-bound NASMs under total internal reflection (TIR)-illumination (see FIG. 13A), or epi-illumination (see FIG. 13B). Upon recognition of target molecules in the presence of an oligonucleotide substrate with a tag (where the tag is capable of binding to a subsequently added fluorescent label via interactions including, but not limited to, biotin/streptavidin, amine/aldehyde, hydrazide, thiol, or other reactive groups) those oligonucleotide substrates hybridized to NASMs will undergo ligation and become covalently bonded to the thereto. In order to maximize the probability of hybridization for a given NASM, oligonucleotide substrate can be added in excess relative to NASM, the temperature of the ambient solution in which the reaction takes place can be kept below room temperature (e.g., 4° C.), and agitation of the reaction vessel can be employed to overcome the kinetic limitation of diffusion-limited transport of species in solution. Given the above conditions, as well as sufficient time for maximal hybridization and subsequent ligation to occur, fluorescent label with the appropriate reactive group to bind the substrate tag is added to the reaction mixture. Again, the degree of substrate-label binding can be maximized through control of label concentration, solution temperature, and agitation. Once the fluorescent label has bound to all available ligated substrate-NASM target complex, the solution temperature can be raised to drive off all of the hybridized but unligated substrate. With TIR-illumination, the spatial extent of the excitation region above the solid substrate surface to which the ribozymes are bound is only on the order of 100 nm. Therefore, the bulk solution above the substrate surface is not illuminated and the detected fluorescent emission will be primarily due to fluorophores which are bound to ligated oligonucleotide substrate-NASM-target molecule complexes tethered to the substrate surface. The fluorescence emission from surface-bound NASM-target molecule complexes in this homogeneous solid phase assay format represents an easily detectable optical signal. In another embodiment, the fluorescence polarization (FP) of the labeled substrate can be monitored, as shown in FIG. 13C. Upon ligation, the steady state fluorescence polarization signal from the substrate-NASM complex will increase detectably relative to the FP signal from the free labeled oligonucleotide substrate in solution, due to the difference in the diffusional rotation rates between the free and ligated forms.

In another embodiment, an unlabeled ligating NASM such as the lysozyme-dependent L1 ligase (see, e.g., Robertson et al., Nucleic Acids Res. 28:1751-1759 (2000)) is bound to a solid surface. In this embodiment, the oligonucleotide substrate is coupled to an enzyme-linked luminescent moiety, such as horseradish peroxidase (HRP) by a tag (where the tag is capable of binding to a subsequently added label via interactions including, but not limited to, biotin/streptavidin, amine/aldehyde, hydrazide, thiol, or other reactive groups). In the absence of target molecule, no luminescent emission is detected from the surface-bound NASMs. Upon recognition of target molecules in the presence of labeled oligonucleotide substrate, those oligonucleotide substrates hybridized to NASMs will undergo ligation and become covalently bonded to the NASMs. After removal of excess, unbound oligonucleotide substrate, the substrate for activation of the enzyme-linked luminescent label is added to the reaction volume. The resulting luminescent signal (e.g., from HRP, luciferase, etc.) is easily detectable using standard luminometers (e.g., the Fusion multifunction plate reader, Packard Bioscience). In a further embodiment, the activated solution can be precipitated, followed by colorimetric detection. In a particular embodiment, the enzyme linked signal amplification, TSA, (sometimes referred to as CARD-catalyzed reporter deposition) is an ultrasensitive detection method. The technology uses turnover of multiple tyramide substrates per horseradish peroxidase (HRP) enzyme to generate high-density labeling of a target protein or nucleic acid probe in situ. Tyramide signal amplification is a combination of three elementary processes: (1) Ligation (or not) of a biotinylated ligase oligonucleotide substrate oligo, followed by binding (or not) of a streptavidin-HRP to the probe; (2) HRP-mediated conversion of multiple copies of a fluorescent tyramide derivative to a highly reactive radical; and (3) Covalent binding of the reactive, short lived tyramide radicals to nearby nucleophilic residues, greatly reducing diffusion-related signal loss.

5) Generating Biosensors

Optical nucleic acid sensor molecules for the detection of a target molecule of interest are generated by first selecting catalytic nucleic acid molecules with catalytic activity modifiable (e.g., activatable) by a selected target molecule. In one embodiment, at least a portion of the catalytic site of the catalytic NASM is then removed and an optical signal generating unit is either added or inserted. Recognition of the target molecule by the nucleic acid sensor molecule activates a change in the properties of the optical signaling unit.

The nucleic acid sensor molecules can be, e.g., those which possess either ligating or cleaving activity in the presence of a target molecule.

One advantage of using nucleic acid sensor molecule arrays as opposed to protein arrays is the relative ease with which nucleic acid sensor molecules can be attached to chip surfaces. Immobilization of nucleic acid sensor molecules on a substrate provides a straightforward mechanism for carrying out multiple arrays in parallel. Initially, the optimal attachment chemistries are determined for use in immobilizing these molecules on a solid substrate. These molecules are further configured such that their activity and allosteric behavior is maintained following immobilization. Generally, the chip is configured such that it may be placed at the bottom of a sample holder and overlaid with sample solution, target and substrate oligonucleotide. Following an incubation to allow target present within the sample to activate catalysis, the sample is washed away and the extent of ribozyme catalysis quantified.

For example, endonuclease nucleic acid sensor molecules are generated by transcription in the presence of γ-thio-GTP (introducing a unique thiol at their 5′-end) and subsequently attached to a thiol-reactive surface (e.g. gold-coated polystyrene as described by Seetharaman et al., Nature Biotech 19:336 (2001)). Attachment methodologies are evaluated on the basis of the following criteria: efficiency, e.g., what is the yield of nucleic acid sensor molecule capture; capacity, e.g., what is the maximum concentration of nucleic acid sensor molecules that can be localized in a given spot size; stability, e.g., are ribozymes efficiently retained under a variety of solution conditions and during long-term storage; detection, e.g., do immobilization chemistries interfere with the ability to generate a detectable signal.

To the extent that activity for immobilized nucleic acid sensor molecules is diminished, three different strategies for reconfiguring ribozymes for activity in solid phase applications are available: 1) immobilization chemistries, a variety of different immobilization chemistries are compared on the basis of their ability to maintain allosteric behavior. To the extent that they leave different surfaces available for protein effectors to interact with, that they tether different ends of the nucleic acid sensor molecules, and that they position the NASM either directly at the surface or displaced from the surface (in the case of streptavidin capture), different behaviors are observed depending upon the immobilization method. Protein-target activated NASMs have been shown to function in both direct and indirect attachment scenarios; 2) blocking chemistries, blocking agents (e.g., carrier proteins) are tested to determine whether losses in allosteric responsiveness are due to non-specific interactions between the allosteric activators and the chip surface; 3) tethers, steric effects may cause decreased catalytic activity upon direct end attachment to a solid support. Arbitrary sequence tethers are added as needed to increase the spacing between the attachment end and the core of the ribozyme.

Immobilized nucleic acid sensor molecules for target are prepared and are assayed for activity by monitoring either retention of end-labeled oligonucleotide substrate (for L1 ligase-based ribozymes) or release of end-labeled ribozyme (for endonucleases as originally described by Seetherman et al., Nature Biotech 19:336 (2001)). Radioactive tracers are used for labeling RNAs and substrates.

In one-embodiment, a biosensor is provided which comprises a plurality of optical nucleic acid sensor molecules labeled with first and second signaling moieties specific for a target molecule. In another embodiment, the optical NASMs are labeled with a single signaling moiety. In one embodiment, the labeled nucleic acid sensor molecules are provided in a solution (e.g., a buffer). In another embodiment, the labeled nucleic acid sensor molecules are attached directly or indirectly (e.g., through a linker molecule) to a substrate. In further embodiments, nucleic acid sensor molecules can be synthesized directly onto the substrate. Suitable substrates which are encompassed within the scope include, e.g., glass or quartz, silicon, encapsulated or unencapsulated semiconductor nanocrystal materials (e.g., CdSe), nitrocellulose, nylon, plastic, and other polymers. Substrates may assume a variety of configurations (including, e.g., planar, slide shaped, wafers, chips, tubular, disc-like, beads, containers, or plates, such as microtiter plates, and other shapes).

Different chemistries for attaching nucleic acid sensor molecules to solid supports include: 1) conventional DNA arrays using aldehyde coated slides and 5′-amino modified oligonucleotides. The attached oligonucleotide serves as a capture tag that specifically hybridizes to a 3′-end extension on the ribozyme. Nucleic acid sensor molecule RNA treated with periodate to specifically introduce an aldehyde modification at the 3′-end. Modified RNA can be used either in a subsequent reaction with biotin hydrazide enables RNA capture on commercially-available streptavidin coated slides or in a subsequent reaction with adipic acid dihydrazide enables RNA capture on commercially-available aldehyde coated slides.

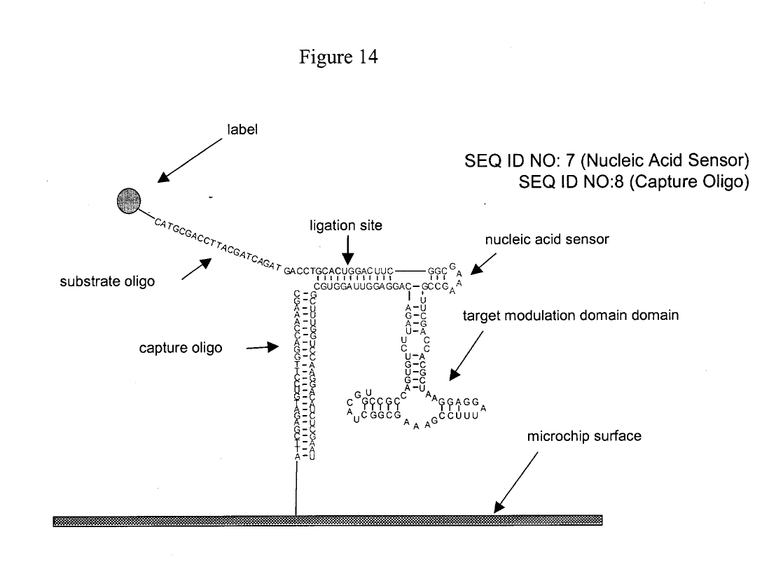

Numerous attachment chemistries, both direct and indirect, can be used to immobilize the sensor molecules on a solid support. These include, e.g., amine/aldehyde, biotin/streptavidin (avidin, neutravidin), ADH/oxidized 3′ RNA. In a particular embodiment, the nucleic acid sensor molecules ligate a substrate in the presence of a target molecule. In this embodiment the ribozymes are bound to a solid substrate via the effector oligonucleotide sequence as shown in FIG. 14.

In one embodiment, larger substrates can be generated by combining a plurality of smaller biosensors forming an array of biosensors. In a further embodiment, nucleic acid sensor molecules placed on the substrate are addressed (e.g., by specific linker or effector oligonucleotide sequences on the nucleic acid sensor molecule) and information relating to the location of each nucleic acid sensor molecule and its target molecule specificity is stored within a processor. This technique is known as spatial addressing or spatial multiplexing. Techniques for addressing nucleic acids on substrates are known in the art and are described in, for example, U.S. Pat. No. 6,060,252; U.S. Pat. No. 6,051,380; U.S. Pat. No. 5,763,263; U.S. Pat. No. 5,763,175; and U.S. Pat. No. 5,741,462.

In another embodiment, a manual or computer-controlled robotic microarrayer is used to generate arrays of nucleic acid sensor molecules immobilized on a solid substrate. In one embodiment, the arrayer utilizes contact-printing technology (i.e., it utilizes printing pins of metal, glass, etc., with or without quill-slots or other modifications). In a different embodiment, the arrayer utilizes non-contact printing technology (i.e., it utilizes ink jet or capillary-based technologies, or other means of dispensing a solution containing the material to be arrayed). Numerous methods for preparing, processing, and analyzing microarrays are known in the art (see Schena et al., Microarray Biochip Technology, ed. pp. 1-18 (2000); Mace et al., Microarray Biochip Technology, ed. pp. 39-64 (2000); Heller et al., Academic Press, Inc. pp. 245-256 (1999); Basararsky et al., Microarray Biochip Technology, ed. pp. 265-284 (2000); Schermer, DNA Microarrays a Practical Approach pp. 17-42 (1999)). Robotic and manual arrayers are commercially available including, for example, the SpotArray from Packard Biosciences, Meriden, Conn., and the RA-1 from GenomicSolutions, Ann Arbor, Mich.

In another embodiment, different nucleic acid sensor molecules are immobilized on a streptavidin-derivatized substrate via biotin linkers. The individual sensor spots can be manually arrayed. For example, NASM can hybridize to a biotin-linked capture oligo, which in turn will bind to a streptavidin coated surface.

Solution measurements of target molecule concentration can be made by bathing the surface of the biosensor array in a solution containing the targets (analytes) of interest. In practice this is accomplished either by incorporating the array within a microflowcell (with a flow rate of ˜25 microliters/min), or by placing a small volume (˜6-10 microliters) of the target solution on the array surface and covering it with a cover slip. Detection and quantification of target concentration is accomplished by monitoring changes in the fluorescence polarization (FP) signal emitted from the fluorescein label under illumination by 488 nm laser radiation. The rotational diffusion rate is inversely proportional to the molecular volume; thus the rotational correlation time for the roughly 20-nucleotide unbound sensor (i.e., in the absence of target molecule) will be significantly less than that for the target-NASM complex. The fluorescence emission from the target-NASM complex will therefore experience greater residual polarization due to the smaller angle through which the emission dipole axis of the sensor fluorophore can rotate within its radiative lifetime. In another embodiment, different surface attachment chemistries are used to immobilize the NASMs on a solid substrate. As previously noted, these include, e.g., interactions involving biotin/streptavidin, amine/aldehyde, hydrazide, thiol, or other reactive groups.

One type of array includes immobilized effector oligonucleotides with terminal amine groups attached to a solid substrate derivatized with aldehyde groups. This array can be used to spatially address (i.e., the sequence of nucleotides for each effector oligonucleotide can be synthesized as a cognate to the effector oligonucleotide binding domain of a nucleic acid sensor molecule specific for a particular target molecule) and immobilize the nucleic acid sensor molecules prior to their use in a solid-phase assay (see, e.g., Zammatteo et al., Anal Biochem 280:143 (2000)).

For example, to attach effector oligonucleotides to aldehyde derivatized substrate, discrete spots of solution containing effector oligonucleotides with amine-reactive terminal groups or linkers with terminal amine groups using microarraying pins, pipette, etc are printed and then allowed to dry to dry for 12 hrs. at room temperature and <30% relative humidity. The substrate is then rinsed twice with dH₂O containing 0.2% SDS for 2 min. with vigorous agitation at room temperature. The substrate is then rinsed once in dH₂O for 2 min. with vigorous agitation at room temperature and transferred to boiling (100° C.) dH₂O for 3 min. to denature DNA. The denatured substrate is then dried by centrifuging at 500×g for 1 min. and then treated with 0.1 M NaBH₄ in phosphate buffered saline (PBS, pH 7) for 5 min. with mild agitation at room temperature. Following NaBH₄ treatment, the substrate is rinsed twice in dH₂O containing 0.2% SDS for 1 min. with vigorous agitation at room temperature and then washed once with dH₂O for 2 min. with vigorous agitation at room temperature. The substrate is again boiled in dH₂O (100° C.) for 10 sec. to denature DNA. The substrate is dried by centrifugation as described above and stored at 4° C. prior to hybridization.

In the case where it is desirable to immobilize an array of NASMs by direct attachment to a solid surface, the nucleic acid sensor molecules are bound to a solid substrate directly via their 3′ termini. The attachment is accomplished by oxidation (using, e.g., Ncc periodate) of the 3′ vicinal diol of the nucleic acid sensor molecule to an aldehyde group. This aldehyde group will react with a hydrazide group to form a hydrazone bond. The hydrazone bond is quite stable to hydrolysis, etc., but can be further reduced (for example, by treatment with NaBH₄ or NaCNBH₃). The use of adipic acid dihydrazide (ADH, a bifunctional linker) to derivatize an aldehyde surface results in a hydrazide-derivatized surface which provides a linker of approximately 10 atoms between the substrate surface and point of biomolecular attachment (see Ruhn et al., J. Chromatography A 669:9 (1994); O'Shaughnessy, J. Chromatography 510:13 (1990); Roberston et al., Biochemistry 11(4):533 (1972); Schluep et al., Bioseparation 7:317 (1999); Chan et al., J. Colloid and Interface Sci. 203:197 (1998)).

A hydrazide-terminated surface can be prepared by ADH treatment of the aldehyde substrate. Briefly, to 50 mL of 0.1 M phosphate buffer (pH 5) 100-fold excess of adipic acid dihydrazide (ADH) relative to concentration of aldehyde groups is added on substrate surface. The substrate is then placed in a 50 mL tube containing the ADH in phosphate buffer and shaken mixture for 2 h. Following incubation, the substrate is washed 4-times with 0.1 M phosphate buffer (pH 7). The free aldehyde groups on the substrate surface are then reduced by treatment with a 25-fold excess of NaBH₄ or NaCNBH₃ in 0.1 M phosphate buffer in a 50 ml conical tube with shaking for 90 min. The substrate is then washed 4-times with 0.1 M phosphate buffer (pH 7) and stored 0.1 M phosphate buffer (pH 7) at 4° C. until use.

Nucleic acid molecules for specific coupling to the ADH-terminated surface via their 3′ termini are prepared by periodate oxidation of the RNA, see, e.g., Proudnikov et al., Nucleic Acid Res. 24(22):4535 (1996); Wu et al., Nucleic Acids Res. 24(17):3472 (1996). Briefly, up to 20 μg RNA in 5 μl of H₂O at 20° C. is treated with 1 ml 0.1 M NaIO₄ (˜20-fold excess relative to RNA). The RNA is incubated with the NaIO₄ for 30 min. in a light-tight tube prior to the addition of 1 ml 0.2 M Na sulphite (˜2-fold excess relative to NaIO₄) to stop the reaction (30 min.; room temperature). The oxidized RNA is then recovered by ethanol precipitation and a spin-separation column.

The specificity of the biosensors and NASMs according to the invention is determined by the specificity of the target modulation domain of the nucleic acid sensor molecule. In one embodiment, a biosensor is provided in which all of the nucleic acid sensor molecules recognize the same molecule. In another embodiment, a biosensor is provided which can recognize at least two different target molecules allowing for multi-analyte detection. Multiple analytes can be distinguished by using different combinations of first and second signaling molecules. In addition to the wavelength/color and spatial multiplexing techniques previously described, biosensors may be used to detect multiple analytes using intensity multiplexing. This is accomplished by varying the number of fluorescent label molecules on each biosensor in a controlled fashion. Since a single fluorescent label is the smallest integral labeling unit possible, the number of fluorophores (i.e., the intensity from) a given biosensor molecule provides a multiplexing index. Using the combination of 6-wavelength (color) and 10-level intensity multiplexing, implemented in the context of semiconductor nanocrystals derivatized as bioconjugates, would theoretically allow the encoding of million different analyte-specific biosensors (Han et al., Nature Biotechnol. 19:631 (2001)).

In one embodiment, multiple single target biosensors can be combined to form a multianalyte detection system which is either solution-based or substrate-based according to the needs of the user. In this embodiment, individual biosensors can be later removed from the system, if the user desires to return to a single analyte detection system (e.g., using target molecules bound to supports, or, for example, manually removing a selected biosensor(s) in the case of substrate-based biosensors). In a further embodiment, nucleic acid sensor molecules binding to multiple analytes are distinguished from each other by referring to the address of the nucleic acid sensor molecule on a substrate and correlating its location with the appropriate target molecule to which it binds (previously described as spatial addressing or multiplexing).

In one embodiment, subsections of a biosensor array can be individually subjected to separate analyte solutions by use of substrate partitions or enclosures that prevent fluid flow between subarrays, and microfluidic pathways and injectors to introduce the different analyte solutions to the appropriate sensor subarray.

Nucleic Acid Sensor Molecule and Biosensor Systems

In one embodiment, a nucleic acid sensor molecule or biosensor system is provided comprising a nucleic acid sensor molecule in communication with a detector system. In a further embodiment, a processor is provided to process optical signals detected by the detector system. In still a further embodiment, the processor is connectable to a server which is also connectable to other processors. In this embodiment, optical data obtained at a site where the NASM or biosensor system resides can be transmitted through the server and data is obtained, and a report displayed on the display of the off-site processor within seconds of the transmission of the optical data. In one embodiment, data from patients is stored in a database which can be accessed by a user of the system.

Data obtainable from the biosensors according to the invention include diagnostic data, data relating to lead compound development, and nucleic acid sensor molecule modeling data (e.g., information correlating the sequence of individual sensor molecules with specificity for a particular target molecule). In one embodiment, these data are stored in a computer database. In a further embodiment, the database includes, along with diagnostic data obtained from a sample by the biosensor, information relating to a particular patient, such as medical history and billing information. Although, in one embodiment, the database is part of the nucleic acid sensor molecule system, the database can be used separately with other detection assay methods and drug development methods.

Detectors used with the nucleic acid sensor molecule systems according to the invention, can vary, and include any suitable detectors for detecting optical changes in nucleic acid molecules. These include, e.g., photomultiplier tubes (PMTs), charge coupled devices (CCDs), intensified CCDs, and avalanche photodiodes (APDs). In one embodiment, an optical nucleic acid sensor molecule is excited by a light source in communication with the biosensor. In a further embodiment, when the optical signaling unit comprises first and second signal moieties that are donor/acceptor pairs (i.e., signal generation relies on the fluorescence of a donor molecule when it is removed from the proximity of a quencher acceptor molecule), recognition of a target molecule will cause a large increase in fluorescence emission intensity over a low background signal level. The high signal-to-noise ratio permits small signals to be measured using high-gain detectors, such as PMTs or APDs. Using intensified CCDs, and PMTs, single molecule fluorescence measurements have been made by monitoring the fluorescence emission, and changes in fluorescence lifetime, from donor/acceptor FRET pairs (see, e.g., Sako, et al., Nature Cell Bio. 2:168 (2000); Lakowicz et al, Rev. Sci. Instr. 62(7):1727 (1991)).

Light sources include, e.g., filtered, wide-spectrum light sources, (e.g., tungsten, or xenon arc), laser light sources, such as gas lasers, solid state crystal lasers, semiconductor diode lasers (including multiple quantum well, distributed feedback, and vertical cavity surface emitting lasers (VCSELs)), dye lasers, metallic vapor lasers, free electron lasers, and lasers using any other substance as a gain medium. Common gas lasers include Argon-ion, Krypton-ion, and mixed gas (e.g., Ar—Kr) ion lasers, emitting at 455, 458, 466, 476, 488, 496, 502, 514, and 528 nm (Ar ion); and 406, 413, 415, 468, 476, 482, 520, 531, 568, 647, and 676 nm (Kr ion). Also included in gas lasers are Helium Neon lasers emitting at 543, 594, 612, and 633 nm. Typical output lines from solid state crystal lasers include 532 nm (doubled Nd:YAG) and 408/816 nm (doubled/primary from Ti:Sapphire). Typical output lines from semiconductor diode lasers are 635, 650, 670, and 780 nm.

Excitation wavelengths and emission detection wavelengths will vary depending on the signaling moieties used. In one embodiment, where the first and second signaling moieties are fluorescein and DABCYL, the excitation wavelength is 488 nm and the emission wavelength is 514 nm. In the case of semiconductor nanocrystal-based fluorescent labels, a single excitation wavelength or broadband UV source may be used to excite several probes with widely spectrally separated emission wavelengths (see Bruchez et al., Science 281:2013 (1998); Chan et al., J. Colloid and Interface Sci. 203:197 (1998)).

In one embodiment, detection of changes in the optical properties of the nucleic acid sensor molecules is performed using any of a cooled CCD camera, a cooled intensified CCD camera, a single-photon-counting detector (e.g., PMT or APD), or other light sensitive sensor. In one embodiment, the detector is optically coupled to the nucleic acid sensor molecule through a lens system, such as in an optical microscope (e.g., a confocal microscope). In another embodiment, a fiber optic coupler is used, where the input to the optical fiber is placed in close proximity to the substrate surface of a biosensor, either above or below the substrate. In yet another embodiment, the optical fiber provides the substrate for the attachment of nucleic acid sensor molecules and the biosensor is an integral part of the optical fiber.

In one embodiment, the interior surface of a glass or plastic capillary tube provides the substrate for the attachment of nucleic acid sensor molecules. The capillary can be either circular or rectangular in cross-section, and of any dimension. The capillary section containing the biosensors can be integrated into a microfluidic liquid-handling system which can inject different wash, buffer, and analyte-containing solutions through the sensor tube. Spatial encoding of the sensors can be accomplished by patterning them longitudinally along the axis of the tube, as well as radially, around the circumference of the tube interior. Excitation can be accomplished by coupling a laser source (e.g., using a shaped output beam, such as from a VCSEL) into the glass or plastic layer forming the capillary tube. The coupled excitation light will undergo TIR at the interior surface/solution interface of the tube, thus selectively exciting fluorescently labeled biosensors attached to the tube walls, but not the bulk solution. In one embodiment, detection can be accomplished using a lens-coupled or proximity-coupled large area segmented (pixelated) detector, such as a CCD. In a particular embodiment, a scanning (i.e., longitudinal/axial and azimuthal) microscope objective lens/emission filter combination is used to image the biosensor substrate onto a CCD detector. In a different embodiment, a high resolution CCD detector with an emission filter in front of it is placed in extremely close proximity to the capillary to allow direct imaging of the biosensors. In a different embodiment, highly efficient detection is accomplished using a mirrored tubular cavity that is elliptical in cross-section. The sensor tube is placed along one focal axis of the cavity, while a side-window PMT is placed along the other focal axis with an emission filter in front of it. Any light emitted from the biosensor tube in any direction will be collected by the cavity and focused onto the window of the PMT.

In still another embodiment, the optical properties of a nucleic acid sensor molecule are analyzed using a spectrometer (e.g., such as a luminescence spectrometer) which is in communication with the biosensor. The spectrometer can perform wavelength discrimination for excitation and detection using either monochromators (i.e., diffraction gratings), or wavelength bandpass filters. In this embodiment, biosensor molecules are excited at absorption maxima appropriate to the signal labeling moieties being used (e.g., acridine at 450 nm, fluorescein at 495 nm) and fluorescence intensity is measured at emission wavelengths appropriate for the labeling moiety used (e.g., acridine at 495 nm; fluorescein at 515 nm). Achieving sufficient spectral separation (i.e., a large enough Stokes shift) between the excitation wavelength and the emission wavelength is critical to the ultimate limit of detection sensitivity. Given that the intensity of the excitation light is much greater than that of the emitted fluorescence, even a small fraction of the excitation light being detected or amplified by the detection system will obscure a weak biosensor fluorescence emission signal. In one embodiment, the biosensor molecules are in solution and are pipetted (either manually or robotically) into a cuvette or a well in a microtiter plate within the spectrometer. In a further embodiment, the spectrometer is a multifunction plate reader capable of detecting optical changes in fluorescence or luminescence intensity (at one or more wavelengths), time-resolved fluorescence, fluorescence polarization (FP), absorbance (epi and transmitted), etc., such as the Fusion multifunction plate reader system (Packard Biosciences, Meriden, Conn.). Such a system can be used to detect optical changes in biosensors either in solution, bound to the surface of microwells in plates, or immobilized on the surface of solid substrate (e.g., a biosensor microarray on a glass substrate). This type of multiplate/multisubstrate detection system, coupled with robotic liquid handling and sample manipulation, is particularly amenable to high-throughput, low-volume assay formats.

In embodiments where nucleic acid sensor molecules are attached to substrates, such as a glass slide or in microarray format, it is desirable to reject any stray or background light in order to permit the detection of very low intensity fluorescence signals. In one embodiment, a small sample volume (˜10 nL) is probed to obtain spatial discrimination by using an appropriate optical configuration, such as evanescent excitation or confocal imaging. Furthermore, background light can be minimized by the use of narrow-bandpass wavelength filters between the sample and the detector and by using opaque shielding to remove any ambient light from the measurement system.

In one embodiment, spatial discrimination of nucleic acid sensor molecules attached to a substrate in a direction normal to the interface of the substrate (i.e., excitation of only a small thickness of the solution layer directly above and surrounding the plane of attachment of the biosensor molecules to the substrate surface) is obtained by evanescent wave excitation. Evanescent wave excitation utilizes electromagnetic energy that propagates into the lower-index of refraction medium when an electromagnetic wave is totally internally reflected at the interface between higher and lower-refractive index materials. In this embodiment a collimated laser beam is incident on the substrate/solution interface (at which the biosensors are immobilized) at an angle greater than the critical angle for total internal reflection (TIR). This can be accomplished by directing light into a suitably shaped prism or an optical fiber. In the case of a prism, the substrate is optically coupled (via index-matching fluid) to the upper surface of the prism, such that TIR occurs at the substrate/solution interface on which the biosensors are immobilized. Using this method, excitation can be localized to within a few hundred nanometers of the substrate/solution interface, thus eliminating autofluorescence background from the bulk analyte solution, optics, or substrate. Target recognition is detected by a change in the fluorescent emission of the nucleic acid sensor, whether a change in intensity or polarization. Spatial discrimination in the plane of the interface (i.e., laterally) is achieved by the optical system.

In one embodiment, a large area of the biosensor substrate is uniformly illuminated, either via evanescent wave excitation or epi-illumination from above, and the detected signal is spatially encoded through the use of a pixelated detector, such as CCD camera. An example of this type of uniform illumination/CCD detection system (using epi-illumination) for the case of microarrayed biosensors on solid substrates is the GeneTAC 2000 scanner (GenomicSolutions, Ann Arbor, Mich.). In a different embodiment, a small area (e.g., 10×10 microns to 100×100 microns) of the biosensor substrate is illuminated by a micro-collimated beam or focused spot. In one embodiment, the excitation spot is rastered in a 2-dimensional scan across the static biosensor substrate surface and the signal detected (with an integrating detector, such as a PMT) at each point correlated with the spatial location of that point on the biosensor substrate (e.g., by the mechanical positioning system responsible for scanning the excitation spot). Two examples of this type of moving spot detection system for the case of microarrayed biosensors on solid substrates are: the DNAScope scanner (confocal, epi-illumination, GeneFocus, Waterloo, ON, Canada), and the LS IV scanner (non-confocal, epi-illumination, GenomicSolutions, Ann Arbor, Mich.). In yet another embodiment, a small area (e.g., 10×10 microns to 100×100 microns) of the biosensor substrate is illuminated by a stationary micro-collimated beam or focused spot, and the biosensor substrate is rastered in a 2-dimensional scan beneath the static excitation spot, with the signal detected (with an integrating detector, such as a PMT) at each point correlated with the spatial location of that point on the biosensor substrate (e.g., by the mechanical positioning system responsible for scanning the substrate). An example of this type of moving substrate detection (using confocal epi-illumination) system for the case of microarrayed biosensors on solid substrates is the ScanArray 5000 scanner (Packard Biochip, Billerica, Mass.).

For example, a TIR evanescent wave excitation optical configuration is implemented, with a static substrate and dual-capability detection system. The detection system is built on the frame of a Zeiss universal fluorescence microscope. The system is equipped with 2 PMTs on one optical port, and an intensified CCD camera (Cooke, St. Louis, Mo.) mounted on the other optical port. The optical path utilizes a moveable mirror which can direct the collimated, polarized laser beam through focusing optics to form a spot, or a beam expander to form a large (>1 cm) beam whose central portion is roughly uniform over the field of view of the objective lens. Another movable mirror can direct the light either to the intensified CCD camera when using large area uniform illumination, or to the PMTs in the scanned spot mode. In spot scanning mode, a polarizing beamsplitter separates the parallel and perpendicular components of the emitted fluorescence and directs each to its designated PMT. An emission filter in the optical column rejects scattered excitation light from either type of detector. In CCD imaging mode, manually adjusted polarizers in the optical column of the microscope must be adjusted to obtain parallel and perpendicular images from which the fluorescence polarization or anisotropy can be calculated. A software program interfaces with data acquisition boards in a computer which acquires the digital output data from both PMTs and CCD. This program also controls the PMT power, electromechanical shutters, and galvanometer mirror scanner, calculates and plots fluorescence polarization in real time, and displays FP and intensity images.

In another embodiment, the detection system is a single photon counter system (see, e.g., U.S. Pat. No. 6,016,195 and U.S. Pat. No. 5,866,348) requiring rastering of the sensor substrate to image larger areas and survey the different binding regions on the biosensor.

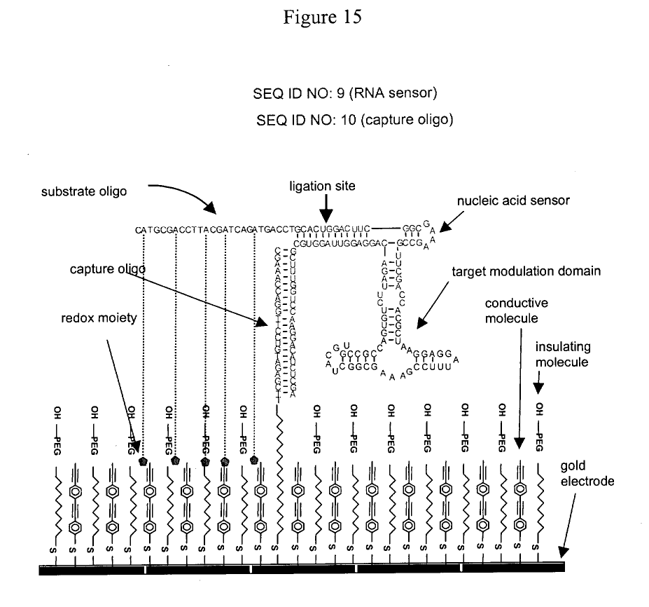

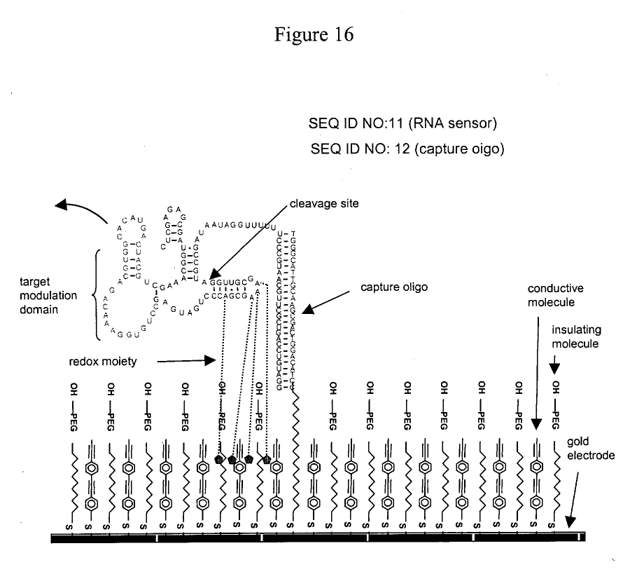

In another embodiment of the invention, the biosensor is used to detect a target molecule through changes in the electrochemical properties of the nucleic acid sensor molecules in close proximity to it which occur upon recognition of the target by the NASM. in a one embodiment, the biosensor system consists of three major components: 1) optical nucleic acid sensor molecules immobilized on an array of independently addressable gold electrodes. The nucleic acid sensor molecules immobilized on each electrode may be modulated by the same or different target molecules, including proteins, metabolites and other small molecules, etc.; 2) an oligonucleotide substrate which acts as a signaling probe, hybridizing to the oligonucleotide substrate binding domain of the ligase sensor and forming a covalent phosphodiester bond with the nucleic acid sensor molecule nucleotide adjacent to its 3′ terminus in the presence of the appropriate target. This oligonucleotide substrate is typically a nucleic acid sequence containing one or more modified nucleotides conjugated to redox active metallic complexes, e.g., ferrocene moieties, which can act as electron donors; and 3) an immobilized mixed self-assembled surface monolayer (SAM), comprised of conductive species separated by insulating species, covering the surface of the electrodes, as shown in FIGS. 15 and 16. Examples of conductive species include thiol-terminated linear molecules, such as oligophenylethyl molecules, while examples of nonconductive thiol-terminated linear molecules, include alkane-thiol molecules terminated with polyethylene glycol (PEG). All immobilized species can be covalently attached to the electrode surface by terminal thiol groups. Upon recognition of the target molecule by the target modulation domain and subsequent ligation of the oligonucleotide substrate, the redox active signaling moieties coupled to the substrate oligo will be brought into close proximity to the conductive surface layer, resulting in a detectable increase in electronic surface signal.

In another preferred embodiment, the biosensor system consists of two major components: (1) Optical nucleic acid sensor molecules immobilized on an array of independent addressable gold electrodes. The nucleic acid sensor molecules immobilized on each electrode may be modulated by the same or different target molecules, including proteins, metabolites and other small molecules, etc. The NASM will contain one or more nucleotides conjugated to redox active metallic complexes, e.g., ferrocene moieties, which can act as electron donors; and (2) an immobilized mixed self-assembled surface monolayer (SAM), comprised of conductive species separated by insulating species, covering the surface of the electrodes. Examples of conductive species include thiol-terminated linear molecules, such as oligophenylethyl molecules, while examples of nonconductive thiol-terminated linear molecules include alkane-thiol molecules terminated with polyethylene glycol (PEG). The SAM-coated molecule can be immobilized via a capture oligonucleotide. In this case, the redox active signaling moieties are coupled to the body of the NASM. Upon recognition of the target molecule by the target modulation domain and subsequent cleavage, the bulk of the NASM, including the nucleotides coupled to the redox active signaling moieties, will dissociate from the surface, resulting in a detectable loss of electronic current signal.

In another embodiment, the array would be subjected, e.g., by an integrated microfluidic flowcell, to an analyte solution containing the target(s) of interest at some unknown concentration. The range of possible sample analyte solutions may include standard buffers, biological fluids, and cell or tissue extracts. The sample solution will also contain the signaling probe at a saturating concentration relative to the immobilized nucleic acid sensor molecule. This ensures that at any given time during analysis, there is a high probability that each nucleic acid sensor molecule will have a signaling probe hybridized to it. In the presence of the target molecules in the sample solution, the nucleic acid sensor molecule will form a covalent phosphodiester bond, i.e., ligate, with the signaling probe, thus immobilizing it with its redox active electron donor species in electrical contact with the conductive molecules within the mixed self-assembled surface monolayer. After some integration time, during which signal probe ligation occurs, it may be necessary to denature the hybridized but unligated signaling probes. This denaturation step, which effectively removes ‘background’ signaling probes and their associated redox moieties from the vicinity of the electrode, can be accomplished by a small temperature increase (e.g., from 21° C. to 25° C.), or by a brief negative voltage spike applied to the sensor electrodes followed by the application of a large positive DC voltage to a separate electrode that would collect unligated signaling. For the case of a sufficiently short hybridization region, e.g., 5 base-pairs, on the signaling probe, a separate denaturation step may not be necessary. In either case, following nucleic acid sensor molecule activation by target molecules, a linear electrical potential ramp is applied to the electrodes. The redox species conjugated to the immobilized signaling probe-nucleic acid sensor molecule will be electrochemically oxidized, liberating one or more electrons per moiety. The conductive molecules within the surface monolayer will provide an electrical path for the liberated electrons to the electrode surface.

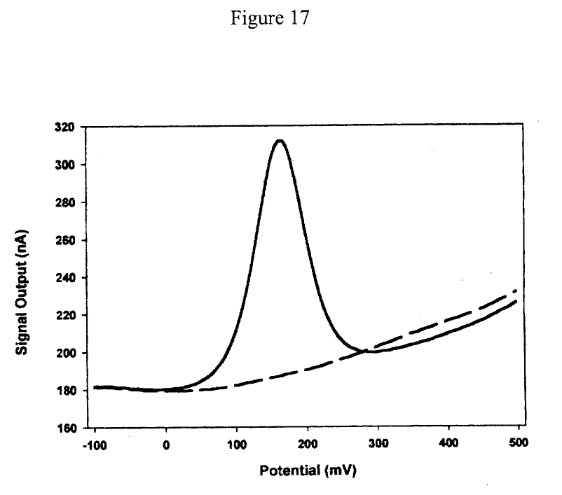

The net electron transfer to or from the electrode will be measured as a peak in the faradaic current, centered at the redox potential of the electron donor species (specified for a given reference electrode) and superposed on top of the capacitive current baseline which is observed in the absence of surface-immobilized signaling probes, as shown in FIG. 17. Quantitative analysis of the sensor signal, and therefore accurate determination of target molecule concentration, is based on the fact that the measured faradaic peak height is directly proportional to number of redox moieties immobilized at the electrode, that is, the number of nucleic acid sensor molecules ligated to signaling probes times the multiplicity of redox moieties per signaling probe molecule. Signal generation by the nucleic acid sensor molecules is thus amplified by virtue of multiple redox species per signaling probe. In addition, if an alternating current (AC) bias voltage is applied (superposed) on top of the DC linear voltage ramp applied to the sensor electrodes, i.e., in the case of AC voltammetry, signal amplification would result from the cyclic repetition of the signal-generating redox reaction.

The system described above for the case of a surface-immobilized nucleic acid sensor molecule which ligates a signaling probe containing one or more modified nucleotides conjugated to redox active species suggests a general method and instrumentation for the detection and quantitation of an arbitrary target molecule in solution in real time. Detection of a particular target would require development of a nucleic acid sensor molecule that recognizes the target molecule. Additionally, nucleic acid sensor molecules have been developed which are activated only in the presence of two different target molecules. Such dual-effector sensors could be used to detect the simultaneous presence of two or more targets, or could be used in conjunction with single-target molecule sensors to form biological logic (i.e., AND, OR, etc.) circuits.

Multiplexed detection of multiple target molecules simultaneously in a complex sample solution could be accomplished by immobilizing nucleic acid sensor molecules against the target molecules of interest on separate electrodes within a two-dimensional array of electrodes. A complex sample solution containing multiple target molecules and a common signaling probe could then be introduced to the array. All nucleic acid sensor molecules would be exposed simultaneously to all targets, with the target-activated nucleic acid sensor molecule response(s) being observed and recorded only at the spatial location(s) known to contain a nucleic acid sensor molecule specific for the target molecules present in the (unknown) sample. The utility of such a nucleic acid sensor molecule array would be greatly enhanced by the integration of a microfluidic sample and reagent delivery system. Such an integrated microfluidic system would allow the application of reagents and samples to the sensor array to be automated, and would allow the reduction of sample volume required for analysis to <1 μL.

The sensor array electrodes may be of any configuration, number, and size. In a preferred embodiment, the sensor and reference electrodes would be circular gold pads on the order of 100-500 μM in diameter, separated by a center-to center distance equal to twice their diameter. Each electrode would be addressed by separate electrical interconnects. The application of electrical signals to the sensor electrodes can be accomplished using standard commercially available AC and DC voltage sources. Detection of faradaic electrical signals from the sensor electrodes can be accomplished easily using standard commercially available data acquisition boards mounted within and controlled by a microcomputer. Specifically, the raw sensor current signals would need to be amplified, and then converted to a voltage and analyzed via a high resolution (i.e., 16 bit) analog to digital converter (ADC). It is possible to reduce the signal background and to increase the signal to noise ratio (SNR) by using the common technique of phase-sensitive detection. In this detection method, an alternating current (AC) bias voltage (at a frequency between, for example, 100 to 1000 Hz) is superposed on top of the DC linear voltage ramp applied to the sensor electrodes. The frequency of the applied bias voltage is called the fundamental frequency. It can be shown that the sensor response signal contains multiple frequency components, including the fundamental frequency and its harmonics (integral multiples of the fundamental frequency). It can further be shown that the nth harmonic signal is proportional to the nth derivative of the signal. Detecting these derivative signals (by means of a lock-in amplifier) minimizes the effects of constant or sloping backgrounds, and can enhance sensitivity by increasing the signal to noise ratio and allowing the separation of closely spaced signal peaks. It should be noted that digital, computer-controlled AC and DC voltage sources (i.e., digital to analog converters, DACs), current preamplifiers, analog to digital converters (ADCs), and lock-in amplifiers are all available as integrated signal generation/acquisition boards that can be mounted within and controlled by a single microcomputer.

In a preferred embodiment, an integrated nucleic acid sensor molecule system with electrochemical detection would include the following elements: one, an independently addressable multielement electrode array with immobilized surface layer composed of conductive species separated by insulating species and sensors; two, optical nucleic acid sensor molecules immobilized on the electrode array; three, an oligonucleotide substrate/signaling probe which ligates with the nucleic acid sensor molecule in the presence of the appropriate target; four, an automated or semi-automated microfluidic reagent and sample delivery system; and five, a reader instrument/data acquisition system consisting of a microcomputer controlling the appropriate voltage sources, current and lock-in amplifiers, data acquisition boards, and software interface for instrument control and data collection.

In another embodiment, the change in activity of the nucleic acid sensor molecule can be detected by watching the change in fluorescence of a nucleic acid sensor molecule when it is immobilized on a chip. A ligase can be attached to a chip and its ligase activity monitored. Ligase nucleic acid sensor molecules, labeled with one fluorophore, e.g., Cy3, are attached via an amino modification to an aldehyde chip. The initial Cy3 fluorescence indicates the efficiency of immobilization of the nucleic acid sensor molecules. Next, the chip is exposed to a substrate labeled with a second fluorophore, e.g., Cy5, with or without the target. In the presence of target, the nucleic acid sensor molecule ligates the substrate to itself, and becomes Cy5-labeled. Without target, the ligation does not occur.

The use of a labeled effector oligonucleotide does not change the rate of ligation of the nucleic acid sensor molecule whether target is present or not. When using nucleic acid sensor molecules in the context of a chip based system, in one embodiment, an effector oligonucleotide is used to attach the nucleic acid sensor molecule to the chip.

In another embodiment, a hammerhead nucleic acid sensor molecule could be used to measure the concentration of an analyte through the use of fluorescence.

Any optical method known in the art, in addition to those described above can be used in the detection and/or quantification of all targets of interest in all sensor formats, in both biological and nonbiological media.

Any other detection method can also be used in the detection and/or quantification of targets. For example, radioactive labels could be used, including ³²P, ³³P, ¹⁴C, ³H, or 125I. Also enzymatic labels can be used including horseradish peroxidase or alkaline phosphatase. The detection method could also involve the use of a capture tag for the bound nucleic acid sensor molecule.

6) ADP Nucleic Acid Sensor Molecules

FIG. 3 illustrates an RNA ribozyme library derived from a hammerhead sequence pool consisting of up to 10¹⁶ variants of randomized sequences appended to the hammerhead ribozyme motif. The starting pool of nucleic acids comprising a target modulation domain (TMD), linker domain (LD) and catalytic domain (CD) was prepared on a DNA synthesizer. Random nucleotides are incorporated during the synthesis to generate pools of roughly 10¹⁶ molecules. Randomized stem region scanning library is designed to identify cis-hammerhead NASMs that are modulated by ADP. The linker library was generated by appending an ADP target modulation domain to the randomized linker domain to create a library of potential ADP-modulated cis-hammerhead NASMs. The linker library of ADP-modulated cis-hammerhead NASMs consists of up to 65,000 variants. Most molecules in the randomized NASM pools are non-functional NASMs. In some libraries, the catalytic site is a known sequence (a ligase site or a hammerhead catalytic core) and is at least a portion of either the 5′ and/or 3′ fixed region (the other portion being supplied by the random sequence), or is a complete catalytic site. However, the catalytic site may be selected along with the target molecule binding activity of oligonucleotides within the oligonucleotide pool.

Sorting among the ADP sensors candidates to find the desired molecules starts from the complex sequence pool, whereby desired ADP-modulated sensors are isolated through an iterative in vitro selection process: in addition to the target-activated NASMs that one desires, the starting pool is usually dominated by either constitutively active or completely inactive ribozymes. The selection process removes both types of contaminants. In a following amplification stage, thousands of copies of the surviving sequences are generated to enable the next round of selection. During amplification, random mutations can be introduced into the copied molecules—this ‘genetic noise’ allows functional NASMs to continuously evolve and become even better adapted as target-activated enzymes. The entire experiment reduces the pool complexity from 10¹⁷ molecules down to around 100 ADP sensor candidates that require detailed characterization.

The nucleic acid sensor molecules identified through in vitro selection comprise a catalytic domain (i.e., a signal generating moiety), coupled to a target modulation domain, (i.e., a domain which recognizes ADP and which transduces that molecular recognition event into the generation of a detectable signal). In general, the target modulation domain is defined by the minimum number of nucleotides sufficient to create a three-dimensional structure which recognizes ADP. In addition, the nucleic acid sensor molecules of the present invention use the energy of molecular recognition to modulate the catalytic or conformational properties of the nucleic acid sensor molecule. The selection process as described in detail in the present invention identifies novel nucleic acid sensor molecules through target modulation of the catalytic core of a ribozyme.

The NASM selection procedures place selective pressure on catalytic effectiveness of potential NASMS by modulating both ADP concentration and reaction time-dependence. Either parameter, when optimized throughout the selection, can lead to nucleic acid molecular sensor molecules which have custom-designed catalytic properties, e.g., NASMs that have high switch factors, and or NASMs that have high specificity.

ADP sensor candidates which are derived from in vitro selection are tested as target modulated biosensors. The pool of ADP sensor candidates is cloned into various plasmids transformed into E. coli. Individual ADP sensor encoded DNA clones are isolated, PCR amplified and the ADP sensor candidate is transcribed in vitro to generate ADP sensor RNA. The ADP sensor RNAs are then tested in target modulation assays which determine the rate or extent of ribozyme modulation. For hammerhead ADP sensor RNAs, the extent of target dependent and independent reaction is determined by quantifying the extent of self cleavage of an oligonucleotide substrate in the absence or presence of ADP. The extent of reaction can be followed by electrophoretic separation of the reaction products on a denaturing PAGE gel, and subsequently analyzed by standard radiometric methods.

Individual ADP sensor clones which display high target dependent switch factor values, or high k_{act} rate values are subsequently chosen for further modification and evaluation. Hammerhead derived NASM clones are then further modified to render them suitable for the optical detection applications that are described in detail below. In brief, these ADP sensors are used as fluorescent biosensors affixed to solid supports, as fluorescent biosensors in homogeneous FRET-based assays.

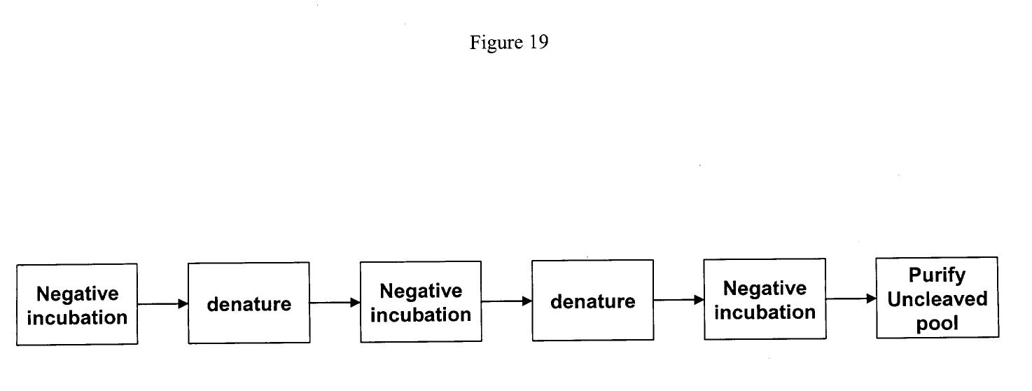

Initial target modulation domains were derived from the minimized ADP aptamer sequence, two pools, designated Pool A and Pool B, were prepared for stem selection. The selection protocol is outlined in FIG. 19. DNA pools were synthesized, purified and transcribed to RNA in preparation for selection round 1. Clones were selected based on the switch factor and were analyzed for the ability to discriminate between ADP and ATP. Representative clones were modified for use in FRET-based assays.

7) Core Uses of ADP NASMS

NASMs have been developed for purposes of target mining and for use in inhibitor studies that demonstrate utility in drug screening and characterization

Target Discovery/Validation by ATPase Mining and Kinase Mining

The ADP nucleic acid compositions according to the invention can be used to detect ADP generation or disappearance associated with a variety of different biological processes, such as various diseases and disorders. In one embodiment, ADP or by inference ATP levels in a cell, tissue or organ sample are associated with a pathological condition, which can be detected using the ADP NASMs of the present invention, and detection of changes in the optical properties of the nucleic acid sensor molecules of the biosensor, and by inference the ADP/ATP level itself provides a means of diagnosing the condition.

Drug Discovery

Generally, methods of drug discovery comprise steps of 1) identifying target(s) molecules associated with a disease; 2) validating target molecules (e.g., mimicking the disease in an animal or cellular model); 3) developing assays to identify lead compounds which affect that target (e.g., such as using libraries to assay the ability of a compound to bind to the target); 4) prioritizing and modifying lead compounds identified through biochemical and cellular testing; 5) testing in animal models; and 6) testing in humans (clinical trials). Through the power of genomics and combinatorial chemistry, large numbers of lead compounds can be identified in high throughput assays (step 3); however, a bottleneck occurs at step 4 because of the lack of efficient ways to prioritize and optimize lead compounds and to identify those which actually offer potential for clinical trials.

The target activatable nucleic acid sensor molecules according to the present invention offer a way to solve this problem by providing reagents which can be used at each step of the drug development process. Most importantly, the nucleic acid compositions according to the present invention offer a way to correlate biochemical data, from in vitro biochemistry and cellular assays, with the effect of a drug on physiological response from a biological assay.

In one embodiment of invention, a method for identifying a drug compound is provided, comprising identifying a profile of ATP consuming-ADP generating biological agents associated with a disease trait in a patient or test sample, administering a candidate compound to the patient, and monitoring changes in activity of the biological agents in the profile. In one embodiment, the ATP consuming-ADP generating biological agents are protein kinases which utilize ATP to phosphorylate partner proteins in a signal transduction cascade, thereby regulating biochemical function of the partner, and the ADP NASM is used to identify inhibitors of kinase activity. In one embodiment of the invention, the ATP consuming-ADP generating biological agents are helicases which utilize ATP to unwind DNA, and the ADP NASM is used to identify inhibitors of helicase activity. In general, it is thought the human proteome is comprised of around two thousand protein kinases and a significantly greater number of proteins with ATPase activity. Hence, in another embodiment of the invention, the ADP NASM is used to identify inhibitors of all enzymes that utilize ATP to generate ADP.

In one embodiment of the present invention, ADP NASM is used to identify, or mine, all proteins in a tissue or patient sample that have ATPase activity or that have kinase activity. Thus, in one embodiment of the invention the ADP NASM is used as a kinase mining (or profiling), or ATPase mining tool. In another embodiment, this kinase or ATPase activity of the monitored profile is compared with a profile of a healthy patient or population of healthy patients, and a compound which generates a profile which is substantially similar to the profile of biological agents in the healthy patient(s) (based on routine statistical testing) is identified as a drug. In a further embodiment, both the profiling and the drug identification step is performed using at least one nucleic acid sensor molecule whose properties change upon binding to a target molecule.

Nucleic acid sensor molecules for Use in Identifying Lead Compounds In one embodiment, the ADP NASM is used to identify the ATP utilizing agent (an ATPase or a protein kinase) as identified as described above, and ATP utilizing agent is provided and are validated by testing against multiple patient samples in vitro to verify that the optical signal generated by these molecules is diagnostic of a particular disease. Validation can also be performed ex vivo, e.g., in cell culture, (using microscope-based detection systems and other optical systems as described in U.S. Pat. No. 5,843,658, U.S. Pat. No. 5,776,782, U.S. Pat. No. 5,648,269, and U.S. Pat. No. 5,585,245) and/or in vivo, for example, by providing a profile biosensor in communication with an optical fiber.

In one embodiment, the same methods which are used to validate the diagnostic value of particular sets of target molecule/nucleic acid sensor molecule combinations are used to identify lead compounds which can function as drugs. Thus, in one embodiment, the effects of a compound on target dependent optical signaling is monitored to identify changes in a signature profile arising as a result of treatment with a candidate compound.

In one embodiment, samples from a treated patient are tested in vitro; however, samples can also be tested ex vivo or in vivo. When the diagnostic profile identified by the biosensor changes from a profile which is a signature of a disease to one which is substantially similar to the signature of a wild type state (e.g., as determined using routine statistical tests), the lead compound is identified as a drug. Target molecules which activate the biosensor can comprise molecules with characterized activity and/or molecules with uncharacterized activity. Because large number of target molecules can be monitored simultaneously, the method provides a way to assess the affects of compounds on multiple drug targets simultaneously, allowing identification of the most sensitive drug targets associated with a particular trait (e.g., a disease or a genetic alteration).

NASMs have been described that directly recognize target proteins of therapeutic interest, such as the MAP kinases. Potentially equally useful are NASMs that recognize the substrates of drug targets. A NASM, based upon the hammerhead, self-cleaving ribozyme, has been developed which emits a fluorescent signal in the presence of ADP, but not ATP or other nucleotides, for assaying ADP-producing enzymes like kinases. Moreover, this sensor has been validated in measurements of ERK kinase activity as a platform for high-throughput screening for small molecule, kinase inhibitors.

The use of the ADP sensor as a target-finding reagent was tested on a prototype “library” of 23 purified proteins. This library consisted of both ATP-dependent and ATP-independent enzymes. The ATP-dependent set further consisted of activated enzymes with robust ATP hydrolysis activities and non-activated enzymes. ATP hydrolysis was performed by incubating triplicate samples of each of these proteins in the presence of ATP. Following quenching of the ATPase reaction, the ADP yield was assayed by measuring the rate of increase in FAM fluorescence upon addition of the ADP NASM.

The ADP NASM is a multi-component sensor consisting of the hammerhead motif, 3′-end-labeled with FAM, hybridized to a 5′-fluorophore-modified “quencher” oligonucleotide. Signaling occurs upon ADP-dependent activation of hammerhead self-cleavage, when the 3′-cleavage product dissociates and diffuses away from the quencher fluorophore. The rate of signal generation over time increases 100-fold in the presence of ADP compared to ATP. However, upon incubation of ATP with phosphorylated ERK, the rate of signal generation is enhanced around 50-fold over background, consistent with the generation of ADP. Similar results are observed with other kinases. Moreover, the signal induced by phosphorylated ERK is concentration-dependent. These results indicate that the ADP NASM faithfully reports the level of ADP due to the presence of an ATP hydrolyzing enzyme.

8) Other Uses of ADP NASMS

The ADP-reactive biological agents are those which consume or generate ADP, where ADP is a starting material, product, or by product of the activity of the biological agent. Examples of such biological agents include kinase, ATPase, and nucleotide triphosphate hydrolases, which generate ADP, or phosphatases, which use ADP as a starting material.

The ADP-recognizing nucleic acid molecules according to the invention can be used to detect ADP generation or disappearance associated with a variety of different biological processes, such as various diseases and disorders. ADP-relevant biological agents include those which are ADP-reactive as well as those which are non-ADP reactive agents. Non-ADP reactive biochemical agents are involved in a biochemical pathway with ADP-reactive biological agents, but do not directly generate or consume ADP. Modulatory compounds are those compounds whose levels, structure, and/or activity can be used to evaluate activity of ADP relevant biological agents.

The activity of such agents can be monitored from any biological fluid, such as bodily fluid, cell culture, and the like. As used herein, “bodily fluid” refers to a mixture of molecules obtained from an organism. This includes, but is not limited to, whole blood, blood plasma, urine, semen, saliva, lymph fluid, meningal fluid, amniotic fluid, glandular fluid, sputum, and cerebrospinal fluid. This also includes experimentally separated fractions of all of the preceding. Bodily fluid also includes solutions or mixtures containing homogenized solid material, such as feces, tissues, and biopsy samples.

In one embodiment, the ADP-relevant agent is associated with a pathological condition and detection of changes in the optical properties of the nucleic acid sensor molecules of the biosensor, or the ADP itself provides a means of diagnosing the condition.

Because signal generation in the NASM biosensor is reversible, washing of the biosensor(s) in a suitable buffer will allow the biosensor(s) to be used multiple times, enhancing the reproducibility of the any diagnostic assay since the same reagents can be used repeatedly. Suitable wash buffers include, e.g., binding buffer without target or, for faster washing, a high salt buffer or other denaturing conditions, followed by re-equilibration with binding buffer.

Re-use of the biosensor is enhanced by selecting optimal fluorophores. For example, Alexa Fluor 488, produced by Molecular Probes, has similar optical characteristics compared to fluorescein, but has a much longer lifetime. Another way to re-use biosensors involves engineering a site recognized by a nuclease proximal to the signal generating site, and sequences comprising signaling moieties are removed from the biosensor and replaced by new sequences, as needed.

In one embodiment of the invention, a method for identifying a drug compound is. provided comprising identifying a profile of ADP-relevant biological agents associated with a disease trait in a patient, administering a candidate compound to the patient, and monitoring changes in activity of the biological agents in the profile.

In another embodiment, the monitored profile is compared with a profile of a healthy patient or population of healthy patients, and a compound which generates a profile which is substantially similar to the profile of biological agents in the healthy patient(s) (based on routine statistical testing) is identified as a drug. In a further embodiment, both the profiling and the drug identification step is performed using at least one nucleic acid sensor molecule whose properties change upon binding to a target molecule.

When the diagnostic profile identified by the biosensor changes from a profile which is a signature of a disease to one which is substantially similar to the signature of a wild type state (e.g., as determined using routine statistical tests), the lead compound is identified as a drug. Target molecules which activate the biosensor can comprise molecules with characterized activity and/or molecules with uncharacterized activity. Samples from a treated patient are tested in vitro, ex vivo, or in vivo. A biosensor to be used ex vivo monitors optical signals in a cell using a microscope based detection system. When an appropriate biosensor is used in vivo to monitor the effects of the compound on the patient, the biosensor is provided in communication with a fiber optic probe inserted into the patient. In another embodiment, an in vivo assay is done by introducing a nucleic acid sensor molecule which retains its catalytic activity into a physiological system (e.g., by injection at a target site in the body, through liposome carriers, and other means of administration routinely used in the art), obtaining cells from the physiological system and detecting the effect of the compound on the catalytic activity of the nucleic acid sensor molecule (e.g., by evaluating the sequence of the nucleic acid sensor molecule) as a means of determining the level, structure, or activity of a drug target, and relating the level, structure, or activity or the target molecules to the efficacy of the drug. The incorporation of biosensors into fiber optic waveguides is known in the art (see, e.g., U.S. Pat. No. 4,577,109; U.S. Pat. No. 5,037,615; U.S. Pat. No. 4,929,561; U.S. Pat. No. 4,822,746; and U.S. Pat. No. 4,762,799). The selection of fluorescent energy transfer molecules for in vivo use is described in EP-A 649848, for example.

A large number of modulatory compounds, both characterized and uncharacterized, can be identified simultaneously using an ADP biosensor. In one embodiment, the modulatory compounds are evaluated in high throughput screening assays, using either solution-based biosensors or substrate-based biosensors, to characterize the biological activity of the modulatory compounds.

For example, in one embodiment, nucleic acid sensor molecules are used to assess levels of ADP appearance or disappearance catalyzed by a particular biological agent, such as an enzyme, in a wild type vs. a disease state in the presence of a known or potential modulatory compound. In this way, components of a pathway that would be affected by a drug acting on that enzyme can be identified. In another embodiment, the levels, structure, and/or activity of all of the modified forms of a modulatory compound, or the active and inactive forms of a biological agent (e.g., a receptor) is determined in a wild type vs. a disease state, to further develop a diagnostic profile of a diagnostic pathway target molecule and to evaluate changes of that profile in the presence of a drug.

In one embodiment, the modulatory compounds are tested in an in vitro biochemical assay to determine compound potency. In this embodiment, a preliminary dosing effect is determined to identify the IC₅₀ of candidate drug. In one embodiment, multiple ADP biosensors are contacted with samples of biological agents from patients exposed to different doses of the candidate drugs identified, to identify candidate drugs with the highest potency (e.g., requiring the least amount of drug to generate a wild type profile or an effective drug profile). Potency can range from picomolar affinity to nanomolar affinity as measured by in vitro IC₅₀ values. The desired selectivity of a drug candidate for its target can vary from 2 to a million-fold, and can be obtained by measuring the potency (IC₅₀) of a drug lead toward the drug target, versus the drug's potency (IC₅₀) values against other pertinent targets (target pertinence is determined by the requirements of the biological system under investigation). A drug lead is deemed optimal when the parameters of potency, selectivity and cellular action are optimized with respect to each other.

In one embodiment, nucleic acid sensor molecules are used in cellular assays where the effect of adding a modulatory compound on cell physiology is known and the researcher wants to determine that the drug is in fact acting on the desired biological agent. Here a candidate drug is added to a physiological system (e.g., cell(s), tissue(s), organ(s), or a patient). Cells from the physiological system are lysed and the ADP is monitored using the nucleic acid sensor molecule either in an ELISA format or other solid support-based format (e.g., a profiling array) or a solution phase format. In another embodiment, cell lysates are contacted with a profiling biosensor specific for a target or pathway of interest to determine the profile of target molecules in the lysed sample. The profile is then compared to the wild type profile and the disease profile to determine if the drug is operating in vivo to restore a cell to its wild type state. Thus, the physiological effect of a candidate drug on a physiological system is correlated with the in vivo mechanism of action of the candidate drug.

In one embodiment, nucleic acid sensor molecules are expressed in vivo or intracellularly using plasmids, viruses or other extra-chromosomal DNA vectors and the cellular nucleic acid sensor molecules are extracted and used to determine the activity of a drug or drug target. These cellular assays can also determine the selectivity of a compound for one target in a pathway relative to other candidate targets in a signal transduction pathway(s) or in another biochemical pathway(s). This data can be used to validate a drug lead or drug target.

Target cells (e.g., tissue(s)) are removed from an animal model of the disease being targeted for treatment and lysed for testing. The lysate is contacted with nucleic acid sensor molecules either in a solid phase assay, a solution phase assay, or in a biosensor array format to assess the in vivo biological activity of a candidate drug identified by any of the previous steps or by some other method, on a target or pathway. Thus, in this embodiment, the physiological effect of a drug on a diseased or normal tissue is correlated with the in vivo mechanism of action of the drug.

In one embodiment, nucleic acid sensor molecules are used in clinical trials to determine the fate of a drug in human or animal models, or used to follow the effect of drug treatment on a target or molecular pathway of choice, as described above. In one embodiment, the nucleic acid sensor molecules, in a solid phase assay (e.g., ELISA format), a solution phase assay, or in a pathway profiling biosensor array format, are used to assess the in vivo biological activity of a drug being tested using lysed cell samples as described above.

The invention is further illustrated in the following non-limiting examples.

### EXAMPLES

### Example 1

RNA Pool Generation and ADP Aptamer Selection

A. JD18.25 RNA Pool Generation

ADP aptamers were derived from a random of pool of RNA aptamers, termed the JD18.25 pool, comprised of RNA molecules of approximately 77 nucleotides in length and having a 5′ oligonucleotide:5′-GGACGGAUCGCGUGAUGA-3′ (SEQ ID NO: 13), a stretch of 40 randomized nucleotides (N₄₀), followed by a 3′ oligonucleotide: 5′-AUCUCACACACC UCCCUGA-3′ (SEQ ID NO: 14). The JD18.25 RNA pool was derived as detailed below.

1. JD18.25 Primer and JD18.25 Pool Preparation

JD18.25A (pool, “DMT-on”) and JD18.25B and JD18.25C (primers, “DMT-off”) were synthesized by solid phase synthesis on an expedite 8909 DNA synthesizer at 1 μmole scale and used for the aptamer pool generation. The JD18.25A template was 94 nucleotides in length, and had a 5′ oligonucleotide :5′-TCAGGGAGGTGTGTGAGAT-3′ (SEQ ID NO: 15), a stretch of 40 randomized nucleotides (N₄₀), followed by a 3′ oligonucleotide: 5′-TCATCACGCGATCCGTCCTATAGTGAGTCGTATTA-3′ (SEQ ID NO: 16) was 94 nucleotides in length including the T7 promoter and was prepared on a 1 μM scale (trityl on). The JD18.25B 5′ primer 5′-TAATACGACTCACTATAGGACGGATCGCGTGATGA-3′; (SEQ ID NO: 17) for PCR was 35 nucleotides in length. The JD18.25C 3′ primer5′-TCAGGGAGGTGTGTGAGAT-3′ (SEQ ID NO: 18) was 19 nucleotides in length. The JD18.25B 5′ primer and JD18.25C 3′ primer were both prepared on a 200 nM scale and had a Tm of approximately 58° C. The JD18.25A pool, as well as the JD18.25B and JD18.25C oligonucleotide primers were deprotected by treatment with 1 ml of concentrated NH₄OH (85° C.; 4 h) and then purified as follows.

The JD18.25B and JD18.25C primers were first centrifuged for 2 min. in a picofuge to remove CPG beads. The supernatant was aspirated from the pelleted CPG beads and transferred to a 15 ml centrifuge. Supernatant was desalted with 11 ml of butanol by thorough mixing with a vortex mixer and the DNA precipitated by centrifugation in a clinical centrifuge for 15 min. The supernatant was decanted away and the purified oligonucleotide primer was dried under vacuum to remove any residual butanol. The purified primers were resuspended in 400 μl 10 mM Tris-HCl, pH 8 containing 0.1 mM EDTA (TE buffer). The concentration of primer was determined spectrophotometrically at OD₂₆₀.

The JD18.25A pool oligonucleotide was purified using two PolyPac2 columns (1 μmole-scale) and reagents commercially obtained from Glen Research (Sterling, Va., USA). Each PolyPac2 column was prepared for sample loading by washing with 4 ml acetonitrile and then washing with 4 ml 2M TEAA buffer. Five hundred microliters of the deprotected JD18.25A pool oligonucleotide was diluted to 2 ml final sample volume with ddH₂O. Diluted sample was loaded onto a PolyPac2 column. Effluent from the column was reapplied onto the column 3 times. The column was washed with 6 ml 1:10 NH₄OH solution. Material was detritylated with 4 ml 2% TFA slowly. The final 0.5 ml of 2% TFA was pushed through the column after waiting 2 min. and observing puffs of smoke from bottom of the column. The column was then washed with 4 ml ddH₂O, washed with 6 ml 1:10 NH₄OH solution, and then washed again with 4 ml ddH₂O. JD18.25A pool oligonucleotide was eluted from the PolyPac2 column with 3 ml 20% acetonitrile. The JD18.25A pool oligonucleotide was precipitated from the eluate by addition of 300 μl 3M NaOAc, pH=5.1, 12 ml 100% ethanol and incubation over night at −20° C. Oligonucleotide was pelleted from the mixture by centrifugation using a clinical (3300 RPM, 4° C., for 30 min). The supernatant was removed from the oligonucleotide pellet. The oligonucleotide pellet was resuspended in 500 μl TE. The concentration of the oligonucleotide pool was determined spectrophotometrically at OD₂₆₀. The purified pool and primer sizes were verified by 15% TBE-Urea gel.

2. Extendibility Assay

The fraction of the synthetic DNA template active for transcription was estimated using the JD18.25A (Pool) and JD18.25B (5′ Primer) which was kinased with gamma ³²P-ATP in a 10 μl reaction mixture containing: 1 μl 5 μM JD18.25A or JD18.25B; 1 μl 10×T4 PNK Kinase Buffer (NEB, 700 mM Tris, pH 7.6, containing 100 mM MgCl₂ and 5 mM DTT); 1 μl NEB PNK Kinase; 1 μl ³²P-γ-ATP; 6 μl ddH₂O. The reaction mixture was incubated at 37° C. for 25 min. The reactions were then purified over a Princeton Separations Centrasep 5 column by adding 800 μl of ddH₂O to the column, vortexing, and allowing the column to hydrate for 30 min. The column was then centrifuged for 1 min (750×g) in a wash tube and the flow through was discarded. The column was washed a second time by this procedure prior to adding the kinase reaction. Purified 5′-³²P-labeled primer was collected by centrifuging the column containing the kinase reaction mixture for 2 min (750×g ) in an Eppendorf tube.

Purified 5′-³²P-labeled primer was then used to extend the synthetic JD18.25A DNA pool template. Two extension reactions were run as follows: The 50 μl extension reaction contained ±4 μM JD18.25A (Pool), 2 μM JD18.25B (5′ Primer), 1 μl ³²P-ATP Kinased JD18.25B (5′ Primer) Centrasep purified, 0.2 mM dNTPs, 2 mM MgCl₂, 1×Taq Buffer (Invitrogen). Annealing was performed at 95° C. The temperature was lowered to 25° C. prior to the addition of add 1 μl Taq polymerase. The reactions were incubated at 75° C. for 20 min. One extension reaction contained the JD18.25A template. Another extension reaction was performed without the JD18.25A template and served as a negative experimental control.

The test reactions were run out on a 15% TBE-Urea gel with extension reaction ±4 μM JD18.25A (Pool) and kinased JD18.25A (Pool) and exposed on phosphor-imager screen. 5′-³²P-JD18.25A served as a size marker in one lane for the location of fully extended DNA in the +template reaction lane, the reaction—template served as a marker for the primer in the second lane, and the reaction +template was in the third lane.

Fraction of active extendible molecules was determined by dividing intensity of full length band in the +template lane by the total exposure in that lane. The fraction of fully extendible molecules was estimated at ˜26%.

3. Large Scale Transcription of the JD18.25 RNA Pool

The JD18.25 DNA template was transcribed by oligonucleotide-directed transcription under the following reaction conditions in a total volume of 20 ml (divided into 4×5 ml reactions). The transcription reaction mixture contained 1×T7 Buffer (25 mM MgCl₂, 40 mM Tris pH 7.8, 0.01% Triton X-100, 1 mM spermidine), 1×NTPs (5 mM each NTP), 15 mM DTT, 0.629 μM JD18.25A (Pool; 2×10¹⁵ fully extendible DNA template molecules), and 1.26 μM JD18.25B (5′ Primer). Large scale transcription of the JD18.25 DNA template was conducted as by adding T7 Buffer, NTPs, DTT and ddH₂O into reaction tubes. The mixture was prewarmed at 37° C. for 15 min prior to the addition of the JD18.25A (Pool) and JD18.25B (5′ Primer) which were then annealed at 85° C. for 3 min. The annealing was terminated by cooling the mixture on ice for 2 min. The JD18.25A (Pool) and JD18.25B (5′ Primer) mixture was then added to the appropriate reaction tubes followed by the addition of 900 μl of T7 Polymerase (JD Prep). The samples were then split into 4×5 ml aliquots in 15 ml conical tubes and incubated overnight in 37° C. water bath. The reactions were terminated with the addition of 1/10 volume of 500 mM EDTA (500 μl per tube). Each sample was then split into 2×50 ml conical tubes and 3 volumes of 100% ethanol was added. Samples were centrifuged in clinical centrifuge for 30 min (4° C.; 3000 RPM) for 30 min. Ethanol was removed from the pellet and the pellets were resuspended in 2 ml TE buffer. Samples were split into 2×1 ml aliquots prior to use. The transcription was verified by examination of the UV shadow of gel electrophoresed test sample (10 μl; 10% TBE-Urea gel).

4. DNAse Treatment of the Transcribed JD18.25 RNA Pool

DNA was removed from the transcribed JD18.25 RNA pool by enzyme treatment with RNAse-free DNAse I prior to in vitro selection of the ADP aptamers. Each 1 ml aliquot of JD18.25 RNA pool sample was enzymatically treated by adding 110 μl 10× Promega DNAse Buffer, 100 μl RNAse-free DNAse I, and 40 μl ddH₂O followed by incubation for 1.5 h. at 37° C. Reactions were then extracted with phenol and chloroform to remove the DNAse by splitting the 2.2 ml of sample into 4×550 μl aliquots, adding 550 μl phenol to each aliquot and vortexing each thoroughly. The aqueous and organic phases were separated by centrifuging the samples in microcentrifuge at maximum speed for 2 min. The top layer containing the RNA was transferred to a fresh tube and re-extracted with phenol and chloroform as before to yield the extracted, transcribed JD18.25 RNA pool.

The extracted, transcribed JD18.25 RNA pool was split into 4×300 μl aliquots. These samples were then ethanol precipitated by addition of 3 volumes of −20° C. ethanol (900 μl to each) and vortexing well. Precipitated RNA was pelleted by centrifugation in a microcentrifuge at maximum speed for 20 min (4° C.). The ethanol was removed and each pellet resuspended in 250 μl TE and 250 μl 2× Loading Dye (no xylene cyanol).

Samples were purified by gel electrophoresis (10% acrylamide gel, 1.5 mM, single comb; run at 25W for 2 h. RNA bands were cut from the gel (travels about the same distance as xylene cyanol). The RNA-containing gel sections were then crushed by passing them through a 20 ml syringe into a 50 ml conical tube. The RNA eluted from the gel fragments into 10 ml TE buffer containing 25 mM EDTA by rotating the tube overnight. After incubation, the gel suspension was passed through a 0.2 μM filter and the filtrate retained. The tube was then rinsed with 15 ml TE which was similarly filtered and then combined with the initial filtrate. The combined filtrates were split into 2×50 ml conical tubes (10 ml/tube). The RNA was precipitated with the addition of 1/3 volume 3 M NaOAc (3.33 ml/tube) and 3 volumes of ethanol (30 ml/tube). The mixture was thoroughly vortexed and the RNA precipitated by incubation at −80 ° C for 1 h.

The precipitated RNA was collected by centrifuging the sample in a clinical centrifuge for 1 h (3000 RPM, 4° C.). The supernatant was aspirated from the pellet, the pellets vacuum dried and then resuspended in 500 μl TE and 500 μl ddH₂O. The concentration of the RNA pool was quantified spectrophotometrically by OD₂₆₀ (E₂₆₀ of the RNA pool=750.9/mM(cm)).

The removal of the original DNA from the transcribed JD18.25 RNA pool was confirmed by PCR. The DNAse treatment removed essentially all the original DNA present in the transcribed JD18.25 RNA pool because subsequent PCR products were only observed in the presence of both reverse transcriptase and DNA polymerase but not DNA polymerase alone.

5. Data Summary for the JD18.25 RNA Pool Generation Procedures

Of the 1.21×10²⁴ (4⁴⁰) DNA molecules possible, 2.67×10¹⁶ DNA molecules were synthesized in the JD18.25A DNA pool. Twenty-six percent (7.06×10¹⁵ DNA molecules) of the DNA molecules synthesized in the JD18.25A DNA pool were active as judged by the extendability assay. That is, 2.00×10¹⁵ DNA molecules present in the JD18.25A DNA pool were active as template for transcription. Reverse transcription of the JD18.25A DNA pool and subsequent purification yielded approximately 3.50×10¹⁶ RNA molecules in the JD18.25 RNA pool. The efficiency of transcription of the JD18.25A pool was approximately 17.5 RNA copies transcribed per JD18.25A DNA template.

B. In vitro Selection of ADP Aptamers

The ADP aptamers were selected from the JD 18.25 aptamer pool using repeated rounds of an affinity column-based selection procedure as detailed below.

1. Affinity Column-Based Selection Procedures

ADP aptamer selection was conducted over 16 rounds of affinity column-based selection. Modifications of made over the course of in vitro selection are summarized in Table 1.

TABLE 1
Modifications in ADP Selection

| | | | ADP | ADP | | |
|---|---|---|---|---|---|---|
| | Buffer | ATP | elutions | elutions | Agarose | ATP |
| Round | washes | washes | done | kept | Precol | Precol |
| 1 | 12 | 0 | 4 | 4 | Y | N |
| 2 | 12 | 0 | 4 | 4 | Y | N |
| 3 | 12 | 0 | 4 | 4 | Y | N |
| 4 | 18 | 0 | 4 | 4 | Y | N |
| 5 | 18 | 0 | 4 | 8 | Y | N |
| 6 | 18 | 10 | 4 | 4 | Y | N |
| 7 | 15 | 15 | 4 | 4 | Y | N |
| 8 | 15 | 20 | 4 | 4 | Y | N |
| 9 | 15 | 19 | 4 | 7 | Y | N |
| 10 | 15 | 10 | 4 | 6 | N | Y |
| 11 | 15 | 15 | 4 | 8 | N | Y |
| 12 | 15 | 15 | 5 | 8 | N | Y |
| 13 | 15 | 15 | 5 | 8 | N | Y |
| 14 | 0 | 15 | 4 | 8 | N | Y |
| 15 | 0 | 20 | 4 | 8 | N | Y |
| 16 | 0 | 20 | 4 | 8 | N | Y |

The general selection buffer used for in vitro selection of the ADP aptamers was 50 mM Hepes, pH 7.4, containing 25 mM MgCl₂ and 150 mM NaCl. Washes which contain ATP used in later rounds of selection) utilized selection buffer supplemented with 4 mM ATP. In turn, selection buffer supplemented with 4 mM ADP was used as an ADP specific elution solution. The affinity column was pre-equilibrated in selection buffer containing 10 μg/ml t-RNA. A small amount (˜1 pmole) of JD18.25 RNA pool was reverse transcribed, PCR amplified and transcribed in the presence of a³²P-UTP to produce radiolabeled RNA to follow the first round of selection. In all subsequent rounds PCR products were transcribed in the presence of α³²P-UTP for the same purpose.

In round 1 of the selection procedure 4×10¹⁵ molecules of gel purified JD18.25 RNA pool and 20 μl of a³²P-UTP labeled RNA from transcription of RT-PCR of RNA pool were diluted to 500 μl final reaction volume with selection buffer and incubated for 10 min to allow the RNA to fold. In subsequent selection rounds, approximately 4×10¹⁴ molecules of α³²P-UTP labeled RNA were used to monitor the selection process. In the round 2 of selection the sample was diluted to 300 μl final reaction volume with selection buffer. In all subsequent selection rounds, the sample was diluted to 200 μl final reaction volume with selection buffer.

2. Affinity Columns

C-8 linked ADP Agarose

RNA aptamer selection was carried out using C-8 linked ADP agarose purchased from Sigma (St Louis, Mo., USA). The concentration of ADP in the resin was ˜1.6 mM. In round 1, 400 μl resin, or 800 μl of a 50% slurry of the resin or 4×10¹⁷ molecules of ADP were used. In subsequent rounds, 200 μl, or 400 μl of a 50% slurry of the resin or 2×10¹⁷ molecules of ADP were used. The resin was hydrated in selection buffer for 30 min before use. Resin was then transferred to a disposable 5 ml column and equilibrated with selection buffer plus tRNA. In round 1, the affinity column was equilibrated with 10 ml selection buffer supplemented with 10 μg/ml tRNA. In subsequent selection rounds the affinity column was equilibrated with 5 ml selection buffer supplemented with 10 μg/ml tRNA.

Adipic Acid Dihydrazide Agarose

An adipic acid dihydrazide agarose pre-column was used in selection to prevent matrix binders. Pre-columns were equilibrated with selection buffer supplemented with 10 μg/ml tRNA exactly as was done for the ADP column. In selection round 1, 600 μl resin or 1.2 ml 50% resin was used. In selection round 2, 300 μl resin or 600 μl 50% resin was used. In selection round 3 through selection round 9, 200 μl resin or 400 μl 50% resin was used.

ATP Precolumn

In later rounds (i.e., 10-16) of ADP aptamer selection, an ATP precolumn (5 mM; Sigma Chemical CO., St. Louis, Mo., USA) was used to increase ADP/ATP discrimination. The affinity column was equilibrated with 5 ml selection buffer supplemented with 10 μg/ml tRNA 30 min before use. In rounds 10 through 16, 200 μl resin, or 400 μl 50% resin was used.

3. In vitro Selection Protocol

FIG. 20 summarizes the selection strategies tested in studies to optimize the ADP aptamer selection protocol. These strategies included washes with selection buffer and then washes with ATP in selection buffer. Washes only with ATP in selection buffer were also tested. The use of an ATP precolumn was tested. Further, start material from the initial elution peak from round 4 was used.

The selection conditions and procedures that yielded the best results were to load the RNA solution onto a pre-column inside of the ADP-affinity column such that the flow through from the pre-column flows directly into the ADP column. In round 1, 500 μl RNA solution was loaded onto the column. In round 2, 300 μl RNA solution was loaded onto the column. In all subsequent selection rounds, 200 μl RNA solution was loaded onto the column.

The flow-through was collected off of ADP column as Wash 1. Sample was incubated on the column for 5 min to allow binding and the columns were then washed with selection buffer (the number of washes in each round varied as detailed in Table 1). After the third wash, the precolumn was removed and the ADP column washed directly. In selection round 1, the wash volume was 600 μl. In selection round 2 the wash volume was 300 μl. In all subsequent selection rounds, the wash volume was 200 μl. Starting with selection round 6, the column was washed with 200 μl of selection buffer containing 4 mM ATP (number of washes in each round varied as detailed in Table 1). Each wash/elution volume was collected in a new tube. The column was then eluted by washing the column with selection buffer containing 4 mM ADP (number of elutions conducted and used varies per round as detailed in Table 2).

The radioactivity in each fraction was measured using a Bioscan instrument. For later rounds, the radioactivity of this starting RNA was quantified using the Bioscan QC4000XER (Bioscan, Inc., Washington, D.C.).

TABLE 2
ADP: Molecules Loaded per Round Calculated by OD260

| | Column | RNA | Fold Excess on |
|---|---|---|---|
| Round | Molecules | Molecules | Column |
| 1 | 4.00E+17 | 4.00E+15 | 100.00 |
| 2 | 2.00E+17 | 7.30E+13 | 2739.73 |
| 3 | 2.00E+17 | 5.78E+13 | 3460.21 |
| 4 | 2.00E+17 | 5.54E+14 | 361.01 |
| 5 | 2.00E+17 | 4.42E+14 | 452.49 |
| 6 | 2.00E+17 | 5.60E+14 | 357.14 |
| 7 | 2.00E+17 | 5.34E+14 | 374.53 |
| 8 | 2.00E+17 | 5.06E+14 | 395.26 |
| 9 | 2.00E+17 | 5.14E+14 | 389.11 |
| 10 | 2.00E+17 | 5.78E+14 | 346.02 |
| 11 | 2.00E+17 | 2.52E+14 | 793.65 |
| 12 | 2.00E+17 | 5.78E+14 | 346.02 |
| 13 | 2.00E+17 | 5.79E+14 | 345.42 |
| 14 | 2.00E+17 | 6.36E+14 | 314.47 |
| 15 | 2.00E+17 | 5.33E+14 | 375.23 |
| 16 | 2.00E+17 | 5.84E+14 | 342.47 |

RNA specifically eluted from the ADP column was ethanol precipitated, reverse transcribed, PCR amplified and ultimately transcribed again into RNA for the next round of selection.

For reverse transcription, the ADP aptamer candidate molecules were reverse transcribed by adding 31 μl of stock 1 solution containing 1 μM JD18.25C (3′ Primer) and 2 mM dNTPs to the pellet from elution ethanol precipitations. The sample was incubated at 65° C. for 2 min and then cooled to 4° C. for 2 min. To this mixture, 19 μl of stock 2 solution (1×1^{st} Strand Buffer (Invitrogen); 10 mM DTT, and 1 μl Superscript II Reverse Transcriptase (Invitrogen)) and the resulting mixture incubated at 42° C. for 1 h.

In order to prevent the appearance of PCR artifacts resulting from over amplification, a small sale PCR was done each round using a small fraction of the reverse transcription reaction. The extent of the test PCR was monitored by running an agarose gel (E-gel from Invitrogen) every few cycles of PCR until a bright band appeared. The full scale PCR was then run for the number of cycles required to observe the desired band in the test reaction plus a few extra cycles to account for less efficient thermocycling in the larger reaction volume. Table 3 summarizes the PCR results from the selection.

TABLE 3
PCR Cycles Per Round

| | Test | Full |
|---|---|---|
| Round | PCR | PCR |
| 1 | 15 | 22 |
| 2 | 15 | 20 |
| 3 | 15 | 22 |
| 4 | 15 | 15 |
| 5 | 15 | 15 |
| 6 | 15 | 21 |
| 7 | 15 | 15 |
| 8 | 13 | 15 |
| 9 | 12 | 15 |
| 10 | 12 | 15 |
| 11 | 15 | 21 |
| 12 | 15 | 20 |
| 13 | 15 | 20 |
| 14 | 15 | 15 |
| 15 | 15 | 20 |
| 16 | 15 | 20 |

To conduct the PCR test, 20 μl of PCR Master Mix (containing 1 μM JD18.25B (5′ Primer), 1 μM JD18.25C (3′ Primer), 0.2 mM dNTPs, 1× PCR Buffer (Invitrogen), 3 mM MgCl₂) was added to 5 μl of reverse transcriptase (RT) reaction. The PCR amplification was conducted in a thermocycler with by cycling at 94° C. (30 s); 58° C. (30 s; number of cycles varied per round as detailed in Table 3); 72° C. (30 s); 72° C. (3 min) and then held at 4° C. until further processing of the sample. The PCR band was then verified on a 2% agarose gel.

In the full-scale PCR amplification, 200 μl PCR Master Mix was added to 45 μl RT reaction. The full-scale PCR amplification was conducted using the same conditions used for the test PCR. The number of cycles varied per round as detailed in Table 3. One half of the PCR reaction was saved and archived. The remainder was used for transcription in preparation for a subsequent round of selection.

One half of the large scale PCR reaction was ethanol precipitated and used as a template for transcription of the next round/pool RNA. Transcription of the pelleted material was initiated with the addition of 100 μl transcription reaction mixture containing 1×T7 Buffer, 1×NTPs, 15 mM DTT, 1 μl α³²P-UTP, and 4.5 μl of T7 Polymerase. The transcription reaction was carried out in a 37° C. water bath overnight (4 h for Round 2) and the transcribed products subsequently collected by ethanol precipitation and gel purification.

4. Studies Monitoring ADP Aptamer Enrichment

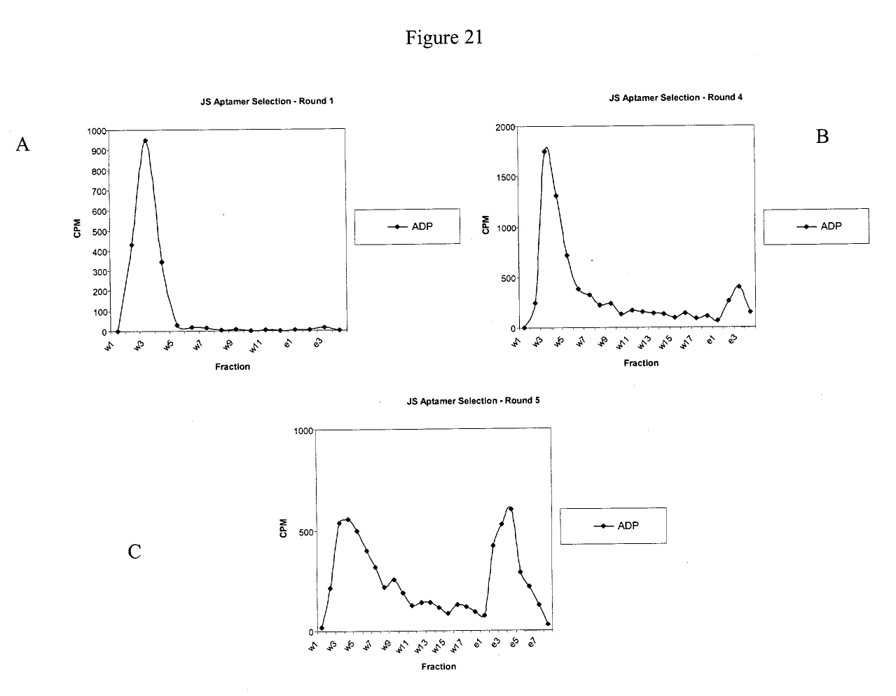

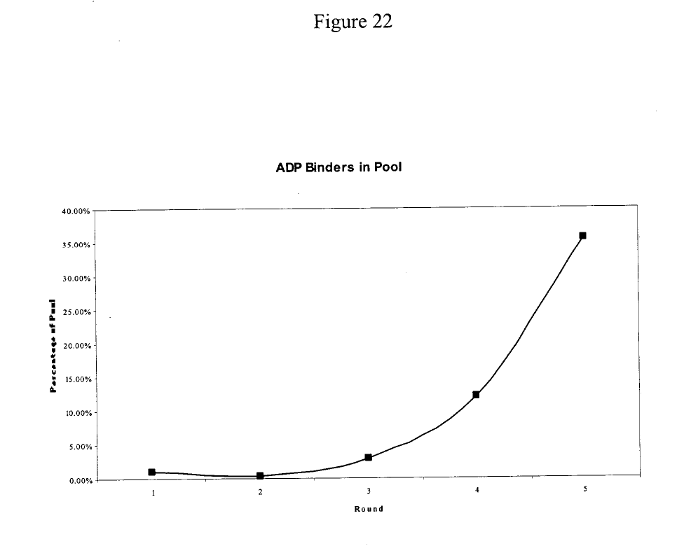

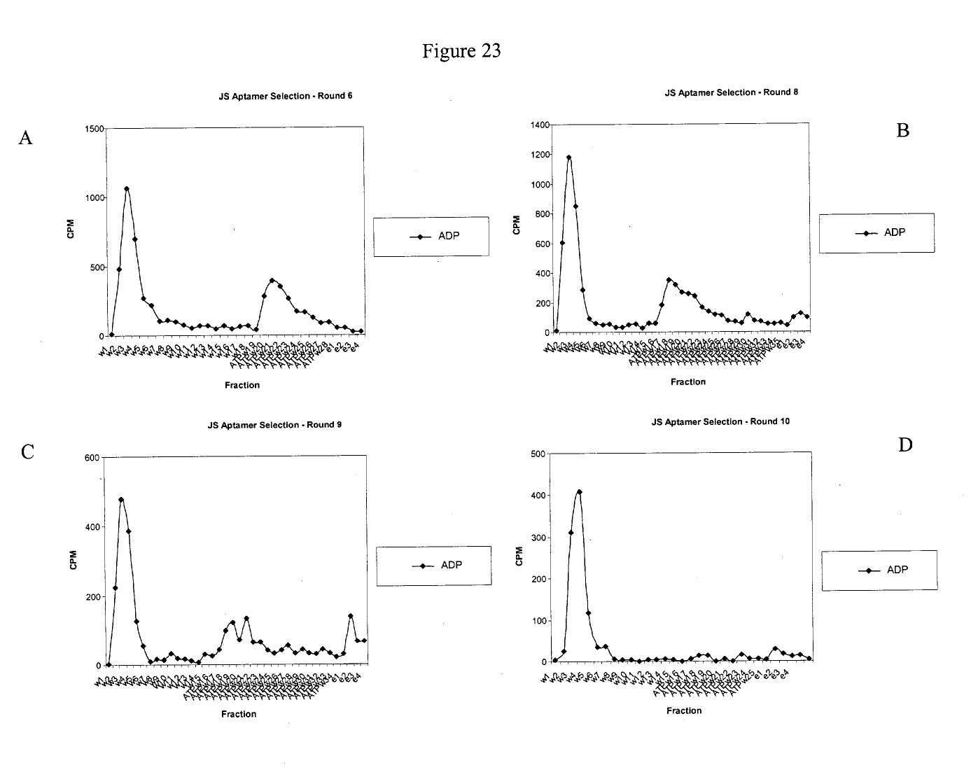

ADP aptamer selection was monitored radiometrically using α³²P-UTP labeled RNA tracer in each selection round. FIG. 21 shows the elution profiles observed in round 1, 4, and 5. The enrichment of RNA retained by the ADP affinity column is indicated by the increasing appearance of a peak corresponding with elution wash 3 (e3) in round 4 and 5 which is not observed in round 1. As shown in FIG. 22, ADP binders are enriched more than 35% at selection round 5.

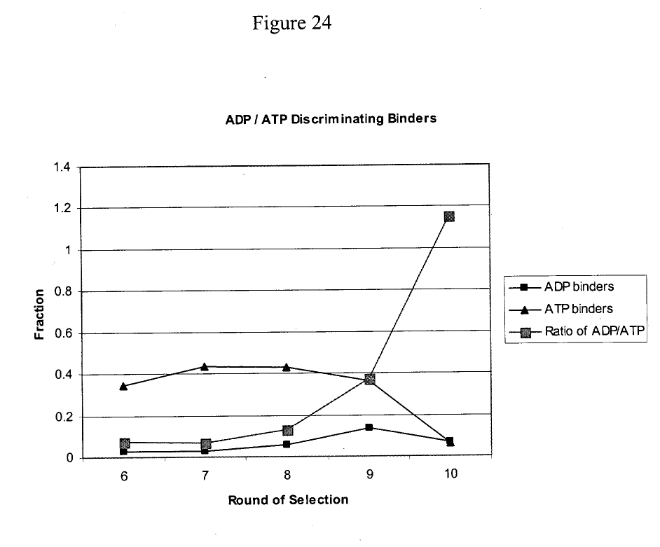

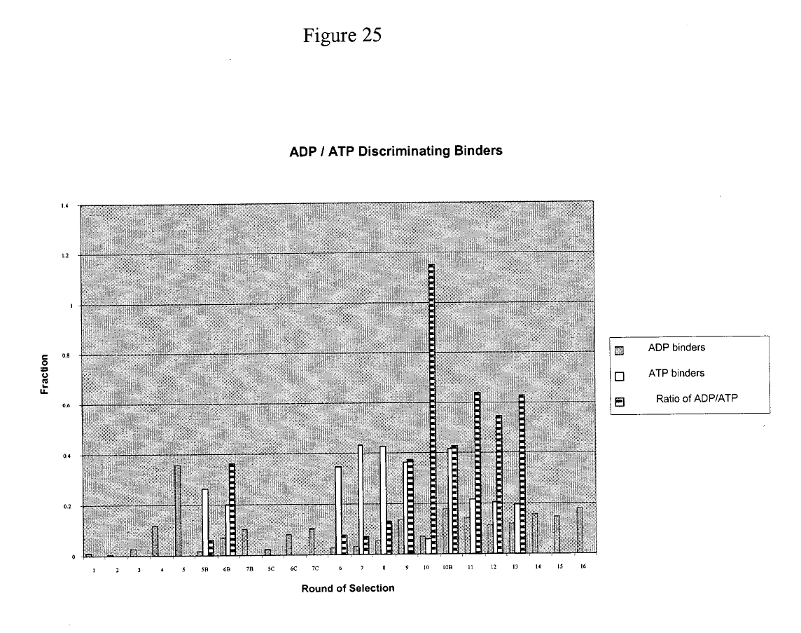

Enrichment of binders selective for ADP over ATP was obtained with the addition of successive ATP washes in rounds 6, 8, and 9 as detailed in FIG. 23 and described above. Coupled with an ATP affinity precolumn in round 10, ATP binders were essentially removed from the ADP aptamer candidate pool (FIG. 23). As detailed in FIG. 24, a 1.2-fold enrichment in the ratio of ADP-to-ATP binders was obtained with the addition of the ATP affinity precolumn prior to the ADP affinity column. As shown in FIG. 25, the increase in the ratio of ADP-to-ATP binders is first observed with the addition of the ATP washing regime in round 6. The ADP-to-ATP ratio is further increased with the addition of the ATP affinity precolumn at round 10. In selection rounds 14 through round 16 no ATP binders were observed in the ADP aptamer candidate pool due to prior rounds of selection. Likewise, in round 5C through 7C where an ATP precolumn was coupled with ATP washes (see, e.g., FIG. 20), no ATP binders were observed in the ADP aptamer candidate pool. As such, material from rounds 6C and 16 were subsequently cloned and further characterized as detailed in Example 3.

### Example 2

Aptamer Characterization and Assays

A. TOPO PCR Cloning of ADP Aptamers from Round 6C and Round 16

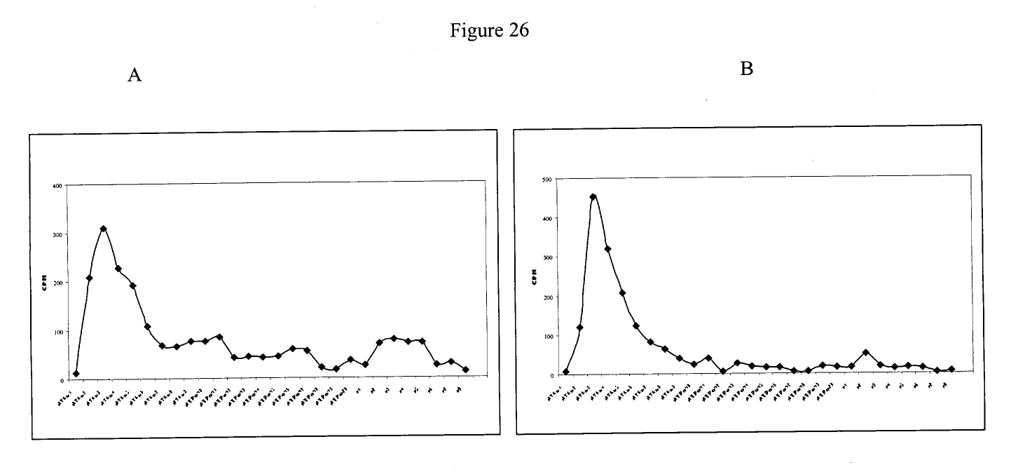

As shown in FIG. 26, Eluates from selection rounds 6C and 16 (FIG. 26) were RTIPCR amplified and cloned and sequenced using the TOPO PCR cloning kit from Invitrogen. Briefly, a 6 μl reaction containing 2 μl PCR reaction (freshly prepared), 1 μl TOPO kit salt solution, 1 μl TOPO vector, and 2 μl ddH₂O. The reaction was mixed by gentle trituration with a pipet and then incubated for 20 min at room temperature. Following incubation 2 μl of this reaction mixture was added to a 50 μl vial of TOPO kit “one shot cells.” The cells and reaction mixture were triturated and then incubated for 10 min on ice. The cells with the reaction mixture were then heated for 30 seconds in a 42° C. water bath. Sample was then immediately transferred to ice and 250 μl SOC medium from TOPO kit was added. The cells were then allowed to recover in a 37° C. water bath for 30 min. One half of the cells containing reaction mixture (150 μl) was plated on LB AMP plates (dried thoroughly in hood). Plates were subsequently sent to Lark Technologies for sequence analysis. Twenty-four of the 25 clones derived from selection round 6C and sent for testing were successfully sequenced and 20 clones had inserts. Forty-three of the 45 clones derived from selection round 16 and sent for testing were successfully sequenced and 33 clones had inserts. A summary of the ADP aptamer sequence data is shown in Table 4.

TABLE 4

ADP Aptamer Sequences Obtained from TOPO Cloning Studies

| | | |
|---|---|---|
| Identifier | Sequence | SEQ ID NO |

| | | |
|---|---|---|
| ARX3P1.C07.6 | GGACGGATCGCGTGATGATACCAGCGATCGCGAGAAGAAAGTAAGAAACGG | 19 |
| | CTGGATCTCACACACCTCCCTGA | |
| ARX3P1.C08.6 | GGACGGATCGCGTGATGATACCAGCGATCGCGAGAAGAAAGTAAGAAACGG | 20 |
| | CTGGATCTCACACACCTCCCTGA | |
| ARX3P1.C09.6 | GGACGGATCGCGTGATGATACCAGCGATCGCGAGAAGAAAGTAAGAAACGG | 21 |
| | CTGGATCTCACACACCTCCCTGA | |
| ARX3P1.D07.6 | GGACGGATCGCGTGATGATACCAGCGATCGCGAGAAGAAAGTAAGAAACGG | 22 |
| | CTGGATCTCACACACCTCCCTGA | |
| ARX3P1.H08.6 | GGACGGATCGCGTGATGATACCAGCGATCGCGAGAAGAAAGTAAGAAACGG | 23 |
| | CTGGATCTCACACACCTCCCTGA | |
| ARX3P1.D08.6 | GGACGGATCGCGTGATGATACCAGCGATCGCGAGAAGAAAGTAAGAAACGG | 24 |
| | CTGGATCTCACACACCTCCCTGA | |
| ARX3P1.E07.6 | GGACGGATCGCGTGATGATACCAGCGATCGCGAGAAGAAAGTAAGAAACGG | 25 |
| | CTGGATCTCACACACCTCCCTGA | |
| ARX3P1.E09.6 | GGACGGATCGCGTGATGATACCAGCGATCGCGAGAAGAAAGTAAGAAACGG | 26 |
| | CTGGATCTCACACACCTCCCTGA | |
| ARX3P1.F09.6 | GGACGGATCGCGTGATGATACCAGCGATCGCGAGAAGAAAGTAAGAAACGG | 27 |
| | CTGGATCTCACACACCTCCCTGA | |
| ARX3P1.G07.6 | GGACGGATCGCGTGATGATACCAGCGATCGCGAGAAGAAAGTAAGAAACGG | 28 |
| | CTGGATCTCACACACCTCCCTGA | |
| ARX3P1.G08.6 | GGACGGATCGCGTGATGATACCAGCGATCGCGAGAAGAAAGTAAGAAACGG | 29 |
| | CTGGATCTCACACACCTCCCTGA | |
| ARX3P1.A07.6 | GGACGGATCGCGTGATGATACCAGCGATCGCGAGAAGAAAGTAAGAAACGG | 30 |
| | CTGGATCTCACACACCTCCCTGA | |
| ARX3P1.F06.6 | GGACGGATCGCGTGATGATACCAGCGATCGCGAGAAGAAAGTAAGAAACGG | 31 |
| | CTGGATCTCACACACCTCCCTGA | |
| ARX3P1.G03 | GGACGGATCGCGTGATGATACCAGCGATCGCGAGAAGAAAGTAAGAAACGG | 32 |
| | CTGGATCTCACACACCTCCCTGA | |
| ARX3P1.H03 | GGACGGATCGCGTGATGATACCAGCGATCGCGAGAAGAAAGTAAGAAACGG | 33 |
| | CTGGATCTCACACACCTCCCTGA | |
| ARX3P1.B05 | GGACGGATCGCGTGATGATACCAGCGATCGCGAGAAGAAAGTAAGAAACGG | 34 |
| | CTGGATCTCACACACCTCCCTGA | |
| ARX3P1.E05 | GGACGGATCGCGTGATGATACCAGCGATCGCGAGAAGAAAGTAAGAAACGG | 35 |
| | CTGGATCTCACACACCTCCCTAA | |
| ARX3P1.F02 | GGACGGATCGCGTGATGATACCAGCGATCGCGAGAAGAAAGTAAGAAACGG | 36 |
| | CTGGATCTCACACACCTGCCCTGA | |
| ARX3P1.D01 | GGACGGATCGCGTGATGATACCAGCGATCGCGAGAAGAAAGTAAGAAACGG | 37 |
| | CTGGATCTCACACACCTCCCAA | |
| ARX3P1.A05 | GGACGGATCGCGTGATGATACCAACGATCGCGAGAAGAAAGTAAGAAACGG | 38 |
| | CTGGATCTCACACACCTCCCTGA | |
| ARX3P1.E01 | GGACGGATCGCGTGATGATACCAGCGATCGCGAGAAGAAAGTAAGAAACGG | 39 |
| | CTGGATCTCACACACCTCCCTGA | |
| ARX3P1.D09.6 | GGACGGATCGCGTGATGACCAGGCAAGCGTGGCCTAGTAATGATCAAAAGG | 40 |
| | ACTCTGATCTCACACACCTCCCTGA | |
| ARX3P1.B07.6 | GGACGGATCGCGTGATGAAGGCCAGCTCTTGGTATCCTAAGCAGAACCAAG | 41 |
| | GTGCGGATCTCACACACCTCCCTGA | |
| ARX3P1.H06.6 | GGACGGATCGCGTGATGAAGGCCAGCTCTTGGTATCCTAAGCAGAACCAAG | 42 |
| | GTGCGGATCTCACACACCTCCCTGA | |
| ARX3P1.B08.6 | GGACGGATCGCGTGATGATGGAGAATAAAAACAACCGGGATATTGCCCCGT | 43 |
| | AAAGTCCATCTCACACACCTCCCTGA | |
| ARX3P1.A08.6 | GGACGGATCGCGTGATGATGGACCAGTTGTCGAGACATCTGGTGGAAGACT | 44 |
| | CTGCATCTCACACACCTCAA | |
| ARX3P1.H07.6 | GGACGGATCGCGTGATGAATGCCAGACCATCAGAAACAGTTTTTTCCCTAA | 45 |
| | ACGAGGCATCTCACACACCTCCCTGA | |
| ARX3P1.E08.6 | GGACGGATCGCGTGATGAGGTTGCAGCAGAGCCGACAACGCGGCTCTGGTG | 46 |
| | GGCATCTCACACACCTCCCTGA | |
| ARX3P1.E06 | GGACGGATCGCGTGATGAGCATAAGGCATAAACCTGTGGATTGTCAATGCG | 47 |
| | CATCATCTCACACACCTCCCTGA | |
| ARX3P1.D05 | GGACGGATCGCGTGATGAAGGGCATGGAAGGTTAAGGAGACCTAAGTGTTC | 48 |
| | ATCTGCATCTCACACACCTCCCTGA | |
| ARX3P1.C05 | GGACGGATCGCGTGATGAAATGTAAACATTGAGCGATGGATAACAAGTTAG | 49 |
| | TTACTATCTCACACACCTCCCTGA | |
| ARX3P1.B01 | GGACGGATCGCGTGATGAAATGTAAACATTGAGCGATGGATAACAAGTTAG | 50 |
| | TTACTATCTCACACACCTCCCTAA | |
| ARX3P1.G04 | GGACGGATCGCGTGATGAGATTAGCGATGCACAAGCAAGACAATAAGACAC | 51 |
| | GGCTAGATCTCACACACCTCCCAA | |
| ARX3P1.B06 | GGACGGATCGCGTGATGAGATTAGCGATGCACAAGCAAGACAATAAGACAC | 52 |
| | GGCTAGATCTCACACACCTCCCTGA | |
| ARX3P1.A02 | GGACGGATCGCGTGATGACTGAGGGGTAATGAACACCCCGGACAATCAGAC | 53 |
| | ACGGTCATCTCACACACCTCCCTGA | |
| ARX3P1.F01 | GGACGGATCGCGTGATGACGAGGGGAATGAACACCCCGGACAATCAGACAC | 54 |
| | GGTCATCTCACACACCTCCCTGA | |
| ARX3P1.G02 | GGACGGATCGCGTGATGACGAGGGGAATGAACACCCCGGACAATCAGACAC | 55 |
| | GGTCATCTCACACACCTCCCTGA | |
| ARX3P1.A03 | GGACGGATCGCGTGATGATAAATCTTTAGCGTGCAGAACGTACAACGAATC | 56 |
| | GGGTCTATCTCACACACCTCCCTGA | |
| ARX3P1.D03 | GGACGGATCGCGTGATGATAAATCTTTAGCGTGCAGAACGTACAACGAATC | 57 |
| | GGGTCTATCTCACACACCTCCCTGA | |
| ARX3P1.G01 | GGACGGATCGCGTGATGATAAATCTTTAGCGTGCAGAACGTACAACGAATC | 58 |
| | GGGTCTATCTCACACACCTCCCTGA | |
| ARX3P1.E02 | GGACGGATCGCGTGATGAGATTTAGCGATGATGCAATGAATGACTGAAGTG | 59 |
| | TCGATCTCACACACCTCCCTGA | |
| ARX3P1.H01 | GGACGGATCGCGTGATGAGATTTAGCGATGATGCAATGGATGACTGAAGTG | 60 |
| | TCGATCTCACACACCTCCCTGA | |
| ARX3P1.H05 | GGACGGATCGCTGTGATGAGATTTAGCGATGATGCAATGAATGACTGAAGT | 61 |
| | GTCGATCTCACACACCTCCCTGA | |
| APX3P1.A04 | GGACGGATCGCGTGATGAGATTTAGCGATGATGCAATGAATGACTGAAGTG | 62 |
| | TCGATCTCACACACCTCCCTGA | |
| ARX3P1.F05 | GGACGGATCGCGTGATGAGATTTAGCGATGATGCAATGAATGACTGAAGTG | 63 |
| | TCGATCTCACACACCTCCCTGA | |
| ARX3P1.B02 | GGACGGATCGCGTGATGAGATTTAGCGATGATGCAATGAATGACTGAAGTG | 64 |
| | TCGATCTCACACACCTCCCTGA | |
| ARX3P1.B03 | GGACGGATCGCGTGATGAGATTTAGCGATGATGCAATGAATGACTGAAGTG | 65 |
| | TCGATCTCACACACCTCCCTGA | |
| ARX3P1.B04 | GGACGGATCGCGTGATGAGATTTAGCGATGATGCAATGAATGACTGAAGTG | 66 |
| | TCGATCTCACACACCTCCCTGA | |
| ARX3P1.C01 | GGACGGATCGCGTGATGAGATTTAGCGATGATGCAATGAATGACTGAAGTG | 67 |
| | TCGATCTCACACACCTCCCTGA | |
| ARX3P1.C02 | GGACGGATCGCGTGATGAGATTTAGCGATGATGCAATGAATGACTGAAGTG | 68 |
| | ACGATCTCACACACCTCCCTGA | |
| ARX3P1.C03 | GGACGGATCGCGTGATGAGATTTAGCGATGATGCAATGAATGACTGAAGTG | 69 |
| | ACGATCTCACACACCTCCCAA | |
| ARX3P1.D06 | GGACGGATCGCGTGATGAGATTTAGCGATGATGCAATGAATGACTGAAGTG | 70 |
| | TCGATCTCACACACCCTCCCTGA | |
| APX3P1.G05 | GGACGGATCGCGTGATNAGATTTANCNTGTGATGCAATGAANGATTAAAGT | 71 |
| | GTNGNTCNNNCANACCTCCCCTGA | |

The ADP aptamer clone identifier and frequency of occurrence is shown in Table 5.

TABLE 5
ADP Aptamer Clone Identifier and Frequency from
Selection Round 6C and 16

| | | Freq obs in | Freq obs in |
|---|---|---|---|
| Clone name | Clone source | R6c | R16 |
| G08.6 | R6C | 13 | 8 |
| D09.6 | R6C | 1 | − |
| B07.6 | R6C | 2 | − |
| B08.6 | R6C | 1 | − |
| A08.6 | R6C | 1 | − |
| H07.6 | R6C | 1 | − |
| E08.6 | R6C | 1 | − |
| E06 | R16 | − | 1 |
| D05 | R16 | − | 1 |
| C05 | R16 | − | 2 |
| G04 | R16 | − | 2 |
| F01 | R16 | − | 3 |
| D03 | R16 | − | 3 |
| D06 | R16 | − | 13 |

Clones were considered to be identical, and thus observed more than once, if they differed by two or fewer nucleotides.

B. SPA Screening of ADP Aptamer Clones

Mini-prepped plasmid DNA and corresponding sequence (Table 4) were obtained from Lark Technologies. Any sequence that differed by more than two nucleotides from all other ADP aptamer clones was considered to be unique for the purposes of screening. “Unique” clones were PCR amplified from purified plasmid DNA using the original 5′-primer used in the selections (JD18.25B; SEQ ID NO: 17) and a new 3′-primer, 5′-CGAAGAAGGGAACAGAACCACGCAAGGTCAGGGAGGTGTGTGAGAT-3′ (JD18.122.A; 200 nM scale, 46-mer) Tm ˜58° C.; SEQ ID NO: 72). This primer adds a 3′-sequence tag to the transcribed RNA molecules which allows the aptamer clones to be immobilized onto the surface of an NEN streptavidin coated flash plate via base pairing with a biotinylated DNA capture oligo (MK08.112A; 5′-biotin-CGAAGAAGGGAACAGAACCACGCAA-3′; SEQ ID NO: 73).

NEN flashplates used in the ADP SPA screen and competition binding studies were prepared by incubating the individual wells with 40 pmol of MK08.112A biotinylated capture probe in 22 μl of PBS Buffer: BupH (0.05% tRNA, 0.025% Tween 20) with and shaking at 650 RPM, 15 min. Excess capture probe was removed from the wells by washing with 1×PBS 3 times and inverting plate with force to remove liquid and blotting them dry on paper towels. Crude transcription of RNA transcribed with capture probe sequence (5 μl) in 25 μl 1×PBS was incubated in designated wells with shaking at 650 RPM for 30 min. Excess aptamer was removed by washing with 1×PBS 3 times and blotting as before. Treated wells were incubated with 30 μl of 1 μM ³H-ADP in 1× Selection Buffer for 30 s with shaking at 650 RPM and then assessed for ADP-mediated signal by quantification on a TopCount, ³H SPA scintillation counter.

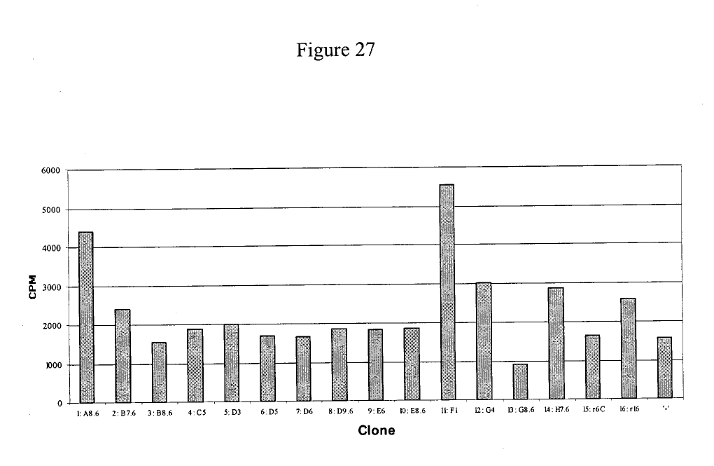

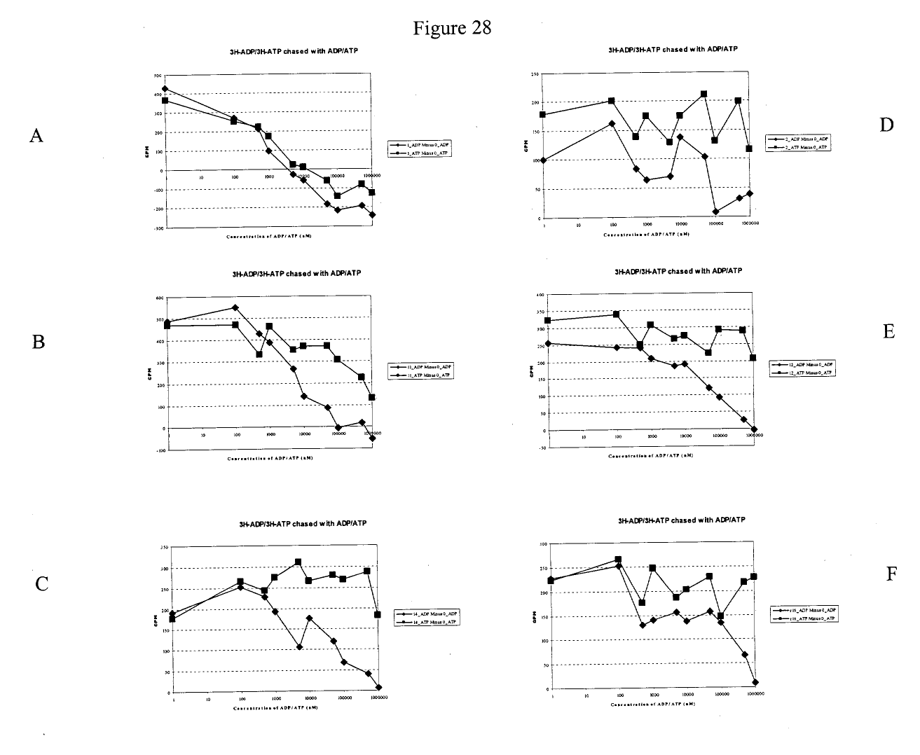

As shown in FIG. 18, surface immobilized aptamer RNA that binds to ³H-ADP will concentrate the tritiated nucleotide on the surface of the flash plate and generate a scintillation proximity signal detectable in a Topcount instrument. Clones were initially assessed for the ability to yield ADP-mediated signal in the ADP SPA. As shown in FIG. 27, ADP aptamer RNA from select ADP aptamer clones bound ³H-ADP, thus yielding an ADP-mediated signal in the SPA screening assay of more than 2.5-fold above background signal (Cf. clone 11:F1 vs. clone R0).

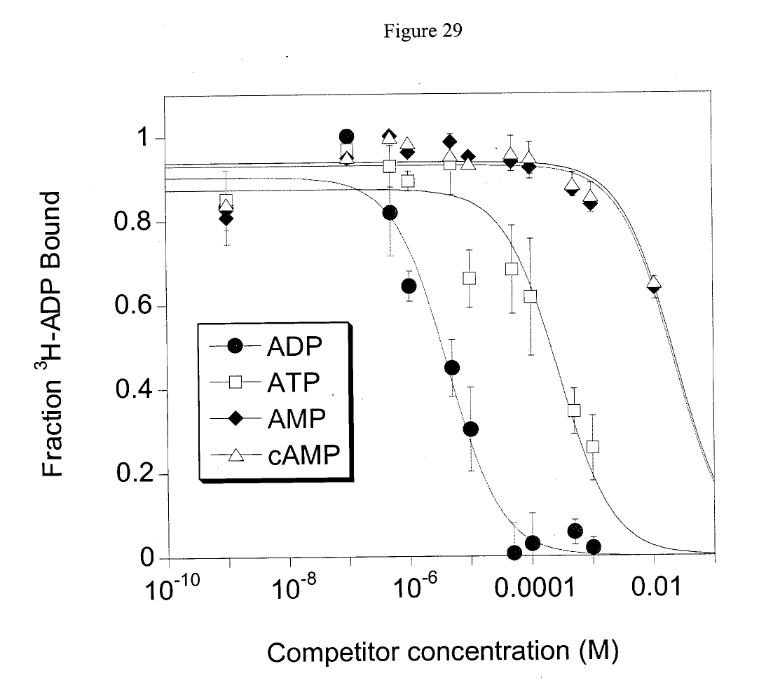

The best ADP binders, e.g., clones 1, 2, 11, 12, 14, and 16 were subsequently tested for ADP/ATP discrimination by competition with either cold ADP or ATP (FIG. 28). Clones such as F01 (clone #11), whose SPA signal was competed off with the lowest concentration of ADP, i.e., the best ADP binder, and correspondingly the highest concentration of ATP, i.e., the worst ATP binder, were considered to be ADP selective. The more detailed competitive binding analysis of ADP aptamer clone F01 shown in FIG. 29 revealed that this aptamer is highly selective for ADP compared to other adenosine nucleoside derivatives, e.g., AMP, ATP, and cAMP.

i) Selectivity of ADP Aptamer F01: SPA KD Determination for Nucleoside Analogs and Mimetics

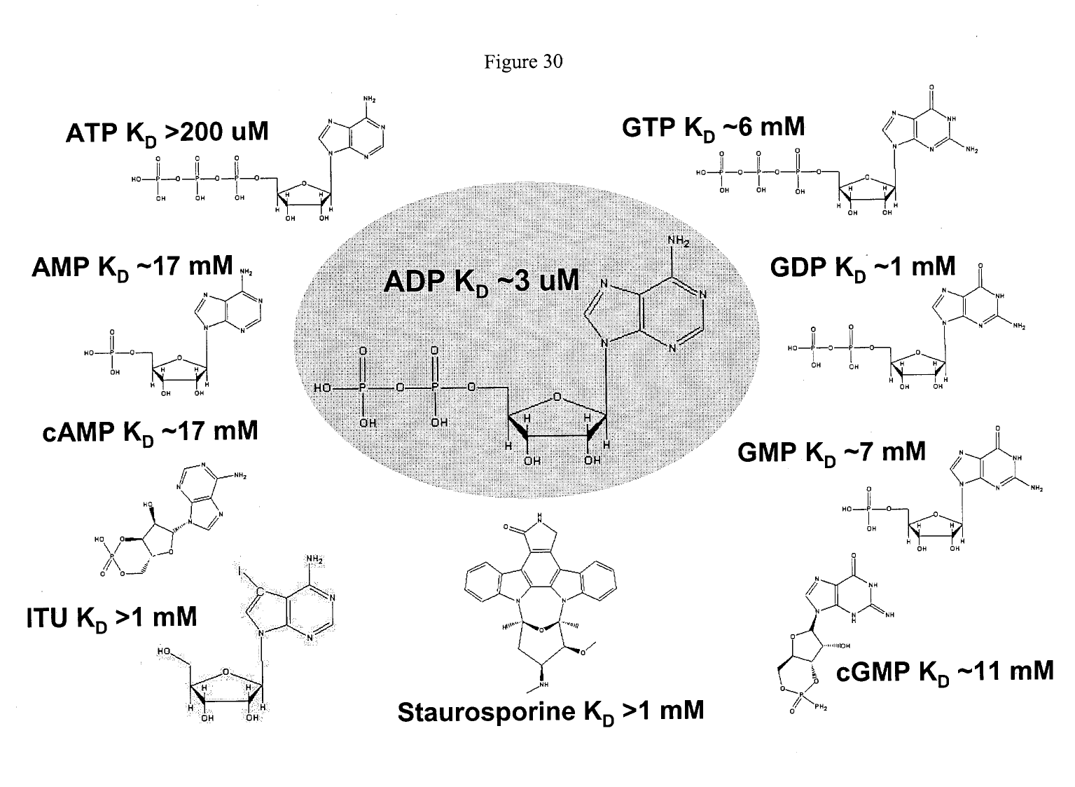

The specificity of the ADP aptamer clone F01 was characterized using a variety of nucleoside analogues and mimetics as shown in FIG. 30. NEN flashplates used in the ADP assay were prepared as described above using 150 pmol ADP clone F01 ( in 20 μl 1×PBS incubated in designated wells with shaking at 650 RPM for 30 min). Excess F01 aptamer was removed by washing with 1×PBS 3-times and blotting as before. Treated wells were incubated with 30 μl of 1 μM ³H-ADP in 1× Selection Buffer for 30 s with shaking at 650 RPM and then assessed for ADP-mediated signal by quantification on a TopCount, ³H SPA scintillation counter. Thereafter, 1 μl of each test compound was added to the appropriate wells and the plate shaken at 650 RPM for 30 s prior to recounting on TopCount, ³H SPA scintillation counter. Titration of each test compound was continued with subsequent 1 μl additions up to 10 mM final concentration. The dissociation constant K_{D} was estimated from the IC₅₀ of the competitive binding curve.

As shown in FIG. 29, ADP aptamer clone F01 showed a high degree of specificity for ADP. The K_{D} for ATP was at least 70-fold higher that that observed for ADP. The other nucleoside derivatives and mimetics tested, e.g., AMP, cAMP, ITU, GTP, GDP, GMP, cGMP, and staurosporine, tested had K_{D} values that exceeded the K_{D} value for ADP by more than 300-fold (FIG. 30).

ii) Effect of ATP Purity on ADP Aptamer Selectivity

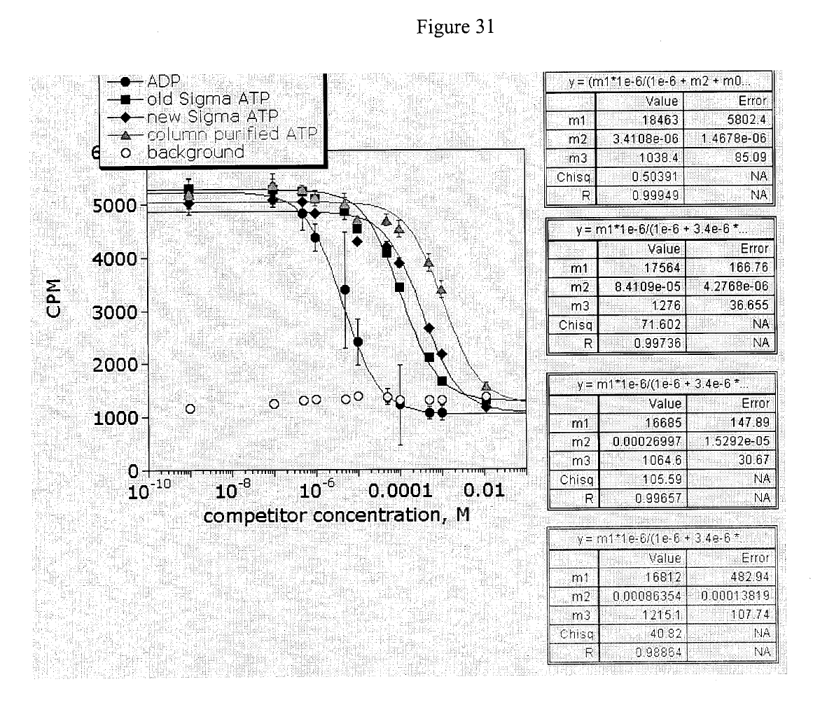

HPLC analysis of commercially obtained ATP preparations revealed 1-2% contamination with ADP. This impurity confounds precise determination of ADP aptamer selectivity by SPA competition. While the initial indicated K_{D} for ATP was >200 μM (FIG. 30), column purification of ATP and reassay with directly biotinylated F01 aptamer revealed the K_{D} for ATP to be approximately 860 μM (FIG. 31). That is, the ADP aptamer is approximately 4-fold more selective for ADP over ATP than preliminary studies indicated.

iii) ADP Aptamer F01-based Kinase SPA

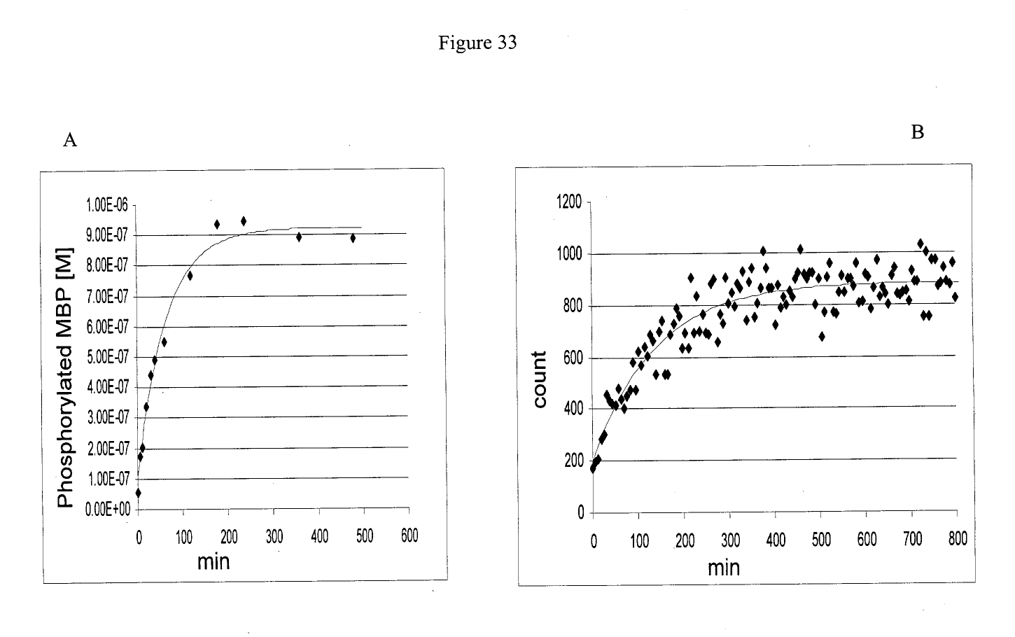

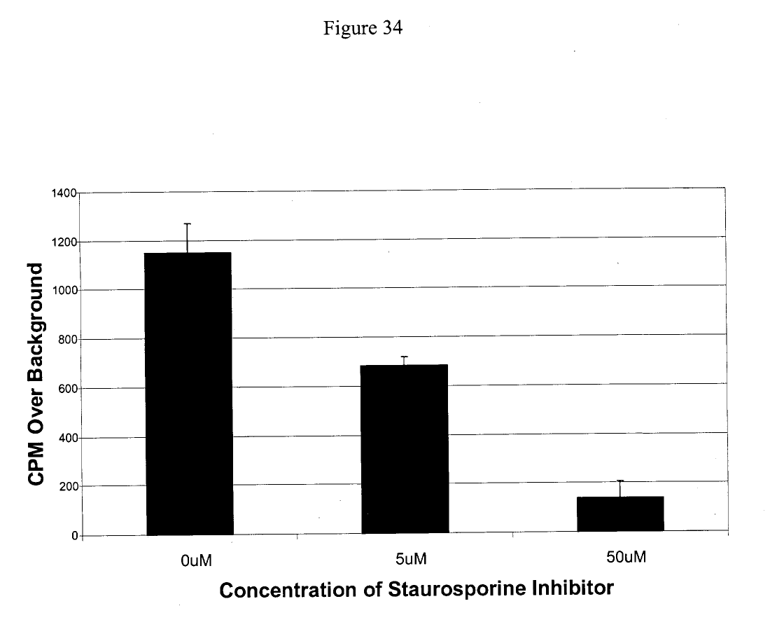

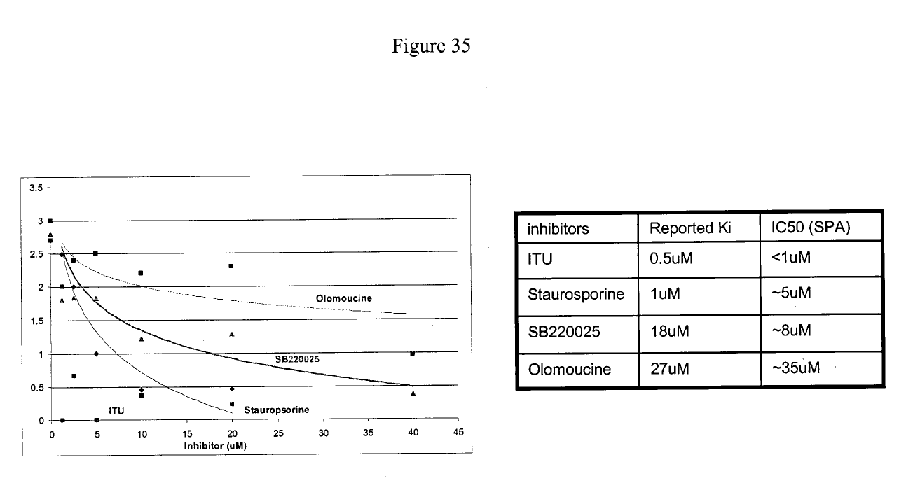

As shown in FIG. 32, surface immobilized aptamer RNA that binds to ³H-ADP may be utilized to measure kinase-mediated protein phosphorylation. An ADP aptamer will concentrate the tritiated ADP released by kinase on the surface of the flash plate and generate a scintillation proximity signal detectable in a Topcount instrument. FIG. 33B shows the use of the ADP F01 aptamer to detect ppERK-mediated phosphorylation of myelin basic protein (MBP) in an ADP SPA. The time-dependent phosphorylation of ppERK determined by direct quantification of ³²P-labeled MBP in a radiometric assay (FIG. 33A) correlated well (within a factor of two) with the time-dependent increase in assay signal observed by ADP SPA incorporating the ADP F01 aptamer. Furthermore, as shown in FIG. 34, concentration-dependent inhibition of ppERK by staurosporine was observed using the ADP SPA incorporating the ADP F01 aptamer, with assayed with ³H-ATP and MBP. Similarly, the ADP F01 aptamer detected the concentration-dependent inhibition of ppERK by the kinase inhibitors, ITU, SB220025, and olomoucine as well (FIG. 35).

A 96-well high throughput screening (HTS) ppERK kinase assay was constructed using the ADP F01 aptamer in an SPA. This assay was performed with 40 nM ppERK, 1.35 μM ³H-ATP, and 10 μM MBP and displayed reproducible kinetics and a signal more that 5-fold over background S/B=5.46, as shown in FIG. 36B (Z′ factor=0.79, Signal/Noise=80.2). Measurements could be obtained within 10 min with approximately 5% deviation between probe preparations. FIG. 36A shows the time course of ADP generation. This HTS assay was used to assess the relative inhibitory activity of 100 test compounds at 10 μM concentration (FIG. 37). This HTS assay was performed three times (FIGS. 37A, 37B and 37C). As shown in FIG. 37A, test compounds showed a spectrum of inhibitory activity and the known inhibitor kinase inhibitors among the test panel, e.g., ITU and staurosporine, significantly decreased the reaction rate observed in the HTS ppERK assay. As seen in FIG. 37B (ADP-HH screening), test compounds showed a spectrum of inhibitory activity and the known inhibitor kinase inhibitors among the test panel, e.g., staurosporine, significantly decreased the reaction rate observed in the HTS ppERK assay. As seen in FIG. 37C (ADP-SPA screening (2^{nd})), test compounds showed a spectrum of inhibitory activity and the known inhibitor kinase inhibitors among the test panel, e.g., staurosporine, significantly decreased the reaction rate observed in the HTS ppERK assay.

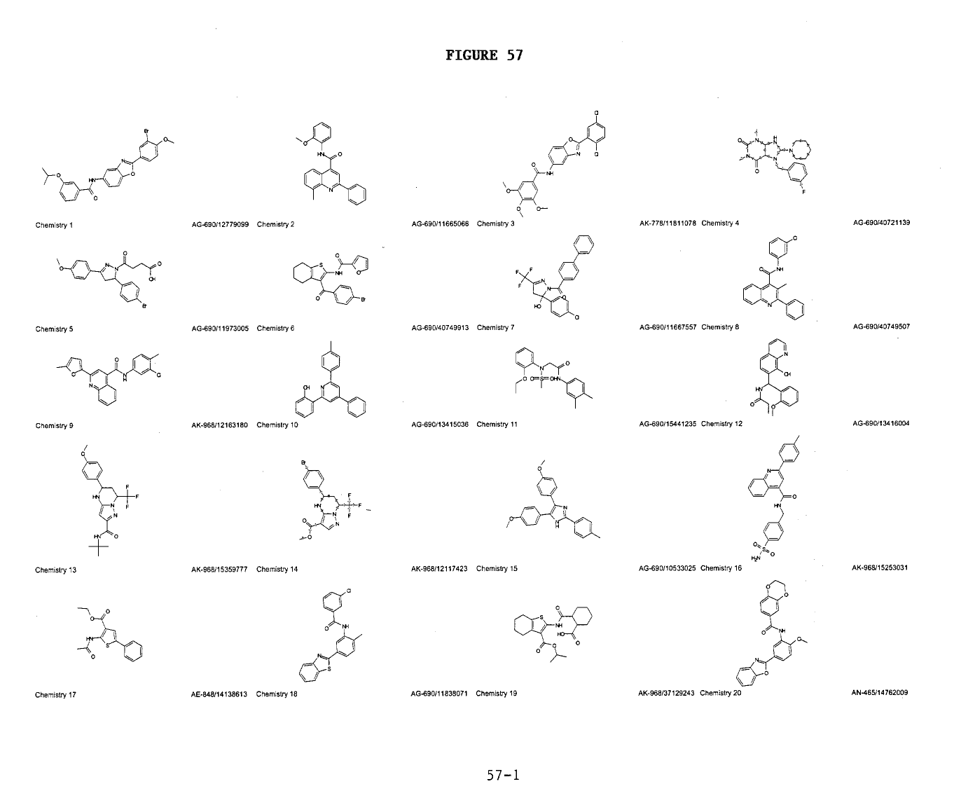

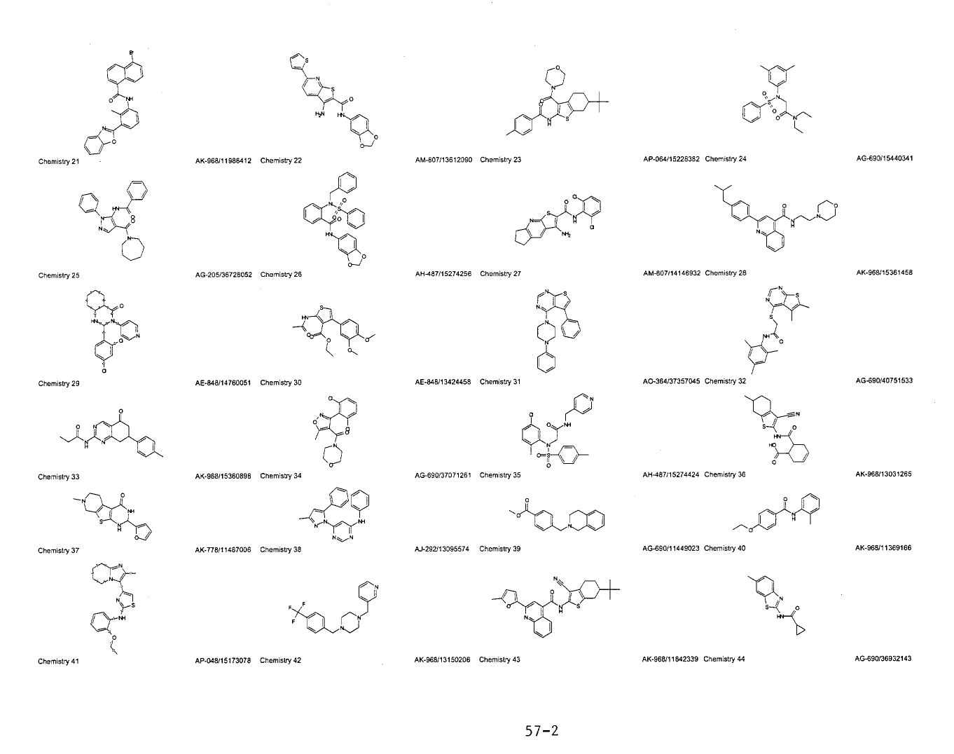

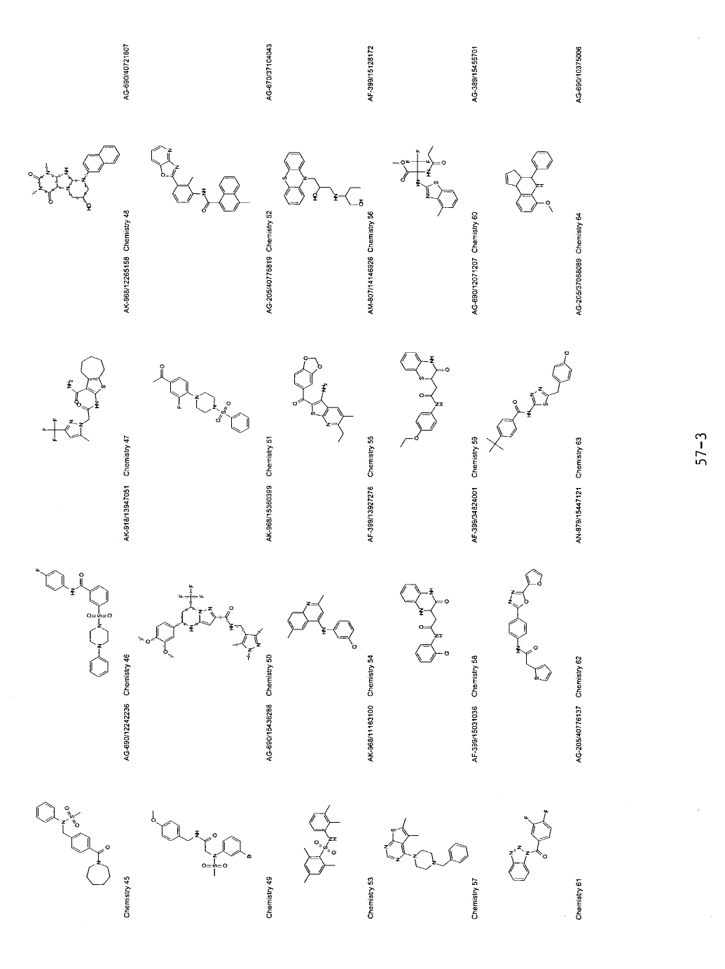

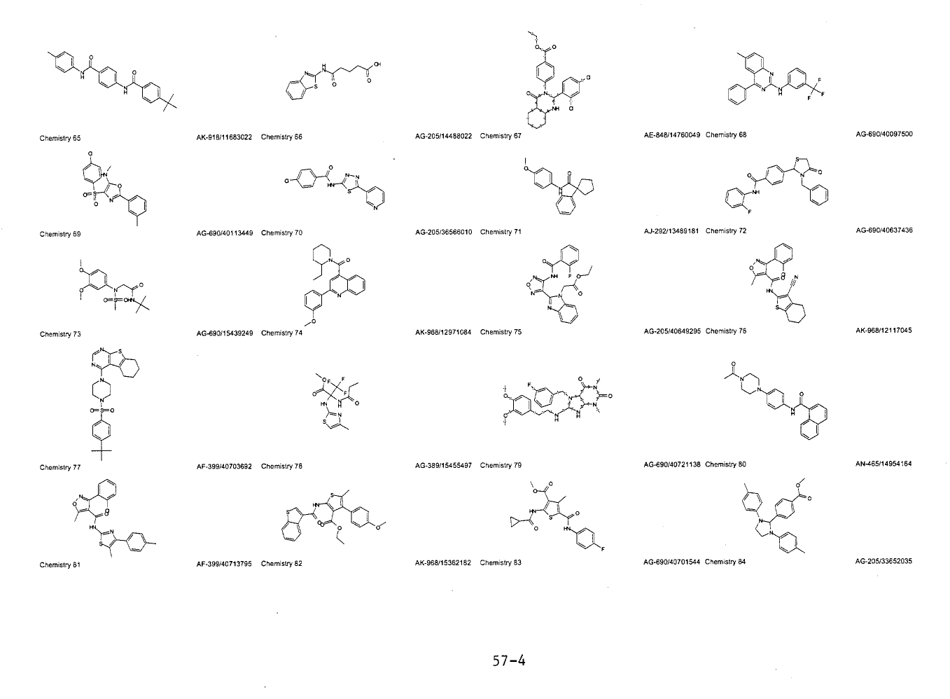

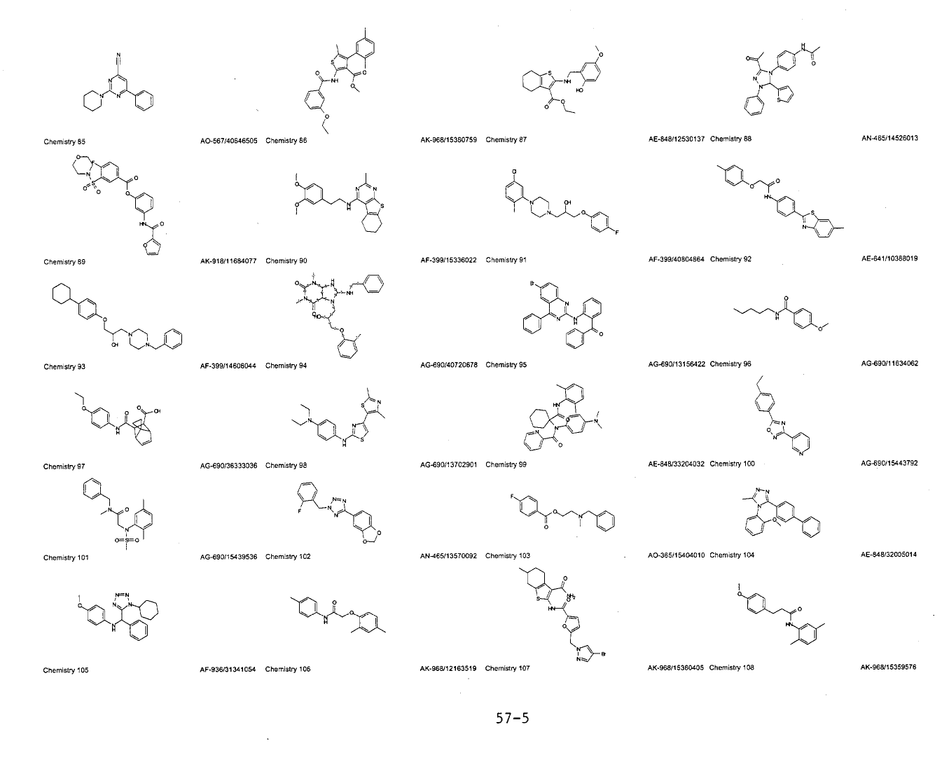

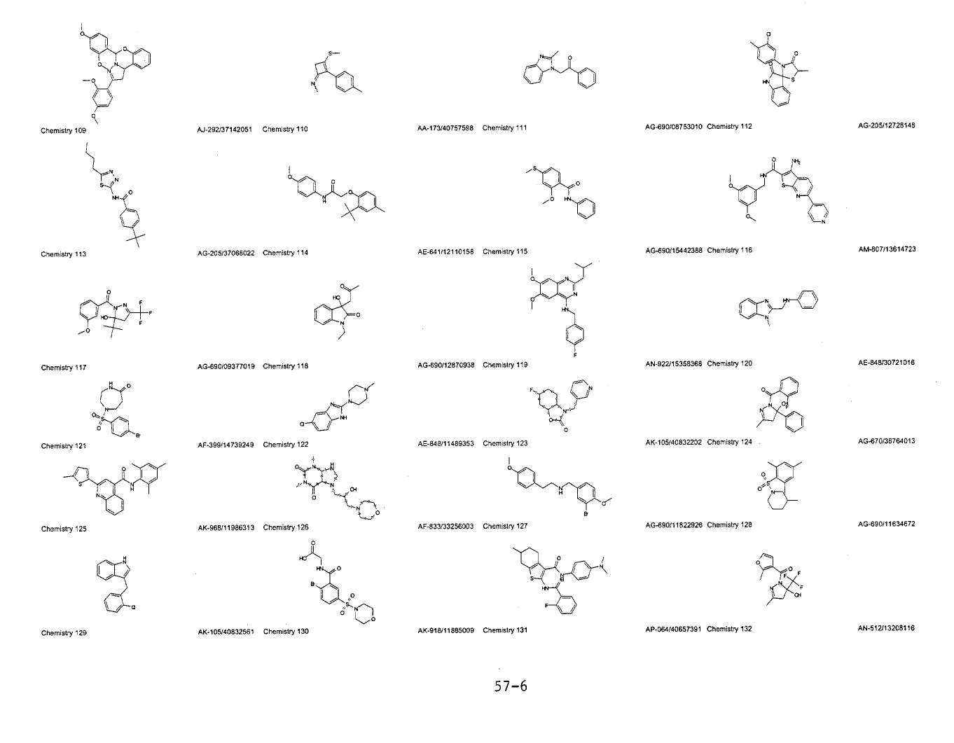

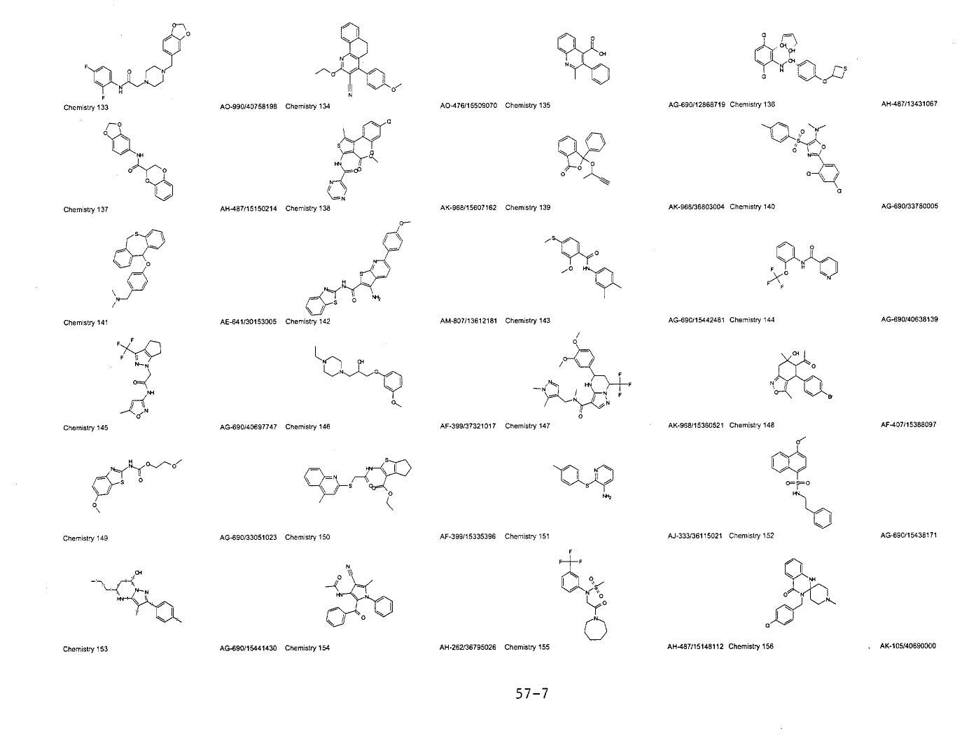

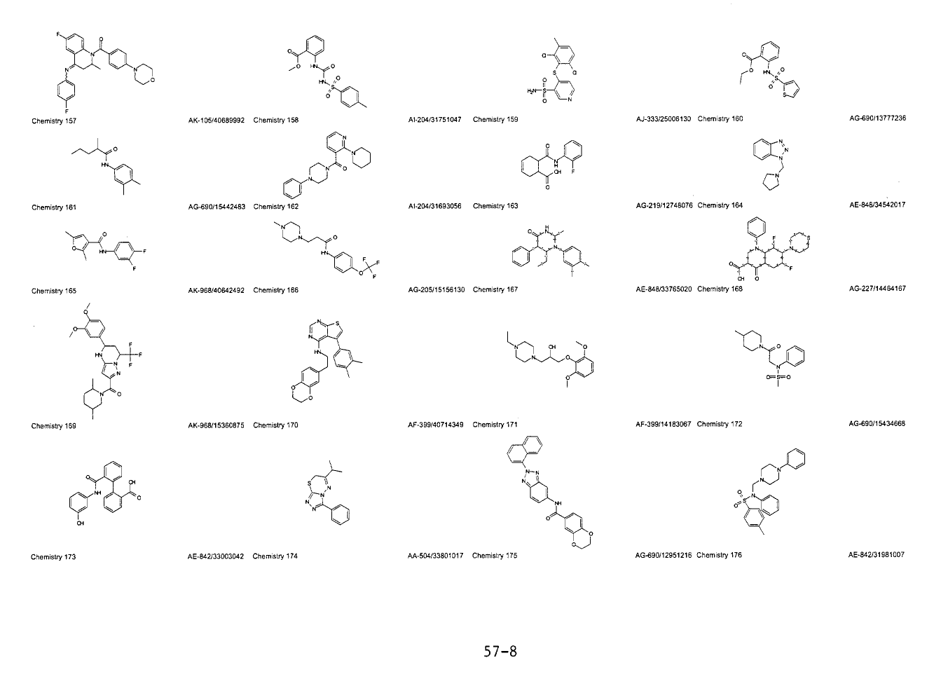

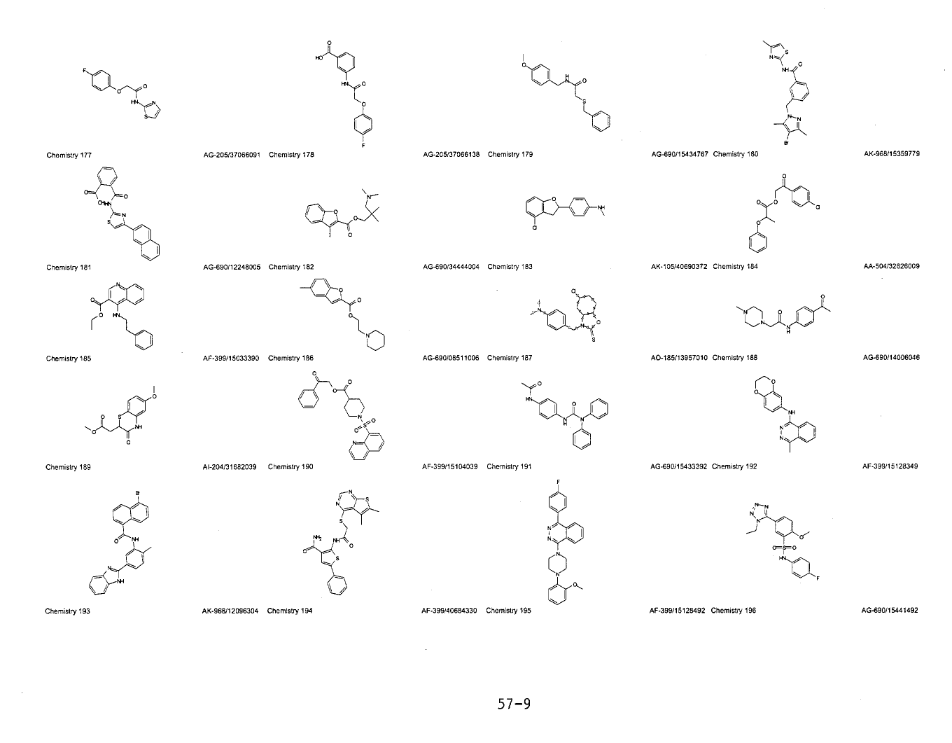

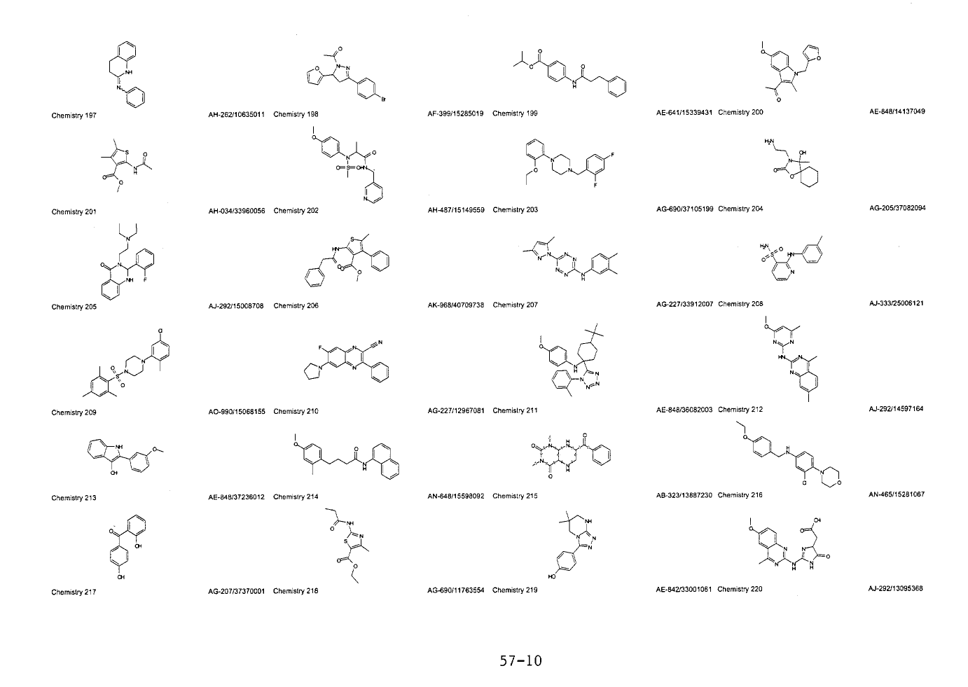

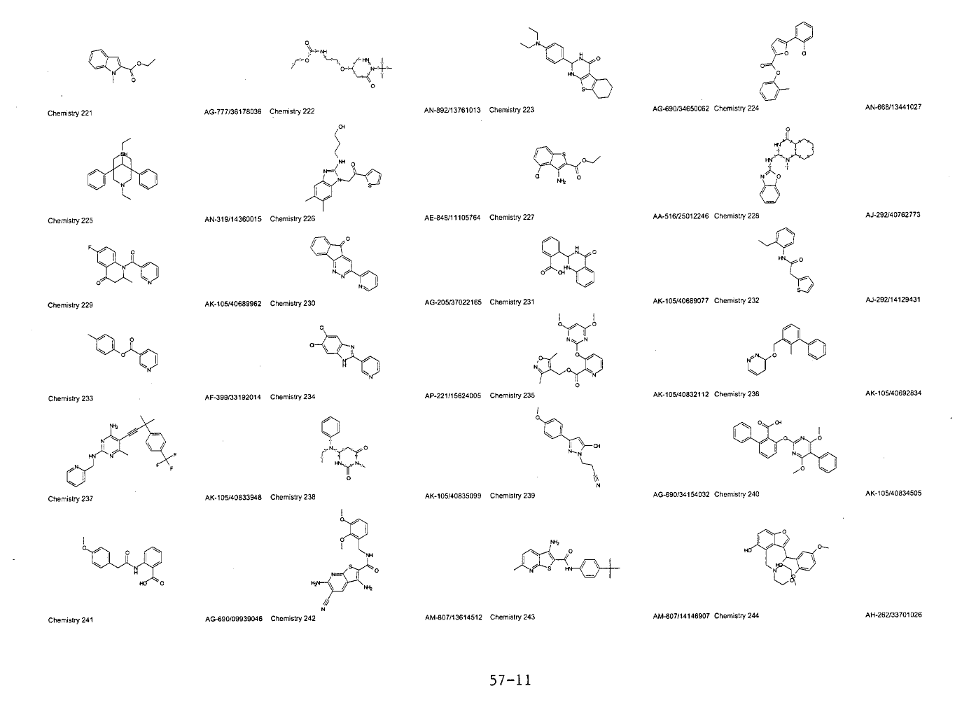

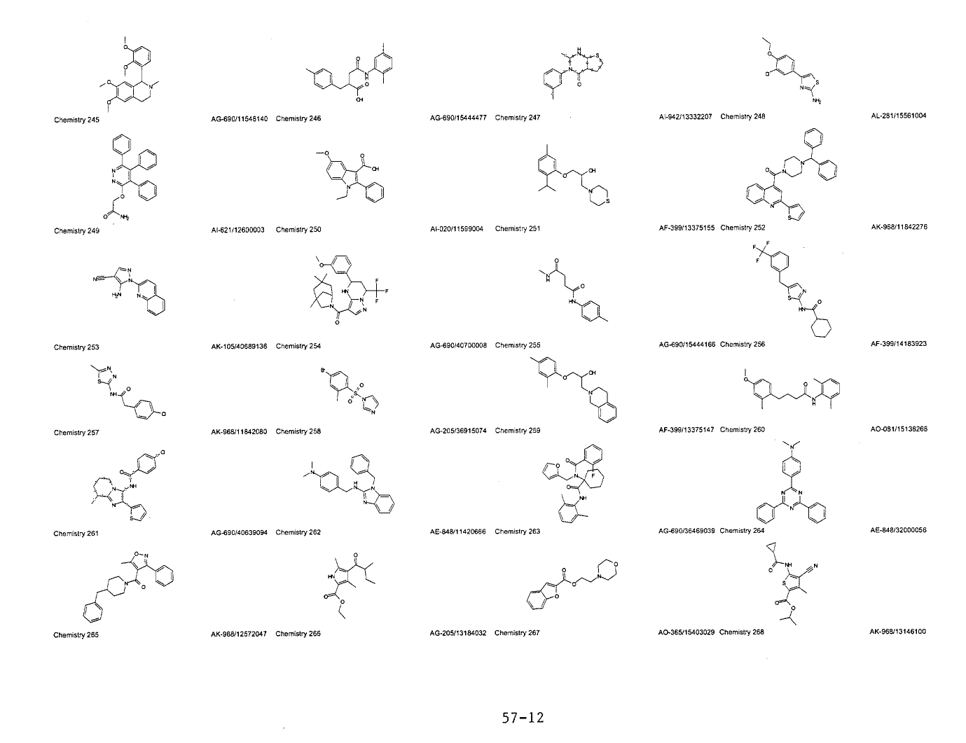

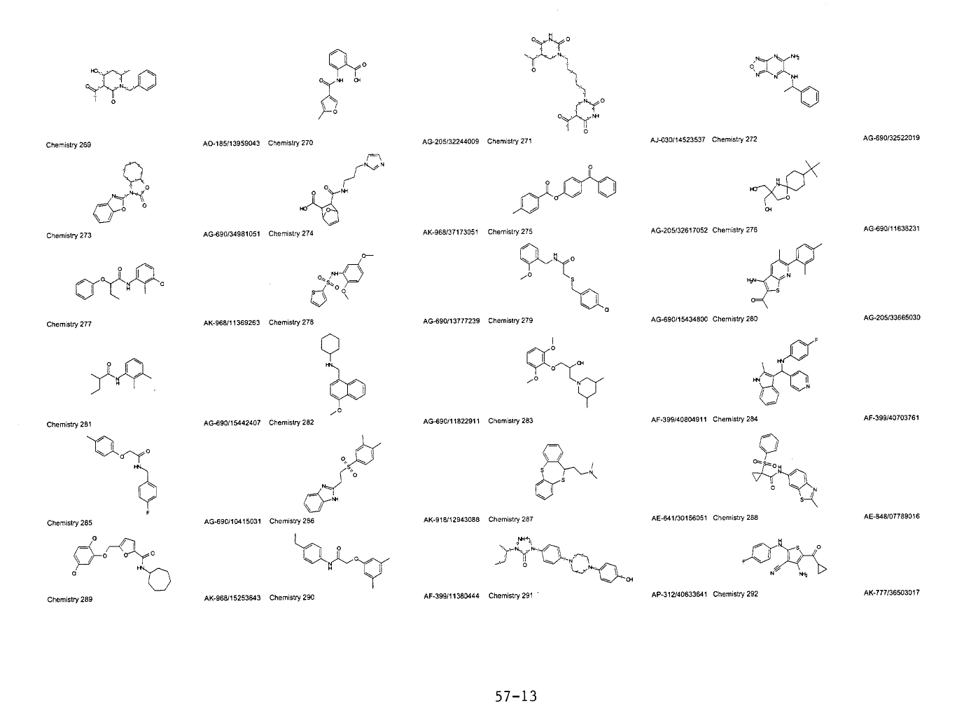

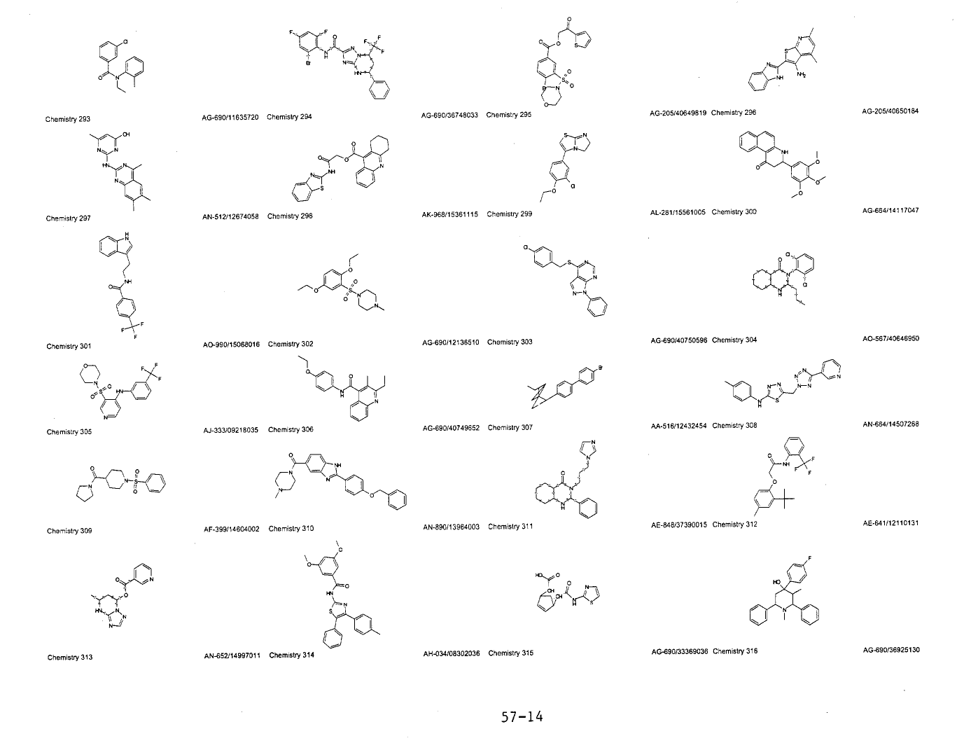

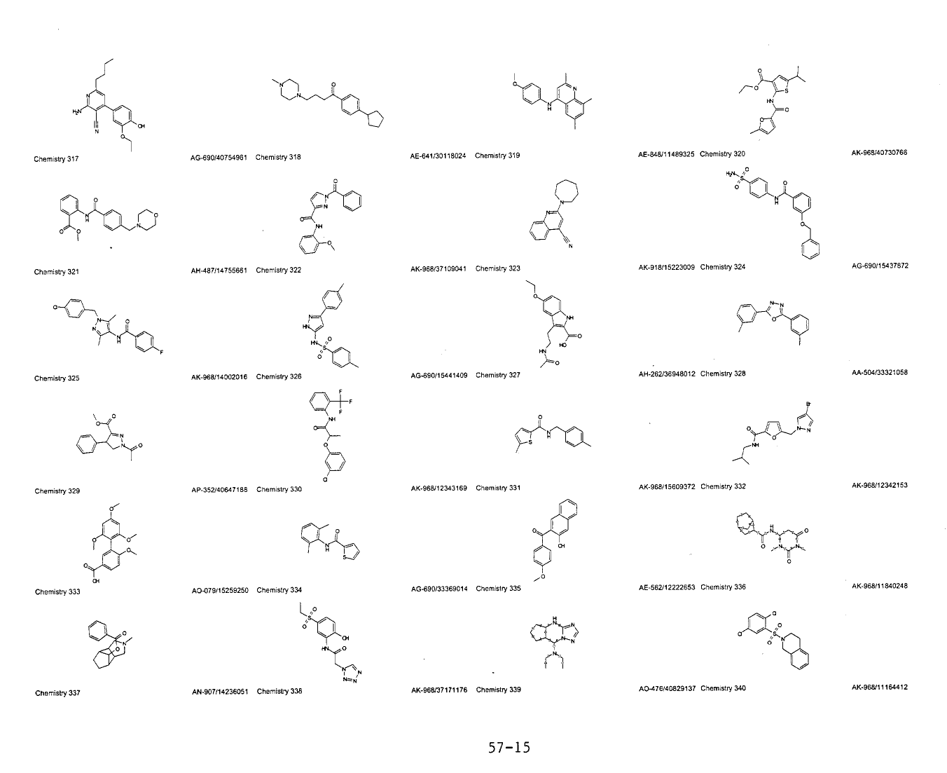

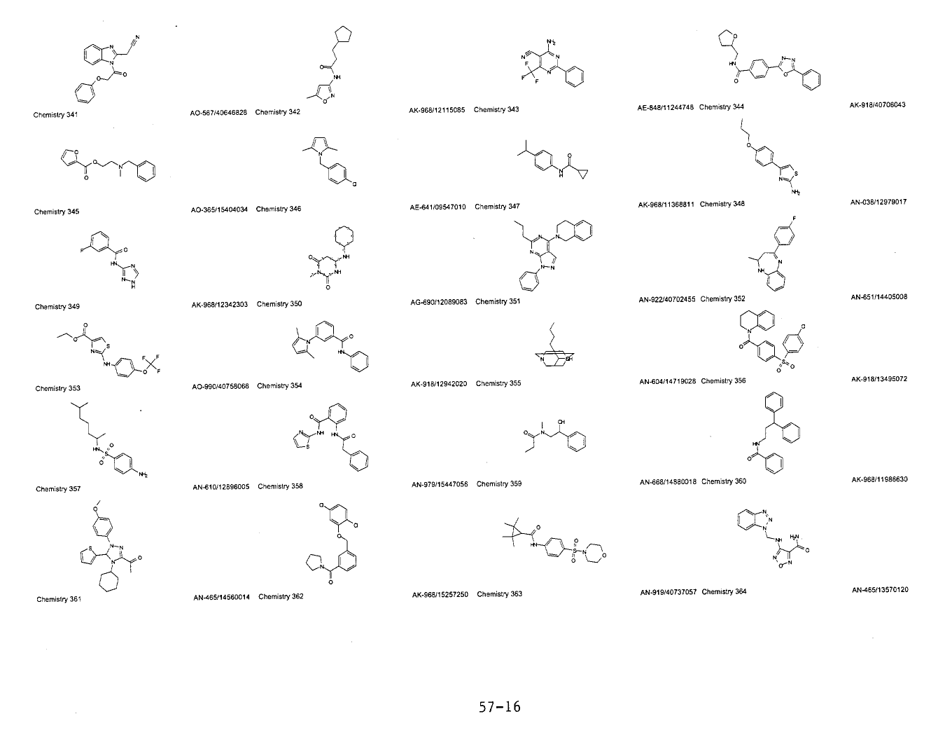

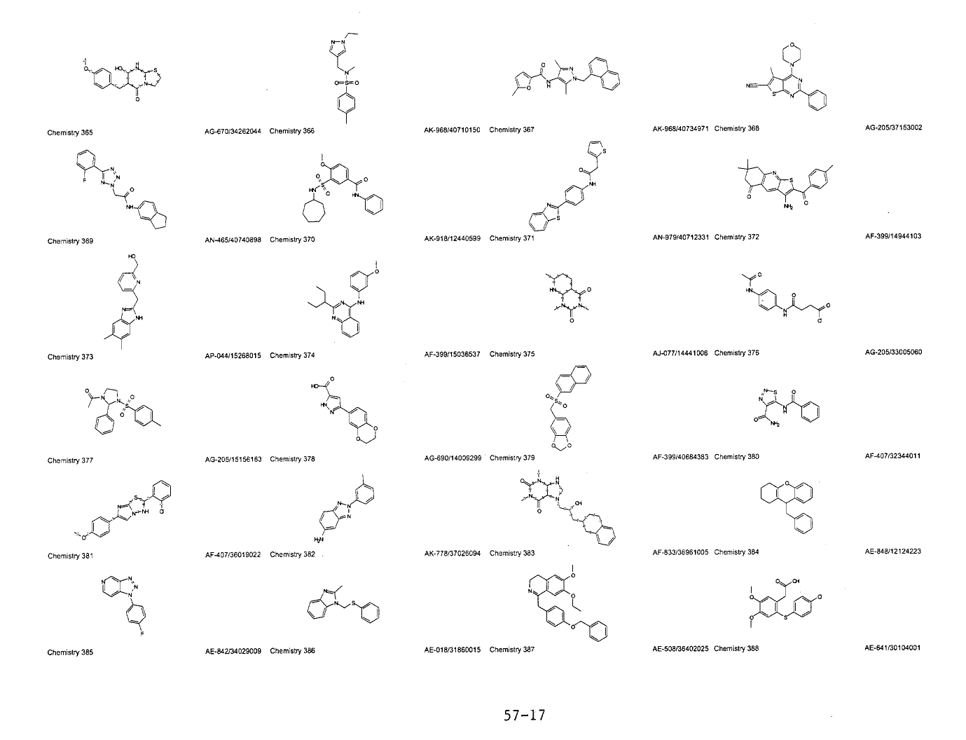

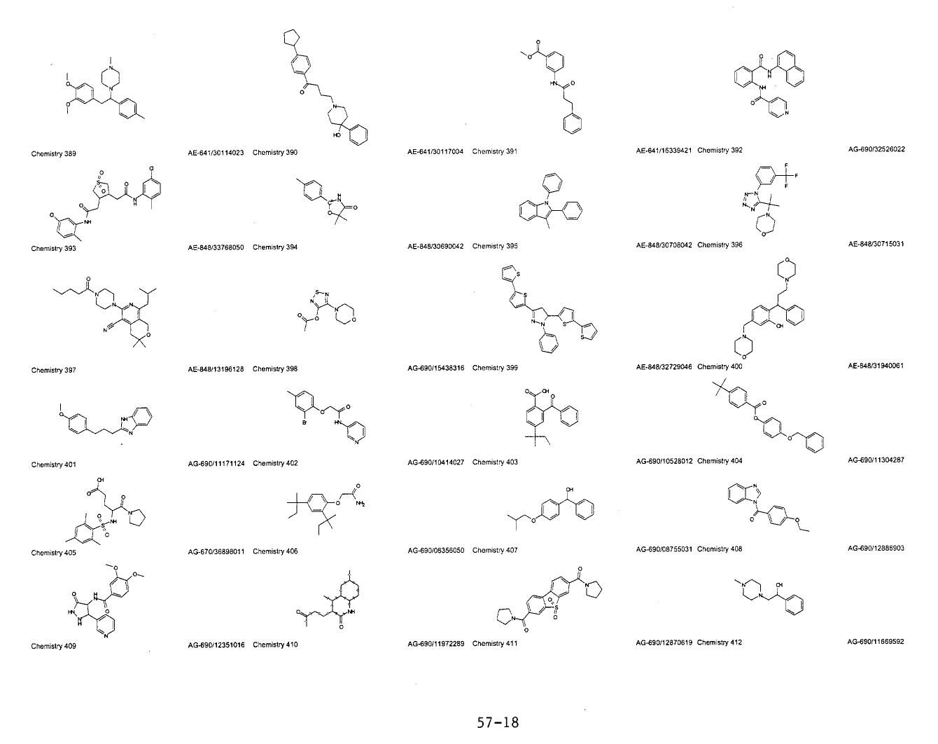

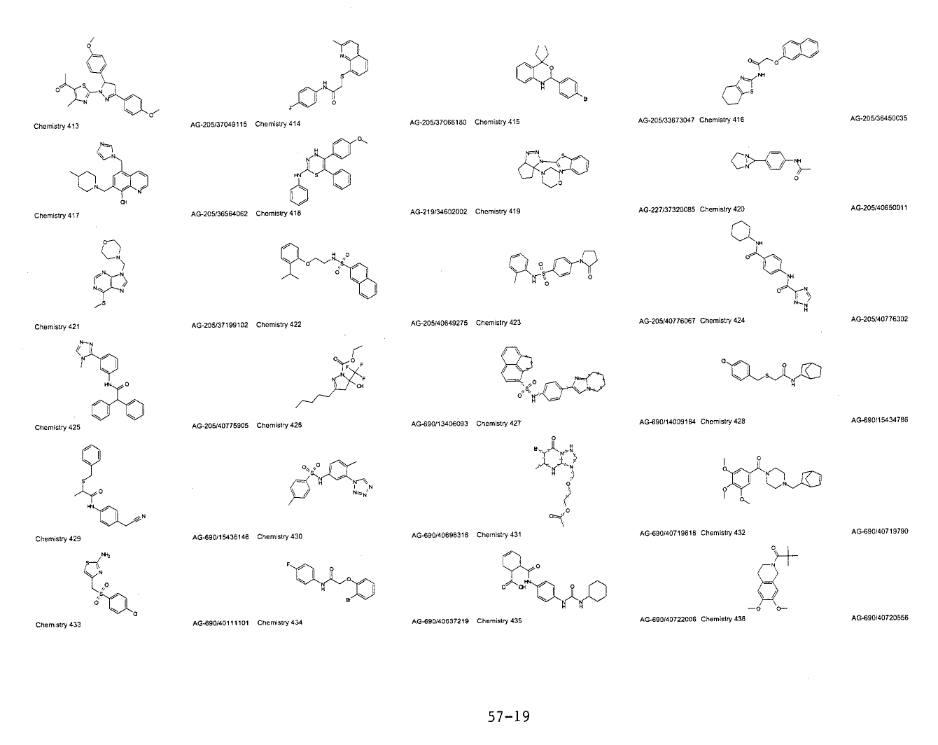

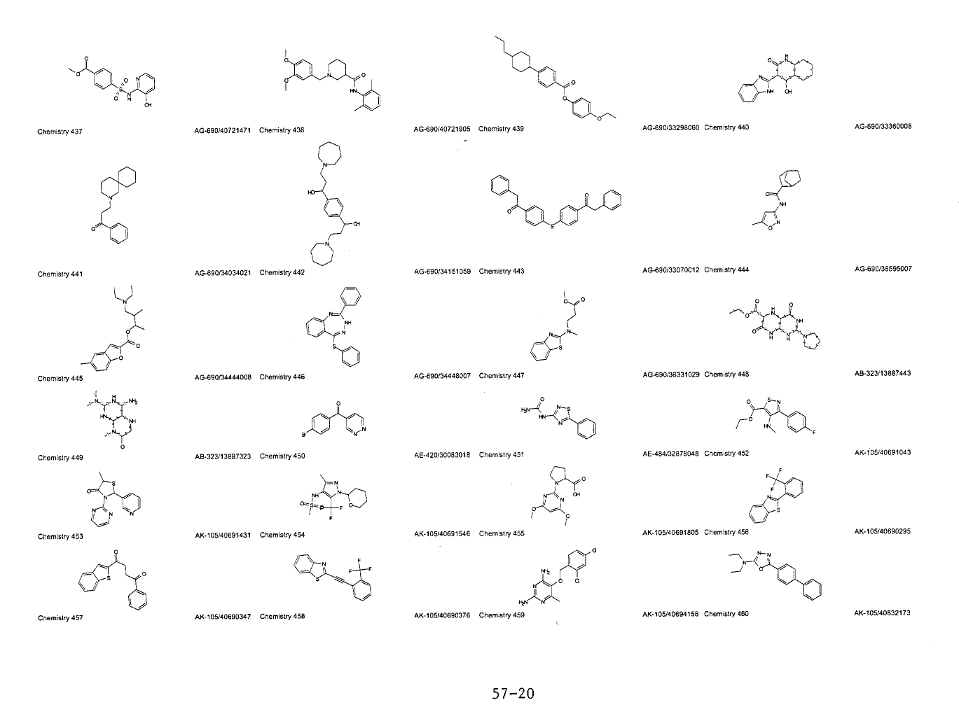

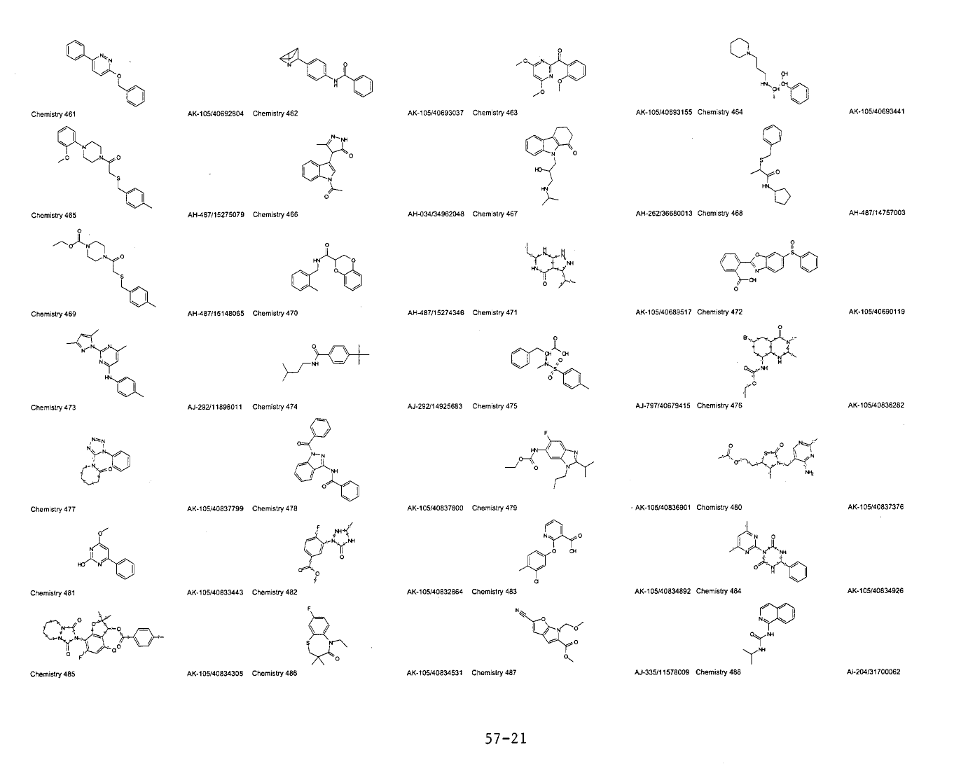

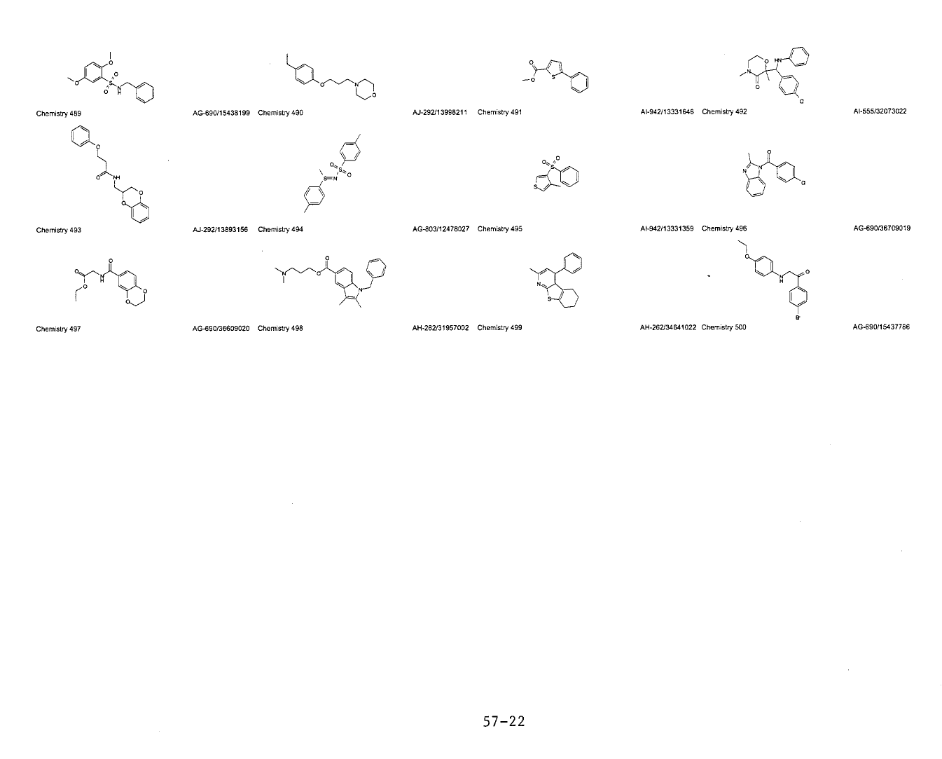

Table A is a chart that correlates the compound number, located on the X axis, of the compounds tested in FIGS. 37A-37C with the name of each compound tested. The structure associated with the name of each compound is presented in FIG. 57.

TABLE A
Correlation of Compound Number (X axis) in FIGS. 37A-37C with
Compound Name

| Compound | | Compound | | Compound | |
|---|---|---|---|---|---|
| Number | Compound | Number | Compound | Number | Compound |
| (FIG. 37A) | Name | (FIG. 37C) | Name | (FIG. 37B) | Name |
| 3 | AG-690/10375006 | 1 | Staurosporine | 1 | Staurosporine 10 uM |
| | | | 10 uM | | |
| 4 | AK-105/40837799 | 2 | Staurosporine | 2 | AK-968/11369166 |
| | | | 10 uM | | |
| 5 | AG-690/11838071 | 3 | Staurosporine | 3 | AK-968/15359576 |
| | | | 10 uM | | |
| 6 | AG-690/08753010 | 4 | Staurosporine | 4 | AE-848/13424458 |
| | | | 10 uM | | |
| 7 | AG-690/15439536 | 5 | Staurosporine | 5 | AK-968/11368811 |
| | | | 0.5 uM | | |
| 10 | AK-105/40693441 | 6 | Staurosporine | 6 | AK-968/15360875 |
| | | | 0.5 uM | | |
| 12 | AK-968/15360898 | 7 | Staurosporine | 7 | AK-968/15359777 |
| | | | 0.5 uM | | |
| 13 | ITU | 8 | Staurosporine | 8 | AK-918/12440599 |
| | | | 0.5 uM | | |
| 15 | AK-968/15253031 | 9 | AH- | 9 | AK-918/13947051 |
| | | | 487/14757003 | | |
| 16 | AN-610/12896005 | 10 | AK- | 10 | AE-848/30715031 |
| | | | 105/40832202 | | |
| 17 | AH-487/15274424 | 11 | AK- | 11 | AG-205/40776067 |
| | | | 968/40734971 | | |
| 18 | AJ-292/15008708 | 12 | AG- | 12 | |
| | | | 690/15436146 | | |
| 19 | AG-690/40721607 | 13 | AG- | 13 | Staurosporine 10 uM |
| | | | 690/12870938 | | |
| 24 | AK-968/37109041 | 14 | AG- | 14 | AE-848/30721016 |
| | | | 690/40111101 | | |
| 26 | STAUROSPORINE1 | 15 | AJ- | 15 | AF-399/14604002 |
| | 0 | | 292/14597164 | | |
| 28 | AG-690/15441430 | 16 | AG- | 16 | AG-207/37370001 |
| | | | 690/0993904616 | | |
| 30 | AG-690/12136510 | 17 | AK- | 17 | AE-848/31940061 |
| | | | 918/4070604317 | | |
| 31 | AG-690/11634062 | 18 | AN- | 18 | AN-651/14405008 |
| | | | 668/1488001818 | | |
| 32 | AP-064/15228382 | 19 | AK- | 19 | AN-465/14762009 |
| | | | 968/1234230319 | | |
| 36 | AG-205/14488022 | 20 | AO- | 20 | AJ-292/13095574 |
| | | | 476/4082913720 | | |
| 37 | AK-105/40832202 | 21 | AG- | 21 | AJ-292/14129431 |
| | | | 690/12868719 | | |
| 38 | AG-690/09939046 | 22 | AG- | 22 | AJ-797/40679415 |
| | | | 690/10375006 | | |
| 39 | STAUROSPORIN20 | 23 | AK- | 23 | AG-205/40776137 |
| | | | 968/15253031 | | |
| 41 | AE-848/11489353 | 24 | AG- | 24 | |
| | | | 690/12248005 | | |
| 42 | AJ-292/11898011 | 25 | AH- | 25 | Staurosporine 10 uM |
| | | | 262/10635011 | | |
| 42 | AK-105/40690295 | 26 | AG- | 26 | AJ-333/36115021 |
| | | | 690/11822911 | | |
| 44 | AK-968/15609372 | 27 | AF- | 27 | AI-942/13332207 |
| | | | 399/15036537 | | |
| 45 | AH-487/15275079 | 28 | Staurosporine | 28 | AG-219/12748076 |
| | | | 1 uM | | |
| 47 | AM-807/14146907 | 29 | | 29 | AK-968/37171176 |
| 49 | AK-968/40734971 | 30 | Staurosporine | 30 | AE-641/10388019 |
| | | | 1 uM | | |
| 51 | AG-690/12248005 | 31 | Staurosporine | 31 | AF-833/33256003 |
| | | | 1 uM | | |
| 52 | SB2003580 | 32 | AK- | 32 | AP-044/15268015 |
| | | | 105/40834926 | | |
| 55 | AK-105/40834531 | 33 | AN- | 33 | AK-105/40689962 |
| | | | 610/12896005 | | |
| 56 | AG-690/14009299 | 34 | AG- | 34 | AO-567/40646505 |
| | | | 690/15441430 | | |
| 57 | AN-038/12979017 | 35 | AG- | 35 | AF-399/40713795 |
| | | | 690/40720556 | | |
| 58 | AG-690/15441235 | 36 | AG- | 36 | |
| | | | 690/11171124 | | |
| 59 | AI-555/32073022 | 37 | AP- | 37 | |
| | | | 312/40633641 | | |
| 62 | AN-668/14880018 | 38 | AG- | 38 | AG-690/12071207 |
| | | | 690/11838071 | | |
| 63 | AH-262/10635011 | 39 | AH- | 39 | AE-562/12222653 |
| | | | 487/15274424 | | |
| 64 | AG-690/40720556 | 40 | AK- | 40 | AG-690/36709019 |
| | | | 105/40837799 | | |
| 65 | SB220025 | 41 | AE- | 41 | AK-968/12163519 |
| | | | 848/11489353 | | |
| 67 | AH-487/15148065 | 42 | AG- | 42 | AJ-030/14523537 |
| | | | 690/15442388 | | |
| 68 | AN-919/40737057 | 43 | AG- | 43 | AJ-292/40762773 |
| | | | 690/11548140 | | |
| 70 | AG-690/40720678 | 44 | AG- | 44 | AG-205/36566010 |
| | | | 690/08753010 | | |
| 72 | AG-690/11634672 | 45 | AJ- | 45 | AH-487/15274256 |
| | | | 292/15008708 | | |
| 73 | AG-690/12870938 | 46 | AG- | 46 | AH-262/36948012 |
| | | | 690/12136510 | | |
| 74 | AO-476/40829137 | 47 | AJ- | 47 | AO-990/40758 198 |
| | | | 292/11898011 | | |
| 75 | AG-690/11822911 | 48 | AK- | 48 | |
| | | | 105/40690295 | | |
| 76 | AG-690/11171124 | 49 | AN- | 49 | control |
| | | | 979/40712331 | | |
| 77 | AG-690/15442388 | 50 | AG- | 50 | AG-690/36333036 |
| | | | 690/15439536 | | |
| 78 | OLOMOUCINE | 51 | | 51 | AG-690/11763554 |
| 80 | AG-690/15434767 | 52 | Control | 52 | AG-670/36764013 |
| 81 | AG-690/11822926 | 53 | Control | 53 | AE-848/34542017 |
| 82 | AO-990/15068016 | 54 | Control | 54 | AK-777/36503017 |
| 83 | NK-968/37129243 | 55 | AG- | 55 | AG-690/11665066 |
| | | | 690/40721607 | | |
| 84 | AG-205/36564062 | 56 | AG- | 56 | AG-690/40750596 |
| | | | 690/11634062 | | |
| 85 | AG-690/40111101 | 57 | AM- | 57 | AG-690/40637436 |
| | | | 807/14146932 | | |
| 88 | AP-312/40633641 | 58 | AK- | 58 | AF-399/40804911 |
| | | | 105/40834531 | | |
| 89 | AG-690/11548140 | 59 | AH- | 59 | AN-465/40740898 |
| | | | 487/15148065 | | |
| 90 | AN-979/40712331 | 60 | AG- | 60 | |
| | | | 690/15442483 | | |
| 91 | PD98059 | 61 | AG- | 61 | control |
| | | | 690/40719790 | | |
| 93 | AN-979/15447121 | 62 | AP- | 62 | AG-205/37066091 |
| | | | 064/15228382 | | |
| 94 | AK-968/11163100 | 63 | AK- | 63 | AH-262/33701026 |
| | | | 968/15609372 | | |
| 95 | AG-690/15438171 | 64 | AG- | 64 | AK-778/37026094 |
| | | | 690/14009299 | | |
| | | 65 | AN- | 65 | AG-690/11449023 |
| | | | 919/40737057 | | |
| | | 66 | AN- | 66 | AG-690/33369036 |
| | | | 979/15447121 | | |
| | | 67 | AK- | 67 | AE-842/34029009 |
| | | | 105/40837800 | | |
| | | 68 | AG- | 68 | AG-690/40721139 |
| | | | 690/15434767 | | |
| | | 69 | AG- | 69 | AG-690/08755031 |
| | | | 690/40697747 | | |
| | | 70 | AH- | 70 | AK-968/40642492 |
| | | | 487/15275079 | | |
| | | 71 | AN- | 71 | AG-205/40776302 |
| | | | 038/12979017 | | |
| | | 72 | AG- | 72 | |
| | | | 690/15433392 | | |
| | | 73 | AG- | 73 | |
| | | | 690/11822926 | | |
| | | 74 | AM- | 74 | AG-690/15438199 |
| | | | 807/14146907 | | |
| | | 75 | AK- | 75 | AG-690/15434668 |
| | | | 105/40693441 | | |
| | | 76 | AH- | 76 | AG-690/15440341 |
| | | | 487/15274346 | | |
| | | 77 | AG- | 77 | AG-690/15439249 |
| | | | 690/15441235 | | |
| | | 78 | AG- | 78 | AG-205/15156163 |
| | | | 690/40720678 | | |
| | | 79 | AO- | 79 | AE-848/37390015 |
| | | | 990/15068016 | | |
| | | 80 | AK- | 80 | AH-487/14755661 |
| | | | 968/11163100 | | |
| | | 81 | AK- | 81 | AH-262/31957002 |
| | | | 968/11986630 | | |
| | | 82 | AI- | 82 | AG-205/40775819 |
| | | | 204/31700062 | | |
| | | 83 | AF- | 83 | AN-512/12674058 |
| | | | 399/37321017 | | |
| | | 84 | AI- | 84 | |
| | | | 555/32073022 | | |
| | | 85 | AG- | 85 | |
| | | | 690/37105199 | | |
| | | 86 | AK- | 86 | AK-968/15360521 |
| | | | 968/37129243 | | |
| | | 87 | AG- | 87 | AF-399/15128349 |
| | | | 690/15438171 | | |
| | | 88 | AK- | 88 | AK-968/13150206 |
| | | | 968/15360898 | | |
| | | 89 | AK- | 89 | AJ-292/13489181 |
| | | | 968/37109041 | | |
| | | 90 | AG- | 90 | AE-641/30104001 |
| | | | 205/14488022 | | |
| | | 91 | AM- | 91 | AE-641/30118024 |
| | | | 807/13612090 | | |
| | | 92 | AG- | 92 | AN-648/15598092 |
| | | | 690/11634672 | | |
| | | 93 | AG- | 93 | AH-487/15149559 |
| | | | 205/36564062 | | |
| | | 94 | AG- | 94 | AG-205/40775905 |
| | | | 690/33051023 | | |
| | | 95 | AG- | 95 | AG-205115156130 |
| | | | 664/14117047 | | |
| | | 96 | AK- | 96 | |
| | | | 918/15223009 | | |

C. Determination of the Secondary Structure of the ADP Aptamer

Two structural analytical methods were used to determine the minimal secondary structure of the ADP F01 aptamer, 3′-end mapping and doped RNA reselection.

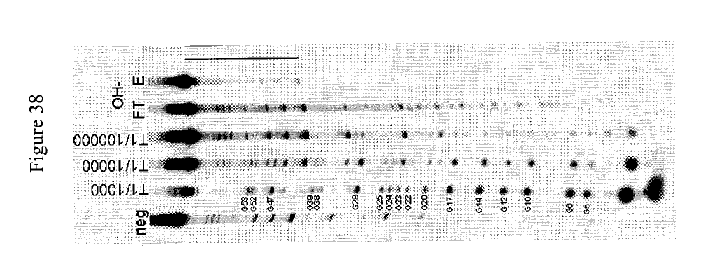

The first structural analytical method used to study ADP F01 aptamer was 3′-end mapping by alkaline hydrolysis (FIG. 38). This technique, uses 5′-³²P-end-labeled RNA. The RNA is partially degraded in mildly basic buffer (NaHCO₃, pH 9.5). The RNA is then subsequently applied to the ADP affinity column. Both the unbound (FT, flow through) and ADP binder RNAs eluted from the column (E) are concentrated by ethanol precipitation and run out on a sequencing gel along with an RNAse T1 (cuts at G) sequencing lane. The shortest fragment observed in the lane with specifically eluted RNA represents the 3′-end of the core of the aptamer. As indicated by the vertical lines in FIG. 38, two possible functional 3′ boundaries were estimated for the ADP F01 aptamer.

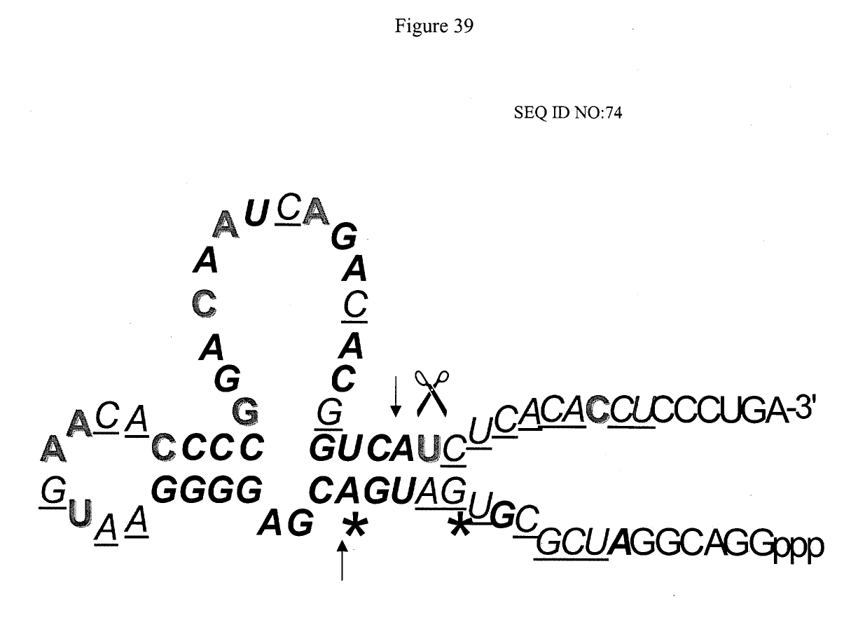

Another structural analytical approach, doped reselection, followed by sequence and covariational analysis is a powerful technique for unambiguously assigning the secondary structure of an aptamer. Doped reselection involves resynthesis of the aptamer clone at the DNA level with the core sequence of the aptamer mutated such that at any given position the nucleotide is 85% likely to be the wild type sequence and 5% likely to be any of the other three nucleotides. Reselection with this mutated RNA reveals regions of the sequence that are highly conserved or not, thus identifying key regions of the aptamer. Furthermore in regions of Watson/Crick base pairing, covariation will appear, e.g., a G becomes an A at one position and a C becomes a T at another position. If covariation is observed in a region of potential Watson/Crick pairings, it is extremely likely that the potential pairings are in fact real and that the proposed helix is an element of the secondary structure of the aptamer. The results of doped reselection for the F01 ADP aptamer are presented in FIG. 39. Bold italic nucleotides were highly conserved, gray shadowed nucleotides were moderately conserved, italic underlined nucleotides were unconserved, and plain nucleotides were undoped. An asterisk (*) indicated basepairs where Watson/Crick covariation was observed, arrows indicate primer boundaries in the initial selection, and scissors indicate the 3′-boundary as determined by alkaline hydrolysis for that pairing.

In the doped reselection analysis of ADP F01 aptamer, a new pool was synthesized based on the F01 clone using the primers shown in Table 6.

TABLE 6

Primers Used to Synthesize the ADP F01 Pool for Doped Reselection

| | | | | |
|---|---|---|---|---|
| | SEQ ID | | Tm | |
| Probe | NO | Length | (° C.) | Sequence |

| | | | | |
|---|---|---|---|---|
| >ADP_11D_5, 5′ | 75 | 34 nt | 58 | 5′-TAATACGACTCACTATAGGACCTG |
| primer for ADP doped | | | | GCTTGGACGG-3′ |
| pool = jd18155u | | | | |
| >ADP_11D_3, 3′- | 76 | 19 nt | 58 | 5′-AGTCCCGAGCACTTCAGGG-3′ |
| primer for ADP doped | | | | |
| pool = jd18155v | | | | |
| >ADP_11D rev comp | 77 | 115 nt | N.D.* | 5′-AGTCCCGAGCACTTCAGGGAGGTG |
| for oligo txn = jd15155w | | | | TGTGAGATGACCGTGTCTGATTGTCCG |
| oligo with trityl on and | | | | GGGTGTTCATTCCCCTCGTCATCACGC |
| 85% wt/5% | | | | GAT CCGTCCAAGCCAGGTCCTATAGT |
| doping at bold or | | | | GAGTCGTATTA-3′ |
| numbered positions | | | | |

*Not Determined

All the methods used to generate, select and clone the F01 pool were carried out essentially as previously described for ADP selection round 16 except that the selection buffer was modified to better fit with kinase assay conditions. That is, the F01 pool was PolyPacII purified as ADP selection; a 5 ml large scale transcription and gel purification was done as ADP selection; selection round 1 started with 10¹⁴ molecules; three rounds of doped selection were conducted before cloning and sequencing. However, the selection buffer used was different in doped reselection to be more compatible with buffers used in kinase assays. Specifically, the selection buffer used in these studies was 50 mM Hepes, pH 7.5, containing 10 mM MgCl₂, 10 mM MnCl₂, 100 mM NaCl, 1 mM DTT and 1% DMSO. The results of the doped reselection analysis of ADP aptamer F01 is shown in Table 7.

TABLE 7

Summary of Doped Reselection Analysis of ADP Aptamer F01

| | | |
|---|---|---|
| Sequences | | |
| from Doped | | SEQ ID |
| Reselection: | Nucleic acid Sequence | NO: |

| | | |
|---|---|---|
| >ARX3P1.F01 | GGACGGATCGCGTGATGACGAGGGGAATGAACACCCCGGACAATCAGACACG | 78 |
| | GTCATCTCACACACCTCCCTG | |
| >ARX7P1.A02 | GGACCTGGCTTGGACGGATCGAGTGATGACGAGGGGACTGAACACCCCAGAC | 79 |
| | AATCAGACACGGTCACCTCACATACCTCCCTGAAGTGCTCGGGACT | |
| >ARX7P1.A03 | GGACCTGGCTTGGACGGCTTGCGTGGTGACGAGGGGAATGAACATCCCGGAC | 80 |
| | AATCAGAAACGGTCATCACACATCCACCCCTGAAGTGCTCGGGACT | |
| >ARX7P1.A04 | GGACCTGGCTTGGACGGATNGCGTGATGACGAGGGGCANNATTAACCCGGAC | 81 |
| | AATCGGACACGGTCATAACNNACACCTCCCTGAAGTGCTCGGGACT | |
| >ARX7P1.A05 | GGACCTGGCTTGGACGGANCNAGTGATGACGAGGGGAATGAACACCCCGGAC | 82 |
| | AATTAGACACGGTCATCTCAGCTAGCTCCCTGAAGTGCTCGGGACT | |
| >ARX7P1.A06 | GGACCTGGCTTGGACGGATCAAGTGATGACGAGGGGAGCGACCACCCCGGAC | 83 |
| | AATCAGACACGGTCATCACACACACATCCCTGAAGTGCTCGGGACT | |
| >ARX7P1.B01 | GGACCTGGCTTGGACGGATTGCGTGATGACGAGGGGAGTCAACCCCCCAGAA | 84 |
| | ACTCAGAAACGGTCATATCACACACCTCCCTGAAGTGCTCGGGACT | |
| >ARX7P1.B02 | GGACCTGGCTTGGACGGATTGCGTGATGACGAGGGGAATAAACACCCCGGAA | 85 |
| | AATCAGAAACGGTCATCTCAGACACCTCCCTGAAGTGCTCGGGACT | |
| >ARX7P1.B03 | GGACCTGGCTTGGACGGATCGCGTGATGACGAGGGGAATGAACACCCCGGAC | 86 |
| | AATTAGAAACGGTCATTTCACATACCGCCCTGAAGTGCTCGGGACT | |
| >ARX7P1.B06 | GGACCTGGCTTGGACGGATCGCGTGATGTCGAGGGGCATGAAAACCCCGGAC | 87 |
| | AATCAGACACGGACATCTATCACTCCGCCCTGAAGTGCTCGGGACT | |
| >ARX7P1.C01 | GGACCTGGCTTGGACGGATCGCGTGATGACGAGGGGAATGATCACCCCGGAC | 88 |
| | AATCAGACACGGTCATCTCTCACAACGCCCTGAAGTGCTCGGGACT | |
| >ARX7P1.A01 | GGACCTGGCTTGGACGGATCGCAAGATGACGAGGGGAATGAACGCCCCGGAC | 89 |
| | AATAAGACACAGTCATCTCACACACCTCCCTGAAGTGCTCGGGACT | |
| >ARX7P1.C03 | GGACCTGGCTTGGACGGTTTGCGTGATGACGAGGGGAATTAGCACCCCGGAC | 90 |
| | AATTAGACACGGTCATCTCGCATACATCCCTGAAGTGCTCGGGACT | |
| >ARX7P1.C04 | GGACCTGGCTTGGACGGATCGCGTGAAGACGAGGGGAATGGACACCCCGGAC | 91 |
| | AATCAGAAACGGTCATCTCACGCAGTTCCCTGAAGTGCTCGGGACT | |
| >ARX7P1.C05 | GGACCTGGCTTGGACGGACNGCGNNATGACGAGGGGAATGAACACCCCGGAC | 92 |
| | AATCAGACACAGTCATCTCACTCANCNCCCTGAAGTGCTCGGGACT | |
| >ARX7P1.D04 | GGACCTGGCTTGGACGGAACGAGTCATGACGAGGGGAATGAACACCCCGGAC | 93 |
| | CGTAAGACACTGTCATCTCACACACCTCCCTGAAGTGCTCGGGACT | |
| >ARX7P1.D05 | GGACCTGGCTTGGACGGATGGNGTGATGACGAGGGGAATGAANACCCCGGAC | 94 |
| | AATCAGANACGGTCATCTCACNCACATCCCTGAAGTGCTCGGGACT | |
| >ARX7P1.D06 | GGACCTGGCTTGGACGGATCGCGTGATGACGAGGGGAATGAAAGCCCCGGAC | 95 |
| | AATCAGACACGGTCATCACACACACGTCCCTGAAGTGCTCGGGACT | |
| >ARX7P1.E01 | CGGTCATTTCACACACCTCCCTGAAGTGCTCGGGACT | 96 |
| >ARX7P1.E02 | GGACCTGGCTTGGACGGATCGCGTGTTGACGAGGGGAATGTACACCCCGGAC | 97 |
| | AATCAGACACAGTCAACCTGACGCACCTCCCTGAAGTGCTCGGGACT | |
| >ARX7P1.E05 | AACTAGTGATGACGAGGGGAATAAACTCCCCGGACAATCAGAAACGGTCATC | 98 |
| | ACAAACCCGTCCCTGAAGTGCTCGGGACT | |
| >ARX7P1.E06 | GGACCTGGCTTGGACGGATCGCGTGATGACGAGGGGAAGGAACACCCCGGAC | 99 |
| | AATCGGATACGGTCATCGCACACTCCTCCCTGAAGTGCTCGGGACT | |
| >ARX7P1.F01 | GGACCTGGCTTGGACGGATCGCGTGATGACGAGGGGATTGAACACCCCGGAC | 100 |
| | AATAAGACACGGTCATATTACACAGCTCCCTGAAGTGCTCGGGACT | |
| >ARX7P1.F02 | GGACCTGGCTTGGACGGATCGCGTGATGACGAGGGGAACGAACCCCCGGACA | 101 |
| | ATAAGAAACGGTCATCTCATCCATCCCCCTGAAGTGCTCGGGACT | |
| >ARX7P1.F05 | GGACCTGGCTTGGACGGATTGCGTGATGACGAGGGGAATGAACACCCCGGAC | 102 |
| | AATCAGACACAGTCATCTCACCAATCGCCCTGAAGTGCTCGGGACT | |
| >ARX7P1.F06 | GGACCTGGCTTGGACGGACAGGGTGATGACGAGGGGAATGAACACCCCGGAC | 103 |
| | AATCAGACACGGTCATCTCCCAGCCCTCCCTGAAGTGCTCGGGACT | |
| >ARX7P1.G01 | GGACCTGGCTTGGACGGATATGGTGATGACGAGGGCAATGAACAACCCGGAC | 104 |
| | AATCAGAAACAGTCATCTCACATCCACCCCTGAAGTGCTCGGGACT | |
| >ARX7P1.G02 | GGACCTGGCTTGGACGGATCGTGTGGTGACGAGGGGAATCAACAACCCGGAC | 105 |
| | AATGAGACACGGTCATCTCACCCCCTGAAGTGCTCGGGACT | |
| >ARX7P1.G03 | GGACCTGGCTTGGACGGATNGCGTGATGACGAGGGGNNTCNAGACCCCGGAC | 106 |
| | AATAAGACACGGTCATCTNACANCCGNCTCCCTGAAGTGCTCGGGACT | |
| >ARX7P1.G04 | GGACCTGGCTTGGACGGAAACGTAATGACGAGGGGAACGAATACCCCGGACA | 107 |
| | ATGAGAAACGGTCATTTTACCCACTTCCCTGAAGTGCTCGGGACT | |
| >ARX7P1.G05 | GGACCTGGCTTGGACGGATCGCGTGGTGACGAGGGGAAAGAAAACTCCGGAC | 108 |
| | AATTAGACACAGTCATCTCACACTCCTCCCTGAAGTGCTCGGGACT | |
| >ARX7P1.G06 | GGACCTGGCTTGGACGGATCGCGGGATGACGAGGGGCATGAACACCTCGGAC | 109 |
| | AATCAGACACGGTCATCTCGCAACACTCCCTGAAGTGCTCGGGACT | |
| >ARX7P1.H01 | GGACCTGGCTTGGACGGATNNCGTGATGACGAGGGGNATGANCACCCCGGAC | 110 |
| | AATTAGAAACGGTCATCTCACACACNTCCCTGAAGTGCTCGGGACT | |
| >ARX7P1.H03 | GGACCTGGCTTGGACGGATNGCGTGATGACGAGGGGAATGAACACCCCGGAC | 111 |
| | AATAAGACACGGTCATCNCGCACNCCTCCCTGAAGTGCTCGGGACT | |
| >ARX7P1.H04 | GGACCTGGCTTGGACGGATAGGCTCATGACGAGGGGGATGAACACCCCGGAC | 112 |
| | AATCAGACACGGTCAGCTCTAACACCGCCCTGAAGTGCTCGGGACT | |
| >ARX7P1.H05 | GGACCTGGCTTGGACGGATAGCGGGATGATGAGGGGATTGAACGCCCCGGAC | 113 |
| | AATCAGAAACGGTCATCTTACGCACGTCCCTGAAGTGCTCGGGACT | |
| >ARX7P1.H06 (1) | GGACCTGGCTTGGACGGATCCCGGCATGACGAGGGGAATGAACACCCCGGAC | 114 |
| | AATAAGACACGGTCATGCTAGACACCTCCCTGAAGTGCTCGGGACT | |

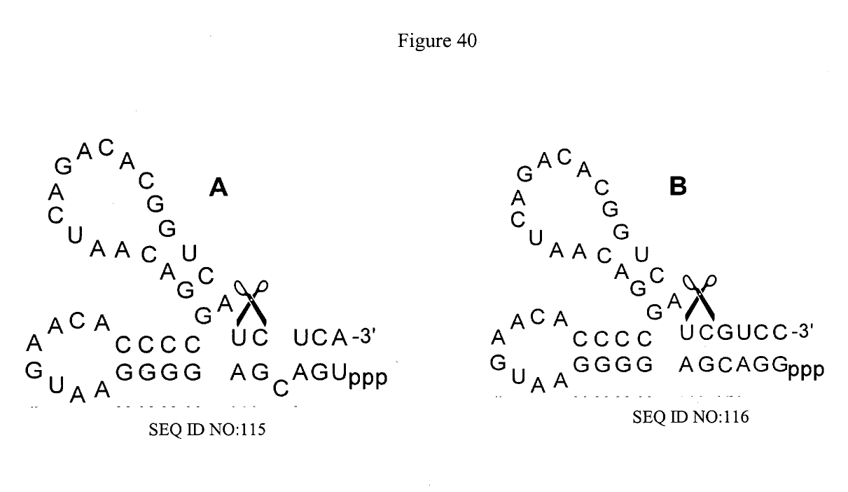

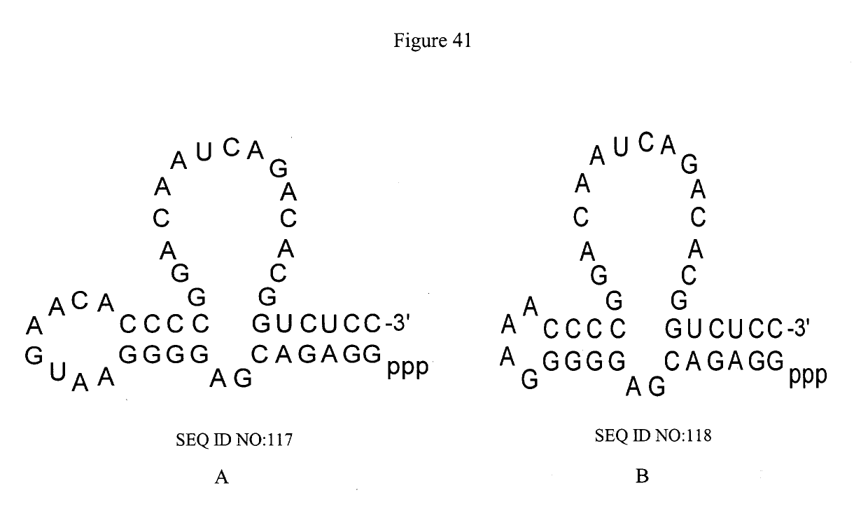

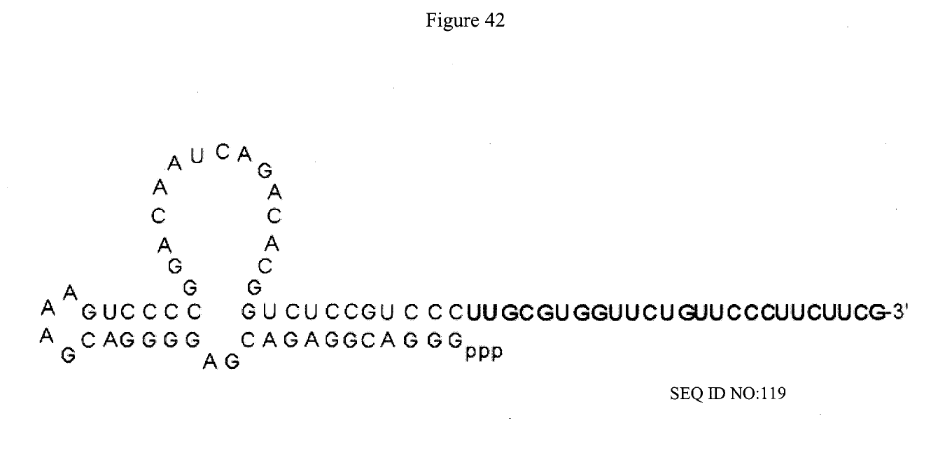

The predicted structure as shown in FIG. 39 differed from that predicted by computational finding models as shown in FIG. 40A. However, the computationally predicted structure engineered from the F01 clone as shown in 40B to increase its stability, however, did not bind ADP. Once the aptamer secondary structure was determined, minimal constructs derived therefrom were tested for their ability to bind to the ADP column. The minimal aptamer structures shown in FIG. 41 were experimentally confirmed to bind to an ADP affinity column. The minimal aptamer was then resynthesized and transcribed with the 3′-capture sequence such that binding experiments could be performed (FIG. 42).

D. Minimized ADP Aptamer F01-based Kinase SPA

As shown in FIG. 32, surface immobilized aptamer RNA that binds to ³H-ADP may be utilized to measure kinase-mediated protein phosphorylation. An ADP aptamer will concentrate the tritiated ADP released by kinase on the surface of the flash plate and generate a signal detectable in a Topcount scintillation counter. For the study presented, minimized ADP aptamer (JD37.98A) (FIG. 42, SEQ ID NO: 1 19) was directly 3′-biotinylated and immobilized onto NEN streptavidin flashplates. Specifically, select assay wells were incubated with 5 pmol of JD37.98A directly 3′-biotinylated aptamer in 25 μl of PBS Buffer:BupH (0.05% tRNA, 0.025% Tween 20) with shaking at 650 RPM for 30 min. Unbound biotinylated aptamer probe was rinsed from the plate with three 1×PBS washes and dried as previously described.

FIG. 43 shows the use of the minimized ADP aptamer to detect ppERK-mediated phosphorylation of Myelin basic protein (MBP) in an ADP SPA. The phosphorylation of ppERK is reflected in the time-dependent and ppERK-dependent increase in assay signal observed using the ADP SPA incorporating the minimized, directly biotinylated ADP aptamer. Furthermore, as shown in FIG. 43, concentration-dependent inhibition of ppERK by staurosporine using this ADP SPA is consistent with the known kinase inhibitory activity of staurosporine.

In following experiments the production of ³H-ADP from ³H-ATP was detected from ppERK using streptavidin coated flash plate wells that were incubated with 60 pmol of MK08.112A biotinylated capture probe in 30 μl of PBS Buffer:BupH (0.05% tRNA, 0.025% Tween 20) for 15 min with shaking at 650 RPM. Unbound probe was removed from the plate with three 1×PBS washes and blotted dry as described above. JD37.98A (150 pmol; not biotinylated) minimized ADP aptamer transcribed with capture probe sequence was added to select wells in 30 μl 1×PBS and incubated for 30 min with shaking at shaking at 650 RPM, Unbound probe was removed with three PBS washes and the plates were blotted dry as described above. Kinase reactions were carried out in a 30 μl final sample volume as follows.

Stock 15 nM ppERK enzyme solution was prepared in selection buffer and incubated for 10 min at room temperature. Kinase test reactions were initiated by adding 1 μl test sample, e.g., inhibitor, to 14 μl selection buffer containing 1 mM DTT, 50pM MBP, and 2.7 μM ³H-ATP and then combining this mixture with an equal volume of 15 nM ppERK enzyme solution. Kinase test reaction was immediately transferred to the appropriate test well and the reaction monitored on a TopCount, ³H SPA scintillation counter over 900 min.

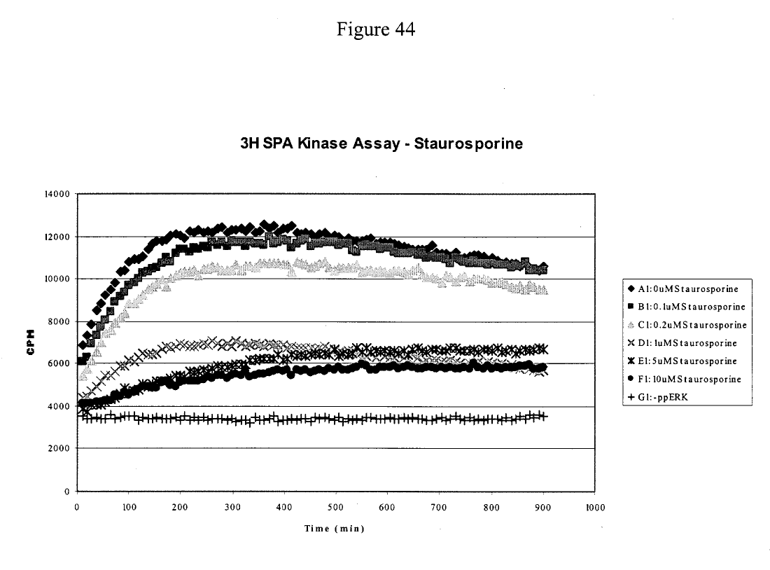

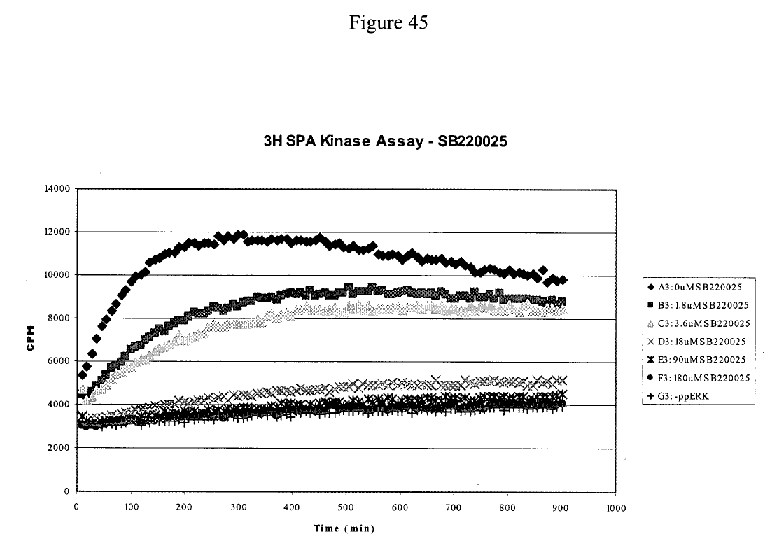

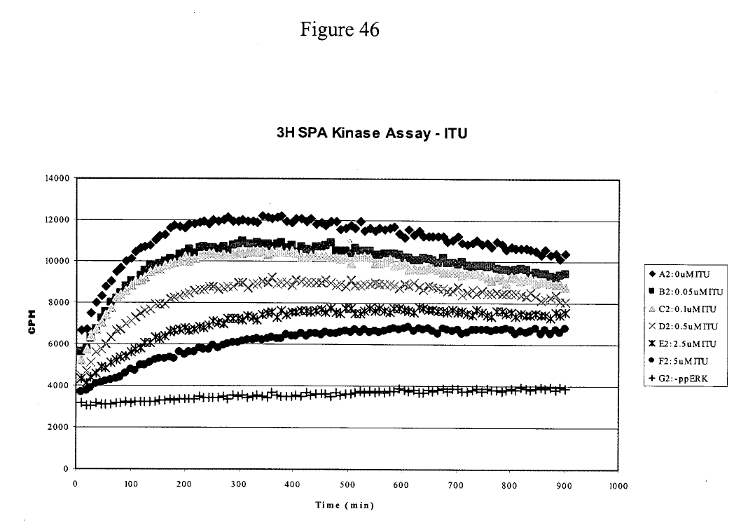

Immobilization of the non-biotinylated minimized ADP aptamer (FIG. 44) gave similar results to those obtained using a directly biotinylated minimized ADP aptamer (FIG. 43). Furthermore, the addition of known kinase inhibitors, e.g., staurosporine (FIG. 44), SB220025 (FIG. 45), or ITU (FIG. 46), yielded concentration-dependent inhibition of ppERK activity as judged by decreased signal observed in the ADP SPA. The IC₅₀ calculated from the inhibition curves obtained in studies treating ppERK with staurosporine (FIG. 44), SB220025 (FIG. 45), and ITU (FIG. 46) are summarized in Tables 8, 9, and 10, respectively.

TABLE 8
IC₅₀ Data Summary for Staurosporine Inhibition of ppERK

| | | | | | | | |
|---|---|---|---|---|---|---|---|
| Saturation | 217 | 262 | 352 | 262 | 550 | 550 | 901 |
| Time (min) | | | | | | | |
| 20% Reacted | 43.4 | 52.4 | 70.4 | 52.4 | 110 | 110 | 901 |
| Time (min) | | | | | | | |
| Closest Real | 46 | 55 | 73 | 55 | 109 | 109 | 901 |
| Data Point to | | | | | | | |
| 20% (min) | | | | | | | |
| Initial Rate | 57.022 | 45.419 | 40.907 | 23.708 | 10.855 | 5.9375 | 0 |
| Concentration | 0 | 0.1 | 0.2 | 1 | 5 | 10 | −ppERK |

TABLE 9
IC₅₀ Data Summary for SB220025 Inhibition of ppERK

| | | | | | | | |
|---|---|---|---|---|---|---|---|
| Saturation | 307 | 460 | 505 | 586 | 640 | 900 | 901 |
| Time (min) | | | | | | | |
| 20% Reacted | 61.4 | 92 | 101 | 117.2 | 128 | 180 | 901 |
| Time (min) | | | | | | | |
| Closest Real | 55 | 91 | 100 | 118 | 127 | 181 | 901 |
| Data Point to | | | | | | | |
| 20% (min) | | | | | | | |
| Initial Rate | 61.013 | 23.883 | 20.427 | 4.716 | 2.8722 | 2.3262 | 0.9597 |
| Concentration | 0 | 1.8 | 3.6 | 18 | 90 | 180 | −ppERK |

TABLE 10
IC₅₀ Data Summary for ITU Inhibition of ppERK

| | | | | | | | |
|---|---|---|---|---|---|---|---|
| Saturation | 280 | 289 | 298 | 298 | 415 | 568 | 901 |
| Time (min) | | | | | | | |
| 20% Reacted | 56 | 57.8 | 59.6 | 59.6 | 83 | 113.6 | 901 |
| Time (min) | | | | | | | |
| Closest Real | 55 | 55 | 55 | 55 | 82 | 109 | 901 |
| Data Point to | | | | | | | |
| 20% (min) | | | | | | | |
| Initial Rate | 50.213 | 46.949 | 48.324 | 36.486 | 19.275 | 10.772 | 0.9191 |
| Concentration | 0 | 0.05 | 0.1 | 0.5 | 2.5 | 5 | −ppERK |

### Example 3

Generating ADP Nucleic Acid Sensor Molecules

A. Generation of ADP Sensors

Using TMDs derived from the minimized ADP aptamer sequence, two pools, designated Pool A and Pool B, were prepared for stem selection. DNA pools were synthesized, purified and transcribed to RNA in preparation for selection round 1. The sequences of pools A and B are shown in FIG. 47.

Selections were initiated with 4×10¹⁴ RNA molecules. Selection buffer used for the ADP sensor selections was 50 mM Hepes, pH 7.5, containing 10 mM MgCl₂, 10 mM MnCl₂, 100 mM NaCl, 1 mM DTT, 1% DMSO, and 0.01% Bovine γ-globulin. A 10× concentrate of the selection buffer (minus γ-globulin) was stored at 4° C. shielded from light. Fresh 2× selection buffer was prepared for each round of selection (500 μl 10× buffer, 250 μl γ-globulin, 4.25 ml H₂O).

After purification on a 10% denaturing acrylamide gel, pool RNA was resuspended in DEPC treated H₂O. To initiate the negative selection, pool RNA was combined with 2× selection buffer to yield a final 1× buffer concentration in 250 μl. The reaction mixture was incubated at room temperature for a fixed period, and then quenched with 50 mM EDTA. Finally, 300 mM NaOAc, and 1.5 vol 2:1 isopropanol:ethanol were generally added to precipitate.

After precipitation, pool RNA was subjected to a denaturation step. In rounds 1-4, a chemical denaturation protocol was used. The pool pellet was resuspended in 90 μl H₂O followed by the addition of 10 μl 100 mM NaOH. The tube was lightly vortexed, then 12 μl NaOAc was added and the material was isopropanol:ethanol precipitated. In rounds 5 and 6 a temperature denaturation protocol was used. After quenching the negative reaction with EDTA, the sample was heated at 90° C. for 2 min, followed by brief cooling on ice, addition of 300 mM NaOAc and isopropanol:ethanol precipitation. During each negative selection step, two denaturation steps were performed (FIG. 19). After the final negative incubation step, samples were precipitated and the uncleaved pool molecules were purified on a 10% denaturing polyacrylamide gel followed by electroelution.

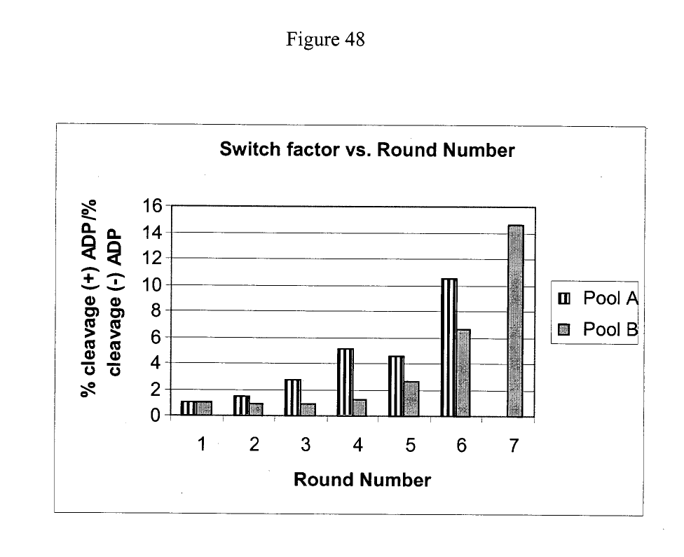

The next step consisted of two components: positive selection and assay. Approximately 20% of the material was used for assay reactions in which the negatively selected pool was incubated in 1× buffer at room temperature for a fixed period of time in the presence and absence of 1 mM ADP (ADP solution made up fresh in 1× buffer immediately prior to use). The remaining 80% of the RNA was incubated in 1× buffer at room temperature for the same period of time in the presence of 1 mM ADP. All reactions were quenched with 50 mM EDTA, and precipitated with 300 mM NaOAc and isopropanol:ethanol. The progress of selection was monitored by measuring the extent of cleavage in the assay reactions plus ADP (+) vs. in the reactions minus ADP (−) (referred to as the switch factor). The results of each round of selection are shown in FIG. 48 and summarized in Table 11.

TABLE 11
Summary of ADP Sensor Selection Rounds

| Round | (−ADP) | (+ADP) | Switch factor | Switch factor |
|---|---|---|---|---|
| Number | minutes | minutes | Pool B | Pool A |
| 1 | 193 | 30 | 1 | 1 |
| 2 | 60 | 30 | | |
| 3 | 180 | 20 | 0.9 | 1.5 |
| 4 | 192 | 20 | 1.3 | 2.7 |
| 5 | 108 | 20 | 2.6 | 5.2 |
| 6 | 90 | 32 | 6.6 | 4.6 |
| 7 | 118 | 60 | 14.6 | 10.5 |

Note:
in round 7, Pool B became contaminated with Pool A.

Pools were subsequently cloned using the TOPO TA cloning kit after round 7. Ninety-six colonies were isolated and inserts amplified by PCR. Twelve clones from each pool were transcribed, purified on a 10% denaturing acrylamide gel, and assayed for ADP-dependent cleavage. RNA was incubated in 1× selection buffer for 30 min at room temperature in the presence or absence of 1 mM ADP. Reactions were quenched and analyzed as described for the assays conducted during selection.

Switch factor for select clones was determined in a single time point 30 min assay using a gel-based readout. Clones were tested in a reaction mixture consisting of 50 mM Hepes, pH 7.5, containing 10 mM MgCl₂, 10 mM MnCl₂, 100 mM NaCl, 1 mM DTT, 1% DMSO, and 0.01% γ-globulin, with or without 1 mM ADP. As summarized in Table 12, 22 of 23 clones had a switch factor greater than 1 and 11 clones had a switch factor ratio (% cleavage (+)/(−)) greater than 10.

TABLE 12
Summary of Clones from ADP Sensor Selection

| clone | % cl. (+)/(−) |
|---|---|
| SCK.46.58.A3 | 21 |
| SCK.46.58.B2 | 13.5 |
| SCK.46.58.C5 | 16 |
| SCK.46.58.D4 | 10.2 |
| SCK.46.58.E3 | 3 |
| SCK.46.58.E4 | 5.8 |
| SCK.46.58.H4 | 4.7 |
| SCK.46.58.G6 | 16 |
| SCK.46.58.G3 | 2.1 |
| SCK.46.58.F6 | 15 |
| SCK.46.58.F5 | 4.3 |
| SCK.46.58.F1 | 25.5 |
| SCK.46.58.B7 | 20.2 |
| SCK.46.58.B8 | 3.7 |
| SCK.46.58.C8 | 9 |
| SCK.46.58.C10 | 1 |
| SCK.46.58.D10 | 12 |
| SCK.46.58.E8 | 6.7 |
| SCK.46.58.E9 | 2.8 |
| SCK.46.58.F7 | 3.7 |
| SCK.46.58.F8 | 19.8 |
| SCK.46.58.G10 | 1.2 |
| SCK.46.58.H10 | 12 |

Table 13 summarizes the sequence identifiers and switch factors for select ADP sensors.

TABLE 13
Sequence Identifiers and Switch Factors for ADP Sensors

| Clone identifier | Clone identifier | Switch |
|---|---|---|
| (LARK) | (internal) | Factor |
| ARX19P1.B01 | SCK.46.58.A3 | 21 |
| ARX19P1.F03 | SCK.46.58.C5 | 16 |
| ARX16P1.G08 | SCK.46.58.F1 | 25.5 |
| ARX19P1.G05 | SCK.46.58.F8 | 19.8 |
| ARX19P1.C09 | SCK.46.58.E8 | 6.7 |
| ARX19P1.G06 | SCK.46.58.G6 | 16 |

Table 14 summarizes the sequences of select ADP sensors, wherein highlighted material represents a random region of the stem selection.

[image]

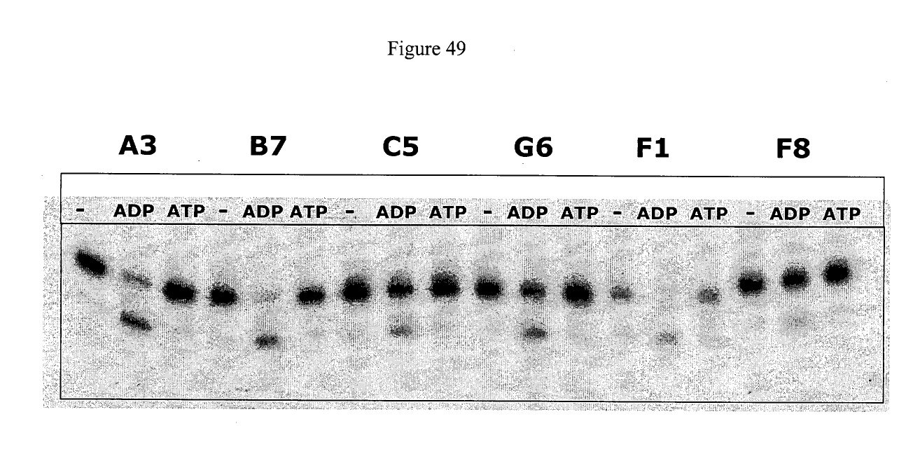

Cleavage assays were performed using radiolabeled RNA and analytical denaturing polyacrylamide gel electrophoresis (PAGE) (gel-based assays). Assays were performed upon 6 selected clones to measure their ability to discriminate between ADP and ATP. Gel-based assay transcription was performed in the presence of α-³²P-labelled UTP, and the resultant transcripts were gel-purified using denaturing PAGE. Assay mixtures containing the test clone were incubated in with either 1× selection buffer, 1× selection buffer plus 250 μM ADP or 1× selection buffer plus 250 μM ATP at room temperature for 60 min. The resultant samples were quenched by the addition of EDTA and the relative extents of cleavage measured by comparison of the intensity of the corresponding bands on a denaturing PAGE observed by reading a phosphorimager plate that had been exposed to the gel. As shown in FIG. 49 and summarized in Table 15, each of the clones discriminated between ADP and ATP, with reactions containing ATP displaying essentially background activity.

TABLE 15
ADP Selectivity of ADP Sensor Clones

| clone | (+/−) ADP | ADP/ATP | (+/−) ATP | % cleaved (+) ADP |
|---|---|---|---|---|
| SCK.46.58.A3 | 13 | 13 | 1 | 65 |
| SCK.46.58.B7 | 22.7 | 8.5 | 2 | 68 |
| SCK.46.58.C5 | 4.1 | 4.7 | 0.9 | 33 |
| SCK.46.58.G6 | 13.7 | 13.7 | 1 | 41 |
| SCK.46.58.F1 | 6.9 | 6.3 | 1 | 76 |
| SCK.46.58.F8 | 16 | 5.3 | 3 | 16 |

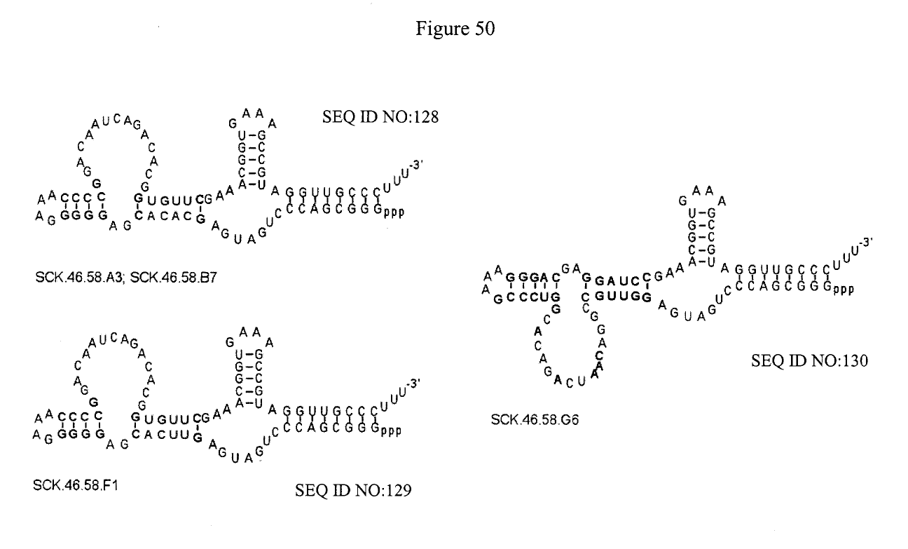

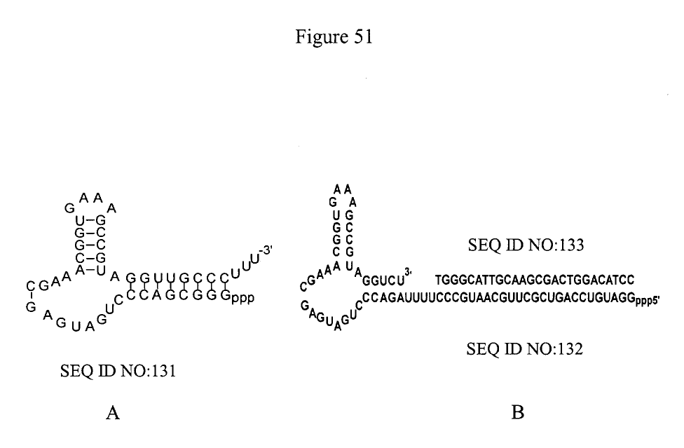

The secondary structure of select ADP sensor clones (wild type) is shown in FIG. 50. The 5 best discriminating clones were modified via PCR to install sequences required for the stem 1 FRET configuration. The general structure of the wild type and stem 1 FRET ADP sensor is shown in FIG. 51. The FRET versions were transcribed, purified, and tested for activation by ADP (30 min, room temperature, 1 mM ADP). As shown in Table 16, each of the clones was activated by ADP. In addition, inefficient separation of full length and cleavage products during purification could lead to higher background.

TABLE 16
Summary of ADP Sensitivity of Select ADP Sensor Clones
Prepared for FRET

| Clone | (−) ADP | % cl (+) ADP |
|---|---|---|
| SCK.46.66.A3 | 2.6 | 50 |
| SCK.46.66.B7 | 2.6 | 47 |
| SCK.46.66.G6 | 3.3 | 23 |
| SCK.46.66.F1 | 1.5 | 25 |
| SCK.46.66.F8 | 2.6 | 46 |

### Example 4

Use of ADP Sensors in FRET-based Assays

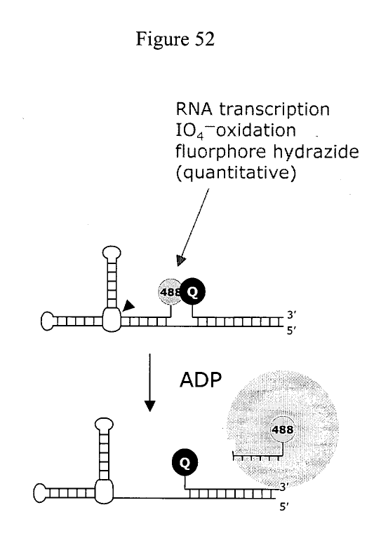

Selected clones were configured for solution-based FRET assays as schematically represented in FIG. 52. Fluorescein labeled sensor RNAs were prepared in a three step procedure. The RNA was oxidized at the 3′ end by incubation on ice with 300 mM sodium acetate (NaOAc), pH 5.4, and 10 mM sodium periodate (NaIO₄) for one hour shielded from light. The reaction was precipitated by the addition of 200 μl isopropanol followed by centrifugation. The oxidized RNA was then reacted with 3 mM fluorescein thiosemicarbazide (Molecular Probes) in 256 mM NaOAc, pH 5, at room temperature for two hours. The reactions were precipitated with one volume of isopropanol followed by precipitation. The RNAs were purified on a 1.5 mm denaturing polyacrylamide gels (8 M urea, 10% acrylamide; 19:1 acrylamide:bis-acrylamide) followed by Elutrap® apparatus (Schleicher and Schuell) at 225V for 1 hour in 1×TBE (90 mM Tris, 90 mM boric acid, 0.2 mM EDTA). The typical yield was approximately 6 nmole fluorescein labeled RNA.

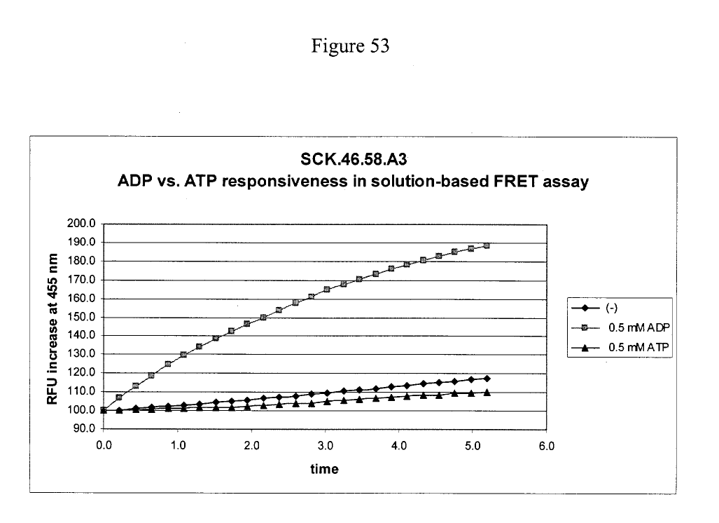

The NASMs were tested for both their ability to discriminate between ADP and ATP and the upper and lower limits of ADP detection in selection buffer (10 mM MgCl₂, 10 mM MnCl₂, 50 mM Hepes, pH 7.5, 100 mM NaCl, 1 mM DTT, 1% DMSO, 0.01% Bovine γ-globulin). For example, STC.46.58.A3 (1.2 μM) was annealed to MK.08.87.B (5′-Dabcyl-dT GGGATTGCAAGCGACTGGACATCC 3′; SEQ ID NO: 134) (6 μM) by heating to 80° C. for 2 min in annealing buffer (50 mM Tris pH 7.4, 50 mM NaCl) followed by cooling for 10 min at room temperature. The resulting complex was then brought up in selection buffer (minus γ-globulin) containing either no effector, 500 μM ADP or 500 μM ATP. The final concentration of sensor was 150 nM. The fluorescence of the reaction mixture at 455 nm was measured over the course of 5 min in a Fusion™ α-FP plate reader (Packard). The plot in FIG. 53 shows that SCK.46.58.A3 reacts essentially at background levels in the presence of 500 μM ATP while cleavage is stimulated in the presence of 500 μM ADP.

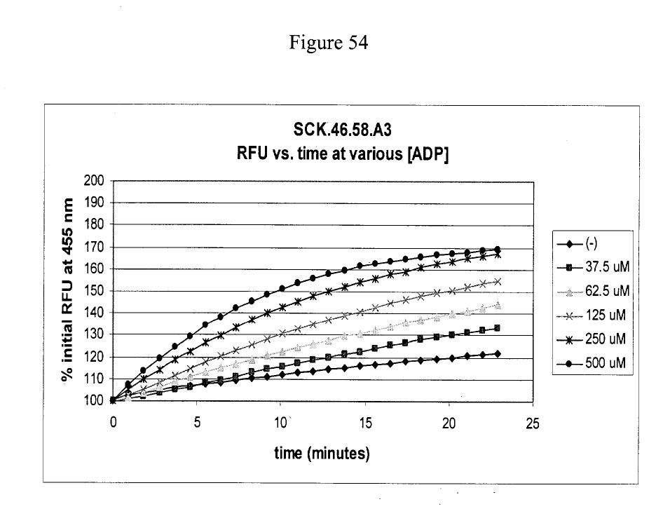

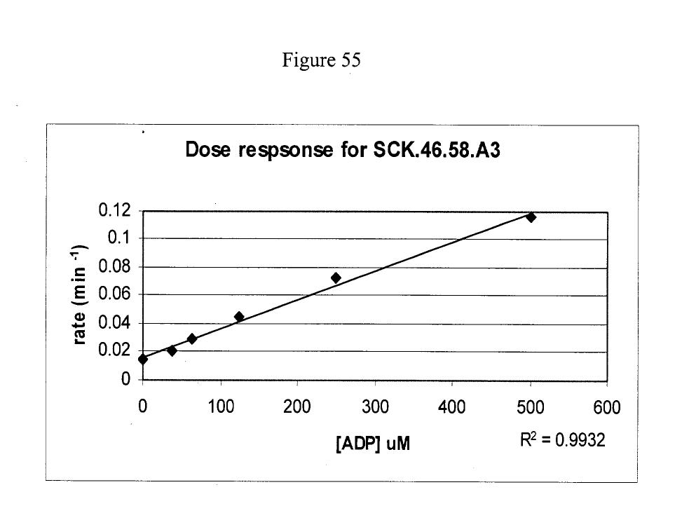

A similar assay was used to measure the response of STC.48.58.A3 over a range of ADP concentrations from 0 to 500 μM (FIG. 54). The rate constant at various ADP concentrations was obtained by fitting the RFU vs. time curve with a pseudo-first order rate equation (y=A(1−e^{-kt})+NS) where k is the first order rate constant, t is the time, NS is the signal amplitude at t=0 and A is the maximum signal amplitude. Under the conditions tested (buffer, reaction time, etc.) ADP can be measured at concentrations as low as 50 μM (FIG. 55).

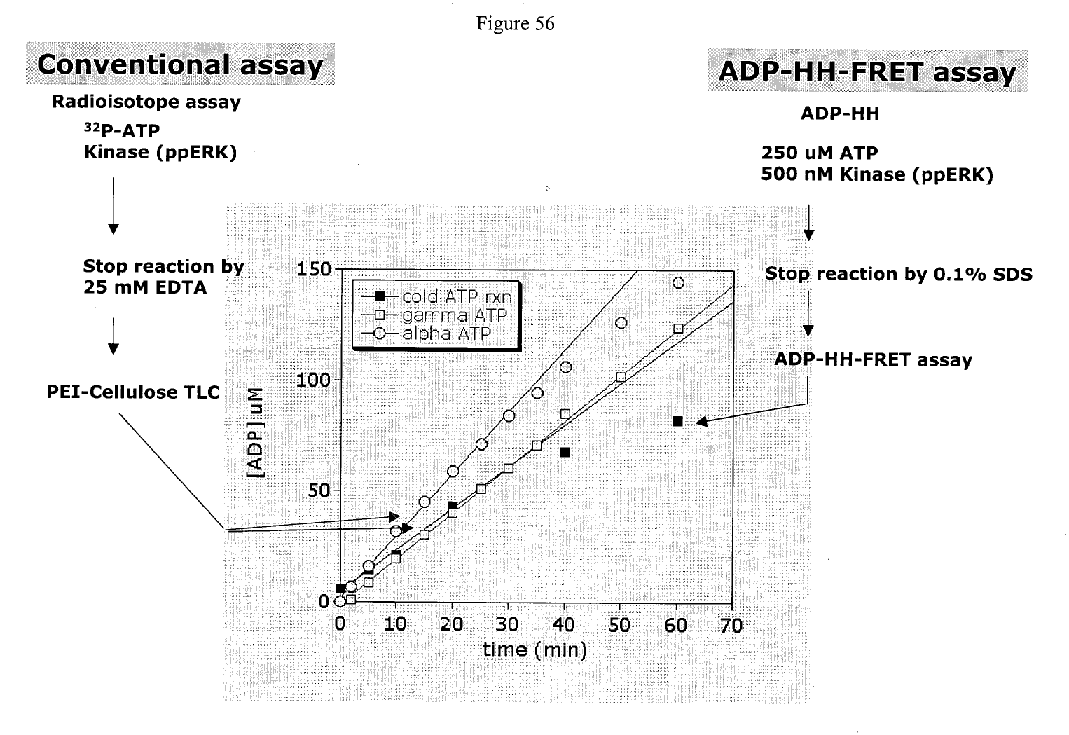

The ADP NASMs were also used to measure ppERK mediated phosphorylation of myelin basic protein (MBP, FIG. 56). As shown in FIG. 56, a time-dependent increase in ADP concentration was observed using an ADP sensor in a FRET assay measuring ppERK activity. The kinetics of ADP generation observed in the FRET assay was comparable to conventional radiometric measurement of ppERK-mediated phosphorylation of MBP.

A small prototype panel of mitogen-activated kinases (MAPK) was prepared to demonstrate one application of the FRET assay as a tool for “target mining”. Target mining refers to the screening of uncharacterized protein samples (e.g., from expression libraries or fractionated biological samples) for novel molecules with the desired activity. Table 17 illustrates a plate map exemplifying the use of ADP NASMs in an ATPase screen to identify MAPK activation pairs.

In mitogen-stimulated signaling cascades, activation of MAPK-catalyzed, downstream phosphorylation, depends upon interactions and/or reactions with upstream partners (e.g., a cell-surface receptor, or another MAPK). In order to demonstrate the utility of NASM-based screening as an approach to identify molecules with MAPK-stimulating activity, the ADP NASM in FRET format (SCK.46.58.A3) was used to detect increases in MAPK catalysis associated with binary mixtures of potential signaling partners. In a prototype screen (Table 17), six samples containing either purified MAPK proteins (Erk2, Mek1. P.386, Jnk3, Mek 6 or buffer (no kinase)) were dispensed in a microtiter plate to generate a 6×6 pairwise matrix. For simplicity, water was used as the phosphoacceptor for MAPK-catalyzed phosphotransfer, although peptide acceptors could also be used.

In each well, ATP hydrolysis was performed by incubating 1-2 μM purified protein (1 μM Kinase+1 μM kinase2 or buffer) with 1 mM ATP for two hours at 37° C. (50 mM Hepes, pH 8, 10 mM MgCl₂, 100 mM NaCl, 1 mM DTT). The relative ADP yield in each well was assayed from the initial rate of fluorescence increase (535 nm) upon the addition of 240 nM NASM RNA (SCK.46.58.A3) and 1.2 μM quencher oligo (MK.08.87.B). In the majority of wells containing 2 μM total protein, the initial rate ranged from 21 to 81 RFU/min (Table 17, unshaded cells; avg 55±19 RFU/min). However, in wells containing know activation pairs (Mek1/Erk2 and Mek6/p386), substantially enhanced fluorescence was observed (Table 17, shaded cells; 150-300 RFU/min), indicating activation of ATPase activity associated with MAPK stimulation.

TABLE 17
Target Mining: Identification Of Mapk Activation Pairs

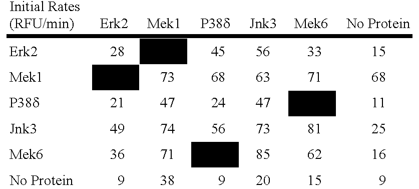

Variations, modifications, and other implementations of what is described herein will occur to those of ordinary skill in the art without departing from the spirit and scope as claimed. Accordingly, the invention is to be defined not by the preceding illustrative description but instead by the spirit and scope of the following claims.

We claim:

1. A nucleic acid sensor molecule comprising:
(a) a target modulation domain, wherein said target modulation domain recognizes ADP;
(b) a linker domain; and
(c) a catalytic domain.

2. The nucleic acid sensor molecule of claim 1 wherein the catalytic domain comprises an optical signal generating unit.

3. The nucleic acid sensor molecule of claim 2, wherein said optical signal generating unit comprises at least one optical signaling moiety.

4. The nucleic acid sensor molecule of claim 2, wherein said optical signal generating unit comprises at least a first optical signaling moiety and a second optical signaling moiety.

5. The nucleic acid sensor molecule of claim 4, wherein said first and second signaling moieties change proximity to each other upon recognition of a target by the target modulation domain.

6. The nucleic acid sensor molecule of claim 5, wherein said first and second signaling moieties comprise a fluorescent donor and a fluorescent quencher, and recognition of a target by the target modulation domain results in an increase in detectable fluorescence of said fluorescent donor.

7. The nucleic acid sensor molecule of claim 5, wherein said first signaling moiety and said second signaling moiety comprise fluorescent energy transfer (FRET) donor and acceptor groups, and recognition of a target by the target modulation domain results in a change in distance between said donor and acceptor groups, thereby changing optical properties of said molecule.

8. The nucleic acid sensor molecule of claim 3, wherein said optical signaling moiety changes conformation upon recognition of a target by the target modulation domain, thereby resulting in a detectable optical signal.

9. The nucleic acid sensor molecule of claim 1, further comprising a detectable label.

10. The nucleic acid sensor molecule of claim 9 wherein the detectable label comprises at least one radioactive moiety.

11. The nucleic acid sensor of claim 9, wherein the detectable label comprises a fluorescent label.

12. The nucleic acid sensor of claim 11, wherein said fluorescent label is fluorescein, DABCYL, or a green fluorescent protein (GFP) moiety.

13. The nucleic acid sensor of claim 1, wherein said nucleic acid sensor further comprises an affinity capture tag label.

14. The nucleic acid sensor molecule of claim 1 or 2, wherein said nucleic acid sensor molecule includes at least one modified nucleotide.

15. The nucleic acid sensor molecule of claim 1 or 2, wherein said catalytic domain comprises an endonucleolytic ribozyme.

16. The nucleic acid sensor molecule of claim 15, wherein said endonucleolytic ribozyme is a cis-endonucleolytic ribozyme or a trans-endonucleolytic ribozyme.

17. The nucleic acid sensor molecule of claim 15, wherein said endonucleolytic ribozyme is a hammerhead ribozyme.

18. The nucleic acid sensor molecule of claim 1 or 2, wherein said catalytic domain comprises a self-ligating ribozyme.

19. The nucleic acid sensor molecule of claim 18, wherein said self-ligating ribozyme is a cis-ligase ribozyme or a trans-ligase ribozyme.

20. The nucleic acid sensor molecule of claim 18, wherein said self-ligating ribozyme is a 1 -piece ligase, 2-piece ligase or 3-piece ligase.

21. The nucleic acid sensor molecule of claim 1 or 2, wherein said nucleic acid sensor molecule comprises RNA, DNA, or both RNA and DNA.

22. The nucleic acid sensor molecule of claim 1, wherein the nucleic acid sensor molecule is as shown in SEQ ID NO: 120 or SEQ ID NO: 121.

23. The nucleic acid sensor molecule of claim 1, wherein the nucleic acid sensor molecule is as shown in any one of SEQ ID NOs: 122-127.

24. A composition comprising the nucleic acid sensor molecule of any one of claims 1-23 and a buffer.

25. The composition of claim 24, further comprising an RNase inhibitor.

26. The composition of claim 25, wherein said RNase inhibitor is selected from the group consisting of Va-riboside, vanadyl, tRNA, polyU, RNaseln and RNaseOut.

27. The composition of claim 25 or 26, wherein said composition is substantially RNase-free.

28. A composition comprising at least one nucleic acid sensor molecule according to any one of claims 1-23, affixed to a substrate.

29. The composition of claim 28, wherein said substrate is glass, gold or other metal, silicon or other semiconductor material, nitrocellulose, nylon, or plastic.

30. The composition of claim 28, wherein the nucleic acid sensor molecule is covalently attached to said substrate.

31. The composition of claim 28, wherein the nucleic acid sensor molecule is non-covalently attached to said substrate.

32. The composition of claim 28, wherein the nucleic acid sensor molecule is immobilized to the substrate via hybridization of a terminal portion of the nucleic acid sensor molecule to an oligonucleotide that is bound to the surface of the substrate.

33. The composition of claim 28, wherein said composition comprises a plurality of nucleic acid sensor molecules immobilized to the substrate via hybridization of a terminal portion of the nucleic acid sensor molecule to an array of oligonucleotides bound to the substrate at spatially discrete regions.

34. The substrate of claim 28, wherein said substrate comprises at least 50 nucleic acid sensor molecules.

35. The substrate of claim 28, wherein said substrate comprises at least 250 nucleic acid sensor molecules.

36. A system for detecting ADP, comprising a composition according to any one of claims 28-35 and a detector in communication with said composition, wherein said detector is capable of detecting a signal generated upon recognition of a target molecule by a nucleic acid sensor molecule.

37. The system of claim 36, further comprising a light source in optical communication with said composition.

38. The system of claim 36, further comprising a processor for processing optical signals detected by the detector.

39. A method of identifying or detecting ADP in a sample, the method comprising:
contacting a sample suspected of containing ADP with a nucleic acid sensor molecule according to any one of claims 2-35, wherein a change in the signal generated by the optical signal generating unit or detectable label indicates the presence of ADP in said sample.

40. The method of claim 39 further comprising quantifying the change in signal generated by the optical signal generating unit or detectable label to quantify the amount of ADP in the sample.

41. The method of claim 39 or 40 wherein the sample is selected from the group consisting of: environmental samples, biohazard materials, organic samples, drugs and toxins, flavors and fragrances, and biological samples.

42. The method of claim 39 or 40 wherein the sample is a biological sample selected from the group consisting of cells, cell extracts, cell lysates, tissues, tissue extracts, bodily fluids, serum, blood, and blood products.

43. A diagnostic system for identifying or detecting ADP, the diagnostic system comprising a nucleic acid sensor molecule according to any one of claims 2-35 and a detector in communication with said nucleic acid sensor molecule, wherein said detector detects changes in the signal generated by the optical signal generating unit or detectable label of said nucleic acid sensor.

44. The diagnostic system of claim 43, further comprising a processor for processing signals detected by the detector.

45. A method of detecting the activity of a biological agent that produces or consumes ADP in a reaction, the method comprising:
contacting a sample containing the biological agent with a nucleic acid sensor molecule according to claim 1,
wherein a change in the signal generated by the optical signal generating unit detectable label indicates activity of the biological agent in said sample.

46. The method of claim 45, further comprising quantifying the amount of signal generated by the optical signal generating unit detectable label to quantify the activity of the biological agent in the sample.

47. The method of claim 45, wherein said biological agent consumes ADP in a reaction.

48. The method of claim 47, wherein said biological agent is an ATP synthase.

49. The method of claim 45, wherein said biological agent produces ADP in a reaction.

50. The method of claim 49, wherein said biological agent is a kinase or an ATPase.

51. The method of claim 50, wherein said kinase is a MAP kinase (MEK), a MAP Kinase Kinase (MEKK), or a MAP Kinase Kinase Kinase, (MEKKK).

52. The method of claim 51, wherein said MAP kinase is ERK1, ERK2, JNK, or P38 MAP kinase.

53. The method of claim 50, wherein said kinase is a RAF kinase.

54. A method of identifying a modulator of activity of a biological agent that produces or consumes ADP in a reaction, the method comprising:
contacting a test agent with a biological agent and nucleic acid sensor molecule according to claim 1, wherein said nucleic acid sensor molecule has a target recognition domain that recognizes ADP.

55. The method of claim 54, wherein said biological agent consumes ADP in a reaction.

56. The method of claim 55, wherein said biological agent is an ATP synthase.

57. The method of claim 54, wherein said biological agent produces ADP in a reaction.

58. The method of claim 57, wherein said biological agent is a kinase or an ATPase.

59. The method of claim 58, wherein said kinase is a MAP kinase (MEK), a MAP Kinase Kinase (MEKK), or a MAP Kinase Kinase Kinase, (MEKKK).

60. The method of claim 59, wherein said MAP kinase is ERK1, ERK2, JNK, or P38 MAP kinase.

61. The method of claim 58, wherein said kinase is a RAF kinase.

62. The method of claim 45 or 54, wherein the catalytic domain of said nucleic acid sensor molecule comprises a cis-ligase ribozyme or a trans-ligase ribozyme.

63. A nucleic acid sensor molecule that is 100 times more specific for ADP than ATP.

64. A nucleic acid sensor molecule that is 1000 times more specific for ADP than ATP.

65. An ADP-specific nucleic acid sensor molecule that recognizes ADP in a 100 fold excess of ATP.

66. An ADP-specific nucleic acid sensor molecule that recognizes ADP in a 1000 fold excess of ATP.

67. An ADP-specific aptamer.

68. A composition comprising an ADP-specific aptamer and a buffer.

69. The composition of claim 68 further comprising an RNase inhibitor.

70. The composition of claim 68 or 69, wherein said composition is substantially RNase-free.

71. A composition comprising at least one ADP-specific aptamer affixed to a substrate.

72. The composition of claim 71, wherein said substrate is glass, gold or other metal, silicon or other semiconductor material, nitrocellulose, nylon, or plastic.

73. The composition of claim 71, wherein said substrate is a multiwell plate containing a scintillant imbedded in the surface of the plate.

74. The composition of claim 71, wherein the ADP-specific aptamer is covalently attached to said substrate.

75. The composition of claim 71, wherein the ADP-specific aptamer is non-covalently attached to said substrate.

76. The composition of claim 71, wherein the ADP-specific aptamer is immobilized to the substrate via hybridization of a terminal portion of the ADP-specific aptamer to an oligonucleotide that is bound to the surface of the substrate.

77. The composition of claim 71, wherein the ADP-specific aptamer is biotinylated and the surface is coated with streptavidin.

78. The composition of claim 71, wherein said composition comprises a plurality of ADP-specific aptamers immobilized in wells of a multiwell plate containing a scintillant imbedded in the surface of the plate.

79. The composition of claim 71, comprising at least 50 ADP-specific aptamers.

80. The composition of claim 71, comprising at least 250 ADP-specific aptamers.

81. A system for detecting ADP comprising a composition according to any one of claims 71-80 and a detector in communication with said composition.

82. The system of claim 81, further comprising a processor for processing signal detected by the detector.

83. A method of detecting ADP in a sample, the method comprising:
contacting a sample containing detectably labeled ADP with a composition according to any one of claims 71-82, wherein detection of the signal generated by the detectable label indicates the presence of ADP in said sample.

84. The method of claim 83 further comprising quantifying the change in signal generated by the detectable label to quantify the amount of ADP in the sample.

85. The method of claim 83 or 84 wherein the sample is selected from the group consisting of environmental samples, biohazard materials, organic samples, drugs, toxins, flavors, fragrances, and biological samples.

86. A diagnostic system for identifying or detecting ADP, the diagnostic system comprising:
a composition comprising an ADP aptamer according to any one of claims 71-82 in contact with detectably labeled ADP;
and a detector in communication with said composition, wherein said detector detects a signal generated by the detectable label of ADP upon binding to the aptamer.

87. The diagnostic system of claim 86, further comprising a processor for processing signals detected by the detector.

88. A method of detecting the activity of a biological agent that produces ADP in a reaction, the method comprising:
contacting a sample containing the biological agent and detectably labeled ATP with an ADP aptamer according to claim 71,
wherein detection of the signal generated by the detectable label indicates activity of the biological agent in said sample.

89. A method of detecting the activity of a biological agent that consumes ADP in a reaction, the method comprising:
contacting a sample containing the biological agent and detectably labeled ADP with an ADP aptamer according to claim 71,
wherein detection of a signal generated by the detectable label indicates activity of the biological agent in said sample.

90. The method of claim 88 or 89, further comprising quantifying the amount of signal generated by the detectable label to quantify the activity of the biological agent in the sample.

91. The method of claim 88, wherein said biological agent is a kinase or an ATPase.

92. The method of claim 89, wherein said biological agent is an ATP synthase.

93. The method of claim 91, wherein said kinase is a MAP kinase (MEK), a MAP Kinase Kinase (MEKK), or a MAP Kinase Kinase Kinase, (MEKKK).

94. The method of claim 93, wherein said MAP kinase is ERK1, ERK2, JNK, or P38 MAP kinase.

95. The method of claim 91, wherein said kinase is a RAF kinase.

96. A method of identifying a modulator of activity of a biological agent that produces or consumes labeled ADP in a reaction, the method comprising:
contacting a test agent with a biological agent and an aptamer according to claim 71, wherein said aptamer recognizes ADP, wherein recognition of the ADP by the aptamer results in a change in the signal generated by the detectable label, and further wherein changes in the signal generated by the detectable label in the presence and absence of said test agent indicates the test agent is a modulator of said activity of the biological agent.

97. The method of claim 96, wherein said biological agent is an ATP synthase.

98. The method of claim 96, wherein said biological agent is a kinase or an ATPase.

99. An ADP-specific aptamer comprising the aptamer shown in any one of SEQ ID NOS. 19-71.

100. An ADP-specific aptamer comprising the aptamer shown in any one of SEQ ID NOS. 78-114.
